(19) United States
(12) Patent Application Publication
(10) Pub. No.: US 2023/0302423 A1
(43) Pub. Date: Sep. 28, 2023

(54) RNA SCAFFOLDED WIREFRAME ORIGAMI AND METHODS THEREOF

(71) Applicant: Massachusetts Institute of Technology, Cambridge, MA (US)

(72) Inventors: Mark Bathe, Cambridge, MA (US); Tyson Shepherd, Louisville, CO (US); Molly Frances Parsons, Cambridge, MA (US)

(21) Appl. No.: 18/191,723

(22) Filed: Mar. 28, 2023

Related U.S. Application Data
(60) Provisional application No. 63/324,538, filed on Mar. 28, 2022.

(51) Int. Cl. B01J 19/00; B82Y 30/00

(52) U.S. Cl. CPC B01J 19/0046; B82Y 30/00; B01J 2219/00603; B01J 2219/00722

(57) ABSTRACT

Methods for designing scaffolded RNA nanostructures of desired shape are described. In some forms, the methods design nucleic acid “staple” sequences that hybridize to a user-defined RNA scaffold and fold it into the desired shape based on A-form helical nucleic acid geometry. In some forms, the methods implement asymmetry in nucleotide positions across two helices of an edge to account for A-form nucleic acid geometry. In preferred forms, crossover asymmetry is implemented in the staples. In other forms, crossover asymmetry is implemented in the RNA scaffold. In other forms, the methods do not introduce crossover asymmetry. Scaffolded RNA nanostructures produced according to the methods including messenger RNAs, replicating RNAs, functional RNAs and other RNA species within the scaffold, staples, or both scaffold and staples are provided. Modified nanostructures including chemically modified nucleotides are also described.

### CROSS-REFERENCE TO RELATED APPLICATION

This application claims the benefit of and priority to U.S. Provisional Application No. 63/324,538 filed Mar. 28, 2022, which is hereby incorporated by reference in its entirety.

### STATEMENT REGARDING FEDERALLY SPONSORED RESEARCH

This invention was made with government support under N00014-16-1-2953 and N00014-20-1-2084 awarded by the Office of Naval Research. The government has certain rights in the invention.

### REFERENCE TO SEQUENCE LISTING

The Sequence Listing submitted as a text file named “MIT_24153_US_ST26.xml,” created on Mar. 27, 2023, and having a size of 308,552 bytes is hereby incorporated by reference pursuant to 37 C.F.R § 1.834(c)(1).

### FIELD OF THE INVENTION

The present invention relates to the design of arbitrary 2D or 3D geometries using scaffolded RNA, and in particular to the design of RNA-based nanostructures having a desired geometric form and enhanced stability.

### BACKGROUND OF THE INVENTION

Nucleic acid nanotechnology offers unique capabilities for diverse applications ranging from therapeutics and enzyme nanoreactors, to patterning and lithography (Timm, et al. Angewandte Chemie (International ed. in English) 54, (2015); Gallego, et al. Adv Mater 29, (2017); Douglas, et al., Science 335, 831-834 (2012); and Li, et al., Nat Biotechnol 36, 258-264 (2018)). The predictability of Watson-Crick-Franklin base-pairing together with crossover motifs derived from the Holliday junctions renders nucleic acids amenable to fabricating a wide variety of 2D and 3D structures. Scaffolded DNA origami, in particular, has been demonstrated to enable the fabrication of nearly arbitrary 2D and 3D dense, bricklike as well as porous, meshlike wireframe objects by folding a single-stranded DNA scaffold to user-specified geometries via annealing with short staple strands (Rothemund, Nature, 440, 297-302 (2006); Benson, et al., Angew Chem Int Ed Engl, 55, 8869-8872 (2016); Benson, et al., Nature 523, 441-444 (2015); Dietz, et al., Science 325, 725-730 (2009). Douglas, et al. Nucleic Acids Res 37, 5001-5006 (2009); He, et al. Nature 452, 198-201 (2008); Jun, et al. ACS Nano doi: 10.1021/acsnano.8b08671 (2019); Jun, et al. Sci Adv 5, eaav0655 (2019); Zhang, et al. Nat Nanotechnol 10, 779-784 (2015); and Veneziano, et al. Science 352, 1534 (2016)).

In contrast, RNA origami has been explored considerably less, largely utilizing RNA/RNA interactions, with only some studies probing the ability to fabricate hybrid RNA/DNA origami (Endo, et al., Chemistry 20, 15330-15333 (2014); Afonin, et al. Nat Nanotechnol 5, 676-682 (2010); Afonin, et al. Nano Lett 12, 5192-5195 (2012); Geary & Andersen, Science 345, 799-804 (2014); Han, et al. Science 358, (2017); Hoiberg, et al., Biotechnol J 14, e1700634 (2019); Kozyra, et al., ACS Synth Biol 6, 1140-1149 (2017); Severcan, et al. Nat Chem 2, 772-779 (2010); Severcan, et al., Nano Lett 9, 1270-1277 (2009); Sparvath, et al., Methods Mol Biol 1500, 51-80 (2017); Ke, et al. Nucleic Acids Res, 47, 1350-1361 (2019); Monferrer, et al., Nat Commun 10, 608 (2019); Wang, et al., Chem Commun (Camb) 49, 5462-5464 (2013); and Zhou, et al. Nanoscale Advances 3, 4048-4051 (2021)).

Importantly, full control over both RNA/DNA composition and 3D geometry of target nucleic acid origami structures would offer additional applications in nanoscale materials synthesis and therapeutics that are not currently offered by either RNA/RNA or DNA/DNA origami alone (Hong, et al. Nano Lett 18, 4309-4321 (2018); Johnson, et al. Small 13, (2017); and Ohno, et al. Curr Opin Biotechnol 58, 53-61 (2018)). Further, automated, top-down sequence design algorithms that have greatly aided the dissemination of scaffolded DNA origami remain sparse (Geary, et al., Nature Chemistry 13, 549-558 (2021)).

Similar to DNA, RNA has a 4-base code that includes common bases of adenine (A), cytosine (C), and guanine (G), with the exception of uracil (U) rather than thymine (T) as a fourth base. Chemically, RNA carries an additional 2′-hydroxyl group on the sugar that forces a C3′-endo form, leading to an A-form double helix (11 base pairs per helical turn, 2.6 Å axial rise per base pair, 23 Å helical diameter), when hybridized with either DNA or RNA, rather than the canonical B-form of duplex DNA (10.5 base pairs per helical turn, 3.4 Å axial rise per base pair, 20 Å helical diameter) (Rich, Journal of Biological Chemistry 281, 7693-7696 (2006)). Unlike DNA, single-stranded RNA complex tertiary folds with Hoogsteen and sugar edge base interactions, thereby rendering reliable de novo tertiary structure prediction and programmability challenging (Miao, et al., Annual review of biophysics 46, (2017); and Shapiro, et al. Methods 103, (2016)).

Nevertheless, knowledge gained from 3D RNA tertiary structures has been used to generate RNA nanoparticles by engineering RNA fragments to assemble into programmed higher-order geometries, using, for example, tRNAs and multi-way junctions, to create complex shapes (Shu, et al., Nature Nanotechnology 6, 658-667 (2011); and Jasinski, et al., ACS Nano 11, 1142-1164 (2017)). However, absolute control over the programmability of the final, target shape has been constrained by the required sequence space of underlying folds.

Beyond the preceding tectonics approach, RNA nanotechnology has recently seen significant advances in programmed folding of long single-stranded RNA objects, with predefined tertiary junctions used to assemble complex nanoparticle shapes. Progress in hybrid RNA/DNA origami folding with high yield and purity has been reported (Ko, et al., Nature Chemistry 2, 1050-1055 (2010)), however no studies have yet realized the design and fabrication of arbitrary dual-duplex (DX) wireframe structures based on RNA scaffolds of varying sequence and length, prior works focusing principally on brick-like or single-duplex-edged structures.

One challenge with respect to scaffolded DNA wireframe origami sequence design is the need to account for the greater pitch and twist of the A-form duplex geometry to realize folding and stability, requiring that adjacent crossovers of distinct scaffold and staple strands be spaced asymmetrically along the helices (Geary, & Andersen, “Design Principles for Single-Stranded RNA Origami Structures”, in DNA Computing and Molecular Programming (eds. Murata & Kobayashi) 1-19 Springer International Publishing, 2014). Further, despite the long-standing goal of understanding the impact of base-level origami design rules on global and local structure and stability, no study to date has comprehensively characterized tertiary structure simultaneously with chemical footprinting to determine a base-level secondary structure, beyond simple 3-way and 4-way DNA junctions characterized using hydroxy radical footprinting (Wang & Seeman, Biochemistry 34, 920-929 (1995)).

Therefore, robust methods for producing RNA-scaffolded origami in three dimensions rendered as wireframe polyhedra composed of dual duplex edges are needed.

It is an object of the invention to provide effective and highly-customizable design principles for structurally-stable RNA-scaffolded origami in three dimensions.

It is also an object of the invention to provide structurally stable and highly reproducible rigid RNA-based nanostructures having user-defined three dimensional polyhedral morphology.

It is a further object of the invention to provide structurally stable, rigid nanoscale DNA/RNA hybrid assemblies.

It is a further object of the invention to provide scaffolded-RNA nanostructures as vehicles for in vivo delivery of RNA-based payloads, such as functional RNAs and mRNAs.

### SUMMARY OF THE INVENTION

Design principles for RNA-scaffolded origami in three dimensions rendered as wireframe polyhedra composed of dual duplex edges have been developed. Methods of using long RNAs as a scaffold to fabricate diverse 3D wireframe origami having use-defined three-dimensional shapes with enhanced structurally stability are provided. Nucleic acid nanostructures with tetrahedral morphology folded from scaffolded RNA, as well as hybrid DNA/RNA nanostructures are also provided.

Methods for designing a scaffolded RNA nanostructure having a geometric shape include (a) determining the geometric parameters of an input, where the input includes a 3D polyhedral or 2D polygon geometric shape and optionally one or more of its physical dimensions; (b) identifying a route for a single-stranded RNA scaffold that traces throughout the geometric shape based on A-form helical nucleic acid geometry; and (c) generating the sequences of the single-stranded RNA scaffold and optionally the nucleic acid sequence of staple strands that combine to form a scaffolded RNA nanostructure having the geometric shape. In some forms, identifying a route for a single-stranded RNA scaffold that traces throughout the geometric shape in step (b) is based on staple crossover asymmetry with 11 nucleotides per helical turn. For example, in some forms, the staple crossover asymmetry includes a difference in the nucleotide position across two helices of an edge of from one to ten nucleotides, inclusive. In preferred forms, the staple crossover asymmetry includes a difference in the nucleotide position across two helices of an edge of four nucleotides. In other forms, identifying a route for a single-stranded RNA scaffold that traces throughout the geometric shape in step (b) is based on scaffold crossover asymmetry with 11 nucleotides per helical turn. For example, in some forms, scaffold crossover asymmetry includes a difference in the nucleotide position across two helices of an edge of from one to ten nucleotides, inclusive. In particular forms, the scaffold crossover asymmetry includes a difference in the nucleotide position across two helices of an edge of six nucleotides. In other forms, identifying a route for a single-stranded RNA scaffold that traces throughout the geometric shape in step (b) is based on no crossover asymmetry, with 11 nucleotides per helical turn.

Generally, the input in step (a) also includes one or more of the geometric shape's physical dimensions. In some forms, the input in step (a) further includes specifying an even number of parallel or anti-parallel helices within each edge of the nanostructure. In some forms, each edge includes four or more parallel or anti-parallel helices arranged in square cross-sectional morphology, or six or more parallel or anti-parallel helices arranged in honeycomb lattice morphology. In some forms, the input in step (a) further includes specifying for each vertex of the nanostructure that two or more edges come together in an aligned angle to create a bevel at the vertex. Typically, the geometric shape does not have spherical topology.

In some forms, the input in step (a) further includes a template RNA scaffold sequence, or the sequence of one or more staples, or a template RNA scaffold sequence and the sequence of one or more staples. In some forms, the input in step (a) further includes the length of one or more of the edges spanning two vertices of the target structure. Typically, the length of each edge is between 22 base pairs and 1,100 base pairs, inclusive.

In some forms, the crossover type is anti-parallel crossover, the length of each edge is expressed as a multiple of 11 base pairs, and the length of each edge is between 22 base pairs and 1,100 base pairs, inclusive. For example, in some forms, the length of each edge is 44 base pairs, 55 base pairs, 66 base pairs, or 77 base pairs. In some forms, the staples are DNA. In other forms, the staples are RNA. In some forms, the nanostructures include one or more RNA staples and one or more DNA staples.

In some forms, the input in step (a) includes providing geometric parameters including vertex, face and edge information determined from a polygonal or polyhedral wire-mesh model of the target shape.

In some forms, identifying a route for a single-stranded RNA scaffold that traces throughout the geometric shape in step (b) includes the steps of:
(i) rendering the geometric shape as a closed surface polyhedral network or an open surface polygonal network; (ii) determining a spanning tree of the network, wherein the vertices and lines of the graph are the nodes and edges of the network, respectively; (iii) classifying each edge of the network based on its membership in the spanning tree, where edges that are members of the spanning tree do not have a scaffold double crossover, and edges that are not members of the spanning tree have a scaffold double crossover; (iv) splitting each edge that is not a member of the spanning tree into two edges, each containing a pseudo-node at the point of the scaffold crossover; (v) splitting each node at each of the vertices into two pseudo-nodes; and (vi) calculating the Euler cycle of the network, wherein the Euler cycle represents the route of a single-stranded RNA scaffold that traces once along each edge in both directions throughout the entire geometric shape. In some forms, the crossover type is parallel crossover, and the length of each edge is between 22 base pairs and 1,100 base pairs, inclusive. In some forms, identifying a route for a single-stranded RNA scaffold that traces throughout the geometric shape in step (b) includes the steps of:
(i) rendering the geometric shape as a closed surface polyhedral network or an open surface polygonal network; (ii) calculating a spanning tree of the network, wherein the vertices and lines of the graph are the nodes and edges of the network, respectively; (iii) classifying each edge of the network as one of four types based on its membership in the spanning tree and on whether it employs anti-parallel or parallel crossovers; edges that are members of the spanning tree have each scaffold portion start and end at different vertices, and edges that are not members of the spanning tree have each scaffold portion start and end at the same vertex; (iv) splitting each edge that is not a member of the spanning tree into two edges, each containing a pseudo-node at the point of the scaffold crossover; (v) splitting each node at each of the vertices into two pseudo-nodes; and (vi) calculating the Euler cycle of the network, wherein the Euler cycle represents the route of a single-stranded nucleic acid scaffold by superimposing and connecting units of partial scaffold routing within an edge based on its classification and length.

In some forms, identifying a route for a single-stranded RNA scaffold that traces throughout the geometric shape in step (b) includes:
(i) rendering the geometric shape as a closed surface polyhedral network or an open surface polygonal network; (ii) rendering each helix in the network as a line, based on the target cross-section of each edge; (iii) calculating a loop-crossover structure, wherein two or more adjacent lines are connected to form loops and all possible double-crossover locations between two loops are calculated; (iv) calculating a dual graph of the loop-crossover structure, wherein the loops and double-crossover locations of the network are converted to nodes and edges of the dual graph, respectively; (v) calculating a spanning tree of the dual graph network; (vi) calculating which of the locations of double-strand crossovers will be used, wherein a single double-strand crossover is placed at each edge that is the part of the spanning tree of the dual graph; and (vii) calculating the Euler cycle of the network, wherein the Euler cycle represents the route of a single-stranded nucleic acid scaffold that traces once through each duplex throughout the entire geometric shape.

In some forms, the spanning tree of the network is determined using a breadth-first search or depth-first search. In some forms the spanning tree is calculated using Prim's formula or Kruskal's formula. Typically, the Euler circuit is the A-trail Euler circuit. In some forms, rendering the geometric shape as polyhedral network includes producing a node-edge network of the three-dimensional structure.

In some forms, the methods also include the step of: (d) predicting the three-dimensional structure of the scaffolded RNA nanostructure.

In some forms, the methods also include the step of: (e) assembling the scaffolded RNA nanostructure.

In some forms, the methods also include the step of: (f) validating the scaffolded RNA nanostructure. In an exemplary form, the scaffolded RNA nanostructure is validated by comparison with a predicted three-dimensional structure.

Polyhedral scaffolded RNA nanostructure designed according to the methods are also described. In some forms, the polyhedral scaffolded RNA nanostructures include two nucleic acid anti-parallel helices spanning each edge of the structure, where the three-dimensional structure is formed from single stranded nucleic acid staple sequences hybridized to a single stranded RNA scaffold sequence, where the RNA scaffold sequence is routed through the Euler cycle of the network defined by vertices and lines of a node-edge network of the polyhedral structure, and where the nanostructure includes at least one edge having a double-strand crossover, where the location of the double-strand crossover is determined by the spanning tree of the network of the polyhedral structure, where the staple sequences are hybridized to the vertices, edges and double strand crossovers of the scaffold sequence to define the shape of the nanostructure, where the staples hybridized to the edges implement crossover asymmetry that includes a difference in the nucleotide position across two helices of the edge of from one to ten nucleotides, inclusive.

In some forms, the polyhedral scaffolded RNA nanostructures include two nucleic acid parallel helices spanning each edge of the structure, where the three-dimensional structure is formed from a single stranded RNA scaffold sequence hybridized to itself and may also hybridize to single stranded nucleic acid staple sequences, the RNA scaffold sequence is routed through the Euler cycle of the network defined by vertices and lines of a node-edge network of the polyhedral structure, where the RNA scaffold sequence hybridizes to itself in at least one edge using parallel crossovers, where the staple sequences, if any, are hybridized to the edges and double strand crossovers of the scaffold sequence to define the shape of the nanostructure, and where the staples hybridized to the edges implement crossover asymmetry that includes a difference in the nucleotide position across two helices of the edge of from one to ten nucleotides, inclusive.

In some forms, the polyhedral or polygonal scaffolded RNA nanostructure includes four or more nucleic acid anti-parallel helices spanning each edge of the structure, where the three-dimensional structure is formed from single stranded nucleic acid staple sequences hybridized to a single-stranded RNA scaffold sequence that is routed through the Euler cycle of the network defined by vertices and lines of a node-edge network of the polyhedral structure, where the nanostructure includes at least one edge including a double strand crossover, where the location of the double strand crossover is determined by a spanning tree of the dual graph of the network of the polyhedral or polygonal structure, where the helices including an edge are arranged as a square lattice of four or more helices, or honeycomb lattice of six or more helices, where the helices meeting at a vertex can be beveled or non-beveled, and where the staple sequences are hybridized to the vertices, edges and double strand crossovers of the scaffold sequence to define the shape of the nanostructure.

In some forms, the scaffolded RNA nanostructures include one or more molecules selected from PNA, protein, lipid, carbohydrate, a small-molecule, a dye, and RNA. The molecule is typically covalently or non-covalently bound to, or complexed with, or encapsulated within the nanostructure. In some forms, the scaffolded RNA nanostructures include a therapeutic, diagnostic or prophylactic agent.

Methods of using the polyhedral scaffolded RNA nanostructures are also provided. In some forms, the methods use the nanostructures for the delivery of therapeutic, diagnostic or prophylactic agents to a subject by administering the nanoparticle to the subject.

Methods of programming 3D geometries of arbitrary compositions of one or more molecules selected from PNA, protein, lipid, carbohydrate, a small-molecule, a dye, and RNA, are also provided. Typically the one or more molecules are conjugated to an underlying scaffolded RNA nanostructure, where the 3D geometry of the one or more molecules is determined by the 3D geometry of the underlying scaffolded RNA nanostructure, where the scaffolded RNA nanostructure is designed according to the described methods for the top-down design of nanostructures. In some forms, assembling the scaffolded RNA nanostructure includes synthesizing the RNA scaffold sequence by a method including in vitro transcription. In some forms, single-stranded or double-stranded nucleic acid overhang sequences extend from nick positions from the oligonucleotide staple strands. In some forms, the nucleic acid overhang sequences that extend from nick positions from the oligonucleotide staple strands form duplex reinforcements along one or more edges of the structure, or span between two vertices of the structure. In some forms, the single-stranded or double-stranded nucleic acid overhangs include one or more sequences of nucleic acids that is complementary to a target RNA or DNA sequence. In some forms, the single-stranded or double-stranded nucleic acid overhangs include one or more sequences of nucleic acids that interact with DNA binding proteins or RNA-binding proteins. Typically, the edge length and nanoparticle geometry is greater than the size of the target molecule that is to be captured, to allow for 1, 2, 3, or more than 3 molecules to be bound independently of any other.

In some forms, the RNA scaffold expresses or encodes one or more selected from messenger RNA (mRNA), replicating RNA (repRNA), guide-strand RNA (gsRNA), ribosomal RNA (rRNA), transfer RNA (tRNA), genomic transcript RNA, aptamer RNA or other functional RNA(s). For example, in some forms, the RNA scaffold includes messenger RNA (mRNA) encoding one or more polypeptides or proteins. In particular forms, the messenger RNA (mRNA) encodes one or more polypeptide or protein antigens. Exemplary antigens include viral antigens, bacterial antigens, protozoan antigens, environmental allergens, food allergens and tumor antigens. In other forms, the messenger RNA (mRNA) encodes one or more enzymes, fluorescent proteins or antigen-binding proteins. In particular forms, the messenger RNA (mRNA) encodes the prokaryotic green fluorescent protein (GFP) protein. In other forms, the RNA scaffold includes or encodes a functional RNA selected from antisense molecules, silencing RNA (siRNA), micro RNA (miRNA), ribozymes, riboswitches, short hairpin RNA (shRNA), triplex forming RNA, and interfering RNA (RNAi).

In some forms, the RNA scaffold and/or staple sequences include one or more modified nucleotides. In some forms, the modified nucleotides reduce or prevent degradation of the modified RNA by RNAse enzymes.

Exemplary modified nucleotides include 2′-fluorinated deoxy-uridine, 2′-fluorinated deoxy-cytosine, and 5-methoxyuridine. In some forms, the scaffolded RNA nanostructure includes one or more RNA/DNA hybrid regions. For example, in some forms, the RNA/DNA hybrid regions facilitates release of the scaffold RNA and/or one or more cargo molecules in the presence of an RNA/DNA hybrid specific nuclease.

Vaccines including scaffolded RNA nanostructures where the RNA scaffold includes messenger RNA (mRNA) that is, or encodes or expresses one or more antigens are also provided. Exemplary vaccines include, express or encode one or more of viral antigens, bacterial antigens, protozoan antigens, environmental allergens, food allergens or tumor antigens.

### BRIEF DESCRIPTION OF THE DRAWINGS

FIG. 1 is a schematic illustrating the workflow for top-down sequence design of different polyhedral RNA origami objects using different RNA species as scaffolds, including mRNA, genomic transcripts and ribosomal RNA. The scaffold is routed through each shape and staples are assigned for (i) edges with no scaffold crossover, (ii) edges with a scaffold crossover, and (iii) vertices. An atomic model structure is then produced. The structure is folded using the staple sequences calculated by the program and the folding/structure are evaluated and characterized by gel shift assay, DMS-MaPseq and Cryo-EM.

FIGS. 2A-2C show characterization of EGFP mRNA scaffolded origami, including a gel mobility shift assay for evaluation of folding with lanes including Marker (M), unfolded RNA, filter-purified folded rT66 structure, and unpurified folded rT66, respectively (FIG. 2A); box plots showing normalized DMS reactivity (mean log₁₀ DMS over segment), corresponding to how frequently a nucleotide is unpaired for the rT66 structure folded without (left), or with (right) staples, respectively. Each data point corresponds to a nucleotide in the scaffold sequence. Whiskers indicate the furthest points within a distance 1.5 times that of the interquartile distance from the box limits, with outliers shown as dots (FIG. 2B); The input target geometry and predicted DX wireframe atomic model for the rT66, followed by an example micrograph (scale bar: 50 nm), two 2D class averages (insets), and two views of the reconstructed density map (scale bars: 5 nm). Arrow indicates modest edge bowing (FIG. 2C).

FIGS. 3A-3B show characterization of M13 RNA transcript scaffolded origami, including a gel mobility shift assay for evaluation of folding with lanes including Marker (M), unfolded RNA, filter-purified folded rO44 structure, and unpurified folded rO44, respectively (FIG. 3A); The input target geometry and predicted DX wireframe atomic model for the rO44, followed by an example micrograph (scale bar: 50 nm), two 2D class averages (insets), and two views of the reconstructed density map (scale bars: 5 nm). Arrows indicate evidence of twist between the two helices of each edge (FIG. 3B).

FIGS. 4A-4F show characterization of 23s rRNA-scaffolded origami. FIG. 4A shows a micrograph of gel mobility shift assay, including lanes (left to right): marker (1 kb plus DNA ladder, NEB), unfolded 23s rRNA fragment scaffold, spin filter-purified folded rO66, and unpurified folded rO66. FIG. 4B shows a micrograph of gel mobility shift assay, including lanes (left to right): unpurified folded rPB66, marker (1 kb plus DNA ladder, NEB), unfolded 23s rRNA fragment scaffold. FIG. 4C and FIG. 4D are box plots of normalized DMS Reactivity (mean login DMS over segment), corresponding to how frequently a nucleotide is unpaired, for the 23s rRNA fragment scaffold, folded without (left) or with (right) staples for the origami (rPO66 in (FIG. 4C) and rPB66 in (FIG. 4D)). Each data point corresponds to a nucleotide in the scaffold sequence. Whiskers indicate the furthest points within a distance 1.5 times that of the interquartile distance from the box limits, with outliers shown as dots. FIG. 4E shows a diagram of the input target geometry and predicted DX wireframe atomic model for the rO66, followed by an example micrograph (scale bar: 50 nm), two 2D class averages (insets), and two views of the reconstructed density map (scale bars: 5 nm). Arrows indicate 1. Slight bowing in the DX edge, 2. Apparent twist in the vertex due to offset helical ends, and 3. Twist in the DX edge. FIG. 4F shows the input target geometry and predicted DX wireframe atomic model for the rPB66, followed by an example micrograph (scale bar: 50 nm), two 2D class averages (insets), and two views of the reconstructed density map (scale bars: 5 nm). Arrows indicate 1. The offset in the ends of helices due to the A-form pitch, 2-3. Indications of twist in the DX edges.

FIGS. 5A-5F show DMS-MaPseq-derived insights into origami per-base stability. FIGS. 5A-5B are schematics of a DX edge, indicating the names for structural features. Structural features including staple DX, staple SX and scaffold DX (FIG. 5A) and staple nick, staple single terminal, and vertex (FIG. 5B) are noted on the scaffold strand (outer), even when the feature is in the staple strand (inner) hybridized with the scaffold, because all chemical probing was measured on the scaffold. FIG. 5C is an array of box plots of stability in proximity to a structural feature showing the quartiles of base 10 logarithms of DMS reactivities of adenine (A) and cytosine (C) residues at each position from 6 bp upstream (−6 to −1) to 6 bp downstream (1 to 6) of each structural feature including scaffold DX, staple DX, staple SX, staple nick, staple single terminus, and vertex, respectively; outliers are more than 1.5 interquartile ranges from the box. P-values refer to the difference between the median DMS reactivity at each position to the median DMS reactivity at all positions further from the structural feature (one-sided Mann Whitney U test). FIG. 5D is a graph of the relationship between the predicted melting temperature of an unbroken double helical segment and the mean DMS reactivity over all A and C residues in the segment, showing mean log₁₀ DMS reactivity (interior) over predicted melting temperature for each of scaffold DX (x), staple DX (+), staple SX (Y), staple nick (O), staple single term (⋄), and vertex ( [symbol] ), respectively. Each segment corresponds to one point whose color and shape are determined by the structural features at its 3′ and 5′ ends, respectively. FIG. 5E is a graph of influence of structural features on the stabilities of 5′ and 3′ ends of double helical segments, showing log 10 ratio of end to interior (−1 to 2) for each of scaffold DX, staple DX, staple SX, staple nick, staple single term, and vertex, respectively for each of features at 5′ ends and 3′ ends. For each structural feature, each point corresponds to one segment. The y coordinate is the log ratio of the DMS reactivity of the base at the 5′ or 3′ end of the segment to the mean DMS reactivity of all interior A and C residues in the segment. The mean ratio for each feature is marked by a horizontal black bar. The P-value for each feature (scatterplot, above) indicates the significance that the bases next to the structural feature are more DMS reactive than interior bases (one tailed Wilcoxon signed-rank test). The curves connecting pairs of structural features indicate the significance that the features have different median reactivities (two-tailed Mann-Whitney U test). Only pairs for which P<0.01 are shown; *P<10-2, **P<10-3, ***P<10-4, ****P<10-5. FIG. 5F is a graph of differential stabilities of A-T and C-G pairs at each structural feature, showing log₁₀ ratio of end to interior for each of scaffold DX, staple DX, staple SX, staple nick, staple single term, and vertex, each at 5′ and at 3′ respectively. For each segment, the ratio is computed as in panel D, except that either only As or only Cs are used. For each structural feature, the black arrow points from the mean ratio among C residues (tail) to the mean ratio among A residues (point of the head), and its length indicates the magnitude of the difference. P-values refer to the significance that this difference is not zero (two-tailed Mann-Whitney U test).

FIGS. 6A-6D are schematic representations of each of four forms of scaffold routing design strategies, including B-form (DAEDALUS;) (FIG. 6A), A-form with scaffold crossover asymmetry (FIG. 6B), A-form with staple crossover asymmetry (FIG. 6C) and Hybrid-form scaffold and staple edge and vertex routings (FIG. 6D). The scaffold crossover asymmetry routing is referred to in the main text as “alternative A-form.”

FIGS. 7A-7D are photomicrographs of electrophoresis gels. FIGS. 7A-7B each show a series of three gels imaged using different exposures of a RNA-scaffolded tetrahedron (scaffold crossover asymmetry design) with 66-bp edge lengths, including lanes with marker (M), RNA Scaffold (Sc) as well as titration series of 0-500 mM KCl (FIG. 7A) or 0-500 mM NaCl, (FIG. 7B), respectively. FIGS. 7C-7D are gels showing a comparison of alternative folding conditions, with HEPES as compared to Tris-HCl pH 8.1 for the long folding protocol (FIG. 7C); and HEPES with the modified folding protocol (“Control fold”) was tested against fast-folding protocol in magnesium, showing near equivalent yields when adjusted for loading.

FIGS. 8A-8B are triplicate graphs of Dynamic light scattering showing Number (0-25%) over Diameter (nm) for monodisperse objects for each of scaffold asymmetry designed RNA-scaffolded tetrahedron with 66 bp per edge (FIG. 8A); and scaffold asymmetry designed RNA-scaffolded pentagonal bipyramid with 66 bp per edge (FIG. 8B), respectively.

FIGS. 9A-9C show characterization of staple-asymmetry-designed A-form tetrahedron with EGFP mRNA scaffold, including a 2D class average micrograph (FIG. 9A), two views of a 2D NMR reconstruction at a resolution of 12 Å (FIG. 9B); and a graph showing Fourier shell correlation (FSC) over spatial frequency (1/A) (FIG. 9C), respectively.

FIGS. 10A-10C show characterization of staple-asymmetry-designed A-form octahedron with four helical turns per edge with M13 RNA transcript scaffold, including a 2D class average micrograph (FIG. 10A), two views of a 2D NMR reconstruction at a resolution of 17 Å (FIG. 10B); and a graph showing Fourier shell correlation (FSC) over spatial frequency (1/Å) (FIG. 10C), respectively.

FIGS. 11A-11C show characterization of staple-asymmetry-designed A-form octahedron with six helical turns per edge and 23s rRNA fragment scaffold, including a 2D class average micrograph (FIG. 11A), two views of a 2D NMR reconstruction at a resolution of 13 Å (FIG. 11B); and a graph showing Fourier shell correlation (FSC) over spatial frequency (1/Å) (FIG. 11C), respectively.

FIGS. 12A-12C show characterization of staple-asymmetry-designed A-form pentagonal bipyramid with 23s rRNA fragment scaffold, including a 2D class average micrograph (FIG. 12A), two views of a 2D NMR reconstruction at a resolution of 19 Å (FIG. 12B); and a graph showing Fourier shell correlation (FSC) over spatial frequency (1/Å) (FIG. 12C), respectively.

FIGS. 13A-13B are schematic representations of RNA-scaffolded tetrahedron with a 66-bp edge lengths folded using A-form geometry staples (FIG. 13A); or Hybrid-form geometry staples (FIG. 13B), respectively.

FIGS. 14A-14B show Cryo-EM micrograph of RNA-scaffolded pentagonal bipyramid with 66-bp edge lengths folded using scaffold asymmetry design A-form geometry staples (FIG. 14A), and two views of a 2D NMR reconstruction (FIG. 14B), respectively.

FIGS. 15A-15B show characterization of a regular RNA-scaffolded tetrahedron showing the distinct wireframe structure designed with scaffold crossover asymmetry. FIG. 15A shows a micrograph of gel mobility shift assay, including lanes (left to right): marker (1 kb plus DNA ladder, NEB), unfolded RNA scaffold, and folded scaffold. FIG. 15B shows two views of a 2D NMR reconstruction of the regular RNA-scaffolded tetrahedron.

FIGS. 16A-16B show characterization of a regular RNA-scaffolded pentagonal bipyramid with 66-bp edge lengths showing the wireframe structure designed with scaffold crossover asymmetry. FIG. 16A shows a micrograph of gel mobility shift assay, including lanes (left to right): marker (1 kb plus DNA ladder, NEB), unfolded RNA scaffold, and folded scaffold. FIG. 16B shows two views of a 2D NMR reconstruction of the regular RNA-scaffolded pentagonal bipyramid. A notable twist is seen along the edge, which disrupts the electron density at the vertices, as shown.

FIGS. 17A-17B show views of a 2D NMR reconstruction for scaffold asymmetry-designed regular octahedra with four helical turns per edge (44 bp) (FIG. 17A); or six helical turns per edge (66 bp) (FIG. 17B), respectively.

FIGS. 18A-18C show characterization of biochemical stability of the scaffold-asymmetry-designed EGFP mRNA-scaffolded tetrahedron with 66-bp edge length. FIG. 18A shows a molecular structure of canonical unmodified nucleotides, and a micrograph of an electrophoresis gel, showing lanes including Marker (M), unfolded scaffold, folded nucleic acid structure, and structure treated with RNAse A (A) or RNAse H (H), respectively. Nanoparticles folded with canonical unmodified nucleotides were subjected to treatment with RNase A and RNase H for 5 minutes at 37° C., showing complete degradation of the scaffold and release of the DNA staples. FIG. 18B shows a molecular structure of 2′-fluorinated deoxy-uridine and cytosine, and a micrograph of an electrophoresis gel, showing lanes including Marker (M), unfolded scaffold, folded nucleic acid structure, and structure treated with RNAse A (A), respectively. RNA including 2′-fluorinated deoxy-uridine and cytosine was folded using the A-form geometry staples, and additionally subjected to RNase A treatment, showing no detected degradation in the five minutes it took the unprotected nanoparticle to degrade, though a notable accumulation in the well suggests RNase binding without cleavage. FIG. 18C shows a molecular structure of 5-methoxyuridine, and a micrograph of an electrophoresis gel, showing lanes including Marker (M), unfolded scaffold, and folded nucleic acid structure, respectively. 5-methoxyuridine can also fold to a single band using A-form staples.

FIGS. 19A-19B are images of 10 scaffolded RNA origami objects designed with the pyDAEDALUSX software, including (FIG. 19A) Input geometries, and (FIG. 19B) output 3D structure predictions, including four Platonic, two Archimedean, two Johnson, and two Catalan polyhedra, respectively, generated using top-down design procedures. Objects are not drawn to scale; space-filling and ribbon cartoons are shown for each model.

FIGS. 20A-20B are schematics showing folding of a 3D-structure according to the methods of transcription and folding with staples without (FIG. 20A) and with (FIG. 20B) an insert that is designed to fold into the interior or exterior of the structure, such as a single-stranded loop region. FIG. 20C is a schematic showing a scaffold with an extra RNA sequence incorporated, in which the inserted RNA protrudes from the scaffold and hangs off the folded nanostructure. The scaffold is shown in blue, staples in grey, and the extra RNA sequence is shown in darker blue.

### DETAILED DESCRIPTION OF THE INVENTION

### I. Definitions

The term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The terms “oligonucleotide” or a “polynucleotide” are synthetic or isolated nucleic acid polymers including a plurality of nucleotide subunits.

The terms “nucleic acid,” “polynucleotide,” and “oligonucleotide” are interchangeable and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid). In general and unless otherwise specified, an analogue of a particular nucleotide has the same base-pairing specificity; i.e., an analogue of A will base-pair with T. When double-stranded DNA is described, the DNA can be described according to the conformation adopted by the helical DNA, as either A-DNA, B-DNA, or Z-DNA. The B-DNA described by James Watson and Francis Crick is believed to predominate in cells and extends about 34 Å per 10 bp of sequence; A-DNA extends about 23 Å per 10 bp of sequence, and Z-DNA extends about 38 Å per 10 bp of sequence.

In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some embodiments the set of characters is (A, C, G, T, U) for adenosine, cytidine, guanosine, thymidine, and uridine respectively. In some embodiments the set of characters is (A, C, G, T, U, I, X, Ψ) for adenosine, cytidine, guanosine, thymidine, uridine, inosine, uridine, xanthosine, pseudouridine respectively. In some embodiments the set of characters is (A, C, G, T, U, I, X, Ψ, R, Y, N) for adenosine, cytidine, guanosine, thymidine, uridine, inosine, uridine, xanthosine, pseudouridine, unspecified purine, unspecified pyrimidine, and unspecified nucleotide respectively. The modified sequences, non-natural sequences, or sequences with modified binding, may be in the genomic, the guide or the tracr sequences.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

The terms “cleavage” or “cleaving” of nucleic acids, refer to the breakage of the covalent backbone of a nucleic acid molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered “sticky” ends. In certain embodiments cleavage refers to the double-stranded cleavage between nucleic acids within a double-stranded DNA or RNA chain.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or MEGALIGN (DNASTAR) software is used to align sequences. Those skilled in the art can determine appropriate parameters for measuring alignment, including any formulas needed to achieve maximal alignment over the full-length of the sequences being compared. When sequences are aligned, the percent sequence identity of a given sequence A to, with, or against a given sequence B (which can alternatively be phrased as a given sequence A that has or includes a certain percent sequence identity to, with, or against a given sequence B) can be calculated as: percent sequence identity=X/Y100, where X is the number of residues scored as identical matches by the sequence alignment program's or formula's alignment of A and B and Y is the total number of residues in B. If the length of sequence A is not equal to the length of sequence B, the percent sequence identity of A to B will not equal the percent sequence identity of B to A. Mismatches can be similarly defined as differences between the natural binding partners of nucleotides. The number, position and type of mismatches can be calculated and used for identification or ranking purposes.

The term “endonuclease”, refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCI, EcoRI, BglII, and AlwI. Endonucleases include also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a homing endonuclease, a mega-nuclease, a chimeric Zinc-Finger nuclease (ZFN) or TAL effector nuclease (TALEN) resulting from the fusion of engineered zinc-finger domains or TAL effector domain, respectively, with the catalytic domain of a restriction enzyme such as FokI, other nuclease or a chemical endonuclease including CRISPR/Cas9 or other variant and guide RNA.

The term “exonuclease”, refers to any wild type or variant enzyme capable of removing nucleic acids from the terminus of a DNA or RNA molecule, preferably a DNA molecule. Non-limiting examples of exonucleases include exonuclease I, exonuclease II, exonuclease III, exonuclease IV, exonuclease V, exonuclease VI, exonuclease VII, exonuclease VII, Xm1, and Rat1.

In some cases an enzyme is capable of functioning both as an endonuclease and as an exonuclease. The term nuclease generally encompasses both endonucleases and exonucleases, however in some embodiments the terms “nuclease” and “endonuclease” are used interchangeably herein to refer to endonucleases, i.e., to refer to enzyme that catalyze bond cleavage within a DNA or RNA molecule.

As used herein, the term “ligating” refers to enzymatic reactions in which two double-stranded DNA molecules are covalently joined, for example, catalyzed by a ligase enzyme.

As used herein, the terms “aligning” and “alignment” refer to the comparison of two or more nucleotide sequence based on the presence of short or long stretches of identical or similar nucleotides. Several methods for alignment of nucleotide sequences are known in the art, as will be further explained below.

The terms “scaffolded RNA”, “scaffolded RNA nanostructure”, “RNA nanostructure”, “RNA-based nucleic acid nanostructure”, and “RNA-based nanostructure” are used interchangeably. They refer to nano-scale 3D or 2D nucleic acid structures formed from a single-stranded RNA “scaffold” that is folded into the 3D or 2D shape. The structures can be folded using one or more short single strands of nucleic acids (staple strands) to direct the folding of the longer, single strand of RNA polynucleotide (scaffold strand) into desired shapes on the order of about 10-nm to a micron or more, and the structures form therefrom. The staple strands can be DNA, or RNA, or any other form of nucleic acid, or nucleic acid variant, such as PNA. In some forms, the RNA scaffold is folded in the absence of any staple strands.

The term polyhedron refers to a three-dimensional solid figure in which each side is a flat surface. These flat surfaces are polygons and are joined at their edges.

The terms “staple strands” or “helper strands” are used interchangeably. “Staple strands” or “helper strands” refer to oligonucleotides that hold the scaffold RNA in its three-dimensional wireframe geometry. Additional nucleotides can be added to the staple strand at either 5′ end or 3′ end, and those are referred to as “staple overhangs”. Staple overhangs can be functionalized to have desired properties such as a specific sequence to hybridize to a target nucleic acid sequence, or a targeting element. In some instances, the staple overhang is biotinylated for capturing the RNA nanostructure on a streptavidin-coated bead. In some instances, the staple overhang can be also modified with chemical moieties. Non-limiting examples include Click-chemistry groups (e.g., azide group, alkyne group, DIBO/DBCO), amine groups, and Thiol groups. In some instances, some bases located inside the oligonucleotide can be modified using base analogs (e.g., 2-Aminopurine, Locked nucleic acids, such as those modified with an extra bridge connecting the 2′ oxygen and 4′ carbon) to serve as linker to attach functional moieties (e.g., lipids, proteins). Alternatively RNA-binding proteins or guide RNAs can be used to attach secondary molecules to the RNA scaffold.

The term “geometry” or “geometric parameters” refer to the angles and/or relative distances that describe any two connected edges of a shape, such as those that define the relative position of faces, and the properties of the vertices and edges that form the three-dimensional solid.

The term “arbitrary geometry” refers to a non-specific three-dimensional shape, for example, any desired three-dimensional closed surface that can be rendered as a polyhedral wire mesh.

The term “network” is a representation of the lines and vertices that define the relations between the line and vertices within the objects. In some embodiments, vertices are represented as nodes and lines are represented as edges in a graph. The degree (or valency) of a vertex of a graph is the number of edges incident to the vertex.

The term “spanning tree” refers to a subset of edges and all the nodes in a graph, such as the graphical node-edge network corresponding to the lines and vertices of a polyhedral shape. Typically, spanning trees include all the vertices. Different spanning trees for a given network can cover different edges. A breadth-first spanning tree includes the maximum number of branches.

The term “Euler Path”, “Eulerian Trail”, or “Eulerian Path” refer to a trail in a graph which visits every edge exactly once. The terms “Euler circuit”, “Euler Cycle”, “Eulerian Cycle” or “Eulerian Circuit” are used interchangeably and refer to a trail in a graph which visits every edge exactly once, and which starts and ends on the same vertex. For the existence of Eulerian cycles it is necessary that every vertex has even degree, and all of its vertices with nonzero degree belong to a single connected component.

The term “loop-crossover structure” refers to 3D structure in which endpoints are joined such that every duplex becomes part of a loop, and positions of possible scaffold double crossovers are found between two loops.

The term “dual graph” refers to the graph by converting each loop to a node and each double crossover to edge.

The terms “DX crossover”, or “antiparallel crossover”, or “DX motif” are used interchangeably, and refer to an antiparallel double crossover nucleic acid motif including of two four-arm Holliday junctions, joined by two helical arms at two adjacent arms. The antiparallel orientation of the nucleic acid helical domains in antiparallel DX motifs implies that the major grooves of one nucleic acid helix faces the minor groove of the other engaged helices come together in each turn.

The term “PX crossover”, or “parallel crossover”, or “PX motif” are used interchangeably to refer to a four-stranded RNA motif wherein two parallel double helices are joined by reciprocal exchange (crossing over) of strands of the same polarity at every point where the strands come together (see Seeman, Nano Letters 1, (1), pp. 22-26 (2001); Wang, PNAS, V. 107 (28), pp. 12547-12552 (2010)). No strand breakage and rejoining is needed, because two double helices can form PX-RNA merely by inter-wrapping. The reciprocal exchange between two double stranded nucleic acid helices can occur between two helices having either the same or opposite stand polarity. An exemplary PX motif is the “paranemic crossover”. PX motifs are usually followed by a pair of numbers, e.g. PX65 motif, that describe the number of base pairs in the major groove and minor groove of the double helices, respectively, between parallel crossovers. The number of base pairs in the major groove is typically greater than that in the minor groove. Exemplary, PX motifs include PX65, PX75, PX85, PX95, PX64, PX74, and PX66 (Maiti, et al., Biophysical Journal. 90, 1463-1479 (2006); Shen, et al., J. Am. Chem. Soc. 126, 1666-1674 (2004)).

The term “bait sequence” refers to a single-stranded nucleic acid sequence that is complementary to any fragment of a target nucleic acid sequence, such as an RNA, for capturing the target nucleic acids. Typically, bait sequences are appended to or otherwise are present as part of the staple sequence of a nucleic acid nanostructure, for example, as an “overhang” sequence of nucleic acids. In some embodiments, bait sequences are complementary to loop regions or single-stranded regions of target RNAs for capturing the RNAs. Alternatively, the bait sequences tether proteins or other ligands that target binding regions to capture a structured RNA or DNA assembly of interest via avidity enhancement.

The term “nucleic acid capture” refers to binding of any nucleic acid molecule of interest having complementary nucleic acid sequences to the bait sequences on the RNA nanostructures, or having affinity for the capture bait probe employed, and being immobilized or attached to the RNA nanostructures via hybridization to the bait sequence, or binding. For example, “RNA capture” refers to binding of any ribonucleic acid molecule of interest to the bait sequences on the RNA nanostructures. Nucleic acids of interest can bind to the inside or outer surface of a nucleic acid nanostructure.

The phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M-1. Affinities greater than 10⁸ M-1 are preferred.

The term “targeting molecule” refers to a substance which can direct a nanoparticle to a receptor site on a selected cell or tissue type, can serve as an attachment molecule, or serve to couple or attach another molecule. The term “direct” refers to causing a molecule to preferentially attach to a selected cell or tissue type. This can be used to direct cellular materials, molecules, or drugs, as discussed below.

The terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes a bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny, et al., J. Immunol., 148, 1547-1553 (1992).

The terms “epitope” or “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10, amino acids, in a unique spatial conformation. Methods of determining spatial conformation of epitopes include, for example, x-ray crystallography and 2-dimensional nuclear magnetic resonance.

The term “small molecule,” as used herein, generally refers to an organic molecule that is less than about 2,000 g/mol in molecular weight, less than about 1,500 g/mol, less than about 1,000 g/mol, less than about 800 g/mol, or less than about 500 g/mol Small molecules are non-polymeric and/or non-oligomeric.

### II. Methods for Design of Scaffolded RNA Nanostructures

Systems and methods for the automated, step-wise design of scaffolded RNA nanostructures having arbitrary geometries have been established. The systems and methods generally involve rendering the geometric parameters of a desired polyhedral form as a node-edge network, and determining the RNA scaffold route and staple design parameters necessary to form the desired polyhedral structure based on A-form helical nucleic acid geometry. Therefore, methods for generating the sequences of the single-stranded RNA scaffold and/or the nucleic acid sequence of staple strands that combine to form a scaffolded RNA nanostructure having the desired shape are provided. An exemplary method for designing scaffolded RNA nanostructure having a desired polyhedral form includes selecting a desired 3D polyhedral or 2D polygon form as a target structure; providing geometric parameters and physical dimensions of the a target structure for a selected 3D polyhedral or 2D polygon form; identifying the route of a single-stranded RNA scaffold that traces throughout the entire target structure; and generating the sequences of the single-stranded RNA scaffold and/or the nucleic acid sequence of staple strands that combine to form a scaffolded RNA nanostructure having the desired shape.

Generally, the scaffolded RNA nanostructures having the desired shape are produced by folding a long single stranded RNA polynucleotide, referred to as a “scaffold strand”, into a desired shape or structure using a number of small “staple strands” as glue to hold the scaffold in place. Typically, the number of staple strands will depend upon the size of the scaffold strand, the complexity of the shape or structure, the types of crossover motifs employed, and the number of helices per edge. For example, for relatively short scaffold strands (e.g., about 150 to 1,500 base in length) and/or simple structures the number of staple strands are small (e.g., about 5, 10, 50 or more). For longer scaffold strands (e.g., greater than 1,500 bases) and/or more complex structures, the number of staple strands can be several hundreds to thousands (e.g., 50, 100, 300, 600, 1,000 or more helper strands). Using parallel crossover motifs, however, the number of staples can be reduced, even to zero. The choice of staple strands and, in some instances, the programmed self-hybridization of the scaffold strand, determine the pattern. In some embodiments, a software program is used to identify the staple strands needed to form a given design.

Typically, the methods include one or more of the following steps:
(a) determining the geometric parameters of an input, wherein the input includes a 3D polyhedral or 2D polygon geometric shape and optionally one or more of its physical dimensions;
(b) identifying a route for a single-stranded RNA scaffold that traces throughout the geometric shape based on A-form helical nucleic acid geometry; and
(c) generating the sequences of the single-stranded RNA scaffold and optionally the nucleic acid sequence of staple strands that combine to form a scaffolded RNA nanostructure having the geometric shape.

In some forms, identifying a route for a single-stranded RNA scaffold that traces throughout the geometric shape in step (b) is based on staple crossover asymmetry, with 11 nucleotides per helical turn. In some forms of the methods, step (b) includes staple routing.

Typically, crossover asymmetry includes a difference in the nucleotide position between two helices of an edge.

In some forms, the staple crossover asymmetry includes a difference in the nucleotide position across two helices of an edge of from one to ten nucleotides, inclusive. For example, in some forms, the staple crossover asymmetry includes a difference in the nucleotide position across two helices of an edge of one nucleotide, two, three, four, five, six, seven, eight, nine or ten nucleotides, inclusive. In particular forms, the staple crossover asymmetry includes a difference of four nucleotides in the nucleotide position across two helices of an edge.

In some forms, identifying a route for a single-stranded RNA scaffold that traces throughout the geometric shape in step (b) is based on scaffold crossover asymmetry, with 11 nucleotides per helical turn.

Typically, the scaffold crossover asymmetry includes a difference in the nucleotide position across two helices of an edge of from one to ten nucleotides, inclusive. For example, in some forms, the scaffold crossover asymmetry includes a difference in the nucleotide position across two helices of an edge of one nucleotide, two, three, four, five, six, seven, eight, nine or ten nucleotides, inclusive. In particular forms, the scaffold crossover asymmetry includes a difference of six nucleotides in the nucleotide position across two helices of an edge.

In some forms, generating the sequences of the single-stranded RNA scaffold in step (c) includes staple crossover asymmetry, with 11 nucleotides per helical turn.

In other forms, identifying a route for a single-stranded RNA scaffold that traces throughout the geometric shape in step (b) is based on no crossover asymmetry with 11 nucleotides per helical turn.

Typically, the route of the scaffold RNA is identified by
(i) Determining edges that form the spanning tree of the node-edge network (for example, using the Prim's Formula);
(ii) Bisecting each edge that does not form the spanning tree to form two split edges;
(iii) Determining an Eulerian circuit that passes twice along each edge of the spanning tree. The direction of the continuous scaffold sequence is reversed at the bisecting point of the node-edge network in a DX-anti-parallel crossover, and the Eulerian circuit defines the route of a single-stranded nucleic acid scaffold sequence that passes throughout the entire structure. Staple strands are located at the vertices and edges of the route of the single-stranded nucleic acid scaffold sequence determined in (d).

Typically, for the scaffolded RNA nanostructures that incorporate parallel crossovers, the route of the scaffold nucleic acid is identified by determining an Eulerian circuit that passes twice or more than twice along each edge of the wireframe. Based on the length and spanning tree classification, units of partial scaffold routing are superimposed and connected to complete the circuit.

In some embodiments the methods further include the steps by
(i) Detaching and scaling each edge of the initial geometry to represent the number of helixes as lines indicating their lengths and endpoints;
(ii) Generating the loop-crossover structure joining endpoints and finding double crossovers between two loops;
(iii) Generating the dual graph of the loop-crossover by converting each loop to a node and each double crossover to edge;
(iv) Computing the spanning tree of the dual graph of the loop-structure (for example, using the Prim's Formula);
(v) Inverting the dual graph back to the loop-crossover structure but without the double-crossovers corresponding to the non-member's spanning tree; Edges that are members of the spanning tree correspond to the subset of crossovers required to complete the Eulerian circuit.

In some embodiments the methods further include the step of
(f) Modelling the 3-Dimensional co-ordinates of each nucleic acid according to the parameters determined in (c) and (d).

In further embodiments the methods further include the step of
(g) Assembling and optionally purifying the nucleic acid nanostructures designed by the methods of any of the steps (a) through (d).

Each of these steps is discussed in more detail, below.

The method described herein is a “top-down approach” of the structure (i.e., only input is a “shape” and the number and geometry of helices per edge). Nothing else is required, except for optional selection of a size and an input sequence (otherwise, default parameters can be used for both).

Default parameters for input scaffold size, nucleic acid type, input scaffold sequence, edge length, number of helices per edge, cross-sectional morphology of edges and vertex geometry (i.e., beveled or non-beveled edges) can be used as necessary to generate the sequences of staples and/or scaffold nucleic acid when no value is specified. Typically, the default nucleic acid is A-form RNA, and the default edge-length is measured in units of 11 bases, with 2 helices per edge. In some embodiments, the default nucleic acid scaffold sequence is the 792-nucleotide (nt) prokaryotic EGFP mRNA, or 660-nt or 924-nt de Bruijn RNA sequences. In some embodiments, when the number of helices per edge is specified, but the vertex morphology is not specified, the default vertex geometry is to use honeycomb morphology with beveled edges.

This is fundamentally different from bottom-up design methods. For example, the “bottom-up approach” does not produce the sequences of staple strands, but requires manual intervention via an heuristic approach, using multiple duplex arms combined together to form the structure (i.e., may not use a single scaffold sequence throughout). The top-down methods start with the desired output, i.e. the final structure and the use of a specific scaffold, and generate the sequences required to synthesize that output, using a single RNA scaffold that is routed throughout the entire structure. The scaffold can be a user-defined scaffold sequence, and the staple sequences are varied accordingly.

The formula uses a maximum-breadth spanning tree to determine positions of the scaffold crossovers for the scaffold routing. Any spanning tree, however, will lead to a valid scaffold routing. The nanostructures themselves are distinct in having a continuous single stranded nucleic acid sequence routed through each edge of the structure. Typically, the methods produce scaffolded RNA nanostructures based on A-form nucleic acid helical geometry having equivalent stability in solution as a corresponding scaffolded DNA nanostructure based on B-form nucleic acid helical geometry and having similar morphology and dimensions. In some forms, the methods produce scaffolded RNA nanostructures based on A-form nucleic acid helical geometry having greater stability in solution than a corresponding scaffolded DNA nanostructure based on B-form nucleic acid helical geometry and having similar morphology and dimensions.

A. Providing a Target Structure

Methods for the step-wise design of a scaffolded RNA nanostructure, based on the arbitrary wireframe geometry of the desired (target) structure as a starting model have been developed. The methods are useful to provide design parameters, such as the sequence of a single-stranded RNA nucleic acid “scaffold”, as well as corresponding single-stranded edge and vertices “staple” sequences necessary to form a nucleic acid nanostructure having the shape of the input structure.

1. Selection of Target Structure

The methods require geometric parameters that define the target shape as input. Therefore, the starting point for the design process is the selection of a target shape. Any arbitrary geometric shape that can be rendered as a “wireframe” model can be selected as input for the design of nucleic acid assemblies.

Exemplary target shapes include three-dimensional structures, including, but not limited to, Platonic polyhedrons, Archimedean polyhedrons, Johnson polyhedrons, Catalan solids, or asymmetric three-dimensional structures. In some embodiments, the target structure has a programmed geometry that is topologically equivalent to that of a sphere. In other embodiments, the target structure has a programmed geometry that is topologically distinct to that of a sphere. For example, target structures including nested structures, and toroidal structures can be designed using the described methods. In other embodiments, the target structure has a programmed geometry that is topologically equivalent to a plane. For example, target structures including triangular mesh, square mesh, or other mesh.

Target structures can be selected based upon one or more design criteria, or can be selected randomly. In some embodiments, structures are selected based on existing ‘natural’ 3-dimensional organizations (e.g., virus capsids, antigens, toxins, etc.). Therefore, in some embodiments, target shapes are designed for use directly or as part of a system to mediate a biological or other responses which are dependent upon, or otherwise influenced by 3D geometric spatial properties. For example, in some embodiments, all or part of a structure is designed to include architectural features known to elicit or control one or more biological functions. In some embodiments, structures are designed to fulfill the 3D geometric spatial requirements to induce, prevent, stimulate, activate, reduce or otherwise control one or more biological functions. Typically, the desired shape defines a specific geometric form that will constrain the other physical parameters, such as the absolute size of the particle. For example, the minimum size of nucleic acid nanostructures designed according to the described methods will depend upon the degree of complexity of the desired shape.

i. 2-Dimensional Wireframe Structures

Target structures can be any solid in two dimensions. Therefore, target structures can be a grid or mesh or wireframe topologically similar to a 2D surface or plane. The grid or mesh can be composed of regular or irregular geometries that can be tessellated over a surface.

Exemplary target structures include triangular lattices, square lattices, pentagonal lattices, or lattices of more than 5 sides. 2D structures can be designed to have varied length and thickness in each dimension. In some embodiments, the edges of 2D nanostructures include a single nucleic acid helix. In other embodiments, the edges of 2D nanostructures include two or more nucleic acid helices. For example, in some embodiments, each edge of the 2D nanostructure includes 2 helices, 4 helices, 6 helices or 8 helices, or more than 8 helices, up to 100 helices per edge, although theoretically unlimited in number.

ii. Polyhedral Structures

Target structures can be any solid in three dimensions that can be rendered with flat polygonal faces, straight edges and sharp corners or vertices.

Exemplary basic target structures include cuboidal structures, icosahedral structures, tetrahedral structures, cuboctahedral structures, octahedral structures, and hexahedral structures. In some embodiments, the target structure is a convex polyhedron, or a concave polyhedron. For example, in some embodiments, the methods design nucleic acid assemblies of a uniform polyhedron that has regular polygons as faces and is isogonal. In other embodiments, the methods design nucleic acid assemblies of an irregular polyhedron that has unequal polygons as faces. In further embodiments, the target structure is a truncated polyhedral structure, such as truncated cuboctahedron.

Platonic polyhedrons include polyhedrons with multiple faces, for example, 4 faces (tetrahedron, (1)), 6 faces (cube or hexahedron (2), 8 faces (octahedron), 12 faces (dodecahedron), and 20 faces (icosahedron).

In some embodiments, the target structure is a nucleic acid assembly that has a non-spherical geometry. Therefore, in some embodiments, the target structure has a geometry with “holes”. Exemplary non-spherical geometries include toroidal polyhedra and nested shapes. Exemplary toroidal polyhedra include a torus and double torus. Exemplary topologies of nested shapes include nested cube and nested octahedron. Exemplary polyhedral forms are depicted in FIG. 19.

In other embodiments, target structures can be a combination of one or more of the same or different polyhedral forms, linked by a common contiguous edge.

iii. Reinforced Polyhedral Structures

In some embodiments, the target structure is a reinforced structure. Reinforced structures are structures that share the same polyhedral form as the equivalent, non-reinforced structure, and include one or more additional edges spanning between two vertices. Typically, the reinforced structure contains at least one or more edges than the corresponding non-reinforced structure. In some embodiments, additional structural elements that appear as “cross-bars” spanning between two vertices are introduced.

In some embodiments, a structure is reinforced by the addition of one or more edges passing internally within the space enclosed by the structure. Therefore, in some embodiments reinforced structures encapsulate a smaller volume than the corresponding non-reinforced structure. In other embodiments, a structure is reinforced by the addition of one or more edges that connects vertices by spanning a face of the polyhedron. In further embodiments, a polyhedral nanostructure is reinforced by including one or more additional edges that connect vertices by spanning a face of the polyhedron and one or more additional edges that connect vertices by passing internally within the space enclosed by the structure. In some embodiments, a polyhedral nanostructure is reinforced by addition of one or more edges that bisects a face of the polyhedron and addition of one or more vertices.

iv. Other Structures

In some embodiments the desired structure has a shape that is visually or geometrically similar to a biological structure, such as the shape of a viral particle, or a sub-component of a viral particle; a protein; or a sub-component of a protein.

2. Providing Geometric Parameters of the Target Structure

The methods can include the step of providing the geometric parameters that define the target structure. Geometric parameters include the spatial coordinates of all vertices, the edge connectivity between vertices, and the faces to which vertices belong. Geometric parameters can be determined using any means that represents the form of the target structure. Typically, geometric parameters are determined by rendering the target structure as a wire-frame mesh. In some embodiments the determination of geometric parameters for an input shape is carried out using a computer-based interface. Therefore, in some embodiments geometric parameters of a target shape are determined in silico. Typically, in silico determination of geometric parameters can require input of a target shape, or input of the rendered wire-frame model of the target shape. In some embodiments, the only input is a target shape, or input of the rendered wire-frame model of the target shape. For example, in some embodiments, following input of a target shape and/or geometric parameters corresponding to a target shape, all other steps are performed within an automated system, such as by a computer using software including each of the method steps, optionally incorporating one or more default parameters. In some embodiments, the input is a 2-dimensional shape, or geometric parameters of a 2-dimensional shape. In some embodiments, the target structure is the three-dimensional form corresponding to the 2-dimensional shape. For example, a 3-dimensional cuboidal structure can be inferred from input of the geometric parameters of one or more of the faces of the 3-dimensional structure. In an exemplary embodiment, a single square face is input and the corresponding regular cube is provided as input in wire-frame conformation.

i. Wire-Frame of Arbitrary Geometry

The methods can determine nucleic acid scaffold and staple sequences for scaffolded RNA nanostructures having the shape of any open or closed geometric surface that can be rendered as a polyhedral surface wire-frame model.

Therefore, the methods include reduction of the target structure as a model that represents each edge of the physical object where two or more continuous smooth surfaces meet, or by connecting an object's constituent vertices using straight lines. Typically, a wireframe model of a geometric shape represents the minimum number of characteristic edges and vertices that define the 2D or 3D shape.

Typically, when some or all of the methods are carried out using a computer-based interface, the geometric parameters of a target shape are provided in a standard polyhedral file format. The geometric parameters of any open or closed, orientable surface network can serve as input using any file format that specifies polygonal geometry known in the art, including but not limited to, Polygon File Format (PLY), Stereolithography (STL), or Virtual Reality Modeling Language (WRL). When a standard polyhedral file format is provided, the code includes a parser to convert the standard polyhedral files into the required inputs.

ii. Edge Geometry

In addition to the geometric parameters of a target shape, the cross-sectional shape on all edges is defined. Exemplary cross-sectional forms include two double-stranded nucleic acid helices; a square lattice (minimum four double helices); and honeycomb lattice (minimum six double helices). Each double helical section has an identification number which determines the orientation of the helix along the edge direction. To make antiparallel helixes given in the bundle of helixes, the identification number should be even when the neighboring helix has the odd number.

In some embodiments, one or more edges of a shape is defined as having a square cross-sectional lattice including an even number of double helices. For example, in some embodiments, each edge includes four helices, six helices or more than six double helices, for example, 36, or 64 double helices. The square lattice is composed such that each of helices are arranged with rectangular symmetry across the axis of the edge and such that any one helix can have crossovers along the edge with up to four other helices. In some embodiments, one or more edges of a shape is defined as having a honeycomb cross-sectional lattice including six double helices, eight double helices, ten double helices or more than ten double helices, for example, 12, 24, or 48 double helices arranged in a honeycomb pattern. The honeycomb lattice is composed such that each of the helices are arranged on a hexagon pattern across the axis of the edge and such that each helix can have crossovers with up to three other helices along the edge.

iii. Vertex Geometry

Typically, a vertex of “degree N” has “N” number of edges emerging from it. For example, if a vertex is of degree 4, it is contacted by 4 edges. An Euler circuit through a node-edge network of a given shape is guaranteed when the degree of every vertex is even. Therefore, in preferred embodiments, the degree of every vertex in the node-edge network is even, such that the Eulerian Circuit of the graph passes through each of the edges once in each direction. By choosing to have an even number of duplexes per edge in the wireframe, the vertices of the final RNA nanostructure are technically of even degrees, even if some or all of the vertices in the wireframe input are of odd degrees.

There are several conventions by which to define the inradius of a vertex, which will vary according to the number of edges that combine at the vertex “junction”, and whether the angles between each edge entering or leaving the junction are equal or different relative to one another. For example, the angles at the vertex determine the distance between the vertex and the beginning of the edge. For vertices with equal face angles the distance between the vertex and the beginning of the edge is the inradius of the regular polygon defined by the width of each edge in the junction. For vertices with unequal face angles, the backbone can be stretched, or the vertex can incorporate nucleotide overlap.

For structures including more than 2 double-helices per edge, the cross-geometry of the nucleic acid scaffold and/or staple strands on all vertices is defined for the junctions between each edge, in relation to the interface with the two or more further edges. For example, when an edge is defined as having a honeycomb cross-sectional form, the geometry of each honeycomb lattice edge can be defined as either having a beveled or non-beveled edge at the junction of multiple edges (vertex).

Typically, the geometry assumes A-form nucleic acid helices with 11 nucleotides per helical twist, and asymmetry in the staple crossover positions to account for helical geometry.

In other forms, the geometry assumes A-form nucleic acid helices with 11 nucleotides per helical twist, and asymmetry in the scaffold crossover positions to account for helical geometry.

In other forms, the geometry assumes A-form nucleic acid helices with 11 nucleotides per helical twist, and no asymmetry to account for helical geometry.

In one embodiment of a non-beveled type, between two neighboring edges at a vertex exactly one helix of one edge is involved with exactly one other helix of the other edge by both a scaffold crossover as well as possibly a staple crossover, irrespective of edge lattice type. All other helices on the edge are extended or truncated to the crossover position near to the vertex. Scaffold or staple strands may be unpaired at the vertex, or no unpaired scaffold or staples may be present.

In one embodiment of the beveled type, between two neighboring edges at a vertex one helix, two helices, three helices, or more than three helices from one edge are connected with an equivalent number of helices on the neighboring edge. Thus, for example, three helices on one edge are connected to three helices on a neighboring edge. The edge length of two adjacent edges at the i-th vertex is modified when the angle between two edges is relatively larger than others. At the 4-arm junction, each of four edges denoted by from ‘a’ to ‘d’ is connected at the vertex, i. The minimum angle, θⁱₘᵢₙ at the i-th vertex is found, and the initial off-set distance (apothem), rᵢ is calculated by θⁱₘᵢₙ and the number of arms in i-th vertex. Two cylinders (in case of the DX tile design) are drawn on each edge with initial off-set distance, rᵢ. When two cylinders located in adjacent edges do not contact each other (not close), the new off-set distance, dⁱ_{a,d}, is determined, in which the subscript a,d represents the edge identifier. The new off-set distance, dⁱ_{a,d} can be solved with two given distance mⁱ_{a,d}, and nⁱ_{a,d} and the given angle θⁱ_{a,d}. The helix continues such that each helix will not sterically clash with any other helix, and crossovers of scaffold and staples will occur at closest contact between any two neighboring helices but on different edges.

Thus, the geometry of a flat type will be connected to a neighboring edge by one scaffold and/or staple crossover at the vertex. The geometry of a beveled type will be connected to a neighboring edge by the number of helices of the edge coming into the vertex divided by the number of edges the incoming edge is a neighbor to. In spherical topologies this is defined as the number of helices of the edge divided by 2. Thus, as an example, a beveled edge vertex with an edge composed of six helices total on a honeycomb lattice will share three scaffold and/or staples crossed-over to three helices on a neighboring edge while the other three helices will crossover to helices on the other neighboring edge of the particle. Typically the choice of vertex geometry is chosen by the generator of the design prior to routing and the geometry, and placement of the crossover between the edges is automated based on extending or contracting all other helices to make crossovers at geometric positions without inducing steric clashing.

3. Providing Physical Parameters of the Target Structure

In addition to the geometric form of the target structure, the methods enable design of the physical parameters of the scaffolded RNA nanostructure. Physical parameters that can be specified by the user include size, molecular weight, core nucleic acid sequence, as well as pre-determination of stability. For example, the stability of the nucleic acid nanostructure in one or more solvents can be required. In an exemplary embodiment, a structure that exhibits stability in physiological salt concentrations is designed by the methods.

Therefore, the methods include design of customized nucleic acid nanostructures having a specified size, having a specified molecular weight, having a specified core nucleic acid sequence, and combinations thereof.

i. Size

Methods for the step-wise design of custom scaffolded RNA nanostructure can produce nanostructures of a desired size. Typically, the size of the nanostructures is specified as a function of the length of the edges that form the wire-frame model of the desired structure.

Typically, the desired length of each edge is specified. In preferred embodiments, the lengths of edges obey the natural geometry of RNA. Preferably, the specified length of each edge does not give rise to shape distortions that force deviation from the target geometry. Therefore, in preferred embodiments, the length of each edge is specified as a number of base-pairs (bp) or nucleotides (nt) that is determined to ensure that no over- or under-wind in nucleic acid duplexes occurs. Typically, the length of each edge is a multiple of the unit number of base-pairs that is required to reduce or prevent over- or under-wind in nucleic acid duplexes that form the edges of the desired nucleic acid nanostructure. In some forms, the unpaired nucleotides in the scaffold are used to ensure no-over- or under-wind in nucleic acid multiple duplexes occurs when the length of each edge is not the multiple of 11 bp.

In some forms the length of each edge is a multiple of 11 base pairs (bp). In some forms, the minimum length of any single edge is 33 bp. For example, in some forms, any edge length smaller than 33 bp will create a scaffold crossover near to the end of an edge (in the vertex staple region) and will not yield a large quantity of final folded nanostructure product. Typically, constraining edge lengths to be multiples of 11 bp does not limit or otherwise restrict the selection of the target shape.

In some embodiments the length of the edge is a multiple of 11 bp. In some embodiments the selection of edges having length of 33 bp, 44 bp, 55 bp, 66 bp, 77 bp, 88 bp, or larger than 88 bp. The minimum edge length allowed in this design paradigm is 44 bp for A-form (RNA) DX wireframe structures. The minimum edge length allowed in this design paradigm is 33 bp for B-form (DNA) DX wireframe structures.

In some embodiments, the desired structures have equal edge lengths throughout the geometry. For example, design of Platonic, Archimedean, or Johnson solids includes the selection of edges having a length of 33 bp, 44 bp, 55 bp, 66 bp, 77 bp, 88 bp, 99 bp, or larger than 99 bp.

In some embodiments, desired structures do not have equal edge lengths throughout the geometry. Therefore, in some embodiments rounding of edge lengths is required. When rounding of edge lengths is required, the method can design nanostructures including deviations between the specified target structure and final design. For example, deviations in lengths of edges can occur at one or more edges in a structure. In these cases, the desired minimum edge length (e.g., 33 bp, or 44 bp) is assigned to the shortest edge and the other edges are scaled and rounded appropriately. Therefore, in some embodiments, where deviations between the specified target structure and final design are associated with different edge-lengths, multiple nanostructures can be designed, having deviations at one or more different edges.

Typically, the rounding of edge lengths is carried out automatically, for example, by computer software. When using automated rounding to generate edge lengths, the user is advised to verify that edge lengths are satisfactory before proceeding to the scaffold routing procedure.

The dimensions of edges that are selected are associated with the overall dimensions of the resulting nucleic acid nanostructure. The size of a nanostructure designed by the described methods can be defined as the maximum length of the structure in a single plane. Typically, the methods can design structures having overall dimensions of approximately 10-1,000 nm, inclusive, such as 50-500 nm, 60-200 nm, or 60-100 nm, for example, 60 nm, 70 nm, 80 nm, 90 nm, 100 nm or larger than 100 nm.

The average minimum size of a nanostructure is typically restricted by the complexity of the desired shape. Therefore, in some embodiments, the desired size of the nanostructure is a characteristic that is used in the automated design of target shapes that fulfill the desired maximum and/or minimum size criteria.

ii. Molecular Weight

The custom nucleic acid nanostructures produced according to the disclosed methods have a molecular weight. Typically, the molecular weight of the nanostructures is a function of the mass of the nucleic acids forming each of the edges that form the wire-frame model of the desired structure. Typically, the methods design nucleic acid nanostructures that have a molecular weight of between 200 kilo daltons (kDa) and 1 mega dalton (1 mDa).

The molecular weight of a nanostructure is typically defined by the size and complexity of the desired shape. Therefore, in some embodiments, the desired molecular weight of the nanostructure is a characteristic that is used in the automated design of target shapes that fulfill a desired maximum and/or minimum molecular weight criteria. Thus, the disclosed methods for step-wise design of custom nucleic acid nanostructures can produce nanostructures having a predetermined or preset molecular weight.

iii. RNA Scaffold Template Sequence In some embodiments, the methods design the sequence of staples that give rise to nanostructure having the desired shape based on a corresponding nucleic acid sequence. Therefore, in some embodiments the input also includes providing one or more RNA template sequences.

The nucleic acid template sequence can include natural RNAs or non-RNAs or can include a combination of natural and non-natural RNAs.

In some forms, the RNA nanostructures include a single-stranded RNA scaffold sequence that encodes or expresses a functional nucleic acid, or encodes or expresses a genomic transcript or gene, such as mRNA or replicating RNA (repRNA).

In some forms the RNA scaffold is mRNA or a replicating RNA (repRNA) vector expressing or encoding one or more polypeptides, proteins or functional nucleic acids.

Exemplary mRNAs encode or express genes expression products such as proteins and polypeptides. Exemplary proteins and polypeptides include enzymes. In some forms the mRNA expresses one or more exogenous genes in a target cell. Therefore, in some forms, the mRNA expresses one or more antigens in a target cell.

In some forms, the RNA scaffold is a functional RNA or a vector expressing one or more functional RNAs. Exemplary functional RNAs that can be encoded or expressed by the scaffold RNA include antisense silencing RNA (siRNA), short hairpin RNA (shRNA), micro RNA (miRNA), external guide sequences (EGSs), Piwi-interacting RNA (piRNA), single guide RNA (sgRNA), ribozymes, and aptamers.

In other forms, no input RNA sequence is provided. Therefore, in some embodiments one or more known RNA sequences are used as a default template sequence. In some forms the default template sequence is a sequence or a subset of a sequence corresponding to a 792-nt prokaryotic EGFP mRNA and 660-nt and 924-nt de Bruijn RNA sequences are used.

In other embodiments, a sequence is randomly generated. In further embodiments, the template sequence for the single-stranded RNA scaffold is determined based on the required scaffold length, for example, as determined by the Eulerian circuit corresponding to the desired shape according to the described methods. If the desired sequence is longer than the input sequence template, a sequence is randomly generated. For example, if the default template sequence is prokaryotic EGFP mRNA, and the required sequence is longer than 792 nucleotides, a random single-stranded scaffold template sequence is generated, for example, by a computer.

Typically, the RNA scaffold sequence is between 150 to 15,000 bases in length.

B. Identifying Scaffold Routing for the Target Structure

The methods include identifying the route of the scaffold RNA throughout the target structure, based on the information provided in the corresponding node-edge network of the corresponding polyhedron. Typically, the nodes and lines of the network correspond to the vertices and edges of the desired polyhedron. For example, Prim's formula can be used to find a breadth-first search spanning tree, one with the most branches. The spanning tree formula does not impose restrictions on the topology of the network. Therefore, the methods provide routing information for any arrangement of nodes and edges using a spanning tree to define the placement of scaffold crossovers.

1. Nanostructures with Antiparallel Double Crossover (DX) Motifs

In some embodiments, the methods require including at least one edge having one “DX” (anti-parallel scaffold crossover) motif. The edges with zero DX scaffold crossovers meet the definition of a spanning tree of a network. Therefore, a single DX anti-parallel scaffold crossover is positioned along every edge that does not form part of the spanning tree of the graph, preferably as close to the center of the edge as possible. The RNA scaffold strand is routed by a method that identifies the Eulerian circuit through the entire network, such that the strand enters each vertex from a first edge and exits the vertex from an adjacent edge that shares a face with the first edge. The route of the RNA scaffold strand is determined according to the rules that the scaffold strand does not enter and exit from the same edge, and the scaffold strand does not exit from an edge that is not-adjacent to the edge it enters. Therefore, the scaffold routing process does not allow for the intersection of RNA strands and the process produces only edges that are connected to the vertex.

Each of the steps involved in determining the route of the single-stranded nucleic acid scaffold is described in more detail, below.

a. Determination of the Node-Edge Network

In some embodiments, the wire-frame model of a desired polyhedral structure is rendered as a node-edge network. Typically, the nodes and edges of the network correspond to the vertices and lines of the polyhedron. In certain embodiments, a node-edge network corresponding to a structure can be represented by the planar graph of the corresponding polyhedron, or by other means. For example, in some embodiments the planar graph of the corresponding polyhedron is a Schlegel diagram. The Schlegel diagram is a projection of the desired polyhedral form from Rd into Rd-1 through a point beyond one of its facets or faces. The resulting entity is a polytopal subdivision of the facet in Rd-1 that is combinatorially equivalent to the original polyhedral form. Formulas and methods for generating a Schlegel diagram of a polyhedral form are known in the art. In other embodiments, a node-edge network is calculated for a corresponding structure without the use of a planar graph.

Therefore, in some embodiments, the methods include the step of providing a node-edge network of the target structure. Typically, each of the vertices corresponds to a node in the network, and each line between any two vertices represents an edge in the network.

b. Creating a Spanning Tree

In some embodiments, the node-edge network is used to establish connectivity amongst all of the vertices. An exemplary representation of connectivity through the node-edge network is by producing one or more spanning trees. The spanning tree is the set of edges that connect all nodes within the network without circuits. In some embodiments, the spanning tree is determined using one or more formulas. Formulas for determining the spanning tree for a network are known in the art. An exemplary method for determining the spanning tree for the node-edge network corresponding to the desired shape is Prim's Formula. Therefore, in some embodiments, identifying scaffold routing includes creating one or more spanning trees for the node-edge network. In certain embodiments, the spanning tree is the spanning tree produced using a maximum-breadth search. If, as in this case, all edges are weighted the same, Prim's formula will generate a breadth-first search spanning tree, one with the most branches. Therefore, in some embodiments, identifying scaffold routing includes the selection of one or more spanning trees that have the most branches.

It has been shown that branching trees self-assemble more reliably than more linear trees, however, any spanning tree will provide a valid route.

c. Locating DX Crossovers

The methods include using the spanning tree to identify the route of the RNA scaffold sequences through the target structure. For example, the methods can identify the location of anti-parallel DX cross-overs within the target structure by classifying each edge.

Determination of a spanning tree including all nodes of the network enables the identification of edges that are within the spanning tree and edges that are not within the spanning tree. Therefore, the methods include identifying edges that are within the spanning tree and edges that are not within the spanning tree. Edges within a spanning tree represent continuous stretches for the route of the single-stranded nucleic acid scaffold in both directions (i.e., 5′-3′ and 3′-5′). Edges not within a spanning tree include anti-parallel DX cross-over motifs. Therefore, for each edge that is not in the spanning tree, a pair of pseudo-nodes is added to split the edge into two halves, each corresponding to one side of a scaffold crossover. At each anti-parallel DX cross-over motif, the single-stranded nucleic acid scaffold reverses the direction it travels along.

The methods include assigning anti-parallel DX cross-over motifs at the center of each edge that is not within a spanning tree. Because a single scaffold crossover is assigned to each edge that is not within a spanning tree, and edges with zero scaffold crossovers must connect to every vertex, there can be no cycles of edges with zero scaffold crossovers, meaning that there are V−1 edges with zero scaffold crossovers, where V is the number of vertices, and the rest have one scaffold crossover.

Locating the DX crossovers within each possible spanning tree corresponds to a unique scaffold routing.

d. Identification of the Euler Circuit

The path of the single-stranded nucleic acid scaffold is defined as the Euler Circuit of the node-edge network. Therefore, the methods include converting the spanning tree into an Eulerian circuit. Converting the spanning tree into an Eulerian circuit includes (1) adding a pair of pseudo-nodes to each edge that is identified as including a DX crossover; (2) adding a set of pseudo-nodes at each vertex in the graph, so that each edge is bounded on both ends by pseudo-nodes; (3) removing the original vertex nodes; and (4) defining the Eulerian circuit through which the continuous scaffold strand will be routed.

Typically, a vertex of degree N has N edges emerging from it. An Eulerian circuit is guaranteed when the degree of every vertex is even. Therefore, the methods include creating a scaffold route for which the degree of every vertex in the node-edge network is even. The Eulerian Circuit of the planar graph passes through each of the edges once in each direction.

Therefore, the Eulerian circuit defined by the methods passes twice along each edge of the spanning tree. The route of the scaffold strand identified by the methods ensure (1) the scaffold strand always enters a vertex from a first edge and exits the vertex from an adjacent edge that shares a face with the first edge; and (2) the scaffold strand does not enter and exit a vertex from the same edge. Therefore, the scaffold routing process produces only edges that are connected to the vertex. The scaffold routing process does not allow for the intersection of RNA strands. Therefore, the methods provide a scaffold route that does not include internal scaffold loops that are disconnected from the rest of the scaffold.

The subset of Eulerian circuit that defines the route of the single-stranded RNA scaffold sequence through the entire polyhedral structure is defined as the subset of Eulerian circuits known as A-trails.

The direction of the scaffold is chosen to run counterclockwise around each face, so that for convex vertices (the majority of cage vertices) the major grooves of the duplexes at each vertex point inward to minimize electrostatic repulsion of the backbone. Therefore, the methods include converting the undirected graph into a directed graph to implement this directional choice.

e. Identifying Scaffold Routing for the Target Structure

Automated sequence design can be performed by first representing the target structure as a polyhedral mesh. Each edge is composed of multiple helices, so that the graph of the mesh is modified to represent these helices as multiple lines. These endpoints are then joined so that every duplex becomes part of a loop. By choosing a particular subset of these double crossovers, these discrete loops can connect to form one continuous Eulerian circuit through the entire structure, creating the scaffold routing. The spanning tree of this dual graph is then computed, and the edges that are members of the spanning tree correspond to the subset of crossovers required to complete the Eulerian circuit. Inverting the spanning tree of the dual graph back to the loop-crossover structure reveals the final scaffold routing. Therefore, the methods include converting the undirected graph into a directed graph to implement this directional choice.

f. Identifying the Sequence of the Single-Stranded Nucleic Acid Scaffold and Staple Sequences

The methods include the identification of the nucleic acid sequences of staples corresponding to the sequence of the single-stranded nucleic acid scaffold.

The length of the scaffold sequence is determined from the Eulerian circuit calculated from the input geometry, modified according to the input size, for example, as determined by the user-defined size of one or more of the edges of the structure. Typically, the sequence of the scaffold is based on a template sequence, for example, a user-defined sequence, or a known sequence, such as a prokaryotic EGFP sequence. If the sequence length required to provide the desired structure according to the methods is smaller than that of the default sequence, a subset of the default sequence will be output. Alternatively, if the sequence length required to provide the desired structure according to the methods is larger than that of the default sequence, a sequence will be generated.

The methods include the placement of all staple sequences. After all the staples are placed, each staple is converted to a vector of numbers, each value corresponding to the scaffold nucleotide to which it is base paired. Then, the input or generated scaffold sequence is used, matching a base identity (A, T, G, or C) to a scaffold number. If no sequence is provided, a segment of M13pm18 is used by default if the required scaffold length is less than 7249 nucleotides, and a sequence is randomly generated if the required length is greater. The complementary nucleotide via Watson-Crick base pairing is then be computed and assigned to the corresponding staple nucleotides. Finally, this list of staple sequences is output for synthesis.

i. Orientation of Scaffold Sequence

The methods combine the user-defined desired size (i.e., edge-length) with the spanning tree and pseudo-node addition to determine a scaffold sequence.

The Eulerian circuit is used to identify a scaffold nick position. The scaffold is nicked at a position located on an edge without scaffold crossovers that is located on the duplex at a distance from the DX crossovers. Using Prim's formula, this edge will have Vertex #1 as one of its endpoints, since with the most-branching default all edges connected to Vertex #1 are members of the spanning tree. Marking this 5′-end as scaffold base #1, each of the scaffold bases are subsequently numbered with knowledge of the edge lengths and routing scheme, all while keeping track of their relative position on their edge.

The scaffold is designed to ensure that all staple and scaffold crossovers remain perpendicular to the helical axes. Therefore, the scaffold is designed to ensure the 5′ end overhangs the 3′end by one nucleotide for each edge. The half-edges, namely those edges that are split by the scaffold crossover, have lengths that are pre-determined by some simplifying assumptions. The scaffold crossover is placed as close to the center as possible, with a convention set here to have a preference towards the lower-index vertex if needed. Therefore, the methods determine how long a particular section of a scaffold is on a given edge.

The methods ascribe two pieces of information to each nucleic acid base within the scaffold: (1) an index number to indicate its position on the scaffold strand; and (2) a set of numbers to indicate its spatial location, including the edge, the duplex, and the position from the 5′ end.

Typically edges are numbered according to their order within the Eulerian circuit, starting from the position of the 5′ nick.

ii. Placement of Staple Strands

The methods identify the routing of the staple strands based on the spatial location, including the edge, the duplex, and the position from the 5′ end. For example, information contained within the set of numbers that indicate the spatial location, including the edge, the duplex, and the position from the 5′ end, is used to identify which bases in the staples are paired with which bases in the scaffold, then the former index number is assigned to the staples accordingly.

Typically, the number of staple strands varies depending upon the complexity of the structure. For structures with small scaffold strands that are of minimal complexity, such as simple tetrahedra, cubes, etc., the number of staple strands is typically about 5, 10, 50 or more than 50. For longer scaffold strands (e.g., greater than 1500 bases) and/or more complex structures, the number of staple strands can be several hundreds to thousands. For example, in some embodiments, the number of staple strands is up to 50, 100, 300, 600, 1,000 or more than 1,000.

There are three categories of staple strands, each with their own prescribed pattern: staples on vertices, staples on edges with scaffold crossovers, and staples on edges without scaffold crossovers.

(i). Staples on Edges with Scaffold Crossovers

The edge staples pair with the intermediate nucleotides between vertex staples. For the edges with scaffold crossovers, two 31-32-nt staples are placed across the scaffold crossover, together occupying a 15-16-nt region on either side of the crossover for sufficiently strong binding. The remainder of scaffold has 42-nt staples placed to create staple crossovers every 21 base pairs, with a 20- or 22-nt staple in the case of a 10- or 11-nt remainder. In some forms, the staples on edges with scaffold crossovers are assigned based on staple crossover asymmetry, with 11 nucleotides per helical turn. In other forms, the staples on edges with scaffold crossovers are assigned based on scaffold crossover asymmetry, with 11 nucleotides per helical turn. In other forms, the staples on edges with scaffold crossovers are assigned with no asymmetry.

(ii). Staples on Edges without Scaffold Crossovers

The edges without scaffold crossovers follow the same pattern, filling with as many 42-nt staples that can fit and using a 20- or 22-nt staple when necessary.

g. Output of Staple Sequences

The methods provide the nucleic acid sequences of staple strands corresponding to the desired target sequence, edge size(s) and optionally a template nucleic acid sequence.

After all the staples are placed according to the methods, each staple is a vector of numbers, each value corresponding to the scaffold nucleotide to which it is base paired. Then, the input or generated scaffold sequence is used, matching a base identity (A, T, G, or C) to a scaffold number.

If no sequence is provided, a default sequence is used. For example, in some embodiments, if the required scaffold length is less than 792 nucleotides, a segment of prokaryotic EGFP RNA sequence is used. In other embodiments, a sequence is randomly generated. The methods determine complementary nucleotides via Watson-Crick base pairing and assign sequences to the corresponding staple nucleotides. Typically, the methods produce this list of staple sequences as output. Therefore, in some embodiments the methods also include the step of synthesizing the staple sequences. In some embodiments the methods include the step of synthesizing the scaffold sequence. In some embodiments, the methods include the step of synthesizing the scaffold sequence and the staple sequences. Therefore, the methods include converting the undirected graph into a directed graph to implement this directional choice.

h. Scaffold Sequence Output Based on User-Defined Staples

Methods to generate staple strand sequences given a scaffold sequence can be inverted, so that the user provides staple strand sequences that are used to generate a scaffold sequence.

The methods for custom-design of a nanostructure having desired geometric parameters can also be used to determine the nucleic acid sequence of a scaffold sequence that will fold into the desired shape based on hybridization with one or more user-defined staple sequences. Therefore, in some embodiments, the methods provide the nucleic acid scaffold sequence, based on the input of user-defined staple strands, desired target structure and optionally edge size(s).

The methods provide a custom scaffold sequence that based on user-defined staple sequences. Typically, the number and size of scaffold sequences that are required by the user will vary according to the desired geometry of the nanostructure. In some embodiments, at least one, two or three staple sequences are required as input. In certain embodiments, one or more staple sequences are required as input, and the methods provide the sequence(s) of one or more remaining, or undefined staple sequences.

2. Nanostructures with Parallel Crossover Motifs

In some embodiments, the methods require including at least one edge having one “PX” (parallel paranemic scaffold crossover) motif. Therefore, in some embodiments, there are two double helices per edge oriented in parallel vertically, that is, one of the duplexes is closer to the interior of the object than the other. In some embodiments, the scaffold cannot be an arbitrary sequence, because self-hybridization must occur to complete the structure. Self-hybridizing regions replace the need for staple strands, so in some embodiments one nucleic acid strand can fold and hybridize to itself to form an origami nanostructure without any other oligonucleotides.

The RNA scaffold strand is routed by a method that identifies the Eulerian circuit through the entire network, such that the strand enters each vertex from a first edge and exits the vertex from an adjacent edge that shares a face with the first edge. The route of the scaffold strand is determined according to the rules that the scaffold strand does not enter and exit from the same edge, and the scaffold strand does not exit from an edge that is not-adjacent to the edge it enters. Therefore, the scaffold routing process does not allow for the intersection of RNA strands and the process produces only edges that are connected to the vertex.

Each of the steps involved in determining the route of the single-stranded nucleic acid scaffold is described in more detail, below.

a. Determination of the Node-Edge Network

In some embodiments, the wire-frame model of a desired polyhedral structure is rendered as a node-edge network. Typically, the nodes and edges of the network correspond to the vertices and lines of the polyhedron. In certain embodiments, a node-edge network corresponding to a structure can be represented by the planar graph of the corresponding polyhedron, or by other means. For example, in some embodiments the planar graph of the corresponding polyhedron is a Schlegel diagram. The Schlegel diagram is a projection of the desired polyhedral form from Rd into Rd-1 through a point beyond one of its facets or faces. The resulting entity is a polytopal subdivision of the facet in Rd-1 that is combinatorially equivalent to the original polyhedral form. Formulas and methods for generating a Schlegel diagram of a polyhedral form are known in the art. In other embodiments, a node-edge network is calculated for a corresponding structure without the use of a planar graph.

Therefore, in some embodiments, the methods include the step of providing a node-edge network of the target structure. Typically, each of the vertices corresponds to a node in the network, and each line between any two vertices represents an edge in the network.

b. Creating a Spanning Tree

In some embodiments, the node-edge network is used to establish connectivity amongst all of the vertices. An exemplary representation of connectivity through the node-edge network is by producing one or more spanning trees. The spanning tree is the set of edges that connect all nodes within the network without circuits. In some embodiments, the spanning tree is determined using one or more formulas. Formulas for determining the spanning tree for a network are known in the art. An exemplary method for determining the spanning tree for the node-edge network corresponding to the desired shape is Prim's Formula. Therefore, in some embodiments, identifying scaffold routing includes creating one or more spanning trees for the node-edge network. In certain embodiments, the spanning tree is the spanning tree produced using a maximum-breadth search. If, as in this case, all edges are weighted the same, Prim's formula will generate a breadth-first search spanning tree, one with the most branches. Therefore, in some embodiments, identifying scaffold routing includes the selection of one or more spanning trees that have the most branches.

It has been shown that branching trees self-assemble more reliably than more linear trees, however, any spanning tree will provide a valid route.

c. Classifying Edges

The methods include using the spanning tree to classify the edges, culminating in the final Eulerian circuit the scaffold strand takes through the target structure.

There are four classifications the edges can have, based on choosing between two options for two traits. One trait is the crossover motif of the edge. Each edge can employ either anti-parallel (DX) or parallel (PX) crossovers. The second trait is determined by membership in the spanning tree. Edges that are members of the spanning tree must have each scaffold fragment, that is, the portion of the scaffold strand within the edge, start and end at different vertices. Edges that are not members of the spanning tree must have each scaffold fragment start and end at the same vertices. Note that this is an extension of the classification used for the two-helix-per-edge DX structures; the classifications and choice of scaffold crossover location follow the same start and end rules as described above.

d. Superimposing and Connecting Edges

Based on the classification (crossover motif, spanning tree membership) and the length of the edge, a set of scaffold fragments, and in some embodiments, staple strands, with routing within the edge already determined, is superimposed on the edge. In some embodiments, this is represented by an M×4 matrix, where M is the length of the edge, and each of the four columns represents one strand, e.g. Column 1 represents the nucleotides 3′ to 5′ from the vertex at the top to the vertex at the bottom in the duplex closer to the interior of the object, Column 2 represents the nucleotides 5′ to 3′ from the vertex at the top to the vertex at the bottom in the interior duplex, Column 3 represents the nucleotides 5′ to 3′ from the top vertex to the bottom vertex in the duplex closer to the exterior of the object for PX edges and 3′ to 5′ for DX edges, and Column 4 represents the nucleotides 3′ to 5′ from the top vertex to the bottom vertex in the exterior duplex for PX edges and 5′ to 3′ for DX edges. Nucleotides in Columns 1 and 2 are complementary via Watson-Crick base pairing, and nucleotides in Columns 3 and 4 are complementary in the same manner Nucleotides in the same row are the same interpolated distance between the two vertices.

In some embodiments, the elements of the matrix determine the route of the scaffold and enforce the crossover motif; for PX edges, the major/minor groove pattern is also enforced. Elements that are consecutive in number, e.g., 4 and 5, or i and i+1, represent nucleotides that share a covalent phosphodiester bond, and elements that are in the same row and are in paired columns (1 and 2, 3 and 4) are base paired. For PX edges, the major/minor groove pattern is the number of bases that lie in the major and minor grooves of the double helix. In some embodiments, the number of bases in a major groove can be less than 5, 5, 6, 7, 8, 9, or more than 9, and the number of bases in a minor groove can be less than 4, 4, 5, 6, or more than 6. The major/minor groove pattern also determines where parallel crossovers can occur. In some embodiments, this is reflected in the matrix as when consecutive nucleotides are not in the same column, e.g. nucleotide 4 is in Column 1 and nucleotide 5 is in Column 4.

When all of the edges have been superimposed, the first and last rows of Columns 1 and 2 of each edge matrix represent the 5′ and 3′ ends that must be joined to neighboring edges at the vertex. The connection is enforced by updating each nucleotide's number to uniquely identify its position in the complete scaffold strand, maintaining that consecutive numbers indicate connection along the phosphodiester backbone.

e. Identifying the Sequence of the Single-Stranded Nucleic Acid Scaffold and Staple Sequences

The methods include the identification of the nucleic acid sequences of scaffold and staples corresponding to the hybridization pattern set by the routing described above.

In regions of parallel crossovers, the sequence must be customized such that Watson-Crick base pairing is followed. In regions of anti-parallel crossovers, the scaffold sequence can be arbitrary, and the staple sequences that hybridize to it must follow Watson-Crick base pairing.

In some embodiments, the scaffold nick is chosen to be placed at the end of a farther-from-center duplex. This may be on PX or DX edge. The 5′ end of the nick is marked as base #1, and the 3′ end is the last base of the scaffold. Some scaffold nucleotides may be part of hairpin loops and do not have bases paired to them; the numbering of the scaffold strand remains unchanged, but these regions may be marked as single-stranded nucleic acid strands.

For these custom sequences, in some embodiments a random number generator choosing between 1 and 4 inclusive, which can map to A, C, G, T for DNA and A, C, G, U for RNA can produce the sequences of one member of each base pair, and its partner's sequence is found via canonical Watson-Crick base pairing. If certain staple sequences are to be incorporated, perhaps for example if they have been functionalized and need to bind to the larger origami structure, then those sequences of those regions are determined from the target staple sequences.

With this, the methods ascribe (1) an index number to indicate its position on the scaffold strand; and (2) a set of numbers to indicate its spatial location, including the edge, the duplex, and the position from the 5′ end.

f. Placement of Staple Strands

In edges with anti-parallel crossovers, staples may be necessary to bring together the portions of scaffold within the edge. In some embodiments, the superimposed edges contain regions where the staples lie based on their numbers being non-consecutive with the rest of the bases in the edges. In this embodiment, vertex staples are not required because only one duplex from each edge meets at the vertex.

g. Output of Staple and/or Scaffold Sequences

The methods provide the nucleic acid sequences of scaffold and staple strands corresponding to the desired target edge size(s) and geometry. Unlike the embodiment that only contains DX motifs, the scaffold sequence is, in part or in whole, a custom sequence.

Based on the nucleotide sequences generated in the previous steps, the methods typically produce this list of staple sequences and scaffold sequence as output. Therefore, in some embodiments the methods also include the step of synthesizing the staple sequences. In some embodiments the methods include the step of synthesizing the scaffold sequence. In some embodiments, the methods include the step of synthesizing the scaffold sequence and the staple sequences.

C. Assembling RNA Nanostructures

Typically, following design according to the described methods, the scaffolded RNA nanostructures are synthesized, folded and purified prior to structural validation. Therefore, methods for the design of scaffolded RNA nanostructures having a desired form optionally include the step of producing the scaffolded RNA nanostructure. In some embodiments, producing the nanostructure includes synthesizing nucleic acids having the sequence of the scaffold and staples according to the designed form; hybridizing the staple sequences to the scaffold; folding the nanostructure; purifying the nanostructure; performing structural analysis of the nanostructure; validating the structure; and combinations.

1. Production of Scaffolded RNA Nanostructures

The methods provide the sequences of the single-stranded RNA scaffold and the oligonucleotide staple sequences that can be combined to form complete three-dimensional scaffolded RNA nanostructures of a desired form and size. Typically, the methods convert the information provided as geometric parameters corresponding to the desired form and the desired dimensions into the sequences of oligonucleotides that can be synthesized using any means for the synthesis of nucleic acids known in the art.

a. Single-Stranded Scaffold RNA Sequence

Scaffold RNA sequences and oligonucleotide staple sequences can be synthesized or purchased from numerous commercial sources. Exemplary RNAs include the EGFP RNA sequence, rr1B RNA sequence (23s), M13 O44 RNA sequence, the rsc1218v1_rT55 RNA sequence, rsc1218v1_rT77 RNA sequence. In some forms, a control RNA sequences is also used. An exemplary control sequence is the HIV RRE RNA sequence.

In some forms the EGFP RNA sequence that is used to create an RNA scaffold sequence is

(SEQ ID NO: 1)
GGUAGCUAAGGAGGUAAAUAAUGGUGAGCAAGGGCGAGGAGCUGUUCAC
CGGGGUGGUGCCCAUCCUGGUCGAGCUGGACGGCGACGUAAACGGCCAC
AAGUUCAGCGUGUCCGGCGAGGGCGAGGGCGAUGCCACCUACGGCAAGC
UGACCCUGAAGUUCAUCUGCACCACCGGCAAGCUGCCCGUGCCCUGGCC
CACCCUCGUGACCACCCUGACCUACGGCGUGCAGUGCUUCAGCCGCUAC
CCCGACCACAUGAAGCAGCACGACUUCUUCAAGUCCGCCAUGCCCGAAG
GCUACGUCCAGGAGCGCACCAUCUUCUUCAAGGACGACGGCAACUACAA
GACCCGCGCCGAGGUGAAGUUCGAGGGCGACACCCUGGUGAACCGCAUC
GAGCUGAAGGGCAUCGACUUCAAGGAGGACGGCAACAUCCUGGGGCACA
AGCUGGAGUACAACUACAACAGCCACAACGUCUAUAUCAUGGCCGACAA
GCAGAAGAACGGCAUCAAGGUGAACUUCAAGAUCCGCCACAACAUCGAG
GACGGCAGCGUGCAGCUCGCCGACCACUACCAGCAGAACACCCCCAUCG
GCGACGGCCCCGUGCUGCUGCCCGACAACCACUACCUGAGCACCCAGUC
CGCCCUGAGCAAAGACCCCAACGAGAAGCGCGAUCACAUGGUCCUGCUG
GAGUUCGUGACCGCCGCCGGGAUCACUCUCGGCAUGGACGAGCUGUACA
AGUAACUGCAGGCAUGCAAGCUUGGCGUAAUCAUGGUCAUAGCUGUUUC
CUGUGUGAAAUUGUUAUCCGCUCACAAUUCCACACAACAUACG

In some forms, the rr1B 23s RNA sequence that is used to create an RNA scaffold sequence is

(SEQ ID NO: 2)
GCCCUGUUUUUGCAGUCAGAGGCGAUGAAGGACGUGCUAAUCUGCGAUA
AGCGUCGGUAAGGUGAUAUGAACCGUUAUAACCGGCGAUUUCCGAAUGG
GGAAACCCAGUGUGUUUCGACACACUAUCAUUAACUGAAUCCAUAGGUU
AAUGAGGCGAACCGGGGGAACUGAAACAUCUAAGUACCCCGAGGAAAAG
AAAUCAACCGAGAUUCCCCCAGUAGCGGCGAGCGAACGGGGAGCAGCCC
AGAGCCUGAAUCAGUGUGUGUGUUAGUGGAAGCGUCUGGAAAGGCGCGC
GAUACAGGGUGACAGCCCCGUACACAAAAAUGCACAUGCUGUGAGCUCG
AUGAGUAGGGCGGGACACGUGGUAUCCUGUCUGAAUAUGGGGGGACCAU
CCUCCAAGGCUAAAUACUCCUGACUGACCGAUAGUGAACCAGUACCGUG
AGGGAAAGGCGAAAAGAACCCCGGCGAGGGGAGUGAAAAAGAACCUGAA
ACCGUGUACGUACAAGCAGUGGGAGCACGCUUAGGCGUGUGACUGCGUA
CCUUUUGUAUAAUGGGUCAGCGACUUAUAUUCUGUAGCAAGGUUAACCG
AAUAGGGGAGCCGAAGGGAAACCGAGUCUUAACUGGGCGUUAAGUUGCA
GGGUAUAGACCCGAAACCCGGUGAUCUAGCCAUGGGCAGGUUGAAGGUU
GGGUAACACUAACUGGAGGACCGAACCGACUAAUGUUGAAAAAUUAGCG
GAUGACUUGUGGCUGGGGGUGAAAGGCCAAUCAAACCGGGAGAUAGCUG
GUUCUCCCCGAAAGCUAUUUAGGUAGCGCCUCGUGAAUUCAUCUCCGGG
GGUAGAGCACUGUUUCGGCAAGGGGGUCAUCCCGACUUACCAACCCGAU
GCAAACUGCGAAUACCGGAGAAUGUUAUCACGGGAGACACACGGCGGGU
GCUAACGUCCGUCGUGAAGAGGGAAACAACCCAGACCGCCAGCUAAGGU
CCCAAAGUCAUGGUUAAGUGGGAAACGAUGUGGGAAGGCCCAGACAGCC
AGGAUGUUGGCUUAGAAGCAGCCAUCAUUUAAAGAAAGCGUAAUAGCUC
ACUGGUCGAGUCGGCCUGCGCGGAAGAUGUAACGGGGCUAAACCAUGCA
CCGAAGCUGCGGCAGCGACGCUUAUGCGUUGUUGGGUAGGGGAGCGUUC
UGUAAGCCUGCGAAGGUGUGCUGUGAGGCAUGCUGGAGGUAUCAGAAGU
GCGAAUGCUGACAUAAGUAACGAUAAAGCGGGUGAAAAGCCCGCUCGCC
GGAAGACCAAGGGUUCCUGUCCAACGUUAAUCGGGGCAGGGUGAGUCGA
CCCCUAAGGCGAGGCCGAAAGGCGUAGUCGAUGGGAAACAGGUUAAUAU
UCCUGUACUUGGUGUUACUGCGAAGGGGGGACGGAGAAGGCUAUGUUGG
CCGGGCGACGGUUGUCCCGGUUUAAGCGUGUAGGCUGGUUUUCCAGGCA
AAUCCGGAAAAUCAAGGCUGAGGCGUGAUGACGAGGCACUACGGUGCUG
AAGCAACAAAUGCCCUGCUUCCAGGAAAAGCCUCUAAGCAUCAGGUAAC
AUCAAAUCGUACCCCAAACCGACACAGGUGGUCAGGUAGAGAAUACCAA
GGCGCUUGAGAGAACUCGGGUGAAGGAACUAGGCAAAAUGGUGCCGUAA
CUUCGGGAGAAGGCACGCUGAUAUGUAGGUGAAGCGACUUGCUCGUGGA
GCUGAAAUCAGUCGAAGAUACCAGCUGGCUGCAACUGUUUAUUAAAAAC
ACAGCACUGUGCAAACACGAAAGUGGACGUAUACGGUGUGACGCCUGCC
CGGUGCCGGAAGGUUAAUUGAUGGGGUUAGCGCAAGCGAAGCUCUUGAU
CGAAGCCCCGGUAAACGGCGGCCGUAACUAUAACGGUCCUAAGGUAGCG
AAAUUCCUUGUCGGGUAAGUUCCGACCUGCACGAAUGGCGUAAUGAUGG
CCAGGCUGUCUCCACCCGAGACUCA

In some forms, the M13 O44 RNA sequence that is used to create an RNA scaffold sequence is

(SEQ ID NO: 3)
GGGUCUCACUGGUGAAAAGAAAAACCACCCUGGCGCCCAAUACGCAAAC
CGCCUCUCCCCGCGCGUUGGCCGAUUCAUUAAUGCAGCUGGCACGACAG
GUUUCCCGACUGGAAAGCGGGCAGUGAGCGCAACGCAAUUAAUGUGAGU
UAGCUCACUCAUUAGGCACCCCAGGCUUUACACUUUAUGCUUCCGGCUC
GUAUGUUGUGUGGAAUUGUGAGCGGAUAACAAUUUCACACAGGAAACAG
CUAUGACCAUGAUUACGAAUUCGAGCUCGGUACCCGGGGAUCCUCUAGA
GUCGACCUGCAGGCAUGCAAGCUUGGCACUGGCCGUCGUUUUACAACGU
CGUGACUGGGAAAACCCUGGCGUUACCCAACUUAAUCGCCUUGCAGCAC
AUCCCCCUUUCGCCAGCUGGCGUAAUAGCGAAGAGGCCCGCACCGAUCG
CCCUUCCCAACAGUUGCGCAGCCUGAAUGGCGAAUGGCGCUUUGCCUGG
UUUCCGGCACCAGAAGCGGUGCCGGAAAGCUGGCUGGAGUGCGAUCUUC
CUGAGGCCGAUACUGUCGUCGUCCCCUCAAACUGGCAGAUGCACGGUUA
CGAUGCGCCCAUCUACACCAACGUGACCUAUCCCAUUACGGUCAAUCCG
CCGUUUGUUCCCACGGAGAAUCCGACGGGUUGUUACUCGCUCACAUUUA
AUGUUGAUGAAAGCUGGCUACAGGAAGGCCAGACGCGAAUUAUUUUUGA
UGGCGUUCCUAUUGGUUAAAAAAUGAGCUGAUUUAACAAAAAUUUAAUG
CGAAUUUUAACAAAAUAUUAACGUUUACAAUUUAAAUAUUUGCUUAUAC
AAUCUUCCUGUUUUUGGGGCUUUUCUGAUUAUCAACCGGGGUACAUAUG
AUUGACAUGCUAGUUUUACGAUUACCGUUCAUCGAUUCUCUUGUUUGCU
CCAGACUCUCAGGCAAUGACCUGAUAGCCUUUGUAGAUCUCUCAAAAAU
AGCUACCCUCUCCGGCAUUAAUUUAUCAGCUAGAACGGUUGAAUAUCAU
AUUGAUGGUGAUUUGACUGUCUCCGGCC.

In some forms, the rsc1218v1_rT55 RNA sequence that is used to create an RNA scaffold sequence is

(SEQ ID NO: 4)
GGGCCUAGUGAGUGCAUUAGAGACUCAAGCCAUGUAUCCAUGACCAGAA
GAGGGGACCCUAGGCCAAGGACAUGAGGGGCUCUAUGCACUAAUGUCUA
GUCAGGCCUUCACCCUAGUCAUCUCUAUGUGGAAUACAAUUGGGAUUCA
UGAGAGAAUCAUGCAAGCCAAAGUCAGCAGGUAGCCUUAUUUGAUGGAG
UUAACCAACAGCCAAAUUUAUUGCCCAGAGCCCAUUAACCAUGCUAGAG
GAGGGUUUCUCCUGAUGACUGGCUCCCUGACAUCUGUGUCUGGGCCUCA
AAUUGGUUUGAAGCUGAUGGGCUAUGCAUAAUCCCAUCACUAAGACCUU
GGUCUUGCUUGCUAGAACUUUUCAUUACACCACUAGCUGAAGUUUGUUA
UGACUGCCCCUAAUGGCAGGCUCCUCAGGGACAGAAGGAACUGGCCAAC
CCCAUAUCUAAUGUUGUUGGGCUUGCUGUAGGGUACCCAUCUAUGAGGA
GGCUUGUAACUGUGAUGCUCCCUUAGCUACUUUUGAUGUAUAUGCACAG
UGUUGGCAGUGGACCUUGCUCCAUGCAAUUCAUAUGGCUAUCUUGCAGU
CCAUGGCCCACCCCAGACUUCAUUGCUGCCCUGUGACACCAUUCAUUAG
AACCUGGUAUGGAAGUGUACCAC.

In some forms, the rsc1218v1_rT77 RNA sequence that is used to create an RNA scaffold sequence is

(SEQ ID NO: 5)
GGGCCUAGUGAGUGCAUUAGAGACUCAAGCCAUGUAUCCAUGACCAGAA
GAGGGGACCCUAGGCCAAGGACAUGAGGGGCUCUAUGCACUAAUGUCUA
GUCAGGCCUUCACCCUAGUCAUCUCUAUGUGGAAUACAAUUGGGAUUCA
UGAGAGAAUCAUGCAAGCCAAAGUCAGCAGGUAGCCUUAUUUGAUGGAG
UUAACCAACAGCCAAAUUUAUUGCCCAGAGCCCAUUAACCAUGCUAGAG
GAGGGUUUCUCCUGAUGACUGGCUCCCUGACAUCUGUGUCUGGGCCUCA
AAUUGGUUUGAAGCUGAUGGGCUAUGCAUAAUCCCAUCACUAAGACCUU
GGUCUUGCUUGCUAGAACUUUUCAUUACACCACUAGCUGAAGUUUGUUA
UGACUGCCCCUAAUGGCAGGCUCCUCAGGGACAGAAGGAACUGGCCAAC
CCCAUAUCUAAUGUUGUUGGGCUUGCUGUAGGGUACCCAUCUAUGAGGA
GGCUUGUAACUGUGAUGCUCCCUUAGCUACUUUUGAUGUAUAUGCACAG
UGUUGGCAGUGGACCUUGCUCCAUGCAAUUCAUAUGGCUAUCUUGCAGU
CCAUGGCCCACCCCAGACUUCAUUGCUGCCCUGUGACACCAUUCAUUAG
AACCUGGUAUGGAAGUGUACCACAGAGCUGCAAGGGGUUGUAGACCACU
UAUGGUGAAAUUUGUAGAAUCCAGGGUGGUGAGCUGUAAAUGAAACUUU
GGAUAACUGGCUUUCUAGAGCCUUCCUCCCACCCUCCCCUGUUUGCCAU
GCCCCAGGGGCAAGCACUGCCCUUUGCACUCUCACUACAGGUAAAGAGU
GUCCCCAGGAGUAAAUUUCUAGCCCCAUAGAAAAGGAAGGUCUAGAGGG
AAAUUGGCAAUGGGCACCUGUCCCAUUAUAAGCAUCUAUUUG.

In some forms, the HIV RRE RNA sequence that is used to create an RNA scaffold sequence is

(SEQ ID NO: 77)
GGAGCUUUGUUCCUUGGGUUCUUGGGAGCAGCAGGAAGCACUAUGGGCG
CAGCGUCAAUGACGCUGACGGUACAGGCCAGACAAUUAUUGUCUGAUAU
AGUGCAGCAGCAGAACAAUUUGCUGAGGGCUAUUGAGGCGCAACAGCAU
CUGUUGCAACUCACAGUCUGGGGCAUCAAACAGCUCCAGGCAAGAAUCC
UGGCUGUGGAAAGAUACCUAAAGGAUCAACAGCUCC.

Typically, scaffold RNA of the desired length is produced using in vitro transcription methodologies. Standard methods for in vitro transcription are known in the art. In some embodiments, the RNA sequences are purchased. Therefore, in some embodiments, the scaffold having a desired length is produced using one or more custom oligonucleotides. When the template scaffold nucleic acid is known, a set of known oligonucleotide forward (for) and reverse (rev) primers can be used.

For example, when the EGFP is used, primers can include:

forEGFP_for:
(SEQ ID NO: 6)
GAATTCTAATACGACTCACTATAGGTAGCTAAGG;
EGFP_rev:
(SEQ ID NO: 7)
CGTATGTTGTGTGGAATTGTGAG;

When the 23SdomIIV is used, primers can include:

23SdomIIV_for:
(SEQ ID NO: 8)
CTTAAGTAATACGACTCACTATAGCCCTGGCAGTCAGAGG;
23SdomIIV_rev:
(SEQ ID NO: 9)
TGAGTCTCGGGTGGAGACAG;

When the M13 for 044 is used, primers can include:

M13o44_for:
(SEQ ID NO: 10)
TAATACGACTCACTATAGGGTCTCGCTGGTGAAAAGAAA;
M13o44_rev:
(SEQ ID NO: 11)
GGCCGGAGACAGTCAAATC;

When the rsc1218v1 is used for rT55 or rT77, primers can include:

rsc1218v1_for:
(SEQ ID NO: 12)
GGCTTATCGAAATTAATACGACTCACTATAGGGCCTAGTGAGTGCATTA
GAG;
rsc1218v1_rT55_rev:
(SEQ ID NO: 13)
GTGGTACACTTCCATACCAGGTTC;
rsc1218v1_rT77_rev:
(SEQ ID NO: 14)
CAAATAGATGCTTATAATGGGACAGGTGC;

When the HIV genome is used, primers can include:

HIV_RRE_for:
(SEQ ID NO: 15)
GGAGCTTTGTTCCTTGGGTTCTTGG;
HIV_RRE_T7_for:
(SEQ ID NO: 16)
GGCTTATCGAAATTAATACGACTCACTA;
and
HIV_RRE_rev:
(SEQ ID NO: 17)
GGAGCTGTTGATCCTTTAGGTATCTTTC.

In some embodiments modified dNTPs (examples of modified dNTPs include, but are not limited to dUTP, Cy5-dNTP, biotin-dNTPs, alpha-phosphate-dNTPs, or ‘2-fluorinated deoxy-uridine, or 2’-fluorinated deoxy-cytosine, or 5-methoxyuridine) are used for production of RNA scaffold.

When nucleic acid scaffold sequences are required to be synthesized, the scaffolds can be synthesized, for example, using GBLOCK® DNA commercially available from Integrated DNA Technologies as a template.

2. Assembly of Scaffolded RNA Nanostructures

The methods include assembly of the RNA scaffold and the corresponding staple sequences into the scaffolded RNA nanostructure of the desired shape and size. Typically, the assembly is carried out by hybridization of the staples to the scaffold sequence. Therefore, in some embodiments, the scaffolded RNA nanostructures are assembled by RNA origami annealing reactions. For example, the oligonucleotide staples are mixed in the appropriate quantities in an appropriate reaction volume. In preferred embodiments, the staple strand mixes are added in an amount effective to maximize the yield and correct assembly of the nanostructure. For example, in some embodiments, the staple strand mixes are added in molar excess of the scaffold strand. In an exemplary embodiment, the staple strand mixes are added at a 10-20×molar excess of the scaffold strand.

Annealing can be carried out according to the specific parameters of the staple and scaffold sequences.

3. Purification of Scaffolded RNA Nanostructures

The methods include purification of the assembled scaffolded RNA nanostructures. Purification separates assembled structures from the substrates and buffers required during the assembly process. Typically, purification is carried out according to the physical characteristics of nanostructures. For example, the use of filters and/or chromatographic processes (FPLC, etc.) is carried out according to the size and shape of the nanostructures.

In an exemplary embodiment, scaffolded RNA nanostructures are purified using filtration, such as by centrifugal filtration, or gravity filtration. In some embodiments, filtration is carried out using an Amicon Ultra-0.5 mL centrifugal filter (MWCO 100 kDa).

Following purification, scaffolded RNA nanostructures can be placed into an appropriate buffer for storage, and/or subsequent structural analysis and validation. Storage can be carried out at room temperature (i.e., 25° C.), 4° C., or below 4° C., for example, at −20° C. Suitable storage buffers include PBS, TAE-Mg²⁺ or DMEM.

D. Predicting 3D Structure

Methods for designing scaffolded RNA nanostructures of a desired shape and size can include steps for validation of the resulting nucleic acid structure based on the output sequences. For example, in some embodiments, the methods also include the step of predicting the 3-dimensional coordinates of the nucleic acids within the nanostructure, based on the output of the system used for positioning scaffold and, when present, staple sequences. When structural information for a scaffolded RNA nanostructure is predicted, the predicted information can be used to validate the nanostructure. Typically, validation of the resulting scaffolded RNA structure includes (1) calculating the positions of each base pair in the structural model; (2) determining the positions of each base pair in the scaffolded RNA nanostructure; and (3) comparing the calculated structural data obtained for the model with that experimentally determined (i.e., observed) for the nanostructure.

1. In Silico Modelling of Structural Data

The 3-dimensional coordinates of a nucleic acid base pair can be calculated by any means known in the art. In a preferred embodiment, the positions of each base pair in the structural model are calculated using computational modelling. Therefore, in some embodiments, in silico modelling is used to predict the three-dimensional structural features of a polyhedral scaffolded RNA nanostructure designed from a target model according to the methods. The parameters used for modelling the 3-dimensional coordinates of a nucleic acid base pair of a given nanostructure designed according to the described methods are determined based upon the presence of antiparallel or parallel crossovers within the structure.

a. In Silico Modelling of Nanostructures Including Antiparallel Crossovers

In some embodiments, in silico modelling is used to predict the three-dimensional structural features of a polyhedral scaffolded RNA nanostructure including anti-parallel cross-overs, designed from a target model according to the described methods.

When in silico modelling is used to predict structural features of scaffolded RNA nanostructures including antiparallel crossovers, in silico modelling can be used to predict the position of each base pair in the structural model by interpolating between the two ends of the edge it resides on, and shifting away perpendicularly from the central axis by 10 Å, half the inter-helical distance for an anti-parallel crossover. The edge is assumed to lie in a plane with a normal vector defined by the sum of the unit normal vectors of the two neighboring faces. There are several ways to define the location of the ends of the edges. The DX-tile edges can be assumed to be two parallel cylinders with combined width 40 Å (20 Å inter-helical distance and 20 Å duplex diameter). This can be further simplified to a rectangle with width 40 Å, with the line of the edge serving as a central axis. In the ideal case, the corners of these rectangles meet, since the scaffold exits and enters the edge from these locations. The widths of the rectangles together would form an N-sided regular polygon, because they have the same sides and have equal angles between them. The perpendicular distance from the center of this polygon and an edge (the beginning of the interpolation) is the inradius of this polygon. From the inradius, the distance between the vertex and the beginning of the DX-tile edge is determined using the sum of the face angles. If the multi-arm DX-tile were flat, this would be equivalent to the inradius.

$$s = \frac{2 π}{{θ}_{tot}} r \qquad ( Eq . 3 )$$

where s is the distance between the vertex and the beginning of the DX-tile edge,
r is the inradius of the polygon formed by the widths of the tiles, and
θₜₒₜ is the sum of all face angles at the vertex.

For regular N-sided polygons,

$$r = \frac{w}{2} cot ( \frac{π}{N} ) \qquad ( Eq . 4 )$$

where w is the combined width of the DX-tile (40 Å).

In some embodiments, in silico modelling is used to predict the co-ordinates of nucleic acids within structures whose edges do not meet at regular angles. Exemplary structures whose edges do not meet at regular angles include the Archimedean solids. In that case, depending on the convention used to define the length of the inradius, there will be backbone stretches or nucleotide overlaps. For the cuboctahedron, a representative Archimedean solid, the size of the object is best fit when backbone stretches are minimized, where the inradius is calculated based on the largest face angle.

$$r = \frac{w}{2} cot ( \frac{{θ}_{max}}{2} ) \qquad ( Eq . 5 )$$

where
θₘₐₓ is the largest face angle. Note that this general equation applies to regular N-sided polygons as well, since θₘₐₓ=2π/N.

For structures with concave vertices, where θₜₒₜ>2π, to obey the convention that all edge axes meet at a single point, s=r is defined, creating a sphere of radius r that defines the edge boundaries.

b. In Silico Modelling of Scaffolded RNA Nanostructures Including Parallel Crossovers Scaffold Sequence Output Based on User-Defined Staples

In some embodiments, in silico modelling is used to predict the three-dimensional structural features of a polyhedral scaffolded RNA nanostructure having parallel crossover motifs, designed from a target model according to the described methods. For example, in silico modelling can be used to predict the position of each base pair in the structural model by interpolating between the two ends of the edge it resides on. If the base pair is part of the interior duplex of the edge, no shifting is necessary; if the base pair is part of the exterior duplex, the position is shifted away along the outward normal of the edge by 20 Å, the inter-helical distance. There are several ways to define the location of the ends of the edges, which are the 5′ and 3′ ends of the interior duplex. The interior duplex can be assumed to be a cylinder with diameter 20 Å. This can be further simplified to a rectangle with width 20 Å. In the ideal case, the corners of these rectangles meet at the vertex since the scaffold exits and enters the edge from these locations. The widths of these rectangles together would form an N-sided regular polygon, because they have the same sides and have equal angles between them. The perpendicular distance from the center of this polygon and an edge (the beginning of the interpolation) is the inradius of this polygon.

Calculating the inradius r and the distance between the vertex and the beginning of the interior duplex s follows the same procedure as described with Eq. 3 to 5, above, except w in this case is the diameter of a duplex, (e.g., 20 Å), instead of the width of a DX-tile (e.g., 40 Å).

2. Validation of Observed Structural Data

For validation, the predicted three dimensional model for a given structure is used as a comparison with the experimentally determined structural data. For example, the in silico prediction of structure(s) for a given input shape, size and optionally a nucleic acid sequence can be compared with actual structural data. Therefore, the methods can include the step of using data obtained by in silico modelling of a virtual structure to validate the structural parameters of a nucleic acid nanostructure designed and synthesized according to the methods. In certain embodiments, a virtual structure prepared by in silico modelling is used as a control for the design and synthesis methods.

Actual structural data corresponding to a scaffolded RNA nanostructure produced according to the methods can be obtained using any method known in the art. Exemplary methods for acquiring and analyzing biophysical data for macromolecular structures include DMS-MaPseq, X-ray crystallography, Nuclear Magnetic Resonance (NMR), Cryo-electron microscopy, Atomic Force Microscopy, Light Microscopy, Small-angle X-ray diffraction, Circular Dichroism, Analytical Ultracentrifugation, chromatographic methods, and combinations.

In some embodiments, differences between the in silico prediction of structural features and actual structural features identify structural deviations, etc.

### III. Systems

A. Computer Implemented Systems

The systems and methods provided herein are generally useful for predicting the design parameters that produce a scaffolded RNA nanostructure having a desired polyhedral shape. In some embodiments, the geometric parameters corresponding to the desired form and the desired dimensions are input using a computer-based interface that allows for the design process to be carried out in a completely in-silico manner. For example, in certain embodiments, the methods are implemented in computer software, or as part of a computer program that is accessed and operated using a host computer. In other embodiments, the methods are implemented on a computer server accessible over one or more computer networks.

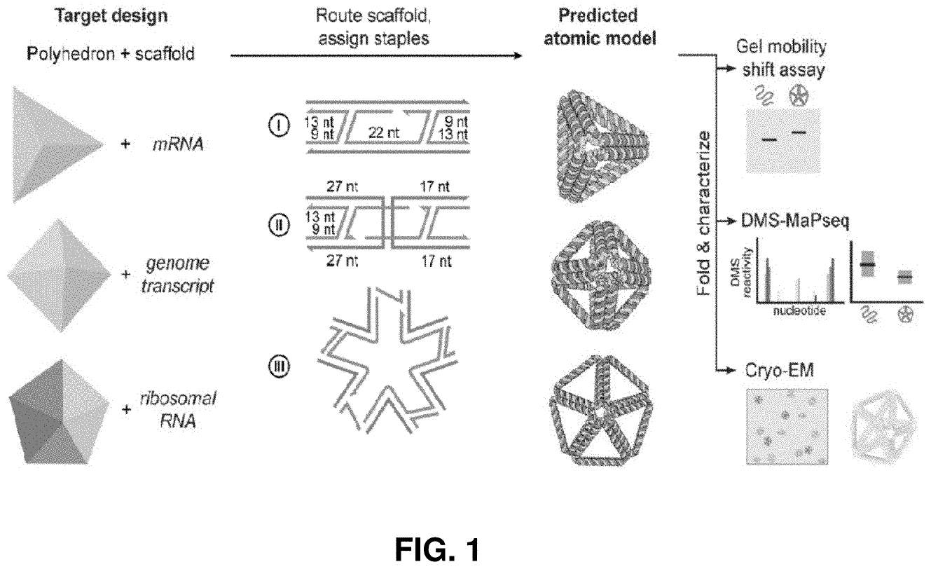

FIG. 1 depicts the work flow of methods that can be implemented. In some embodiments a user accesses a computer system that is in communication with a server computer system via a network, i.e., the Internet or in some cases a private network or a local intranet. One or both of the connections to the network may be wireless. In a preferred embodiment the server is in communication with a multitude of clients over the network, preferably a heterogeneous multitude of clients including personal computers and other computer servers as well as hand-held devices such as smartphones or tablet computers. In some embodiments the server computer is in communication, i.e., is able to receive an input query from or direct output results to, one or more laboratory automation systems, i.e., one or more automated laboratory systems or automation robotics that automate biochemical assays, PCR amplification, or synthesis of PCR primers. See for example automated systems available from Beckman Coulter.

The computer server where the methods are implemented may in principle be any computing system or architecture capable of performing the computations and storing the necessary data. The exact specifications of such a system will change with the growth and pace of technology, so the exemplary computer systems and components should not be seen as limiting. The systems will typically contain storage space, memory, one or more processors, and one or more input/output devices. It is to be appreciated that the term “processor” as used herein is intended to include any processing device, such as, for example, one that includes a CPU (central processing unit). The term “memory” as used herein is intended to include memory associated with a processor or CPU, such as, for example, RAM, ROM, etc. In addition, the term “input/output devices” or “I/O devices” as used herein is intended to include, for example, one or more input devices, e.g., keyboard, for making queries and/or inputting data to the processing unit, and/or one or more output devices, e.g., a display and/or printer, for presenting query results and/or other results associated with the processing unit. An I/O device might also be a connection to the network where queries are received from and results are directed to one or more client computers. It is also to be understood that the term “processor” may refer to more than one processing device. Other processing devices, either on a computer cluster or in a multi-processor computer server, may share the elements associated with the processing device. Accordingly, software components including instructions or code for performing the methodologies of the invention, as described herein, may be stored in one or more of the associated memory or storage devices (e.g., ROM, fixed or removable memory) and, when ready to be utilized, loaded in part or in whole into memory (e.g., into RAM) and executed by a CPU. The storage may be further utilized for storing program codes, databases of genomic sequences, etc. The storage can be any suitable form of computer storage including traditional hard-disk drives, solid-state drives, or ultrafast disk arrays. In some embodiments the storage includes network-attached storage that may be operatively connected to multiple similar computer servers that include a computing cluster.

1. Preparation of Nanostructure Libraries

In some embodiments, nanostructure libraries are designed by automated methods. Automated design programs for generating scaffolded RNA nanostructures allow for a diverse set of geometries to be made, towards the synthesis of a library of objects for applications as diverse as nano-casting, delivery, and structural scaffolding. Libraries of scaffolded RNA nanostructures with diverse sequences and geometries are also useful for diverse applications in memory storage, biomaterials synthesis, controlled nanoscale bioreactors, excitonic materials discover, vaccine development, and therapeutic delivery including cancer immunotherapy. For example, in some embodiments, a library or libraries of scaffolded RNA nanostructures can be constructed with single-strand bait sequences complementary to one or more target molecules. In an exemplary embodiment, the single-strand bait sequences include sequences that are complementary to one or more loops of a target molecule that is RNA.

a. Hi-Throughput Production of Scaffolded RNA Nanostructures and Modifications

Systems for the generation of libraries of scaffolded RNA nanostructures including different modifications can be implemented using automated methods. For example, the methods can provide the sequences of short single-stranded oligonucleotides staple strands of approximately 10-1,000 nucleotides that include “bait” sequences that are complementary in sequence to a region, or regions of a target molecule. In some embodiments the target molecules include RNAs, DNAs, PNAs, LNAs, proteins, lipids, carbohydrate, small molecules, etc. In an exemplary embodiment, the target molecule is a ribonucleic acid. Typically, target molecules interact with bait sequences on nanostructures via covalent or non-covalent linkage to the bait sequence. Exemplary linkages include either chemical conjugation via nucleic acid overhangs with click chemistry/other groups, or hybridization forces. When these staple strands are incorporated into the nanostructures, their position is defined by the design as part of the formation of the nanostructure, where the 5′ end of the staple meets the 3′ end of itself or another staple. Therefore, methods for creating libraries of polyhedral nucleic acid nanostructures for capturing one or more target molecules are provided. In some embodiments, the in silico design of polyhedral nucleic acid nanostructure libraries includes defining ranges for the desired properties of nanostructures within the library pool. Exemplary input ranges include minimum and maximum values for values such as size, vertex geometry, as well as spatial arrangement, and sequence diversity of bait sequences for capturing target molecules.

Typically, computational systems are applied to automate sequence designs of a diverse set of scaffolded RNA nanostructures. Scaffolded RNA nanostructures vary in many ways, including in object geometry (as shown in FIG. 19), edge lengths between vertices, staple nick positions along each edge (including nicks either inwardly facing or outwardly facing from the object), bait sequence orientation on the object (into or out from the edge tile), and the set of bait sequences for capturing the RNA. Different types of edges can provide distinct orientations of bait sequences. Further diversity can be introduced such as using different edge types, including 6-, 8-, 10, or 12-helix bundle. Further topology such as ring structure is also useable for example a 6-helix bundle ring.

Generally, the object geometry, edge length, and sequence topology dictate the scaffold and core staples, which are staple strands found in each class of nanostructure but are not functionalized in the library.

Generally, the high-throughput library generation of scaffolded RNA origami assemblies is achieved via multiple automated steps. Automated design program for generating scaffolded RNA nanostructures allows for a diverse set of geometries to be made towards the synthesis of a library of objects for applications as diverse as nano-casting, delivery, and structural scaffolding.

In some embodiments, a computational approach to generate a set of geometric objects with specific 3D overhangs complementary to single-stranded loops of HIV RNAs, seeking maximum coverage of Euclidian space by the overhangs, to allow for the most number of objects to be tested while being experimentally practical. The number of geometric objections generated in silico is about 10⁵, 2×10⁵, 3×10⁵, 4×10⁵, 5×10⁵, 6×10⁵, 7×10⁵, 8×10⁵, 9×10⁵, 10⁶, 10⁷, or more than 10⁷.

In some embodiments, the object generation approach is automated to attain maximum spatial coverage of the right size order of the overhangs in the fewest possible objects, limiting redundancy of spatial coordination. In some embodiments, a wide diversity of objects is used to ensure maximal coverage across the space of possibilities, such that the final experimental library has near complete spatial coverage.

In preferred embodiments, automated liquid handlers are used for generating these structure mixes. Typically, three high-throughput liquid dispensing steps are used for library generation, involving dispensing of the RNA scaffold, the core staples, and the functionalized staple sequences into designated wells of any suitable multi-well plates.

Generally, automation is preferred for the nanostructure library generation. Using synthesized stocks of staples, in combination with automated liquid handling and a liquid dispenser such as Echo 555 nanofluidic dispenser, high-throughput combinatorial libraries of staples with scaffold are readily generated. Typically, for each structure, there are a scaffold strand and a set of core (i.e. non-functionalized) staples. First, the scaffold and core staples are dispensed to every well of any suitable multi-well plates. Any nano-droplet dispensers having the ability to rapidly dispense 0.5 nL to 100 nL from a source well to a destination well, can be used. In preferred embodiments, an Echo 555 nano-droplet dispenser is used, with the ability to rapidly dispense 2.5 nL from a source well to a destination well.

In some embodiments, the source well contains functionalized oligonucleotide staples at a concentration at about 100 nM, 200 nM, 300 nM, 400 nM, 500 nM, 600 nM, 700 nM, 800 nM, 900 nM, 1 mM, or more than 1 mM. For example, in certain embodiments, 2.5 nL of functionalized oligonucleotide staples is transferred from a source well containing functionalized oligonucleotide staples at a concentration at more than 1 mM. For example, the Echo 555 nano-droplet dispenser is capable of transferring up to 60 droplets per second at a volume of about 2.5 nL.

Using the nano-droplet dispenser system, multiple 384-well plates of distributions of objects are readily generated from a source plate of functionalized staple strands that cover the geometric space allowable by scaffolded RNA origami objects. The methodology is not limited to 384-well plates, any suitable plates that are compatible with high-throughput capability can be used, for example, 96-well plates, 384-well plates, and 1536-well plates. In some embodiments, the concentrations and volume requirement of the nucleic acid scaffold, the core staples, and the functionalized staple strands are taken into consideration when deciding on the plate format.

In some embodiments, the RNA scaffold, the core staples, and the functionalized staple strands are mixed and annealed by slowly changing the temperature down (annealing) over the course of 1 to 48 hours. This process allows the staple strands to guide the folding of the scaffold into the final scaffolded RNA nanostructures. In further embodiments, high-throughput thermocyclers are used to slowly anneal staples and scaffold to generate the target nanostructure library, resulting in six, seven, eight, nine, ten, or more than ten 384-well plates of objects, with maximized utility generated from the computational method. In preferred embodiments, more than ten 384-well plates of nanostructures are generated.

The high-throughput methods allow fast generation so any number of nanostructures is capable of being generated as desired for the library, for example, one thousand, two thousand, three thousand, four thousand, five thousand, six thousand, seven thousand, eight thousand, nine thousand, ten thousand, twenty thousand, thirty thousand, forty thousand, fifty thousand, one hundred thousand, one million, and more than one million nanostructures for assembly. In preferred embodiments, combinatorial libraries of objects with any geometry, size, sequence, and nick placement, allowing for one million, or more than one billion of spatial overhang possibilities.

In some embodiments, liquid handling automation is used to generate approximately 3,000 of these space-covering geometric objects to test against target RNAs. In some embodiments, structural features and thermal stability of these target RNAs are characterized. In further embodiments, detection assays of nanostructure folding and stability using quantitative PCR, high-throughput fast analysis gels, and digestion analysis are used for assessing the scaffolded RNA nanostructures as well as complexes with other molecules, such as RNAs.

In some embodiments, the generated objects have designed staples with 3′ or 5′ single-stranded nucleic acid overhangs distributed over the edges of the wireframe polyhedra with singular bait sequence occurrences per object design per well. In further embodiments, these bait sequences are tested for complementarity within the structure to reduce misfolding.

In some embodiments, the development of chip and single-well technologies in RNA synthesis allows for assembly of nanoscale objects having pools of different sets of staples in each well grown in, for example, a 384-well plate. In each case, purification techniques applied to single structures are applicable to this high-throughput system, typically via filtration and buffer exchange. In further embodiments, high-throughput, rapid-run gel based assays, selective cryo-EM structural studies, and quantitative PCR (qPCR) temperature melting analysis are used for structural analysis, and validation. Additionally, fluorimetric or colorimetric read-outs are feasible using strand-displacement reaction cascades or triggered amplification upon complexing with other molecules. In some embodiments, structure-specific bar-codes or affinity capture tags are included within the scaffold or staple sequences. These tags or codes are used to record and identify desired characteristics, or to select specific nanostructures, or molecules complexed with the nanostructures.

B. Graphical User Interface

In a preferred set of embodiments the computer server receives input submitted through a graphical user interface (GUI). The GUI may be presented on an attached monitor or display and may accept input through a touch screen, attached mouse or pointing device, or from an attached keyboard. In some embodiments the GUI will be communicated across a network using an accepted standard to be rendered on a monitor or display attached to a client computer and capable of accepting input from one or more input devices attached to the client computer. In other embodiments, a phone interface can identify, read and or run entered sequences.

In the exemplary embodiment, the GUI contains a target structure selection region where the user selects the parameters to be input. In this exemplary system a target structure is indicated by clicking, touching, highlighting or selecting one of the structures, or subsets of structures, that are listed. In preferred embodiments, the target structure is selected from a drop-down list. In some embodiments, the overall target structure is selected and then customized to include user-defined features. Customization may include drawing a model, such as a wireframe model, using any computer programs capable of such functions. Other parameters relating to the target structure, such as edge length, molecular weight, overall size, encapsulation volume, wire-frame model topology, etc.

In some embodiments, the GUI enables entering or uploading one or more template or guide sequences, such as nucleic acid sequences. For example, the GUI typically includes a text box for the user to input of one or more parameters. In other embodiments, users may input any sequence or sequences for which they would like to design staple primers. The GUI may additionally or alternatively contain an interface for uploading a text file containing one or more query structures and/or sequences.

In a particular embodiment, a text file contains the geometric parameters of a target shape provided in a standard polyhedral file format. The geometric parameters of any closed, orientable surface network can serve as input using any file format that specifies polygonal geometry known in the art, including but not limited to, Polygon File Format (PLY), Stereolithography (STL), or Virtual Reality Modeling Language (WRL). When a standard polyhedral file format is provided, the code includes a parser to convert the standard polyhedral files into the required inputs.

In embodiments that include both options, the GUI may also contain radio buttons that allow the user to select if the target sequence will be entered in a text box or uploaded from a text file. The GUI may include a button for choosing the file, may allow a user to drag and drop the intended file, or other means of having the file uploaded. Any of the parameters can be entered by hand to further customize.

The GUI also typically includes an interface for the user to initiate the methods based on the input model and/or other parameters. The exemplary GUI embodiment includes a submit button or tab that when selected initiates a search according to the user entered or default criteria. The GUI can also include a reset button or tab when selected removes that user input and/or restores the default settings.

The GUI will in some embodiments have an example button that, when selected by the user, populates all of the input fields with default values. The option selected by the example values may in some embodiments coincide with an example described in detail in a tutorial, manual, or help section. The GUI will in some embodiments contain all or only some of the elements described above. The GUI may contain any graphical user input element or combination thereof including one or more menu bars, text boxes, buttons, hyperlinks, drop-down lists, list boxes, combo boxes, check boxes, radio buttons, cycle buttons, data grids, or tabs.

### IV. RNA-Based Nucleic Acid Nanostructures

RNA-based nucleic acid nanostructures, designed according to the geometric parameters of a desired polyhedral shape based on A-form helical nucleic acid geometry, according to the methods for top-down design of polyhedral nucleic acid assemblies are described. The polyhedral nucleic acid assemblies include a single-stranded RNA scaffold sequence that is routed throughout the entire structure. The polyhedral nucleic acid assemblies optionally include oligonucleotide staple strands that hybridize to the scaffold sequence and create the polyhedral structure. In some forms, the single-stranded RNA scaffold traces throughout the geometric shape based on staple crossover asymmetry with 11 nucleotides per helical turn. In other forms, the single-stranded RNA scaffold traces throughout the geometric shape based on scaffold crossover asymmetry with 11 nucleotides per helical turn. In other forms, the single-stranded RNA scaffold traces throughout the geometric shape based on no crossover asymmetry with 11 nucleotides per helical turn.

When the polyhedral nucleic acid assemblies do not include staple strands, the scaffold sequence hybridizes to itself to create the polyhedral structure. The RNA nanostructures designed according to the described methods include two or more nucleic acid duplexes per edge, and incorporate at least one parallel or anti-parallel crossover motif within at least one edge.

Modified RNA nanostructures are also described. The RNA nanostructures designed and assembled according to the described methods can include one or more modified nucleic acids, such as non-naturally occurring nucleic acids, derivatives and analogs. In some forms, the polyhedral structures are modified to include one or more nucleic acid sequences that are capable of binding or otherwise interacting with one or more non-nucleic acid molecules.

A. Nanostructure Assemblies Produced by Top-Down Design

Scaffolded RNA nanostructures having polyhedral morphology designed and produced according to the described top-down design methods are described. The polyhedral nucleic acid nanostructures include a single stranded nucleic acid scaffold routed through the entire polyhedral structure.

The scaffolded RNA nanostructures can be of any desired shape that can be rendered as a three-dimensional wire-frame mesh with sharp angles and non-curved edges. The scaffolded RNA nanostructures include a single-stranded nucleic acid scaffold that is routed throughout the entire structure. The route of the single-stranded RNA scaffold throughout every face of the structure is the Eulerian circuit through the node-edge network of the planar graph of the structure. Preferably, the Eulerian circuit that defines the path of the single-stranded scaffold sequence throughout the entire structure is the A-trail Eulerian circuit.

In some embodiments, the nanostructures include at least one edge having a DX crossover motif located within the center of the edge. In other embodiments, the nanostructures include at least one edge having a PX crossover motif located within the center of the edge. Typically, the nanostructures include zero or one scaffold crossover structures per edge. The placement of DX scaffold cross-overs is defined using by the maximum-breadth spanning-tree of the node-edge network of the planar graph of the structure. Edges that form part of the maximum-breadth spanning tree are the only edges that do not include a DX scaffold crossover. Edges that form part of the maximum-breadth spanning tree are the only edges that include a single DX scaffold crossover.

In some forms, scaffolded RNA nanostructures produced according to the methods include two nucleic acid anti-parallel helices along each edge to strengthen the rigidity of the structure.

The scaffolded RNA nanostructures are typically less than 1 micron in diameter, for example, 10 nm-1,000 nm, inclusive. In some embodiments, the scaffolded RNA nanostructures have overall dimensions of 50-500 nm, 60-200 nm, or 60-100 nm, for example, 60 nm, 70 nm, 80 nm, 90 nm, 100 nm or leger than 100 nm. The molecular weight of the nanostructure is typically defined by the size and complexity of the polyhedral shape of the nanostructure. Typically, the scaffolded RNA nanostructures have a molecular weight of between 200 kilo daltons (kDa) and 1 mega dalton (1 mDa). The volume encapsulated by the nanostructures is defined by the size and shape of the nanostructures, and can be determined from the dimensions.

Typically the scaffolded RNA nanostructures are stable in physiological concentrations of salt, for example, in PBS, and DMEM.

1. Modified Nucleotides

In some embodiments, the nucleotides of the scaffolded RNA sequences are modified. For example, in some embodiments, one or more of the nucleotides of the staple sequences are modified, or one or more of the nucleotides of scaffold sequence are modified, or both nucleotides in the staple sequences and nucleotides in the scaffold sequence are modified.

When modified nucleotides are incorporated into RNA scaffold strands or oligonucleotide staple strands, the modified nucleotides can be incorporated as a percentage or ratio of the total nucleotides used in the preparation of the nucleic acids. In some embodiments, the modified nucleotides represent 0.1% or more than 0.1% of the total number of nucleotides in the sequence, up to or approaching 100% of the total nucleotides present. For example, the relative amount of modified nucleotides can be between 0.1% and 100% inclusive, such as 0.1%-0.5%, 1%-2%, 1%-5%, 1%-10%, 10%-20%, 20%-30%, 30%-40%, 40%-50%, or more than 50% of the total, up to and including 100%, such as 60%, 70%, 75%, 80%, 85%, 90%, 95% or 99% of the total. In a certain embodiment, a sequence of nucleic acids includes a single modified nucleotide, or two, or three modified nucleotides. In some embodiments, nucleic acid nanostructures contain one, or more than one, up to 100 modified nucleotides in every edge. In other embodiments, the number of modified nucleotides correlates with the size of the nanostructure, or the shape, or the number of faces or edges, or vertices of the nanostructure. For example, in some embodiments, nucleic acid nanostructures include the same or different numbers of modified nucleotides within every edge or vertex. In some embodiments, the modified nucleotides are present at the equivalent position in every structurally-equivalent edge of the nanostructure. In some embodiments, nucleic acid nanostructures include modified nucleotides at precise locations and in specific numbers or proportions as determined by the design process. Therefore, in some embodiments, scaffolded RNA assemblies include a defined number or percentage of modified nucleotides at specified positions within the structure. In some embodiments, scaffolded RNA nanostructures produced according to the described methods include more than a single type of modified nucleic acid. In exemplary embodiments, scaffolded RNA nanostructures include one type of modified nucleic acid on every edge, or mixtures of two or more different modified nucleic acids on every edge. Therefore, when a single type of modified nucleic acid is present at an edge of the structure, each edge can include a different type of modification relative to every other edge.

Examples of modified nucleotides that can be included within the described nanostructures include, but are not limited to, 2′-fluorinated deoxy-uridine, or 2′-fluorinated deoxy-cytosine, or 5-methoxyuridine. diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

In some embodiments phosphorothioate modified backbone on a DNA nucleotide staple or on the RNA scaffold is used to improve stability of the scaffolded RNA nanostructures to degradation by exonuclease. For example, in some embodiments the nucleic acid nanostructures include modified nucleic acids that protect one or more regions of the nanostructure from enzymic degradation or disruption in vivo. In some embodiments, scaffolded RNA nanostructures include modified nucleic acids at specific locations within the structure that direct the timing of the enzymic degradation of specific parts of the structure. For example, modifications can be designed to prevent degradation, or to enhance the likelihood of degradation of one or more edges before or after different edges within the same structure. In this way, modifications that enhance or reduce protection or enzymic degradation of one or more parts of a nanostructure in vivo can drive or facilitate structural changes in the structure, for example, for example to enhance or alter the half-life of a given structure in vivo.

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic Acids Res. 301911-1918 (2002)). In some embodiments, the nucleic acids are synthetic RNA-like high affinity nucleotide analogue, locked nucleic acids. In some embodiments, the scaffolded RNAs are locked nucleic acids. In other embodiments, the staple strands are locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254, 1497-1500 (1991)). It is chemically stable and resistant to hydrolytic (enzymatic) cleavage. In some embodiments, the scaffolded RNAs are PNAs. In other embodiments, the staple strands are PNAs.

In some embodiments PNAs, DNAs, RNAs, or LNAs are used for capture, or proteins or other small molecules of interest to target, or otherwise interact with complementary binding sites on structured RNAs, or DNAs. In other embodiments, a combination of any two or more of PNAs, DNAs, RNAs and/or LNAs is used in the formation of structured nucleic acid nanostructures.

In some embodiments, the structured nanostructures include a combination of any two or more of RNAs, PNAs, DNAs, and/or LNAs. In some embodiments, a combination of any two or more of RNAs, PNAs, DNAs, and/or LNAs is used for the staple strands.

In some embodiments, the nucleic acids produced according to the described methods are modified to incorporate fluorescent molecules. Exemplary fluorescent molecules include fluorescent dyes and stains, such as Cy5 modified CTP.

In some embodiments, scaffolded RNA nanostructures include one or more nucleic acids conjugated to polymers. Exemplary polymers that can be conjugated to nucleic acids include biodegradable polymers, non-biodegradeable polymers, cationic polymers and dendrimers. For example, a non-limiting list of polymers that can be coupled to nucleic acids within the scaffolded RNA nanostructures includes poly(beta-amino esters); aliphatic polyesters; polyphosphoesters; poly(L-lysine) containing disulfide linkages; poly(ethylenimine) (PEI); disulfide-containing polymers such as DTSP or DTBP crosslinked PEI; PEGylated PEI crosslinked with DTSP; Crosslinked PEI with DSP; Linear SS-PEI; DTSP-Crosslinked linear PEI; branched poly(ethylenimine sulfide) (b-PEIS). Typically, the polymer has a molecular weight of between 500 Da and 20,000 Da, inclusive, for example, approximately 1,000 Da to 10,000 Da, inclusive. In some embodiments, the polymer is ethylene glycol. In some embodiments, the polymer is polyethylene glycol. In an exemplary embodiment, one or more polymer are conjugated to the nucleic acids within one or more of the staples. Therefore, in some embodiments, one or more types of polymers conjugated to staple strands are used to coat the nucleic acid nanostructure with the one or more polymers. In some embodiments, one or more types of polymers conjugated to nucleic acids in the scaffold sequence are used to coat the used to coat the scaffolded RNA nanostructure with the one or more polymers.

2. Modified Nanostructures

RNA-based Nucleic acid nanostructures designed and produced according to the described methods can be modified to include nucleic acids having a known function, or molecules other than nucleic acids. Exemplary additional elements include small molecules, proteins, peptides, nucleic acids, lipids, saccharides, or polysaccharides. For example, RNA nanoparticles can be modified to include proteins or RNAs having a known function, such as antibodies or RNA aptamers having an affinity to one or more target molecules. Therefore, the nucleic acid nanostructures designed and produced according to the described methods can be functionalized RNA nanostructures.

RNA nanostructures can include one or more functional molecules at one or more locations on or within the structure. In some embodiments, the functional group is located at one or more staple strands. In other embodiments, the functional moiety is located directly within the scaffold sequence of the nanostructure. In other embodiments, nanostructures include one or more functional moieties located within the scaffold sequence and within one or more staple sequences. When nanostructures include two or more functional moieties, the functional moieties can be the same, or different.

a. Interaction with Functional Molecules

Typically, RNA nanostructures are modified by chemical or physical association with one or more functional molecules. Exemplary methods of conjugation include covalent or non-covalent linkages between the nanostructure and the functional molecule. In some embodiments, conjugation with functional molecules is through click-chemistry. In some embodiments, conjugation with functional molecules is through hybridization with one or more of the nucleic acid sequences present on the nanostructure. In some embodiments, conjugation with functional molecules is through click-chemistry.

i. Modified Staple Sequences

In some embodiments, RNA nanostructures include one or more functional groups located at one or more staple strands. For example, in some embodiments, the RNA nanostructures include modified staple strands include single-stranded overhang sequences. In some embodiments, the overhang sequences are between 4 and 60 nucleotides. In preferred embodiments, the overhang sequences are between 4 and 25 nucleotides. In some embodiments, the overhang sequences contain 4, 5, 6, 7, 8, 9, 10, 11, 12, 13, 14, 15, 16, 17, 18, 19, 20, 21, 22, 23, 24, 25, 30, 35, 40, 45, 50 nucleotides in length.

In some embodiments, nanostructures include oligonucleotide staples extended at either the 5′ or 3′ ends by an unpaired region of nucleic acid, such as DNA, RNA, PNA, or LNA of known sequence. For example, in some embodiments the single-stranded nucleic acid includes a binding site for one or more functional moieties, such as nucleic acids, proteins or small molecules. Therefore, RNA nanoparticles including staple strands extended to include one or more single-stranded nucleic acid binding sites for a functional nucleic acid, protein or small molecule are described. Nucleic acid nanoparticles including functional RNA, small molecules, or proteins are also described. The functionalized nanoparticles can include functional moieties displayed at the surface of the nanoparticle, or located within the inner volume of the nanoparticle. Typically, the location of the functional moiety is determined by the desired biological function of the nanoparticle.

Nucleic acid nanoparticles functionalized with one or more nucleic acid or non-nucleic acid moieties having a known biological function are provided.

In some embodiments, nucleic acid nanoparticles include staple strands extended to include one or more single-stranded nucleic acid sequences that are complementary to the loop region of an RNA, such as an mRNA. Loop regions of mRNA targets can be identified using methods known in the art. When sequences complementary to these loop regions are appended to one or more nanoparticle staple strands, the nanoparticle is capable of capturing the target RNA. Nanoparticles specifically bound to target RNA can be identified from those that are not bound to the target RNA using any assay known in the art, such as by gel mobility shift, and/or imaging by cryo-EM.

ii. RNA Scaffold Sequences

In some forms, the RNA nanostructures include a single-stranded RNA scaffold sequence that encodes or expresses a functional nucleic acid, or encodes or expresses a genomic transcript or gene, such as mRNA or replicating RNA (repRNA). In other forms, the RNA scaffold includes one or more sequences of nucleic acids that bind one or more functional moieties, such as nucleic acids, proteins or small molecules, or that encodes or expresses a protein, or functional nucleic acid molecule. In other forms, the scaffold includes an overhang sequence that includes one or more functionalizing sequences or moieties at the 5′ or 3′ ends. In other embodiments, the scaffold includes an internal functionalizing sequence or moiety, for example, within one or more nucleic acids that form part of an edge of the nanostructure.

(1) mRNA

In some forms the RNA scaffold is mRNA or a replicating RNA vector expressing one or more mRNAs.

Typically, when the RNA scaffold includes mRNA, the scaffold must be unfolded within a target or host cell for expression of the mRNA within the cell. Therefore, in some forms, the nanostructures include an mRNA scaffold that is designed to be released within the cell by unfolding of the nanostructure. Typically, the unfolding includes intracellular enzymic activity, such as removal of one or more of the staples and/or structural components of the nanostructure. In some forms, the RNA nanostructure includes an mRNA scaffold and DNA staples. When the nanostructures include DNA staples, the activity of DNA-specific nucleases can release the scaffold mRNA for expression of one or more antigenic peptides within a host cell. In some forms, the RNA-based nanostructures include an mRNA scaffold sequence that is protected from RNAse activity, for example, by modification of the mRNA scaffold. Exemplary mRNAs encode or express genes expression products such as proteins and polypeptides. Exemplary proteins and polypeptides include enzymes. In some forms the mRNA expresses one or more exogenous genes in a target cell. Therefore, in some forms, the mRNA expresses one or more antigens in a target cell.

Antigens

In some forms the mRNA is, or encodes, or expresses a protein or polypeptide that is an antigenic protein or polypeptide. An antigen can include any protein or peptide that is foreign to the subject organism. Preferred antigens can be presented at the surface of antigen presenting cells (APC) of a subject for surveillance by immune effector cells, such as leucocytes expressing the CD4 receptor (CD4 T cells) and Natural Killer (NK) cells. Typically, the antigen is of viral, bacterial, protozoan, fungal, or animal origin. In some embodiments, the antigen is a cancer antigen. Cancer antigens can be antigens expressed only on tumor cells and/or required for tumor cell survival. Certain antigens are recognized by those skilled in the art as immuno-stimulatory (i.e., stimulate effective immune recognition) and provide effective immunity to the organism or molecule from which they derive.

B cell antigens can be peptides, proteins, polysaccharides, saccharides, lipids, nucleic acids, small molecules (alone or with a hapten) or combinations thereof. T cell antigens are proteins or peptides. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. The antigen may be purified or partially purified polypeptide derived from tumors or viral or bacterial sources. The antigen can be recombinant polypeptide produced by expressing DNA encoding the polypeptide antigen in a heterologous expression system. All or part of an antigenic protein can be encoded by a the scaffold RNA molecule for delivery. Antigens may be provided as single antigens or may be provided in combination. Antigens may also be provided as complex mixtures of polypeptides or nucleic acids.

Viral Antigens

In some embodiments, the antigen is a viral antigen. A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (EBOV) (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A, such as H1N1 strain, and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis. Typically, viral antigens encoded by repRNAs are not derived from the native viral genome from which the repRNA was developed.

In some embodiments, the viral antigen in derived from one or more viruses from the Orthomyxovirus family, for example, the Influenza virus A, Influenza virus B, Influenza virus C, Isavirus, Thogotovirus and Quaranjavirus. Exemplary influenza A virus subtypes include H1N1, H1N2, H3N2, H3N1, H5N1, H2N2, and H7N7. Exemplary influenza virus antigens include one or more proteins or glycoproteins such as hemagglutinin, such as HA1 and HA2 subunits, neurominidase, viral RNA polymerase, such as one or more of PB1, PB2 PA and PB1-F2, reverse transcriptase, capsid protein, non-structured proteins, such as NS1 and NEP, nucleoprotein, matrix proteins, such as M1 and M2 and pore proteins. In some embodiments, Influenza A virus antigens include one or more of the Hemagglutinin (HA) or Neuraminidase (NA) glycoproteins or fragments of the HA or NA, including the antigenic sites of the Hemagglutinin HA1 glycoprotein. In an exemplary embodiment, MDNPs include RNA encoding the influenza A/WSN/33 HA protein.

In some embodiments, the viral antigen in derived from one or more viruses from the genus Ebolavirus, for example, the Zaire ebolavirus (EBOV), Sudan ebolavirus (SUDV), Taï Forest ebolavirus (TAFV), Reston ebolavirus (RESTV), and Bundibugyo ebolavirus (BDBV). In an exemplary embodiment, MDNPs include RNA, such as repRNA, encoding the Zaire ebolavirus glycoprotein (GP), or one or more fragments of the Zaire ebolavirus glycoprotein (GP).

In some embodiments, the viral antigen in derived from one or more viruses from the genus Flavivirus, for example, the Zika virus (ZIKV).

Bacterial Antigens

In some embodiments, the antigen is a bacterial antigen. Bacterial antigens can originate from any bacteria including, but not limited to, Actinomyces sp., Anabaena sp., Bacillus sp., Bacteroides sp., Bdellovibrio sp., Bordetella sp., Borrelia sp., Campylobacter sp., Caulobacter sp., Chlamydia sp., Chlorobium sp., Chromatium sp., Clostridium sp., Corynebacterium sp., Cytophaga sp., Deinococcus sp., Escherichia sp., Francisella sp., Halobacterium sp., Heliobacter sp., Haemophilus sp., Hemophilus influenza type B (HIB), Hyphomicrobium sp., Legionella sp., Leptspirosis sp., Listeria sp., Meningococcus A, B and C, Methanobacterium sp., Micrococcus sp., Myobacterium sp., Mycoplasma sp., Myxococcus sp., Neisseria sp., Nitrobacter sp., Oscillatoria sp., Prochloron sp., Proteus sp., Pseudomonas sp., Phodospirillum sp., Rickettsia sp., Salmonella sp., Shigella sp., Spirillum sp., Spirochaeta sp., Staphylococcus sp., Streptococcus sp., Streptomyces sp., Sulfolobus sp., Thermoplasma sp., Thiobacillus sp., and Treponema sp., Vibrio sp., and Yersinia sp.

Parasite Antigens

In some embodiments, the antigen is a parasite antigen. Exemplary parasite allergens, include but are not limited to, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, the parasite antigen is one or more antigens from a protozoan, such as one or more protozoans from the genus Toxoplasma, for example T. gondii and species from a related genus, such as Neospora, Hammondia, Frenkelia, Isospora and Sarcocystis. Exemplary antigens derived from T. gondii include the GRA6, ROP2A, ROP18, SAG1, SAG2 Å and AMA1 gene products.

Allergens and Environmental Antigens

In some embodiments, the antigen is an allergen or environmental antigen. Exemplary allergens and environmental antigens, include but are not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens.

Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeriaand juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas, e.g., Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

Exemplary food allergens include cow's milk (e.g., lactose), eggs, nuts, shellfish, fish, and legumes (peanuts and soybeans), fruits and vegetables such as tomatoes.

When the antigen is an allergen, the nanoparticles can include one or more immuno-modulatory molecules, or one or more nucleic acids encoding immuno-modulatory molecules to direct the immune response specifically toward a Th1 (cellular) or Th2 (humoral) polarization for the delivered allergen.

Tumor Antigens

In some embodiments, the antigen is a tumor antigen. There are many classes of tumor antigens, including, but not limited to, oncogene expression products, alternatively spliced expression products, mutated gene products, over-expressed gene products, aberrantly expressed gene products, antigens produced by an oncogenic viruses, oncofetal antigens, as well as proteins with altered cell surface glycolipids, and proteins having altered glycosylation profiles.

Exemplary tumor antigens included or encoded by scaffold mRNAs include tumor-associated or tumor-specific antigens, such as, but not limited to, alpha-actinin-4, Alphafetoprotein (AFP), Bcr-Abl fusion protein, Carcinoembryonic antigen (CEA), CA-125, Casp-8, beta-catenin, cdc27, cdk4, cdkn2a, coa-1, dek-can fusion protein, epithelial tumor antigen, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Melanoma-associated antigen (MAGE); Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Me1-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, MUC-1, NB\70K, NY-CO-1, RCAS1, SDCCAG16, TA-90 (Mac-2 binding protein\cyclophilin C-associated protein), TAAL6, TAG72, TLP, tyrosinase, and TPS. An exemplary tumor antigen is the model melanoma tumor antigen Trp 1.

In certain embodiments, the tumor antigen is the gene product of a gene that is normally expressed during embryogenesis, and whose expression in normal adult tissues is limited, such as an “oncofetal” protein, or an alternatively-spliced variant of a normal protein. Oncofetal antigens are proteins which are typically present only during fetal development but are found in adults with certain kinds of cancer. These proteins are often measurable in the blood of individuals with cancer and may be used to both diagnose and follow treatment of the tumors. Therefore, in some embodiments, the MDNPs include or encode one or more oncofetal proteins. An exemplary oncofetal protein is the Hmga2 protein.

(2) Functional RNAs

In certain forms, the RNA scaffold is a functional RNA or a vector expressing one or more functional RNAs.

Exemplary functional RNAs that can be encoded or expressed by the scaffold RNA include antisense silencing RNA (siRNA), short hairpin RNA (shRNA), micro RNA (miRNA), external guide sequences (EGSs), Piwi-interacting RNA (piRNA), single guide RNA (sgRNA), ribozymes, and aptamers.

Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (Kd) less than or equal to 10-6, 10-8, 10-10, or 10-12.

In certain forms, the RNA scaffold is a functional RNA or a vector expressing one or more functional RNAs that is an aptamer. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kds from the target molecule of less than 10-12 M. It is preferred that the aptamers bind the target molecule with a Kd less than 10-6,10-8, 10-10, or 10-12. Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a Kd with the target molecule at least 10, 100, 1000, 10,000, or 100,000 fold lower than the Kd with a background binding molecule. It is preferred when doing the comparison for a molecule such as a polypeptide, that the background molecule be a different polypeptide.

In certain forms, the RNA scaffold is a functional RNA or a vector expressing one or more functional RNAs that is a ribozyme. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are described. Ribozymes that are not found in natural systems, but which have been engineered to catalyze specific reactions de novo are also described. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

In certain forms, the RNA scaffold is a functional RNA or a vector expressing one or more functional RNAs that is a triplex forming oligonucleotide molecule. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12.

In certain forms, the RNA scaffold is a functional RNA or a vector expressing one or more functional RNAs that is an external guide sequence. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

In certain forms, the RNA scaffold is a functional RNA or a vector expressing one or more functional RNAs that induces gene silencing through RNA interference (siRNA). Expression of a target gene can be effectively silenced in a highly specific manner through RNA interference. An RNA polynucleotide with interference activity of a given gene will down-regulate the gene by causing degradation of the specific messenger RNA (mRNA) with the corresponding complementary sequence and preventing the production of protein (see Sledz and Williams, Blood, 106(3):787-794 (2005)). When an RNA molecule forms complementary Watson-Crick base pairs with an mRNA, it induces mRNA cleavage by accessory proteins. The source of the RNA can be viral infection, transcription, or introduction from exogenous sources.

Gene silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism.

In one embodiment, a siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, et al., Nature, 411:494-498 (2001)) (Ui-Tei, et al., FEBS Lett, 479:79-82 (2000)). siRNA can be chemically or in vitro-synthesized or can be the result of short double-stranded hairpin-like RNAs (shRNAs) that are processed into siRNAs inside the cell. For example, WO 02/44321 describes siRNAs capable of sequence-specific degradation of target mRNAs when base-paired with 3′ overhanging ends and is herein specifically incorporated by reference for the method of making these siRNAs. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

In some forms the RNA scaffold includes one or more gene editing components, such as one or more RNAs associated with a CRISPR system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus.

iii. Encapsulation or Structural Enclosure

In some embodiments, scaffolded RNA nanostructures are designed to have a shape or three dimensional form that encloses a volume suitable to contain one or more functional molecules. For example, in some embodiments, the nanostructures are designed to have the shape of a cup, box, vase or other open structure enclosing a volume, into which one or more functional molecules can be loaded or inserted. In some embodiments, insertion or loading of functional molecules to within the inner space of the scaffolded RNA nanostructure is directed through the presence of capture tags within or near the interior space of the structure. In some embodiments, functional molecules that are locate within the inner space of the structure are maintained within the structure by the addition of one or more additional molecules, for example, to “block” or otherwise sterically prevent the release of the contained molecule. Therefore, in some embodiments, scaffolded RNA nanostructures are designed to include a “lid” or other structured nucleic acid form that encapsulates a loaded or “captured” functional molecule with in the inner-space of the nanostructure. In some embodiments the access to the inner space of scaffolded RNA nanostructures is mediated by a structural or conformational change in the structure. Therefore, in some embodiments, the encapsulation of a functional molecule and/or release of the functional molecule from the inner space is controlled by one or more external factors that induce a conformational change in the nanostructure.

b. Functional Molecules

Scaffolded RNA nanostructures including nucleic acid overhang sequences can capture one or more functional moieties, including but not limited to single-guide- or crispr-RNAs (crRNA), anti-sense DNA, anti-sense RNA as well as DNA coding for proteins, mRNA, miRNA, piRNA and siRNA, DNA-interacting proteins such as CRISPR, TAL effector proteins, or zinc-finger proteins, lipids, carbohydrates. In other embodiments, nucleic acid nanoparticles are modified with naturally or non-naturally occurring nucleotides having a known biological function. Exemplary functional groups include targeting elements, immunomodulatory elements, chemical groups, biological macromolecules, and combinations thereof.

In some embodiments, functionalized nucleic acid nanostructures include one or more single-strand overhang or scaffold DNA sequences that are complementary to the loop region of an RNA, such as an mRNA. Nucleic acid nanoparticles functionalized with mRNAs encoding one or more proteins are described. In one exemplary case, a tetrahedron (but could be any other object that can be designed from the procedure) can be functionalized with 3 (or 1 or 2 or more than 3) single-strand overhang DNA sequences that are complementary to the loop region of an RNA, for example an mRNA, for example an mRNA expressing a protein.

i. Targeting Elements

Targeting elements can be added to the staple strands of the scaffolded RNA nanostructures, to enhance targeting of the nanostructures to one or more cells, tissues or to mediate specific binding to a protein, lipid, polysaccharide, nucleic acid, etc. For example, for use as biosensors, additional nucleotide sequences are included as overhang sequences on the staple strands.

Exemplary targeting elements include proteins, peptides, nucleic acids, lipids, saccharides, or polysaccharides that bind to one or more targets associated with an organ, tissue, cell, or extracellular matrix, or specific type of tumor or infected cell. The degree of specificity with which the nucleic acid nanostructures are targeted can be modulated through the selection of a targeting molecule with the appropriate affinity and specificity. For example, antibodies, or antigen-binding fragments thereof are very specific.

Typically, the targeting moieties exploit the surface-markers specific to a biologically functional class of cells, such as antigen presenting cells. Dendritic cells express a number of cell surface receptors that can mediate endocytosis. In some embodiments, overhang sequences include nucleotide sequences that are complementary to nucleotide sequences of interest, for example HIV-1 RNA viral genome.

Additional functional groups can be introduced on the staple strand for example by incorporating biotinylated nucleotide into the staple strand. Any streptavidin-coated targeting molecules are therefore introduced via biotin-streptavidin interaction. In other embodiments, non-naturally occurring nucleotides are included for desired functional groups for further modification. Exemplary functional groups include targeting elements, immunomodulatory elements, chemical groups, biological macromolecules, and combinations thereof.

Typically, the targeting moieties exploit the surface-markers specific to a group of cells to be targeted. Exemplary targeting elements include proteins, peptides, nucleic acids, lipids, saccharides, or polysaccharides that bind to one or more targets associated with cell, or extracellular matrix, or specific type of tumor or infected cell. The degree of specificity with which the delivery vehicles are targeted can be modulated through the selection of a targeting molecule with the appropriate affinity and specificity. For example, antibodies, or antigen-binding fragments thereof are very specific.

(a) Antibodies

In some embodiments, nucleic acid nanostructures are modified to include one or more antibodies. Antibodies that function by binding directly to one or more epitopes, other ligands, or accessory molecules at the surface of cells can be coupled directly or indirectly to the nanostructures. In some embodiments, the antibody or antigen binding fragment thereof has affinity for a receptor at the surface of a specific cell type, such as a receptor expressed at the surface of macrophage cells, dendritic cells, or epithelial lining cells. In some embodiments the antibody binds one or more target receptors at the surface of a cell that enables, enhances or otherwise mediates cellular uptake of the antibody-bound nanostructure, or intracellular translocation of the antibody-bound nanostructure, or both.

Any specific antibody can be used to modify the nucleic acid nanostructures. For example, antibodies can include an antigen binding site that binds to an epitope on the target cell. Binding of an antibody to a “target” cell can enhance or induce uptake of the associated nucleic acid nanostructures by the target cell protein via one or more distinct mechanisms.

In some embodiments, the antibody or antigen binding fragment binds specifically to an epitope. The epitope can be a linear epitope. The epitope can be specific to one cell type or can be expressed by multiple different cell types. In other embodiments, the antibody or antigen binding fragment thereof can bind a conformational epitope that includes a 3-D surface feature, shape, or tertiary structure at the surface of the target cell.

In some embodiments, the antibody or antigen binding fragment that binds specifically to an epitope on the target cell can only bind if the protein epitope is not bound by a ligand or small molecule.

Various types of antibodies and antibody fragments can be used to modify nucleic acid nanostructures, including whole immunoglobulin of any class, fragments thereof, and synthetic proteins containing at least the antigen binding variable domain of an antibody. The antibody can be an IgG antibody, such as IgG1, IgG2, IgG3, or IgG4 subtyes. An antibody can be in the form of an antigen binding fragment including a Fab fragment, F(ab′)2 fragment, a single chain variable region, and the like. Antibodies can be polyclonal, or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The antibodies can also be modified by recombinant means, for example by deletions, additions or substitutions of amino acids, to increase efficacy of the antibody in mediating the desired function. Substitutions can be conservative substitutions.

For example, at least one amino acid in the constant region of the antibody can be replaced with a different residue (see, e.g., U.S. Pat. Nos. 5,624,821; 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In one embodiment, the epitopes are from the same antigen. In another embodiment, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies that target the nucleic acid nanostructures to a specific epitope can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

(b) Capture Tags

In some embodiments, scaffolded RNA nanostructures include one or more sequences of nucleic acids that act as capture tags, or “Bait” sequences to specifically bind one or more targeted molecules. For example, in some embodiments, overhang sequences include nucleotide “bait” sequences that are complementary to any target nucleotide sequence, for example HIV-1 RNA viral genome. In further embodiments, functional groups are present on one or more staple strands to act as capture tags. For example, in some embodiments, one or more biotinylated nucleotides are incorporated into the staple strand. Streptavidin-coated molecules are therefore introduced via biotin-streptavidin interaction. Typically, targeting moieties exploit the surface-markers specific to a group of cells to be targeted. Exemplary targeting elements include proteins, peptides, nucleic acids, lipids, saccharides, or polysaccharides that bind to one or more targets associated with cell, or extracellular matrix, or specific type of tumor or infected cell. Targeting molecules can be selected based on the desired physical properties, such as the appropriate affinity and specificity for the target. Exemplary targeting molecules having high specificity and affinity include antibodies, or antigen-binding fragments thereof. Therefore, in some embodiments, nucleic acid nanostructures include one or more antibodies or antigen binding fragments specific to an epitope. The epitope can be a linear epitope. The epitope can be specific to one cell type or can be expressed by multiple different cell types. In other embodiments, the antibody or antigen binding fragment thereof can bind a conformational epitope that includes a 3-D surface feature, shape, or tertiary structure at the surface of the target cell.

ii. Functional Nucleic Acids

In some embodiments, the scaffolded RNA nanostructures include one or more functional nucleic acids. Functional nucleic acids that inhibit the transcription, translation or function of a target gene are described.

Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the target polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore the compositions can include one or more functional nucleic acids designed to reduce expression or function of a target protein.

Methods of making and using vectors for in vivo expression of the described functional nucleic acids such as antisense oligonucleotides, siRNA, shRNA, miRNA, EGSs, ribozymes, and aptamers are known in the art.

(a) Antisense Molecules

The scaffolded RNA nanostructures can include functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (Kd) less than or equal to 10⁻⁶, 10⁻⁸, 10⁻¹⁰, or 10⁻¹².

(b) Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kd's from the target molecule of less than 10⁻¹² M. It is preferred that the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a Kd with the target molecule at least 10, 100, 1000, 10,000, or 100,000 fold lower than the Kd with a background binding molecule. It is preferred when doing the comparison for a molecule such as a polypeptide, that the background molecule be a different polypeptide.

(c) Ribozymes

The functional nucleic acids can be ribozymes. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are described. Ribozymes that are not found in natural systems, but which have been engineered to catalyze specific reactions de novo are also described. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

(d) Triplex Forming Nucleotides

The functional nucleic acids can be triplex forming oligonucleotide molecules. Triplex forming functional nucleic acid molecules are molecules that can interact with either double-stranded or single-stranded nucleic acid. When triplex molecules interact with a target region, a structure called a triplex is formed, in which three strands of DNA are forming a complex, dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12.

(e) External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

(f) RNA Interference

In some embodiments, the functional nucleic acids induce gene silencing through RNA interference (siRNA). Expression of a target gene can be effectively silenced in a highly specific manner through RNA interference.

Gene silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism.

In one embodiment, a siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, et al., Nature, 411:494-498 (2001)) (Ui-Tei, et al., FEBS Lett, 479:79-82 (2000)). siRNA can be chemically or in vitro-synthesized or can be the result of short double-stranded hairpin-like RNAs (shRNAs) that are processed into siRNAs inside the cell. For example, WO 02/44321 describes siRNAs capable of sequence-specific degradation of target mRNAs when base-paired with 3′ overhanging ends, herein incorporated by reference for the method of making these siRNAs. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

Therefore, in some embodiments, the composition includes a vector expressing the siRNA. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some embodiments, the functional nucleic acid is siRNA, shRNA, or miRNA.

iii. Gene Editing Molecules

In certain forms, the RNA-based nucleic acid nanostructures are functionalized to include gene editing moieties, or to include components capable of binding to gene editing moieties. Exemplary gene-editing moieties that can be included within or bound to nucleic acid nanoparticles are CRISPR RNAs, for the gene editing through the CRISPR/Cas system.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the “target sequence” and the tracrRNA is often referred to as the “scaffold.”

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence (such as CTPS1) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

In an exemplary embodiment, crRNA can be extended 3′ and CRISPR-Cpf1 loaded with this crRNA can be used to capture this protein/RNA complex, as assayed by gel mobility shift and dual staining with a DNA-specific stain and a protein-specific stain.

In another embodiment, CRISPR-Cpf1 complexed with crRNA targeting a sequence in the EGFP gene. The cross-beam was made to be a duplex that contains this specific sequence, but could be homologous to the target sequence with 10, 12, 13, 14, 15, 16, 17, 18, 19, 20, 21, 22, 23 nucleotides (20 nucleotides in the example case). The CRISPR-Cpf1/crRNA complex was found to bind to the nanoparticle as assayed by gel mobility shift and dual staining for DNA and protein material.

iv. Zinc Finger Nucleases

In some embodiments, the RNA-based nucleic acid nanostructures include a nucleic acid construct or constructs encoding a zinc finger nuclease (ZFN). ZFNs are typically fusion proteins that include a DNA-binding domain derived from a zinc-finger protein linked to a cleavage domain.

The most common cleavage domain is the Type IIS enzyme FokI. FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31, 978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys2His2 zinc fingers, each of which generally recognizes three to four nucleotides in the target DNA sequence. The Cys2His2 domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

v. mRNA

In some embodiments, RNA nanostructures are modified by covalent or non-covalent association with an RNA that encodes one or more polypeptides, such as a protein. Therefore, in some embodiments, nucleic acid nanostructures are modified to include one or more messenger RNA molecules (mRNA). The messenger RNA can encode any protein or polypeptide. For example, in some embodiments, nucleic acid nanostructures are modified to include one or more mRNAs, each encoding one or more proteins. In an exemplary embodiment, the mRNA encodes a fluorescent protein or fluorophore. Exemplary fluorescent proteins include mCherry, mPlum, mRaspberry, mStrawberry, tdTomato, GFP, EBFP, Azurite, T-Sapphire, Emerald, Topaz, Venus, mOrange, AsRed2, and J-Red. In some embodiments, nucleic acid nanostructures are modified to include one or more messenger RNA molecules an RNA that encodes one or more polypeptides, such as a protein that is an antigen.

vi. Therapeutic or Prophylactic Agents

In some embodiments, RNA nanostructures are modified by covalent or non-covalent association with a therapeutic agent, or a prophylactic agent, or a diagnostic agent. For example, one or more therapeutic, prophylactic, or diagnostic agents can be associated with the exterior of the RNA nanoparticle, or packaged within the interior space of the RNA nanoparticle, according to the design of the particle and location of the capture tag or site of interaction with the Therapeutic or prophylactic or diagnostic agent. A non-limiting list of active agents that can be encapsulated within, or otherwise associated with the RNA nanoparticle includes anti-infectives, immuno-modifying agents, hormones, antioxidants, steroids, anti-proliferative agents and diagnostic agents. Therapeutic agents can include a drug or modified form of drug such as prodrugs and analogs.

Examples of agents include, but are not limited to, beta-lactam antibiotics (including penicillins such as ampicillin, cephalosporins selected in turn from cefuroxime, cefaclor, cephalexin, cephydroxil and cepfodoxime proxetil); tetracycline antibiotics (doxycycline and minocycline); microlides antibiotics (azithromycin, erythromycin, rapamycin and clarithromycin); fluoroquinolones (ciprofloxacin, enrofloxacin, ofloxacin, gatifloxacin, levofloxacin) norfloxacin, an antioxidant drug includes N-acetylcysteine (NAC); anti-inflammatory drugs, such as nonsteroidal drugs (e.g., indomethacin, aspirin, acetaminophen, diclofenac sodium and ibuprofen); steroidal anti-inflammatory drug (e.g., dexamethasone); antiproliferative agents (e.g., Paclitaxel (Taxol), QP-2 Vincristin, Methotrexat, Angiopeptin, Mitomycin, BCP 678, Antisense c-myc, ABT 578, Actinomycin-D, RestenASE, 1-Chlor-deoxyadenosin, PCNA Ribozym, and Celecoxib) sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase); antimitotic agents (e.g., vincristine, vinblastine, vinorelbine, docetaxel, estramustine); molecularly targeted agents including antibodies, antibody fragments, or carbohydrates/polysaccharides (e.g., imatinib, tretinoin, bexarotene, bevacizumab, gemtuzumab ogomicin and denileukin diftitox); and corticosteroids (e.g., fluocinolone acetonide and methylprednisolone).

vii. Other Modifications In some embodiments, nucleic acid nanostructures include modifications that are not related to the nucleic acid sequence of the staple strands or the scaffold sequence. In some embodiments, the nanostructures include polymers or lipids, for example, surrounding or within the space enclosed by the nanostructure. In a particular embodiment, nanostructures include a complete surface coating, for example, by lipids or other polymers (e.g., polyethylene glycol or phospholipids). Complete surface coating of the nanostructures by lipids or other polymers (e.g., polyethylene glycol or phospholipids) could also be used in order to make these objects able to escape immune defense and enable the capacity of external modification. Therefore, in some embodiments, the surface of the nanostructure includes an amount of lipid or other polymer effective to coat the nanostructure and reduce immune surveillance or immune uptake of the nanostructure. In some embodiments, the surface of the nanostructure includes an amount of lipid or other polymer effective to enable external modification, for example, by insertion of one or more proteins, lipids, nucleic acids, polymers or small molecules into the lipid or polymer layer reconstituted around the nanoparticle. Preferred polymers are biocompatible (i.e., do not induce a significant inflammatory or immune response) and non-toxic.

Examples of suitable hydrophilic polymers include, but are not limited to, poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(amino acids), poly(hydroxy acids), poly(vinyl alcohol), and copolymers, terpolymers, and mixtures thereof.

In preferred embodiments, the one or more hydrophilic polymer segments contain a poly(alkylene glycol) chain. The poly(alkylene glycol) chains may contain between 1 and 500 repeat units, more preferably between 40 and 500 repeat units. Suitable poly(alkylene glycols) include polyethylene glycol, polypropylene 1,2-glycol, poly(propylene oxide), polypropylene 1,3-glycol, and copolymers thereof.

In some, embodiments, amphiphilic proteins or other amphiphilic molecules (e.g., drugs) including targeting moieties, or not including targeting moieties, or combinations, are inserted in a lipid layer reconstituted around the nanoparticles.

Some further non-limiting examples include targeting the therapeutic, prophylactic or diagnostic agent to the disease sites for therapeutic and/or diagnostic purposes.

### V. Uses

RNA nanostructures prepared according to methods described above are suitable for many applications. Some exemplary uses include in drug delivery, in biosensors, in memory storage, in nano-electronic circuitry, etc.

A. Delivery Vehicles

RNA nanostructures are suitable as a delivery vehicle for therapeutic, prophylactic and/or diagnostic agents. Since they are nucleic acid based, RNA nanostructures are entirely biocompatible and elicit minimal immune response in the host. The automated design of any desired geometry of RNA nanostructure further allows manipulation of RNA structure tailored for individual drugs, dose, site of target and desired rate of degradation etc.

Any prophylactic, therapeutic, or diagnostic agent can be incorporated into the RNA origami nanostructures via a variety of interactions, non-covalent or covalent. Some exemplary non-covalent interactions for attachment include intercalation, via biotin-streptavidin interaction, chemical linkers (e.g., using Click-chemistry groups), or via hybridization between complementary nucleotide sequences.

In some embodiments, the agents to be delivered are simply captured inside the RNA origami nanostructures. In these cases, pore size of the RNA polyhedron is a key consideration, i.e., they are small enough so that the agent captured does not leak out. In some embodiments, the RNA polyhedron are assembled in two halves to allow the capture of agent prior to the completion of the polyhedron nanostructures.

1. Delivery of Active Agents

In some forms, therapeutic, prophylactic, toxic, diagnostic or other agents are delivered using the nucleic acid nanoparticles. Exemplary agents to be delivered include proteins, peptides, carbohydrates, nucleic acid molecules, polymers, small molecules, and combinations thereof. In some embodiments, the nucleic acid nanoparticles are used for the delivery of a peptide drug, a dye, an antibody, or antigen-binding fragment of an antibody.

Therapeutic agents can include anti-cancer, anti-inflammatories, or more specific drugs for inhibition of the disease or disorder to be treated. These may be administered in combination, for example, a general anti-inflammatory with a specific biological targeted to a particular receptor. For example, one can administer an agent in treatment for ischemia that restores blood flow, such as an anticoagulant, anti-thrombotic or clot dissolving agent such as tissue plasminogen activator, as well as an anti-inflammatory. A chemotherapeutic which selectively kills cancer cells may be administered in combination with an anti-inflammatory that reduces swelling and pain or clotting at the site of the dead and dying tumor cells. Suitable genetic therapeutics include anti-sense DNA and RNA as well as DNA coding for proteins, mRNA, antisense silencing RNA (siRNA), short hairpin RNA (shRNA), micro RNA (miRNA), external guide sequences (EGSs), Piwi-interacting RNA (piRNA), single guide RNA (sgRNA), ribozymes, and aptamers. In some embodiments, the nucleic acid that forms the nanoparticles include one or more therapeutic, prophylactic, diagnostic, or toxic agents. In some embodiments, the RNA nanoparticle includes one or more siRNAs, or one or more vectors expressing an siRNA. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some embodiments, the functional nucleic acid is siRNA, shRNA, or miRNA.

In some embodiments, therapeutic, prophylactic, toxic, diagnostic or other agents are delivered to a cell or tissue by endogenous uptake of the RNA nanoparticles by the cell or tissue. In some embodiments, the agents are released from the nucleic acid nanoparticles within the blood stream. In other embodiments the agent are released within the gastro-intestinal system, uro-genital system, lymphatic system, central nervous system, or into the skin. The release of agents bound to or otherwise associated with the nucleic acid nanoparticles can occur in vivo, by contact with one or more enzymes, proteins or other factors present in physiological concentrations. Exemplary enzymes include nuclease enzymes, such as exonucleases, endonucleases and other restriction enzymes, proteases, hydrolases and other enzymes. When release of an agent involves a conformational change in the structure of the nucleic acid nanoparticles, the conformational change can occur as a result of exposure to one or more physiological conditions, such as pH, salt concentration or interaction with one or more substances present in the body.

2. Delivery of Scaffold Functional RNAs In Vivo

In some forms the RNA-based nanostructures are used for delivery of one or more functional RNAs into a target or host cell. For example, in some forms, the RNA scaffold is functional RNA, or a vector expressing one or more functional RNAs that is delivered to a host or target cell in the form of a three dimensional RNA nanostructure. Typically, when the RNA scaffold includes functional RNA or a vector expressing one or more functional RNAs, the scaffold must be unfolded within a target or host cell for expression of the mRNA within the cell. Therefore, in some forms, the nanostructures include functional RNA or a vector expressing one or more functional RNAs as a scaffold that is designed to be released within the cell by unfolding of the nanostructure. Typically, the unfolding includes intracellular enzymic activity, such as removal of one or more of the staples and/or structural components of the nanostructure. In some forms, the RNA nanostructure includes a functional RNA or a vector expressing one or more functional RNAs as scaffold and DNA staples. When the nanostructures include DNA staples, the activity of intra cellular DNA-specific nucleases can release the scaffold functional RNA or vector expressing one or more functional RNAs within a host cell. In some forms, the RNA-based nanostructures include functional RNA or a vector expressing one or more functional RNAs as a scaffold sequence that is protected from RNAse activity, for example, by modification of the functional RNA scaffold. Exemplary functional RNAs include antisense silencing RNA (siRNA), short hairpin RNA (shRNA), micro RNA (miRNA), external guide sequences (EGSs), Piwi-interacting RNA (piRNA), single guide RNA (sgRNA), ribozymes, and aptamers.

3. Delivery of Scaffold mRNA In Vivo

In some forms the RNA nanostructures are used for delivery of mRNA or a vector expressing one or more mRNAs into a target or host cell. For example, in some forms, the RNA scaffold is mRNA or a vector expressing one or more mRNAs that is delivered to a host or target cell in the form of a three dimensional RNA nanostructure.

Typically, when the RNA scaffold includes mRNA, the scaffold must be unfolded within a target or host cell for expression of the mRNA within the cell. Therefore, in some forms, the nanostructures include an mRNA scaffold that is designed to be released within the cell by unfolding of the nanostructure. Typically, the unfolding includes intracellular enzymic activity, such as removal of one or more of the staples and/or structural components of the nanostructure. In some forms, the RNA nanostructure includes an mRNA scaffold and DNA staples. When the nanostructures include DNA staples, the activity of DNA-specific nucleases can release the scaffold mRNA for expression of one or more antigenic peptides within a host cell. In some forms, the RNA-based nanostructures include an mRNA scaffold sequence that is protected from RNAse activity, for example, by modification of the mRNA scaffold.

i. Vaccines

In some forms the RNA nanostructures are used as vaccines, for delivery of mRNA that is or encodes or expresses an antigen.

In other forms, RNA nanostructures have bound thereto or are complexed with one or more antigens, for example, at the surface of the nanostructure. 3D organizations of antigens at the surface of a nanostructure can be used to stimulate the immune system by presenting these antigens in geometries that mimic the one or more naturally occurring antigens.

In some forms, specific nucleic acid sequences may be included as adjuvants, with the 3D patterning in geometry and size controlled in an arbitrary manner using the procedure provided here in which the RNA wireframe geometry scaffolds viral proteins or peptides or other active fragments.

Exemplary antigens include viral antigens, parasite antigens, bacterial antigens, allergens or environmental antigens and tumor antigens. In an exemplary embodiment, the antigen is a natural viral capsid structure.

B. Scaffold Structures for Display and Analysis of Molecules

RNA origami nanostructures can act as scaffolds for a variety of molecules including protein, nucleic acid, lipids, or polysaccharides. In some, embodiments, one or more molecules are conjugated to the nanoparticles. For example, in some, embodiments, one or more molecules are conjugated to the outside of the nanoparticle, to the inside of the nanoparticle, or both to the inside and outside of the particle. Any molecules of interest can be conjugated to the nanoparticles. Exemplary categories of molecule that can be conjugated include proteins, lipids, carbohydrates, small-molecules, nucleic acids and combinations.

In some embodiments nanostructures are used to capture and/or restrain molecules in a fixed and known orientation, for example, to assist biophysical analyses, such as structural determination.

C. Sensors

RNA origami nanostructures can act as biosensors for a variety of molecules including protein, nucleic acid, lipids, or polysaccharides. In particular, the RNA origami nanostructures prepared according to the methods described above are capable of adopting any arbitrary shapes, therefore making them ideal sensor for other molecules, or secondary and tertiary structures of other molecules.

In some embodiments, RNA origami nanostructures are used to capture RNA molecules of interest for probing their secondary and tertiary structures. In preferred embodiments, the RNA origami nanostructure:RNA complexes are suitable for further structural analysis for example, particularly using selective 2′-hydroxyl acylation analyzed by primer extension (SHAPE), or cryo-EM analysis.

In some embodiments, RNA origami nanostructures are designed for binding to a particular RNA virus, for example human immunodeficiency virus (HIV), influenza, Ebola, hepatitis C, SARS, and Zika viruses. In some embodiments, RNA origami nanostructures are used as RNA/viral detection sensors for use in the battlefield.

D. Nanoelectronic Circuitry

RNA nanostructures prepared according to methods described above are suitable for use as nanoscale electronic devices. The automation of RNA nanostructure design allows user input of any desired geometry. The staple strands can be functionalized for incorporating any desired functionalities such as anchoring to any surfaces, for incorporating any non-naturally occurring molecules, etc.

In some embodiments, metallization of the RNA template is used for circuit fabrication. In preferred embodiments, the shape of RNA origami nanostructures is maintained after the metallization process.

The present invention will be further understood by reference to the following non-limiting examples.

E. Imaging Probes

RNA nanostructures prepared according to the described methods above are suitable for use as a molecular probe, for example, as a fluorescent probe. Based on the capacity to generate structures with random geometries and size, and the facility of modification on prescribed position determined by the user, fluorescent dyes could be easily conjugated. The number of fluorescent dyes that can be conjugated depends on the structure size and the number of the staple strands that can be modified.

In some embodiments dye-conjugated RNA nanostructures are used for conjugation to specific ligand-binding moieties, such as antibodies, aptamers, protein-binding domains, etc., for example, by integrating chemical groups (e.g., Click-chemistry groups, amine groups, etc.) into the nanostructures. Nanostructures including specific ligand-binding moieties are used for labelling and imaging applications, such as imaging and super-resolution imaging.

F. Light-Harvesting and Excitonic Circuits

RNA nanostructures containing densely or loosely packed aggregates of chromophores can be used as excitonic energy transfer circuits. Chromophores of prescribed types can be organized using the 3D arrays published here to form 1D/2D/3D architectures for exciton funneling and transport in nanoscale energy transport.

The disclosed compositions and methods can be further understood through the following numbered paragraphs.

1. A method for designing a scaffolded RNA nanostructure having a geometric shape including:
(a) determining the geometric parameters of an input, wherein the input includes a 3D polyhedral or 2D polygon geometric shape and optionally one or more of its physical dimensions;
(b) identifying a route for a single-stranded RNA scaffold that traces throughout the geometric shape based on A-form helical nucleic acid geometry; and
(c) generating the sequences of the single-stranded RNA scaffold and optionally the nucleic acid sequence of staple strands that combine to form a scaffolded RNA nanostructure having the geometric shape.

2. The method of paragraph 1, wherein generating the sequences of the single-stranded RNA scaffold in step (c) includes staple crossover asymmetry, with 11 nucleotides per helical turn.

3. The method of paragraph 2, wherein the staple crossover asymmetry includes a difference in the nucleotide position across two helices of an edge of from one to ten nucleotides, inclusive.

4. The method of paragraph 2, wherein the staple crossover asymmetry includes a difference in the nucleotide position across two helices of an edge of four nucleotides.

5. The method of paragraph 1, wherein identifying a route for a single-stranded RNA scaffold that traces throughout the geometric shape in step (b) includes scaffold crossover asymmetry, with 11 nucleotides per helical turn.

6. The method of paragraph 5, wherein the scaffold crossover asymmetry includes a difference in the nucleotide position across two helices of an edge of from one to ten nucleotides, inclusive.

7. The method of paragraph 5, wherein the scaffold crossover asymmetry includes a difference in the nucleotide position across two helices of an edge of six nucleotides.

8. The method of paragraph 1, wherein identifying a route for a single-stranded RNA scaffold that traces throughout the geometric shape in step (b) includes no crossover asymmetry, with 11 nucleotides per helical turn.

9. The method of any one of paragraphs 1-8, wherein the input in step (a) further includes one or more of the geometric shape's physical dimensions.

10. The method of any one of paragraphs 1-9, wherein the input in step (a) further includes specifying an even number of parallel or anti-parallel helices within each edge of the nanostructure.

11. The method of paragraph 10, wherein each edge includes four or more parallel or anti-parallel helices arranged in square cross-sectional morphology, or six or more parallel or anti-parallel helices arranged in honeycomb lattice morphology.

12. The method of any one of paragraphs 1-11, wherein the input in step (a) further includes specifying for each vertex of the nanostructure that two or more edges come together in an aligned angle to create a bevel at the vertex.

13. The method of paragraph 1, wherein the geometric shape does not have spherical topology.

14. The method of any one of paragraphs 1-13, wherein the input in step (a) further includes a template RNA scaffold sequence, or the sequence of one or more staples, or a template RNA scaffold sequence and the sequence of one or more staples.

15. The method of any one of paragraphs 1-14, wherein the input in step (a) further includes the length of one or more of the edges spanning two vertices of the target structure.

16. The method of paragraph 15, wherein the length of each edge is between 22 base pairs and 1,100 base pairs, inclusive.

17. The method of any one of paragraphs 1-16, wherein the crossover type is anti-parallel crossover, wherein the length of each edge is expressed as a multiple of 11 base pairs, and wherein the length of each edge is between 22 base pairs and 1,100 base pairs, inclusive.

18. The method of paragraph 17, wherein the length of each edge is 44 base pairs, 55 base pairs, 66 base pairs, or 77 base pairs.

19. The method of any one of paragraphs 1-17, wherein the staples are DNA.

20. The method of any one of paragraphs 1-17, wherein the staples are RNA.

21. The method of any one of paragraphs 1-20, wherein the input in step (a) includes geometric parameters including vertex, face and edge information determined from a polygonal or polyhedral wire-mesh model of the target shape.

22. The method of any one of paragraphs 1-21, wherein identifying a route for a single-stranded RNA scaffold that traces throughout the geometric shape in step (b) includes the steps of:
(i) rendering the geometric shape as a closed surface polyhedral network or an open surface polygonal network;
(ii) determining a spanning tree of the network, wherein the vertices and lines of the graph are the nodes and edges of the network, respectively;
(iii) classifying each edge of the network based on its membership in the spanning tree,
wherein edges that are members of the spanning tree do not have a scaffold double crossover, and edges that are not members of the spanning tree have a scaffold double crossover;
(iv) splitting each edge that is not a member of the spanning tree into two edges, each containing a pseudo-node at the point of the scaffold crossover;
(v) splitting each node at each of the vertices into two pseudo-nodes; and
(vi) calculating the Euler cycle of the network, wherein the Euler cycle represents the route of a single-stranded RNA scaffold that traces once along each edge in both directions throughout the entire geometric shape.

23. The method of paragraph 22, wherein the crossover type is parallel crossover, and wherein the length of each edge is between 22 base pairs and 1,100 base pairs, inclusive.

24. The method of any one of paragraphs 1-21, wherein identifying a route for a single-stranded RNA scaffold that traces throughout the geometric shape in step (b) includes the steps of:
(i) rendering the geometric shape as a closed surface polyhedral network or an open surface polygonal network;
(ii) calculating a spanning tree of the network, wherein the vertices and lines of the graph are the nodes and edges of the network, respectively;
(iii) classifying each edge of the network as one of four types based on its membership in the spanning tree and on whether it employs anti-parallel or parallel crossovers; edges that are members of the spanning tree have each scaffold portion start and end at different vertices, and edges that are not members of the spanning tree have each scaffold portion start and end at the same vertex;
(iv) splitting each edge that is not a member of the spanning tree into two edges, each containing a pseudo-node at the point of the scaffold crossover;
(v) splitting each node at each of the vertices into two pseudo-nodes; and
(vi) calculating the Euler cycle of the network, wherein the Euler cycle represents the route of a single-stranded nucleic acid scaffold by superimposing and connecting units of partial scaffold routing within an edge based on its classification and length.

25. The method of any one of paragraphs 1-21, wherein identifying a route for a single-stranded RNA scaffold that traces throughout the geometric shape in step (b) includes:
(i) rendering the geometric shape as a closed surface polyhedral network or an open surface polygonal network;
(ii) rendering each helix in the network as a line, based on the target cross-section of each edge;
(iii) calculating a loop-crossover structure, wherein two or more adjacent lines are connected to form loops and all possible double-crossover locations between two loops are calculated;
(iv) calculating a dual graph of the loop-crossover structure, wherein the loops and double-crossover locations of the network are converted to nodes and edges of the dual graph, respectively;
(v) calculating a spanning tree of the dual graph network;
(vi) calculating which of the locations of double-strand crossovers will be used, wherein a single double-strand crossover is placed at each edge that is the part of the spanning tree of the dual graph; and
(vii) calculating the Euler cycle of the network, wherein the Euler cycle represents the route of a single-stranded nucleic acid scaffold that traces once through each duplex throughout the entire geometric shape.

26. The method of any one of paragraphs 22-25, wherein the spanning tree of the network is determined using a breadth-first search or depth-first search.

27. The method of paragraph 26, wherein the spanning tree is calculated using Prim's formula or Kruskal's formula.

28. The method of any one of paragraphs 22-27, wherein the Euler circuit is the A-trail Euler circuit.

29. The method of any one of paragraphs 22-28, wherein rendering the geometric shape as polyhedral network includes producing a node-edge network of the three-dimensional structure.

30. The method of any one of paragraphs 1-29, further including the step of:
(d) predicting the three-dimensional structure of the scaffolded RNA nanostructure.

31. The method of any one of paragraphs 1-30, further including the step of:
(e) assembling the scaffolded RNA nanostructure.

32. The method of paragraph 31, further including the step of:
(f) validating the scaffolded RNA nanostructure.

33. The method of paragraph 32, wherein the scaffolded RNA nanostructure is validated by comparison with a predicted three-dimensional structure.

34. A polyhedral scaffolded RNA nanostructure designed according to the method of any one of paragraphs 1-33.

35. A polyhedral scaffolded RNA nanostructure including two nucleic acid anti-parallel helices spanning each edge of the structure,
wherein the three-dimensional structure is formed from single stranded nucleic acid staple sequences hybridized to a single stranded RNA scaffold sequence,
wherein the RNA scaffold sequence is routed through the Euler cycle of the network defined by vertices and lines of a node-edge network of the polyhedral structure,
wherein the nanostructure includes at least one edge including a double-strand crossover,
wherein the location of the double-strand crossover is determined by the spanning tree of the network of the polyhedral structure,
wherein the staple sequences are hybridized to the vertices, edges and double strand crossovers of the scaffold sequence to define the shape of the nanostructure, and
wherein the staples hybridized to the edges implement crossover asymmetry that includes a difference in the nucleotide position across two helices of the edge of from one to ten nucleotides, inclusive.

36. A polyhedral scaffolded RNA nanostructure including two nucleic acid parallel helices spanning each edge of the structure,
wherein the three-dimensional structure is formed from a single stranded RNA scaffold sequence hybridized to itself and may also hybridize to single stranded nucleic acid staple sequences,
wherein the RNA scaffold sequence is routed through the Euler cycle of the network defined by vertices and lines of a node-edge network of the polyhedral structure,
wherein the RNA scaffold sequence hybridizes to itself in at least one edge using parallel crossovers,
wherein the staple sequences, if any, are hybridized to the edges and double strand crossovers of the scaffold sequence to define the shape of the nanostructure, and
wherein the staples hybridized to the edges implement crossover asymmetry that includes a difference in the nucleotide position across two helices of the edge of from one to ten nucleotides, inclusive.

37. A polyhedral or polygonal scaffolded RNA nanostructure including four or more nucleic acid anti-parallel helices spanning each edge of the structure,
wherein the three-dimensional structure is formed from single stranded nucleic acid staple sequences hybridized to a single-stranded RNA scaffold sequence,
wherein the scaffold sequence is routed through the Euler cycle of the network defined by vertices and lines of a node-edge network of the polyhedral structure,
wherein the nanostructure includes at least one edge including a double strand crossover,
wherein the location of the double strand crossover is determined by a spanning tree of the dual graph of the network of the polyhedral or polygonal structure,
wherein the helices including an edge are arranged as a square lattice of four or more helices, or honeycomb lattice of six or more helices,
wherein the helices meeting at a vertex can be beveled or non-beveled, and
wherein the staple sequences are hybridized to the vertices, edges and double strand crossovers of the scaffold sequence to define the shape of the nanostructure.

38. A polyhedral scaffolded RNA nanostructure including two nucleic acid anti-parallel helices spanning one or more edges of the structure,
wherein the three-dimensional structure is formed from single stranded nucleic acid staple sequences hybridized to a single stranded RNA scaffold sequence,
wherein the RNA scaffold sequence is routed through the Euler cycle of the network defined by vertices and lines of a node-edge network of the polyhedral structure,
wherein the nanostructure includes at least one edge including a double-strand crossover,
wherein the location of the double-strand crossover is determined by the spanning tree of the network of the polyhedral structure,
wherein the staple sequences are hybridized to the vertices, edges and double strand crossovers of the scaffold sequence to define the shape of the nanostructure, and
wherein the staples hybridized to the edges implement crossover asymmetry that includes a difference in the nucleotide position across two helices of the edge of from one to ten nucleotides, inclusive; and
wherein at least one part of the scaffold sequence is not hybridized to staples or itself, and
wherein the non-hybridized scaffold extends from an edge of the polyhedral nanostructure.

39. The polyhedral scaffolded RNA nanostructure of any one of paragraphs 34-38, further including a molecule selected from the group including PNA, protein, lipid, carbohydrate, a small-molecule, a dye, and RNA,
wherein the molecule is covalently or non-covalently bound to, or complexed with, or encapsulated within the nanostructure.

40. The polyhedral scaffolded RNA nanostructure of any one of paragraphs 34-39, further including a therapeutic, diagnostic or prophylactic agent.

41. A method of using the polyhedral scaffolded RNA nanostructure of paragraph for the delivery of the therapeutic, diagnostic or prophylactic agent to a subject, the method including the step of administering the nanoparticle to the subject.

42. A method of programming 3D geometries of arbitrary compositions of one or more molecules selected from the group including PNA, protein, lipid, carbohydrate, a small-molecule, a dye, and RNA,
wherein the molecules are conjugated to an underlying scaffolded RNA nanostructure,
wherein the 3D geometry of the one or more molecules is determined by the 3D geometry of the underlying scaffolded RNA nanostructure, and
wherein the scaffolded RNA nanostructure is designed according to the method of any one of paragraphs 1-33.

43. The scaffolded RNA nanostructure of any one of paragraphs 34-39, wherein single-stranded or double-stranded nucleic acid overhang sequences extend from nick positions from the oligonucleotide staple strands.

44. The scaffolded RNA nanostructure of paragraph 43, wherein the nucleic acid overhang sequences that extend from nick positions from the oligonucleotide staple strands form duplex reinforcements along one or more edges of the structure, or span between two vertices of the structure.

45. The scaffolded RNA nanostructure of any one of paragraphs 43 or 44, wherein the single-stranded or double-stranded nucleic acid overhangs include one or more sequences of nucleic acids that is complementary to a target RNA or DNA sequence.

46. The scaffolded RNA nanostructure of any one of paragraphs 43 or 44, wherein the single-stranded or double-stranded nucleic acid overhangs include one or more sequences of nucleic acids that interact with DNA binding proteins or RNA-binding proteins.

47. The scaffolded RNA nanostructure of any one of paragraphs 42-46, wherein the edge length and nanoparticle geometry is greater than the size of the target molecule that is to be captured, to allow for 1, 2, 3, or more than 3 molecules to be bound independently of any other.

48. The method of any one of paragraphs 1-33, or the scaffolded RNA nanostructure of any one of paragraphs 34-39 or 42-47, wherein the RNA scaffold includes one or more selected from the group including messenger RNA (mRNA), replicating RNA (repRNA), guide-strand RNA (gsRNA), ribosomal RNA (rRNA), transfer RNA (tRNA), genomic transcript RNA, aptamer RNA and functional RNA(s).

49. The method or scaffolded RNA nanostructure of paragraph 48, wherein the RNA scaffold includes messenger RNA (mRNA) encoding one or more polypeptides or proteins.

50. The method or scaffolded RNA nanostructure of paragraph 49, wherein the messenger RNA (mRNA) encodes one or more polypeptide or protein antigens.

51. The method or scaffolded RNA nanostructure of paragraph 50, wherein the antigen is selected from the group including a viral antigen, bacterial antigen, protozoan antigen, environmental allergen, food allergen and tumor antigen.

52. The method or scaffolded RNA nanostructure of paragraph 49, wherein the messenger RNA (mRNA) encodes one or more enzymes, fluorescent proteins or antigen-binding proteins.

53. The method or scaffolded RNA nanostructure of paragraph 52, wherein the messenger RNA (mRNA) encodes the prokaryotic green fluorescent protein (GFP) protein.

54. The method or scaffolded RNA nanostructure of paragraph 48, wherein the RNA scaffold includes a functional RNA selected from the group including antisense molecules, silencing RNA (siRNA), micro RNA (miRNA), ribozymes, riboswitches, short hairpin RNA (shRNA), triplex forming RNA, and interfering RNA (RNAi).

55. The method of any one of paragraphs 1-33, or the scaffolded RNA nanostructure of any one of paragraphs 34-39 or 42-54, wherein the RNA scaffold and/or staple sequences include one or more modified nucleotides.

56. The method or the scaffolded RNA nanostructure of paragraph 55, wherein the modified nucleotides reduce or prevent degradation of the modified RNA by RNAse enzymes.

57. The method or the scaffolded RNA nanostructure of paragraph 55 or 56, wherein the one or more modified nucleotides includes 2′-fluorinated deoxy-uridine, or 2′-fluorinated deoxy-cytosine, or 5-methoxyuridine.

58. The method of any one of paragraphs 1-33, or the scaffolded RNA nanostructure of any one of paragraphs 34-39 or 42-57, wherein nanostructure includes one or more RNA/DNA hybrid regions.

59. The method or the scaffolded RNA nanostructure of paragraph 58, wherein one or more of the RNA/DNA hybrid regions facilitates release of the scaffold RNA and/or one or more cargo molecules in the presence of an RNA/DNA hybrid specific nuclease.

60. A vaccine including the scaffolded RNA nanostructure of paragraph 50 or 51.

61. The method of paragraph 31, wherein assembling the scaffolded RNA nanostructure includes synthesizing the RNA scaffold sequence by a method including in vitro transcription.

The disclosed compositions and methods can be further understood through the following examples.

### EXAMPLES

### Example 1: RNA-Scaffolded 3D Wireframe Origami

Hybrid RNA-DNA origami, in which a long RNA scaffold strand is folded into a target nanostructure via thermal annealing with complementary DNA oligos, has only been explored to a limited extent despite its unique potential for biomedical delivery of mRNA, tertiary structure characterization of long RNAs, and fabrication of artificial ribozymes.

Design strategies for hybrid nucleic acid origami that use DNA oligo staples to fold in vitro-transcribed RNA scaffolds of varying sequences and lengths into seven distinct 3D wireframe polyhedra with DX edges were explored. Impacts of ionic species and strengths on folding were first characterized with gel electrophoresis, with target structures under optimal folding conditions validated using cryo-electron microscopy (cryo-EM) reconstruction. Secondary structure hybridization and stability were assessed at single-nucleotide resolution using DMS-MaPseq, which offered fundamental insights into specific sources of instability in RNA-scaffolded origami design, which might also generalize to DNA-scaffolded origami.

### Methods and Materials

Reagents

Oligonucleotide staples and primers and gBlock synthetic DNA sequences were purchased from Integrated DNA Technologies (IDT, Coralville, IA). HEPES, Trizma base, EDTA, NaCl, KCl, MgCl₂ magnesium acetate, and high-resolution agarose were purchased from Sigma-Millipore (MA). HiScribe T7 RNA polymerase kits and Q5 2×HiFidelity PCR mastermixes were purchased from New England Biolabs (NEB, Ipswitch, MA). Sodium cacodylate solution, pH 7.2, was purchased from VWR. RNAs for use as input to generate RNA scaffolds were provided as Gblocks, or transcribed in vitro as described.

A-Form DX Wireframe Origami Programmed Using pyDAEDALUS

To generalize to A-form helical geometries to allow for RNA/RNA or hybrid RNA/DNA DX-based origami, edge lengths were discretized to multiples of 11 rather than rounded multiples of 10.5, and crossover positions were changed to be compatible with A-form helix crossovers. As described previously (Sparvath, et al., Methods Mol Biol 1500, 51-80 (2017)), scaffold crossover edges, which have adjacent crossovers occurring on different strands (scaffold vs. staple) and thus must occur an odd number of half-twists apart, require that the crossovers on the two helices of the edge be spaced asymmetrically to be compatible with A-form helical geometry. two approaches were investigated to implementing this asymmetry, in addition to testing a design with no asymmetry incorporated (“Hybrid-form”).

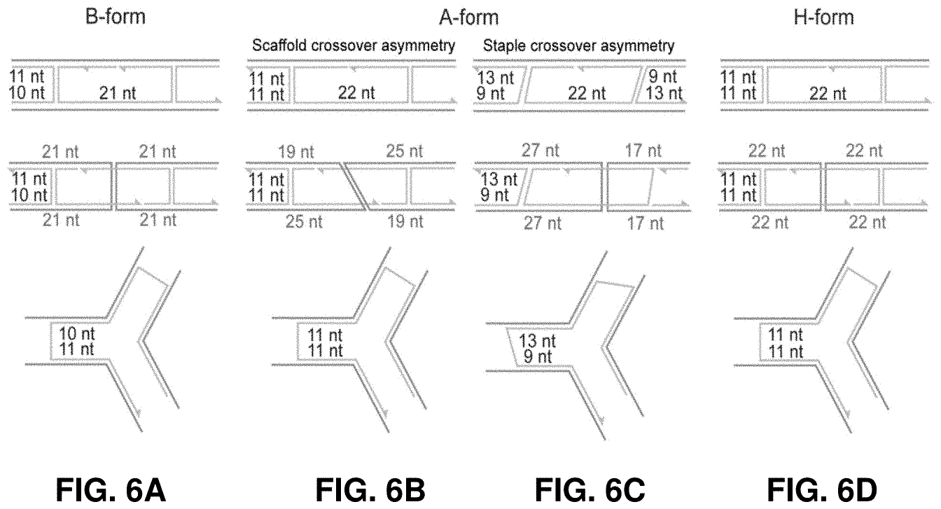

In one approach to A-form, the required asymmetry is incorporated into the staple crossover position calculation (FIG. 1, FIGS. 6A-6D). In this case, staple crossovers are asymmetric across the two helices, with a 4-nt difference between the nucleotide position on the two helices (e.g., the first staple crossover occurs 9 nt from the vertex on the 5′ side and 13 nt from the vertex on the 3′ side).

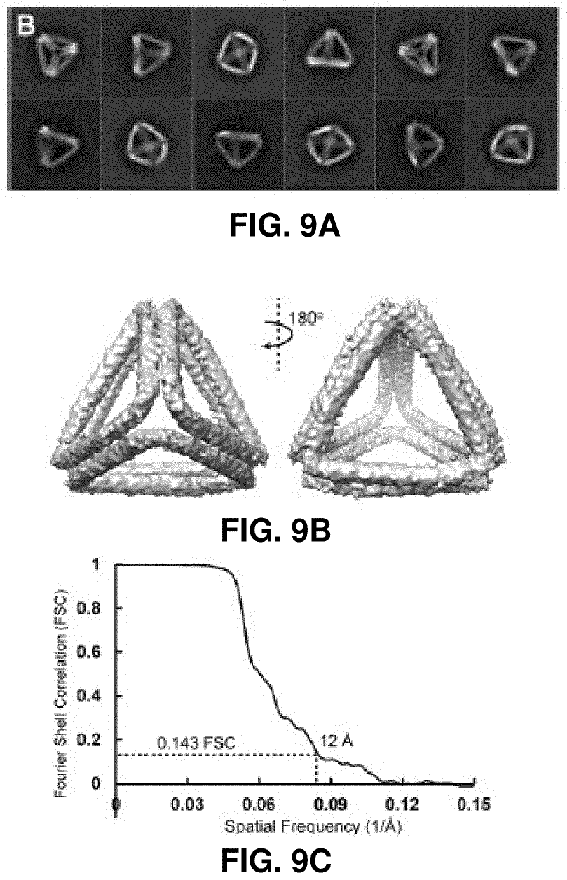

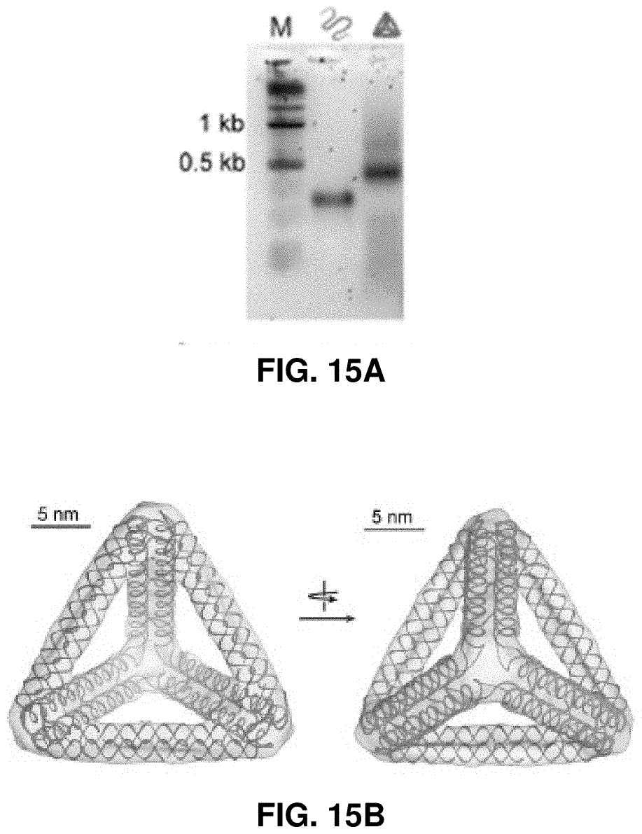

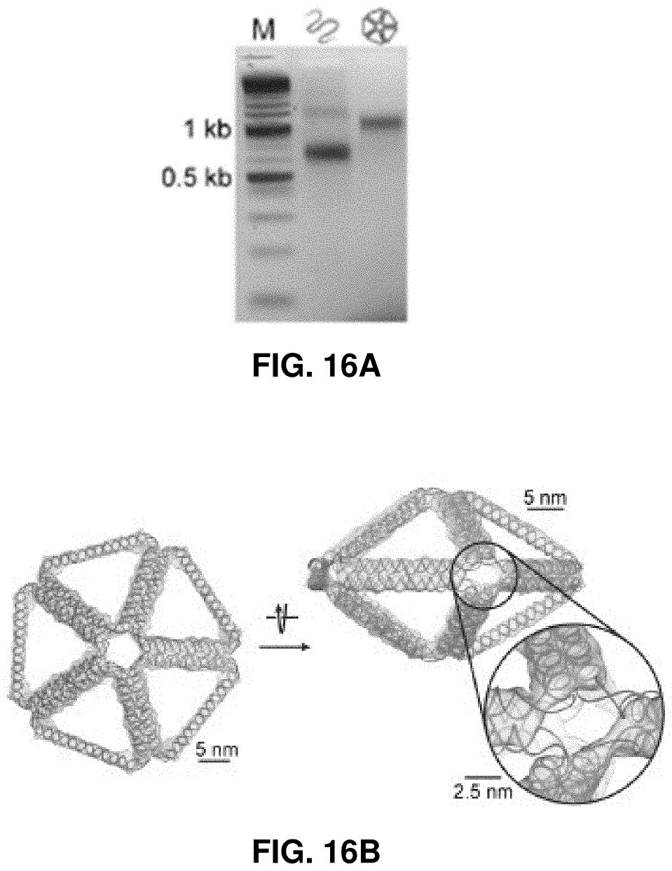

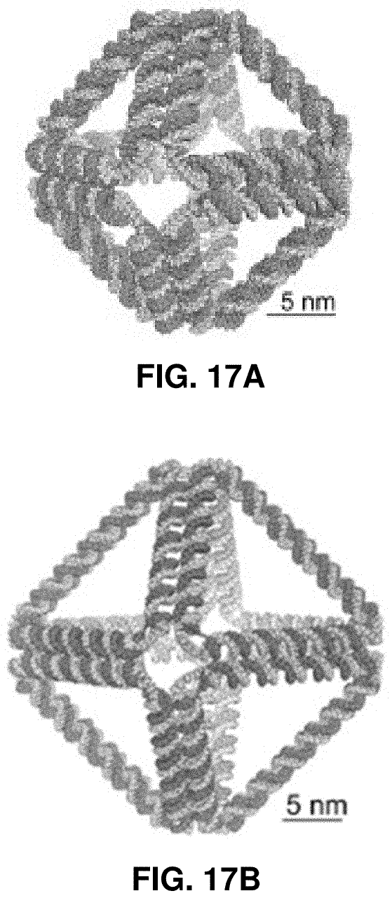

An alternative approach incorporates asymmetry into the scaffold crossover position calculation for A-form (FIG. 6B-6C). In this approach, the scaffold crossover had a 5-nt difference between the nucleotide position on the two helices (e.g., the scaffold crossover on a 33-nt edge would occur 14 nt from the vertex on the 5′ side and 19 nt from the vertex on the 3′ side) (FIGS. 15A-15B, 16A-16B). Manual modifications from B-form designs to the alternative A-form were initially implemented using Tiamat software (Williams, et al. “Tiamat: A Three-Dimensional Editing Tool for Complex DNA Structures. in DNA Computing” (eds. Goel, A., Simmel, F. C. & Sosík, P.) 90-101 (Springer Berlin Heidelberg, 2009)) and subsequently automated. For the tetrahedron and pentagonal bipyramid, an “Hybrid-form” design was also manually generated, which does not have the 5-nt difference in nucleotide position between the two helices, but instead directly crosses over as in the standard DNA design (FIG. 6D, FIGS. 9A-9C).

The A-form design rules with staple crossover asymmetry were implemented in a top-down design algorithm that calculates sequences for folding an input target shape with wireframe DX edges (FIG. 1). The code architecture in pyDAEDALUS (internet site github.com/lcbb/pyDAEDALUSX) mirrors the format and naming conventions of DAEDALUS (Veneziano, et al., Science 352, 1534 (2016)). Briefly, the input target geometry file in the Polygon File Format (PLY) is parsed to identify relevant geometric parameters including coordinates of vertices, edge and face connectivity that form the graph of the shape, and edge lengths. Scaffold routing is achieved by calculating the spanning tree of the graph, and staples are added according to either standard geometric rules for B-form DNA or the A-form design rules, depending on user specification. The resulting outputs of the algorithm are plaintext and Comma-Separated Values (CSV) files that store the routing information, staple sequences, and nucleotide spatial coordinates. The positions and orientations of each nucleotide are represented as vectors following the convention from the software 3DNA (Lu & Olson, Nat Protoc 3, 1213-1227 (2008)). Staple sequences calculated for all structures designed in this study are set forth in Table 1.

While the overall architecture of DAEDALUS is preserved in pyDAEDALUS, several fundamental changes were required. First, the connectivities passed through the functions are stored in pyDAEDALUS as NetworkX Graph objects, rather than sparse matrices as in DAEDALUS.

Second, Prim's algorithm, which is used to generate the spanning trees required to route the scaffold strand, was used in both algorithms as built-in functions. However, for many structures the Python version generates a spanning tree different from the MATLAB version. Although this will affect the scaffold routing and staple sequences, the fidelity of the final design should not be affected, because each possible spanning tree of an object corresponds to a valid scaffold routing. Third, in order to exploit the object-oriented structure that Python enables, the DNAInfo class was introduced, which packages together the many variables associated with the geometry, routing, and structure generated in intermediate sub-functions of the algorithm. To render the code more robust and offer a platform for further development by other contributors, additional frameworks were constructed. A style guide was implemented to help readability of the code, and linting, i.e., automatic checking of adherence to the style guide, is also enforced. In addition, unit tests were introduced to ensure that the functionality of the code is preserved as intended by the original authors. All tests and linting are evaluated automatically on each change to the current version on GitHub, with the results published within the readme.md of the repository.

RNA Transcription

For the RNA scaffolded tetrahedron, the EGFP sequence was generated as a gBlock and cloned with a T7 promoter and Shine-Dalgarno (SD) sequence 5′ of the coding sequence into a pUC19 vector using restriction cloning (EcoRI, Pstl). RNA was transcribed from a Phusion PCR-generated double-stranded DNA (dsDNA) template containing a 5′ T7 promoter, amplified using primers and gel purified. For the pentagonal bipyramid and octahedron with 6 helical turns per edge, primers were chosen flanking Domains I—IV of the rr1B gene encoding the 23S rRNA from the pCW1 plasmid (Weitzmann, et al., Nucleic Acids Res 18, 3515-3520 (1990)). For the octahedron with 4 helical turns per edge, partial M13 DNA template was amplified from mp18 ssDNA (NEB). For the fragment of human immunodeficiency virus 1 (HIV-1) Rev response element (RRE) used as a control in DMS-MaPseq experiments, the sequence was synthesized as a gBlock (IDT) containing a 5′ T7 promoter, then PCR amplified using the Q5 High-Fidelity 2×Master Mix (NEB). For the tetrahedra with 5 and 7 helical turns per edge, a random scaffold sequence was designed with minimal self complementarity (rsc1218v1) and again the sequence was obtained as a synthetic gBlock (IDT), then amplified with Q5 High-Fidelity 2×Master Mix (NEB) to create dsDNA templates for 660 nt and 924 nt scaffolds, each with a 5′ T7 promoter.

Using these dsDNA templates, RNA was transcribed using the manufacturers protocol for HiScribe T7 (NEB) for canonical base RNAs or DuraScribe T7 (Lucigen) for 2′-fluoro-modified base RNAs. RNA was treated with DNase I (NEB), then pre-cleaned on a ZymoClean RNA cleanup kit (RNA Clean-and-concentrator-5). Urea polyacrylamide gel (PAGE) was used to validate purity, and PAGE or HPLC was used to purify the RNAs if byproducts were present. With RNA pre-cleaned using the RNA clean-and-concentrator-5 kit (Zymo), and denatured by addition of 1×RNA loading dye (NEB) and 5-10-minute incubation at 70° C., PAGE purification was performed on a 6% gel containing 8M urea. RNA was sliced from the gel after visualization with SybrSafe (ThermoFisher) and eluted in 300 mM sodium acetate pH 5.2, precipitated in 70% ethanol at −20° C. for >2 hours, and then pelleted at 14,000 RPM for 30 minutes at 4° C.

For HPLC purification, transcribed and column-purified (with ZymoClean RNA clean-andconcentrator-5 kit) RNA was diluted with nuclease-free water and injected into an XBridge Oligonucleotide BEH C18 column (130 Å, 2.5 μm, 4.6 mm×50 mm, Waters) under the following gradient, flowing at 0.9 ml/min: increasing from 38-40% solvent B over 1 minute, increasing to 60% buffer B across 15 minutes, increasing to 66% buffer B across 6 minutes, increasing to 70% buffer B across 30 seconds, reaching 100% buffer B across 30 seconds, maintaining 100% buffer B for 1 minute, decreasing to 38% solvent B over 1 minute, where it was finally held for 2 minutes. Buffer A was a solution containing 0.1 M TEAA, while buffer B included 0.1 M TEAA and 25% (v/v) acetonitrile. All HPLC purification of the RNA scaffold was run at 65° C. to prevent formation of secondary structure. Sodium acetate, pH 5.2, was added to a final concentration of 300 mM in the collected fraction, and the RNA was precipitated in 70% ethanol at −20° C. for >2 hours, then pelleted at 14,000 RPM for 30 minutes at 4° C.

Hybrid RNA/DNA Nanoparticle Folding and Characterization

Using RNase-free buffers and conditions, 20 nM of purified scaffold was mixed with 400 nM individual staples and buffer and salt and brought to 50 μl aliquots for temperature ramping. 10 mM and 50 mM HEPES-KOH pH 7.5 and 50 mM Tris-HCl pH 8.1 were tested, and salt concentrations were tested in 10 mM HEPES-KOH such that the final concentrations of KCl and NaCl individually were 0, 100, 200, 300, 400, and 500 mM, and for MgCl₂: 0, 2, 4, 8, 12, and 16 mM. Folding was performed using a modification of the previously published wireframe origami thermal annealing protocol 14 but with reduced incubation time at high temperatures. Briefly, the folding protocol was 90° C. for 45 s; ramp 85° C. to 70° C. at 45 s/° C.; ramp 70° C. to 29° C. at 15 WV; ramp 29° C. to 25° C. at 10 mrC; 10 m at 37° C.; hold at 4° C. until purification. Folded particles were purified away from excess staples using Amicon Ultra 100 kDa 0.5 ml filter columns and buffer exchanged into the same buffer used for folding.

The size distribution of the origami nanoparticles was measured via DLS using a Zetasizer Nano ZSP (model ZEN5600, Malvern Instruments, UK). Purified nanoparticles were concentrated to 75 nM in 50 μl in 10 mM HEPES-KOH pH 7.5 and 300 mM KCl. The default procedure for DNA was used, only customizing the buffer to include 300 mM KCl. Three serial DLS measurements were performed on the same folded sample at 25° C. The average nanoparticle diameter (nm) and polydispersity index (PdI) were computed using the associated Malvern software (Zetasizer Software v 7.12).

Biochemical stability in the presence of RNases A and H was also tested. 50 nM RNA-scaffolded tetrahedron with 66-bp edge length was incubated for 5 min at 37° C. in the presence of buffer alone, 25 units of RNase H or 3.5 units of RNase A. RNA transcribed with 100% 2′-fluoro-deoxyuridine and cytosine was folded to a tetrahedron with 66-bp edge lengths using the same staples, and also incubated for 5 min at 37° C. in the presence of 3.5 units of RNase A. Reactions were quenched at 4° C. and run at 65V for 180 minutes on a high-resolution 2.5% agarose gel in TBE with 2.5 mM Mg(OAc)2, maintained at 4° C. on ice.

Chemical Probing of Secondary Structure with DMS-MaPseq

The DNA template for each scaffold was generated and amplified as described above. A gene block encoding a 232 nt segment of the HIV-1 Rev Response Element (RRE) with a T7 promoter was ordered from IDT and amplified using Q5 High-Fidelity DNA Polymerase (New England BioLabs) according to the manufacturer's protocol. DNA templates were purified using a QIAquick PCR Purification Kit (Qiagen). The scaffolds and RRE were in vitro transcribed and DNase-treated using the HiScribe T7 High Yield RNA Synthesis Kit (New England BioLabs) according to the manufacturer's protocol, then purified using a Zymo RNA Clean and Concentrator-5 Kit (Zymo Research).

Three denaturing polyacrylamide gels with 6 M urea (2.4 ml 5×Tris-Borate-EDTA (TBE), 4.32 g urea, 1.2 ml 40% 19:1 acrylamide:bis acrylamide, 120 μl ammonium persulfate, 12 μl TEMED, nuclease-free water to 12 ml) were pre-run at 160 volts for 30 min. The RNAs were denatured in 2×RNA Loading Dye (New England BioLabs) at 70° C. for 10 min, immediately placed on ice, and run on the gels at 160 volts for 60 min in 1×TBE in a Mini-PROTEAN Tetra Cell (Bio-Rad). The gels were stained in 1×TBE containing 1×SYBR Safe (Thermo Fisher) for 5 min, and each band of expected molecular weight was excised and transferred to a 0.5 ml tube at the bottom of which a hole had been punctured with a needle. Each 0.5 ml tube was placed in a 1.5 ml tube and spun at 16,000×g for 60 sec to extrude the gel slice into the 1.5 ml tube. Each gel slice was covered with 400 ul of gel elution buffer (250 mM sodium acetate pH 5.2, 20 mM Tris-HCl pH 8.0, 1 mM EDTA pH 8.0, 0.25% w/v sodium dodecyl sulfate) and incubated in a thermomixer at 20° C. for 11 hr while shaking at 500 rpm. The slurries were decanted into Costar Spin-X Centrifuge Tube Filters (Corning) and spun at 16,000×g for 1 min to remove gel particles. To each filtrate, 1 ml 100% ethanol was added, and the tubes were frozen at −80° C. for 1 hr. The tubes were then spun at 12,700×g for 1 hr at 4° C. The pellets were washed with 500 μl 75% ethanol at −20° C. and spun for another 10 min. The supernatants were removed and the tubes uncapped and placed on a 37° C. heat block to dry the pellets for 10-20 min. The pellets containing the RNA were resuspended in 10 μl nuclease-free water.

The gel-purified RNA scaffolds were used to fold nanoparticles in folding buffer (10 mM HEPES-KOH pH 7.5, 300 mM KCl) using 20 nM scaffold and 400 nM for each staple with the temperature steps described above. Each RNA scaffold was also folded using the same protocol but without adding staples. The 23S scaffold was folded in two tubes each containing 85 μl; rT66 without staple 10 was folded in one tube containing 70 μl; all other nanoparticles and scaffolds were folded in two tubes each containing 60 μl.

Folded nanoparticles/scaffolds were purified by five rounds of filtration through Amicon Ultra 100 kDa 0.5 ml filter columns. One Amicon filter for each of the 16 nanoparticle/scaffold samples was first spun at 2,400×g for 30 min at 4° C. with 500 μl of the buffer used to fold the origami. To each pre-spun filter, 350 μl of 300 mM sodium cacodylate pH 7.2 (Electron Microscopy Sciences) was added, followed by 50-150 μl of the pooled folding product of one nanoparticle/scaffold. The samples were spun at 850×g for 30 min at 4° C., after which the filtrate was decanted and 450 μl sodium cacodylate added to the filter, and these steps were repeated for a total of five filtrations.

The fifth filtration was run for 50 min, after which each filter (containing approximately 50 μl) was inverted into a clean collection tube and spun at 1,500×g for 1 min at 4° C. to collect the sample of nanoparticles/scaffold. A 10 μl aliquot of each sample was transferred to a 1.5 ml tube. As a control for normalization of the DMS reactivities, 1.3 μg of gel-purified RRE RNA was denatured in 8 μl of RNase-free water at 95° C. for 60 seconds and immediately placed on ice for 60 seconds. The denatured RRE RNA was mixed with 612 μl of 300 mM sodium cacodylate pH 7.2 (Electron Microscopy Sciences) and incubated at 37° C. for 20 min to refold its structure. A 38.5 μl aliquot of refolded RRE RNA was added into each of 16 tubes containing 10 μl of one nanoparticle/scaffold. To each sample, 1.5 μl of neat dimethyl sulfate (DMS, SUPPLIER) was added (50 μl total volume, 3% DMS v/v), stirred with a pipette tip, and incubated at 37° C. for 5 min in a thermomixer while shaking at 500 rpm. Each reaction was quenched by adding 30 μl neat beta-mercaptoethanol (MilliporeSigma). DMS-modified nucleic acids were purified using a Zymo RNA Clean and Concentrator-5 Kit (Zymo Research) and eluted in 10 μl RNase-free water.

For each RNA sample, 4 μl was reverse transcribed in a 20 μl reaction containing 1 μl pooled reverse primers (10 μM each), 1 μl TGIRT-III enzyme (Ingex), 4 μl 5×First Strand buffer (Invitrogen), 1 μl 10 mM dNTPs (Promega), 1 μl 0.1 M dithiothreitol (Invitrogen), and 1 μl RNaseOUT (Invitrogen). The reactions were incubated at 57° C. in a thermocycler with the lid set to 60° C. for 90 min. The RNA templates were degraded by adding 1 μl of 4.0 M sodium hydroxide (SUPPLIER) to each reaction and incubating at 95° C. for 3 min. Each cDNA was purified using a Zymo Oligo Clean and Concentrator-5 Kit (Zymo Research) and eluted in 10 μl nuclease-free water.

The cDNA from each of the 16 samples was amplified as a set of overlapping amplicons, each 250-556 bp (47 amplicons total), plus one amplicon spanning the entire RRE (16 amplicons total). For each amplicon, 1 μl purified cDNA was amplified with an Advantage HF 2 PCR kit (Takara) in a 25 μl reaction containing 0.5 μl forward primer (10 μM, IDT), 0.5 μl reverse primer (10 μM, IDT), 0.5 μl 50× Advantage-HF 2 Polymerase Mix, 2.5 μl 10× Advantage 2 PCR Buffer, 2.5 μl 10×HF dNTP Mix, and 17.5 μl nuclease-free water. The PCR entailed an initial denaturation step at 94° C. for 60 sec, followed by cycles of 94° C. for 30 sec, 60° C. for 30 sec, and 68° C. for 60 sec, with a final extension at 68° C. for 60 sec. All PCR products were validated using E-Gel EX-Gels with 2% Agarose (Thermo Fisher).

All 47 PCR products from nanoparticles/scaffolds and 5 RRE products were consolidated into 5 pools such that no two amplicons from the same RNA sequence were pooled together. Pools 1-4 contained 6 μl each of 10 PCR products; pool 5 contained 5 μl each of 12 PCR products. For each pool, 30 μl was mixed with 6 μl 6×gel loading dye and run on a 50 ml gel containing 2% SeaKem Agarose (Lonza), 1×Tris-Acetate-EDTA (Boston BioProducts), and 5 μl 10,000×SYBR Safe DNA Gel Stain (Thermo Fisher) at 60 volts for 105 min. Bands at the expected sizes were excised and the DNA extracted using a Zymoclean Gel DNA Recovery Kit (Zymo Research) and eluted in 12 μl 10 mM Tris pH 8.0 (SUPPLIER). DNA libraries were generated and sequenced on an Illumina MiSeq using a 300×300 read length at the MIT BioMicroCenter sequencing core.

Statistical Analysis of DMS Reactivities and Structural Features

DMS-induced mutation rates (“DMS reactivities”) were determined using the Detection of RNA folding Ensembles with Expectation Maximization clustering (DREEM) pipeline (Tomersko, et al. Nature 582, 438-442 (2020)), using the default parameters except for a 90% coverage threshold for clustering. In order to control for variations in DMS treatment among different samples, the DMS reactivities were normalized using a custom script as follows. The median DMS reactivity among the top 50% (n=46) of the 91 adenine (A) and cytosine (C) bases in the spiked-in RRE control was computed for each sample. For each other sample, the ratio of the median DMS reactivity of the RRE to the median DMS reactivity of the RRE in the sample of 23S scaffold without staples (the reference sample) was computed, and the DMS reactivity of the sample was divided by this ratio to normalize it. The software ARIADNE was developed to determine the locations of these structural features using the outputs from pyDAEDALUSX. ARIADNE was written in Python 3.8 and uses NumPy 1.20, Pandas 1.2, Matplotlib 3.3, Seaborn 0.1, and BioPython 1.7. For each origami, ARIADNE was used to generate a table of all nucleotides in the scaffold and staple strands, indicating for each nucleotide the identity of its base (i.e. A, C, G, T, or U) and the identity of the adjacent structural feature (if any), which could be one of seven types: double crossover of the scaffold strand (scaffold DX), 5′ or 3′ terminus of the scaffold stand (scaffold nick), double crossover of the staple strand (staple DX), single crossover (a.k.a. mesojunction) of the staple strand (staple SX), 5′ or 3′ terminus of a staple stand that lies on the same double helix as and adjacent to that stand's 3′ or 5′ terminus (staple nick), 5′ or 3′ terminus of a staple strand that abuts a single crossover of another staple strand (staple single term), or a vertex of the polyhedral origami. Scaffold nicks were excluded from further analysis due to insufficient coverage.

ARIADNE was used to identify all double helical segments, defined as a set of contiguous scaffold-staple base pairs bordered by a structural feature on each end with no structural features in the middle, as in a previous work (Martin & Dietz, Nature Communications 3, 1103 (2012)). The DMS reactivities at the terminal scaffold bases in each segment, the mean DMS reactivity over the interior bases in each segment, their ratios, and the DMS reactivities of bases up to 6 nt upstream and downstream of each structural feature were computed and plotted with a custom Jupyter notebook written in Python 3.8, using Pandas 1.2, Matplotlib 3.3, and Seaborn 0.10. The melting temperature of each segment was predicted using a nearest neighbor model (Sugimoto, et al., FEBS Letters 354, 74-78 (1994)) and salt correction (Wetmur, Critical Reviews in Biochemistry and Molecular Biology 26, 227-259 (1991)) for RNA/DNA duplexes, with 300 mM Na+, implemented in BioPython 1.7. All statistical significance tests were performed using SciPy 1.6 (Virtanen, et al., Nature Methods 17, 261-272 (2020)).

Each analysis was repeated on the DMS reactivities of only adenines and of only cytosines separately. Three microliters of the folded and purified RNA nanostructure solution (approximately 600 nM) was applied onto the glow-discharged 200-mesh Quantifoil 2/1 grid, blotted for four seconds and rapidly frozen in liquid ethane using a Vitrobot Mark IV (Thermo Fisher Scientific). All grids were screened and imaged on a Talos Arctica cryo-electron microscope (Thermo Fisher Scientific) operated at 200 kV at a magnification of 79,000×(corresponding to a calibrated sampling of 1.76 Å per pixel). Micrographs were recorded by EPU software (Thermo Fisher Scientific) with a Gatan K2 Summit direct electron detector in counting mode, where each image is composed of 24 individual frames with an exposure time of 6 s and a total dose −63 electrons per Å2. We used a defocus range of −1.5 to −3 μm to collect images, which were subsequently motion-corrected using MotionCor2 (Zheng, et al. Nat Methods 14, 331-332 (2017)). Single-particle image processing and 3D reconstruction was performed as previously described using the image processing software package EMAN278. All particles were picked manually by e2boxer.py in EMAN2. Resolution for the final maps were estimated using the 0.143 criterion of the Fourier shell correlation (FSC) curve without any mask.

A Gaussian low-pass filter was applied to the final 3D maps displayed in the UCSF Chimera software package (Pettersen, et al., J Comput Chem 25, 1605-1612 (2004)). Correlation of each map with its corresponding atomic model is calculated by the UCSF Chimera fitmap function, with density simulated from the model at the same resolution as the corresponding reconstruction.

Results

Biochemical Characterization to Establish a Folding Protocol

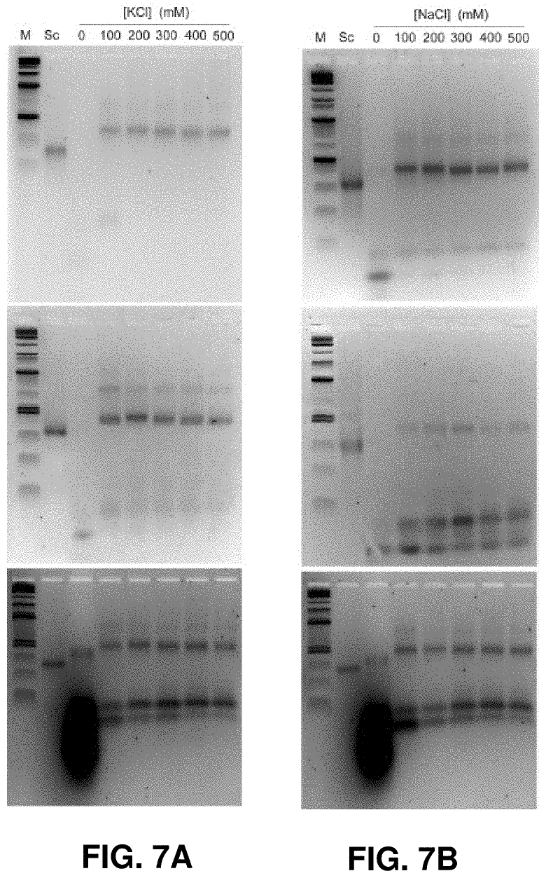

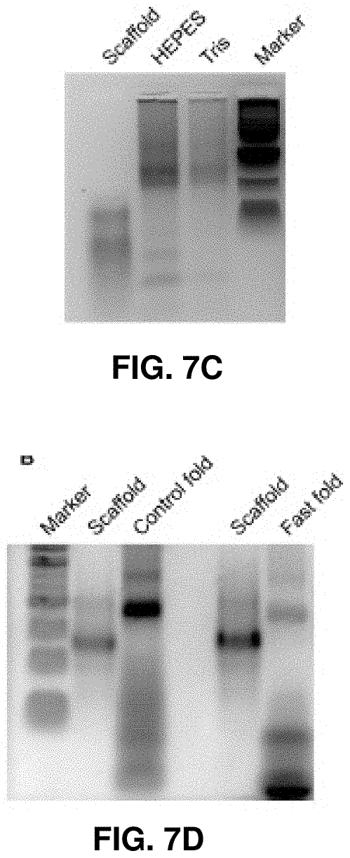

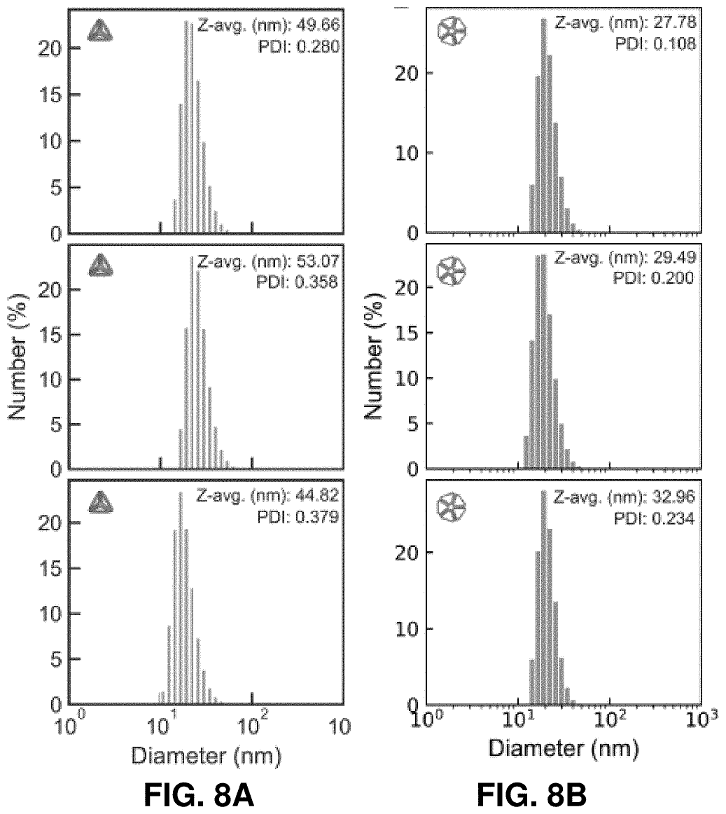

Optimal ionic folding conditions for origami typically vary based on type of design; for example, protocols differ between wireframe and bricklike, six- and two-helix-bundle, and single-stranded RNA- vs. DNA-scaffolded origami (Gerling, et al., Science Advances 4, (2018)). Firstly, the ionic folding conditions for hybrid two-helix-bundle wireframe origami were established. Because the designs presented here are most similar to the DX wireframe designs of DAEDALUS, folding protocols were designed with reduced time at higher temperatures to account for RNA instability. For manual design of an RNA-scaffolded tetrahedron with six helical turns per edge, gel mobility shift assays of KCl and NaCl showed an upward shift of the major band relative to unpaired scaffold, with the band position and discreteness stabilized at 300 mM monovalent salt (FIGS. 7A-7B). These results suggested the origami particles were properly folded in 300 mM monovalent salt and 10 mM HEPES-KOH pH 7.5 using this thermal annealing ramp. No major band was observed when attempting to fold the tetrahedron in magnesium, likely due to RNA degradation from this divalent salt at the high temperatures used during annealing. Consistent with this hypothesis, this effect was mitigated by using previously published fast-folding protocols that use magnesium during folding (FIGS. 7C-7D). Likewise, higher yields of the RNA-scaffolded tetrahedra were achieved in HEPES-KOH pH 7.5 buffer than in Tris-HCl pH 8.1 buffer (FIGS. 7C-7D), possibly due to the combined effects of higher pH and temperature. Dynamic light scattering (DLS) characterization of the tetrahedral origami folded in 300 mM KCl and 10 mM HEPES showed primarily monomeric populations with 33% polydispersity (FIGS. 8A-8B). 300 mM KCl and 10 mM HEPES-KOH pH 7.5 was identified as a suitable buffer for folding hybrid RNA/DNA A-form DX wireframe origami with a 13-hour annealing ramp.

The sequence of the EGFP RNA scaffold was:

(SEQ ID NO: 18)
AAGGGCGAGGAGCUGUUCACCGGGGUGGUGCCCAUCCUGGUCGAGCUGG
ACGGCGACGUAAACGGCCACAAGUUCAGCGUGUCCGGCGAGGGCGAGGG
CGAUGCCACCUACGGCAAGCUGACCCUGAAGUUCAUCUGCACCACCGGC
AAGCUGCCCGUGCCCUGGCCCACCCUCGUGACCACCCUGACCUACGGCG
UGCAGUGCUUCAGCCGCUACCCCGACCACAUGAAGCAGCACGACUUCUU
CAAGUCCGCCAUGCCCGAAGGCUACGUCCAGGAGCGCACCAUCUUCUUC
AAGGACGACGGCAACUACAAGACCCGCGCCGAGGUGAAGUUCGAGGGCG
ACACCCUGGUGAACCGCAUCGAGCUGAAGGGCAUCGACUUCAAGGAGGA
CGGCAACAUCCUGGGGCACAAGCUGGAGUACAACUACAACAGCCACAAC
GUCUAUAUCAUGGCCGACAAGCAGAAGAACGGCAUCAAGGUGAACUUCA
AGAUCCGCCACAACAUCGAGGACGGCAGCGUGCAGCUCGCCGACCACUA
CCAGCAGAACACCCCCAUCGGCGACGGCCCCGUGCUGCUGCCCGACAAC
CACUACCUGAGCACCCAGUCCGCCCUGAGCAAAGACCCCAACGAGAAGC
GCGAUCACAUGGUCCUGCUGGAGUUCGUGACCGCCGCCGGGAUCACUCU
CGGCAUGGACGAGCUGUACAAGUAACUGCAGGCAUGCAAGCUUGGCGUA
AUCAUGGUCAUAGCUGUUUCCUGUGUGAAAUUGUUAUCCGCUCACAAUU
CCACACAA.

Polyhedral Origami Folded with Diverse RNA Scaffolds

To investigate the impact of RNA sequence on 3D wireframe origami folding, the ability of three types of RNA sequences to scaffold A-form DX wireframe origami was examined an mRNA encoding bacterial GFP; a de Bruijn sequence designed to have minimal self-complementarity and repetition; and a synthetic transcript of the M13 viral genome, which is a DNA sequence frequently used to scaffold DNA origami. For an initial test we targeted design and fabrication of a regular tetrahedral geometry, with six edges of equal length and four three-way vertices.

In vitro-transcribed 792-nt prokaryotic EGFP mRNA and 660-nt and 924-nt de Bruijn RNA sequences were used to scaffold tetrahedra with six, five, and seven helical turns per edge, respectively (rT66, rT55, and rT77). To accommodate A-form helical geometry, the DX edges were designed with asymmetry in the staple crossover calculation, and 11 nt per helical turn (FIG. 1, FIG. 6B-6C).

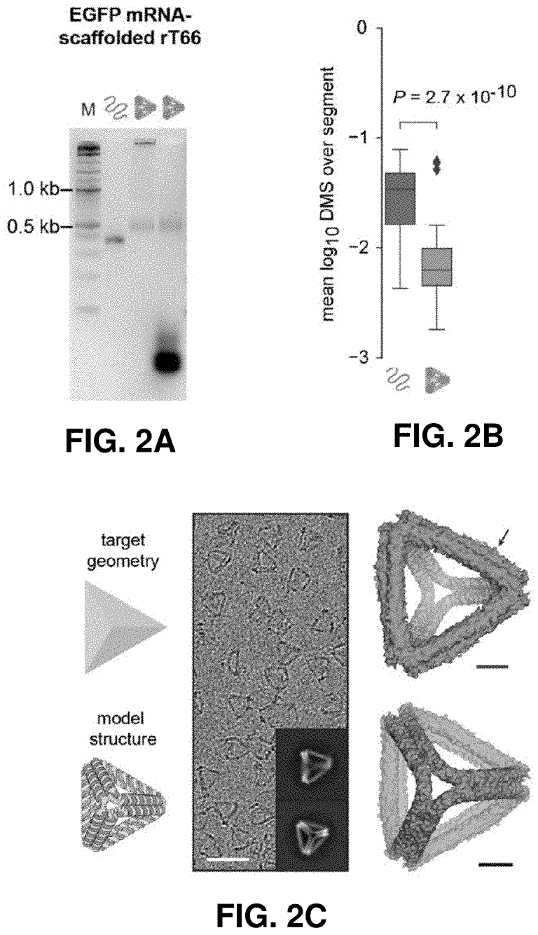

Having determined biochemically that these RNA-scaffolded tetrahedra hybridized to staples and folded compactly, the tertiary structure was characterized with cryo-electron microscopy (cryo-EM). Cryo-EM micrographs for the rT66 (FIG. 2C) showed monodisperse tetrahedral particles, with the reconstruction at 12 Å resolution showing slightly bowed edges, correlating overall with the target atomic model with a correlation coefficient of 0.76.

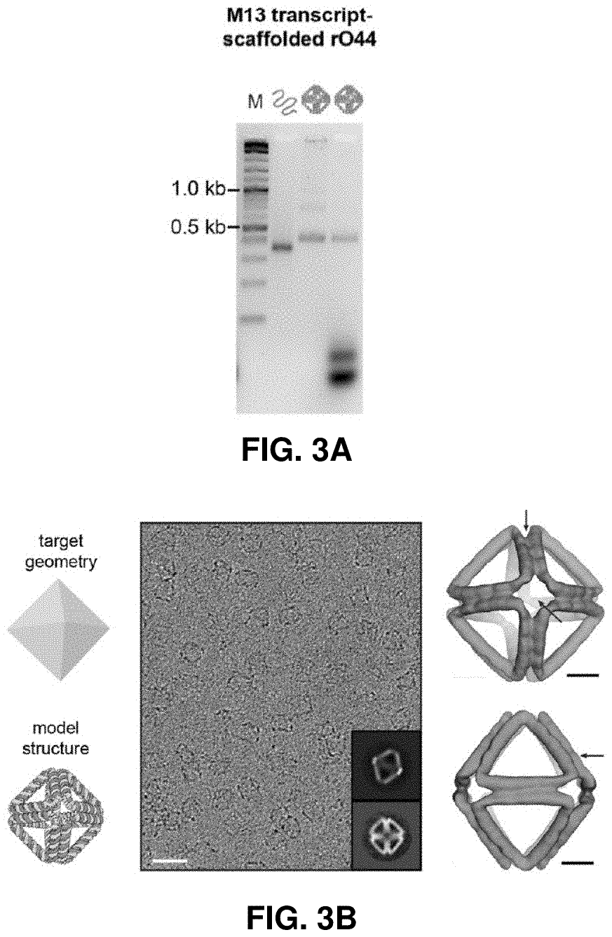

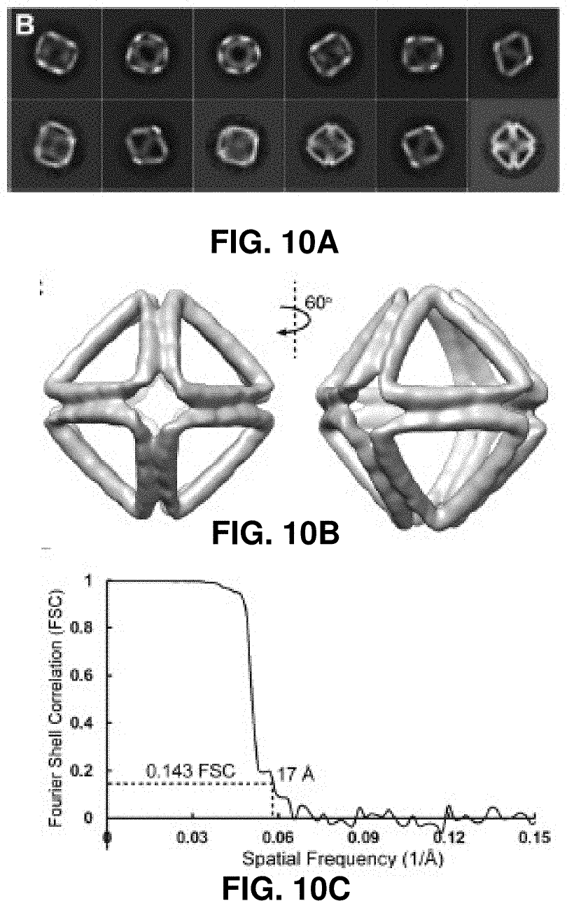

Turning to a more complex geometry with twelve edges of equal length and six four-way vertices, we used a 1056-nt transcript of the M13 phage genome to scaffold a regular octahedron with four helical turns per edge (rO44). The M13 transcript-scaffolded rO44 likewise formed a discrete band with lower electrophoretic mobility than the scaffold in a gel mobility shift assay (FIG. 3A), and cryo-EM screening showed monodisperse octahedral particles (FIG. 3B, FIGS. 9A-9C and 10A-10C). Reconstruction from the cryo-EM data achieved a resolution of 17 Å, and the map had a 0.90 correlation with the predicted atomic model. Unlike the longer-edged rT66, bowed edges were not evident in the reconstructed rO44. Both the rT66 and the rO44 reconstructions suggested edge lengths corresponding to an average helical rise of 0.29 nm/bp (approximately 12.8 nm per edge for the rO44 and 18.9 nm per edge for the rT66, as measured in UCSF ChimeraX (Pettersen, et al., Protein Science 30, 70-82 (2021)), which is 11% larger than the canonical A-form rise of 0.26 nm/bp and 8% larger than the simulated energy-minimized A-form rise of 0.267 nm/bp50. Although still generally consistent with expectations for A-form helices, this increase in average rise might indicate that the helices are slightly underwound (Liebl, et al., Nucleic Acids Research 43, 10143-10156 (2015)), or that the crossover junction geometry creates space that modestly lengthens the edge. For both the rT66 and rO44, we note that the two duplexes in an edge modestly twisted or sheared relative to one another (FIGS. 2A-2C, 3A-3B, 4C and 4E). This distortion might indicate an inability of the A-form twist to relax fully in these structures, which is supported by molecular dynamics simulations (Naskar, et al., Nanoscale 11, 14863-14878 (2019)).

23s-rRNA-Scaffolded Origami

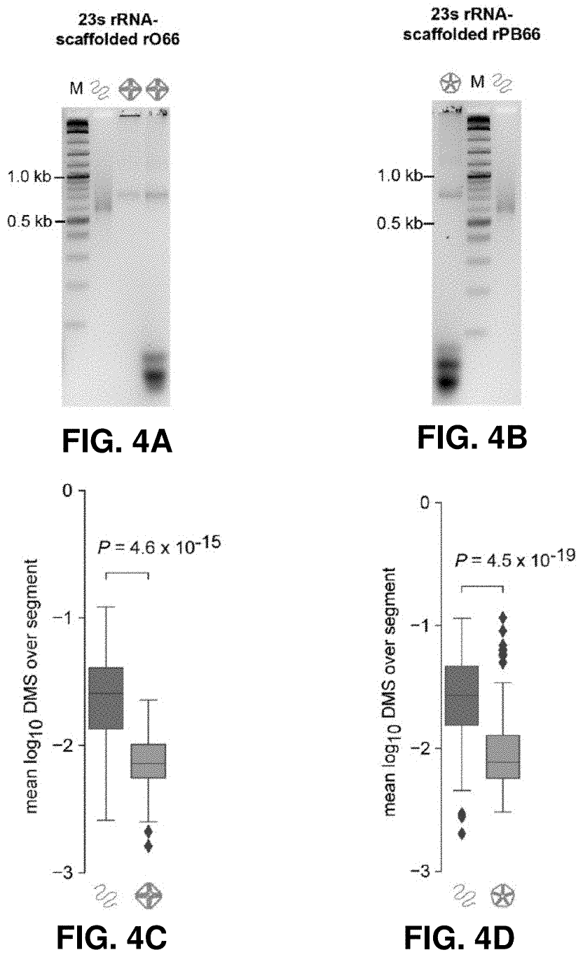

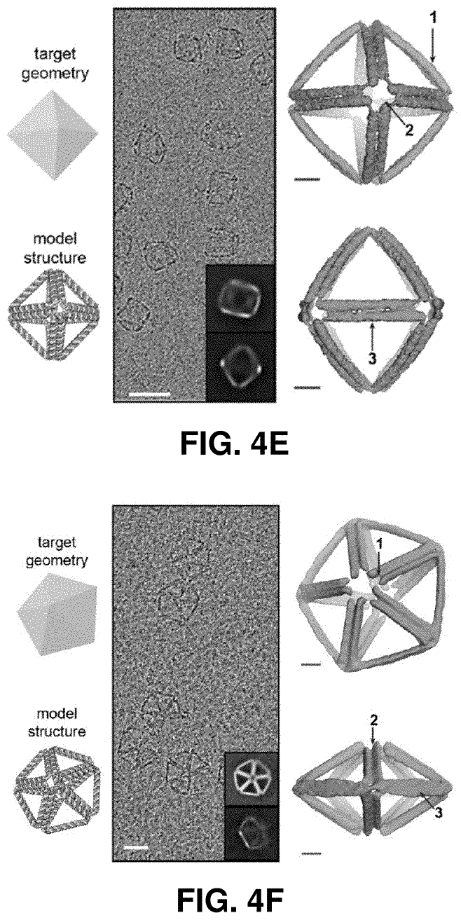

Having validated folding of A-form DX wireframe origami with regular geometries and scaffolds, folding was attempted with the larger, more structurally organized and most abundant naturally occurring scaffold on earth, ribosomal RNA (rRNA) (Palazzo & Lee, Frontiers in Genetics 6, (2015)). Its use as a scaffolding material for wireframe origami may offer both inexpensive scaffold source for scalable synthesis, as well as promise for application to study RNA-mediated catalysis (Wamhoff, et al., Annual Review of Biophysics) doi:10.1146/annurev-biophys-052118-115259 (2019)). The ability of an in vitro transcribed 1980-nt fragment of the E. Coli 23s rRNA to scaffold two different A-form DX wireframe origami objects of varying complexity was therefore tested: a regular octahedron and a pentagonal bipyramid, each with six helical turns per edge (rO66 and rPB66, respectively), as well as a pentagonal bipyramid with five helical turns per edge (rPB55). Unlike the other geometries tested, the rPB66 has multiple vertex types, with both four-way and five-way vertices and corresponding variation in dihedral angles, making it a more complex target geometry in addition to using the longer, inherently structured scaffold. Gel mobility shift assays showed each folded object formed a discrete band shifted slightly upwards from the scaffold band, suggesting compactly folded Objects (FIGS. 4A and 4B). Gel mobility shift assays were also used to assess folding of RNA-scaffolded origami with odd edge lengths, all with the staple asymmetry design. The Pentagonal bipyramid with five helical turns per edge (rPB55), folded from 23s rRNA fragment scaffold, formed a smear in agarose gels with salt, likely due to the formation of secondary structure, but showed a single band on denaturing PAGE gels. The tetrahedron with five helical turns per edge, folded from with ‘rsc1218v1_T55’ synthetic RNA fragment scaffold showed a shift, as did the Tetrahedron with seven helical turns per edge, folded with ‘rsc1218v1_T77’ synthetic RNA fragment scaffold.

As additional evidence of proper folding of the 23s-scaffolded origami, the median normalized DMS reactivities among their double helical segments was lower than that of the 23s fragment scaffold folded without staples (72% lower for rO66, 71% lower for rPB66) (FIGS. 4C and 4D), indicating that the scaffolds hybridized to their staples in the folded origami. In further support of specific staple hybridization, the rO66 design only has staples hybridized to the first 1584 nucleotides of the scaffold, and we determined that the DMS reactivities in the excess scaffold region were well-correlated with those in the same region for the scaffold folded without staples (Pearson Correlation Coefficient=1.0), rather than being depressed.

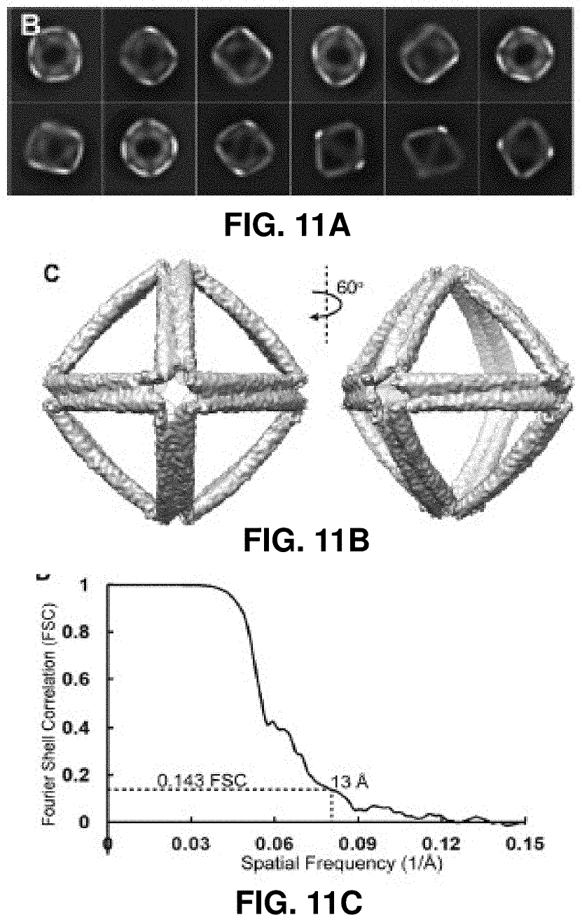

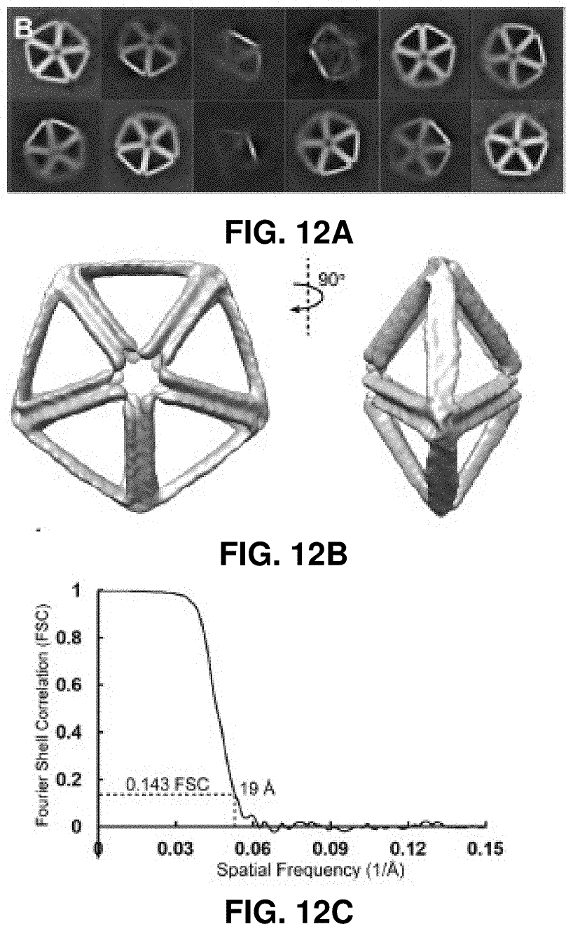

With biochemical evidence of folding, we proceeded to use cryo-EM to characterize the tertiary structures of the rRNA-scaffolded origami. The cryo-EM micrographs for each particle showed well-folded, monodisperse particles (FIGS. 4E-4F, 11A-11C and 12A-12C).

The rO66 reconstruction achieved 13 Å resolution and had a correlation of 0.85 with the predicted atomic model. The reconstruction of the rPB66 achieved 19 Å resolution, and the resulting density map fits with the predicted atomic model with a correlation of 0.92. In the cryo-EM reconstructions for both objects, a slight twisting or shearing of the two duplexes that make up an edge was again observed. The rO66 edges showed some outward bowing, which is not apparent in the rPB66 density map. The edges of the rO66 were approximately 17.9 nm long, as measured in UCSF ChimeraX, corresponding to an average rise of 0.271 nm/bp. The rPB66 has edges averaging approximately 17.8 nm long, corresponding to an average rise of 0.269 nm/bp. These values are consistent with the canonical A-form rise of 0.26 nm/bp and the simulated energy-minimized A-form rise of 0.267 nm/bp50. In both the rO66 and rPB66 reconstructions, an offset in the helical ends of a DX edge at vertices was observed, likely corresponding to the pitch of A-form helices and the asymmetry in staple crossover design.

Alternative Routing Designs

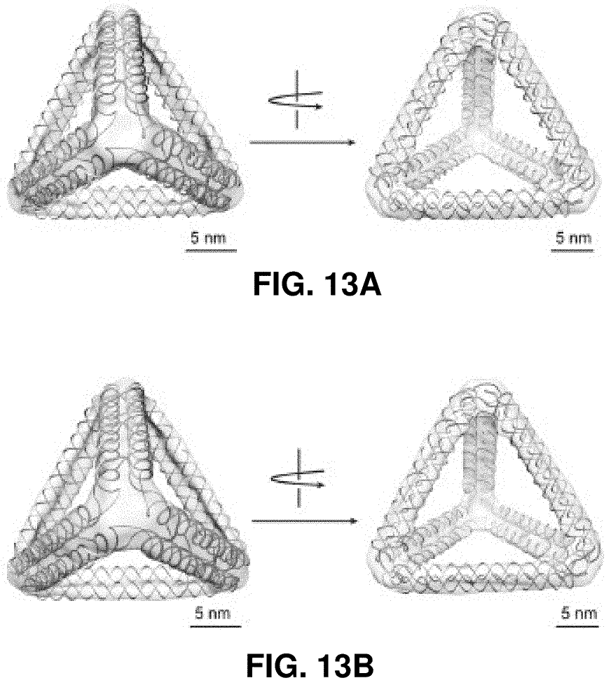

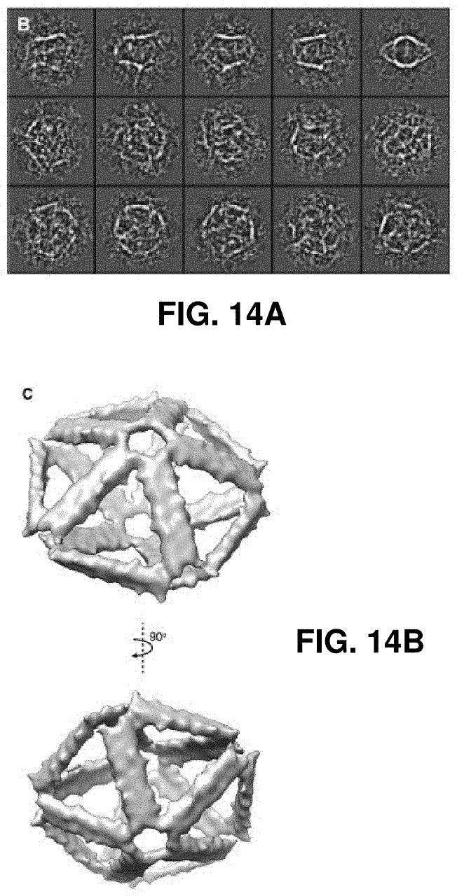

Aside from the A-form design implemented for the origami described above, with 11 nt per helical turn and asymmetry in the staple crossover positions, we tested additional routing schemes, similar to those applied in prior hybrid RNA-DNA origami contexts, for the RNA-scaffolded DX wireframe origami: a B-form design, with no crossover asymmetry and 10.5 bp per helical turn; an “Hybrid-form” design with no crossover asymmetry and 11 nt per helical turn; and an alternative A-form design, with asymmetry incorporated into the scaffold crossover calculation and 11 nt per helical turn (FIG. 6B-6C). The latter design maintained the asymmetrical spacing between adjacent scaffold and staple crossovers on neighboring helices that was suggested from models and implementations of A-form DX routing in RNA-scaffolded designs 43, with the asymmetry incorporated into the scaffold instead of the staple crossover positions. The EGFP-mRNA-scaffolded Hybrid-form rT66 showed high folding yield, with cryo-EM micrographs showing well-formed tetrahedral particles, and a reconstruction yielding a density map with 0.96 alignment correlation with the predicted model (FIGS. 13A-B). However, folding the EGFP mRNA scaffold using staples designed for a B-form fold showed a notably higher gel band shift, and cryo-EM micrographs did not show folded tetrahedron-shaped particles. The alternative A-form rT66 and the rPB66 designed with the scaffold crossover asymmetry behaved similarly to their staple asymmetry A-form designs above, with the same electrophoretic mobility and median DMS reactivity, and showing well-folded particles in cryo-EM imaging (FIGS. 14A-14B, 15A-15B, 16A-16B).

Base-Pair-Level Insight into Origami Stability Using DMS-MaPseq

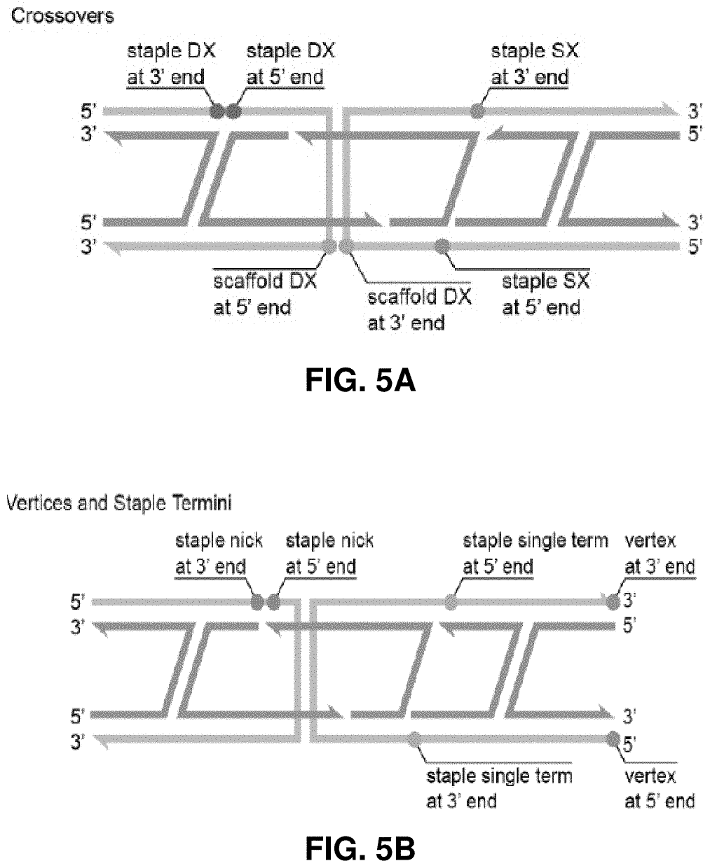

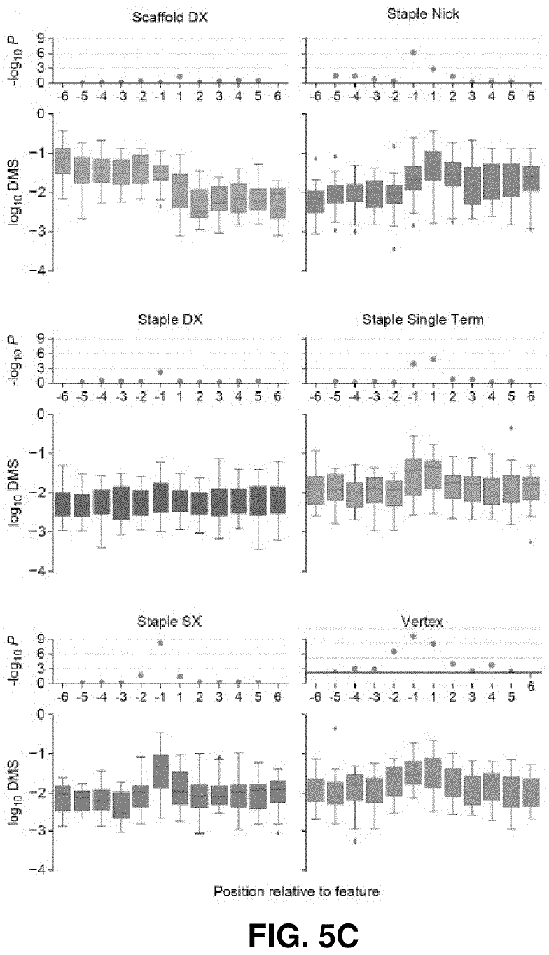

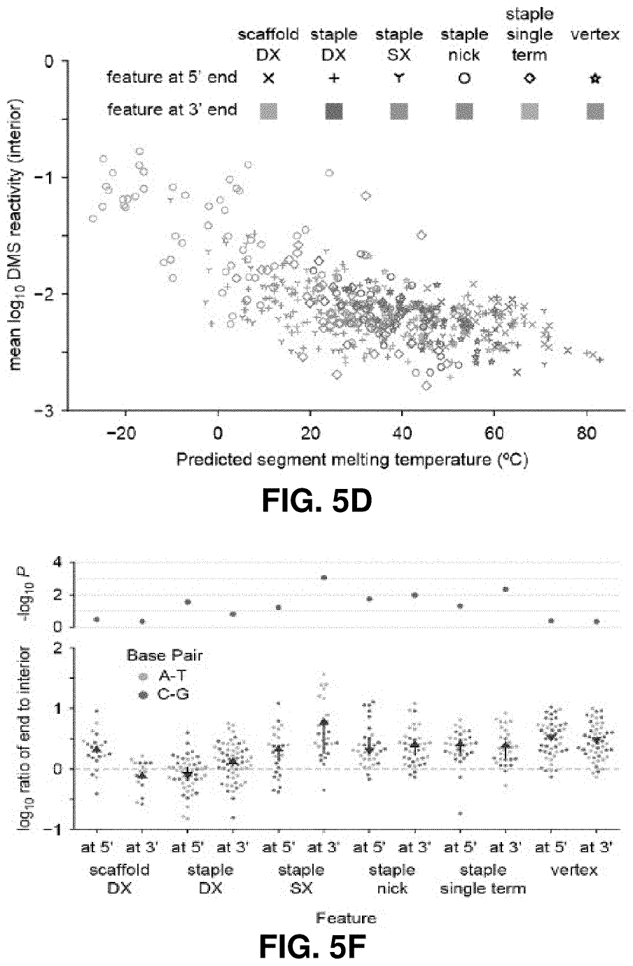

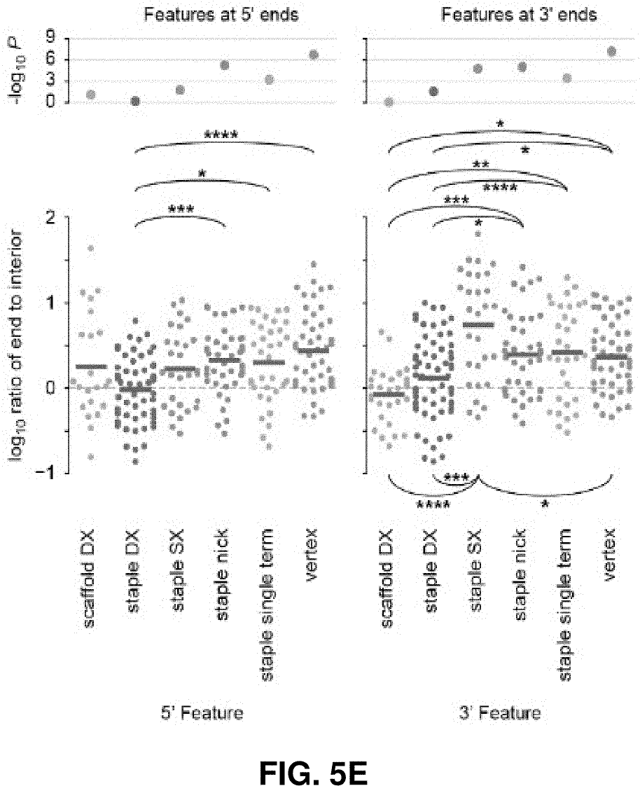

To gain base-level insight into how the placement of crossovers, strand termini, and vertex designs impact RNA scaffolded origami stability, DMS-MaPseq data with base-level resolution were analyzed (FIGS. 5A-5B). For each type of structural feature, we identified all instances of the structural feature among five characterized origami objects (rT55, rT66, rT77, rO66, and rPB66), then computed the distribution of DMS reactivities at each position from 6 bp upstream to 6 bp downstream of the feature instance (FIG. 5C). 6 bp was chosen because it was the length of the shortest contiguous double helical segment in the origami objects examined here. For double and single staple crossovers, staple termini, and vertices, at least one of the bases immediately upstream or downstream of the structural feature (positions −1 and +1, respectively) was more DMS reactive than the corresponding bases further upstream or downstream (positions −6 to −2 and +2 to +6, respectively) at a significance level of 0.01 (one-tailed Mann Whitney U test).

However, by the same criteria for significance, none of these more distant bases was more reactive than corresponding bases further away from the structural feature, except for bases two positions upstream of vertices (P=5×10-5, one-tailed Mann Whitney U test). Thus, crossovers and staple termini destabilized only the immediately adjacent bases, and vertices only bases within two positions.

To account for any biases caused by differences in innate DMS reactivities between adenines and cytosines, we repeated the analysis on each type of residue separately. For both adenines alone and cytosines alone, bases further than 1 nt from a structural feature did not have elevated DMS reactivities, except for bases 2 nt upstream of vertices. For adenines alone, the reactivities of bases adjacent to structural features were generally larger and the P-values generally smaller than for cytosines alone. This result suggests that stabilities of A-T base pairs depend more on their proximities to structural features than do C-G base pairs.

Uniquely among the structural features, scaffold double crossovers showed elevated DMS reactivities at all 6 positions upstream, although bases immediately upstream (position −1) were no more reactive than those further upstream (FIG. 5C). All scaffold double crossovers were placed downstream of a staple nick by 6 or 7 bp—the shortest distance between two structural features in the origami. Thus, we hypothesized that the low melting temperatures of these short double helical segments resulted in the lower tendencies to hybridize. In support of this hypothesis, among all contiguous double stranded segments in the origamis, mean DMS reactivity among the interior bases (excluding the 5′ and 3′ ends of the segment) was moderately anticorrelated with predicted melting temperature (p=−0.58, P=3×10-45; two-tailed one-sample t-test) (FIG. 5D), as well as with segment length (p=−0.41, P=3×10-21) and GC content (p=−0.41, P=1×10-21) (Supplementary FIG. 19). Furthermore, in longer segments with staple nicks at their 5′ ends, interior bases had low DMS reactivities (FIG. 4D), suggesting that the 5′ staple nick was not the main cause of the high DMS reactivities in the 6 or 7 bp segments. We could not deconvolute the contributions of 3′ scaffold crossovers and short segment lengths, but given that the effects of all other structural features were limited to a one- or two-nucleotide vicinity (FIG. 5C) and that segments with 5′ scaffold crossovers generally had low DMS reactivities, we suspect that the 3′ scaffold crossovers are not the primary cause of the increased DMS reactivities of the interior nucleotides.

The relative stabilities of different structural features were then investigated. To determine the effect of each structural feature on local stability when located at the 5′ (or 3′) end of a segment, while controlling for factors that could affect the entire segment (e.g., melting temperature), we divided the DMS reactivity of the base immediately downstream (or upstream) of the structural feature by the mean DMS reactivity among the interior bases in the adjacent segment (FIG. 5E). Bases adjacent to staple single crossovers, staple termini, and vertices, as well as immediately upstream of staple double crossovers, all tended to be more DMS reactive than the interiors of the adjacent segments at a significance level of 0.05 (one-tailed Wilcoxon signed-rank test). Staple termini (both nicks and single termini) and vertices all destabilized local hybridization approximately equally, and more than staple and scaffold double crossovers (FIG. 5E). Previous work with DNA origami motifs has shown that meso-junctions, the geometry adopted by single crossovers in DX edges, are less thermally stable than the conventional junctions of double crossover motifs (Schneider & Dietz, Science Advances 5, (2019); Wang & Seeman, Biochemistry 34, 920-929 (1995)). The data also support this, as DMS reactivities (relative to interior bases) were greater for bases immediately upstream and downstream of staple single crossovers than double crossovers (P=2×10⁻⁵ and P=1×10⁻², respectively, two-tailed Mann-Whitney U test).

To glean insights for optimizing the sequences of the RNA scaffolds, differences between adenines and cytosines adjacent to each structural feature were investigated (FIG. 5F). Most notably, for segments with a 3′ staple single crossover, adenine residues at the crossover were nearly 4 times as DMS reactive (relative to interior adenines) than cytosine residues were (relative to interior cytosines) (P=1×10⁻³, two-tailed Mann-Whitney U test). Similarly, terminal adenines (relative to interior adenines) were more reactive than terminal cytosines (relative to interior cytosines) for segments with 3′ staple single termini and staple nicks. This finding suggests that A-T pairs immediately upstream of staple single crossovers, single termini, and nicks are particularly unstable, and that the origami could be stabilized by replacing them with C-G pairs. Conversely, terminal cytosines were approximately 2-fold more reactive than terminal adenines (relative to interior residues of the same type) for segments with 5′ staple nicks and 5′ staple double crossovers. Relative to interior bases of the same type, there were no significant differences in the reactivities of adenines versus cytosines adjacent to scaffold double crossovers and vertices. These results show that, after controlling for the type of residue and DMS reactivity of each segment, structural features destabilize adjacent A-T and C-G base pairs to different extents, which has implications for optimizing the sequence of the scaffold.

Top-Down A-Form DX Wireframe Origami Design Software

The staple asymmetry design for DX wireframe origami was incorporated as the A-form option in the open-source pyDAEDALUSX software, which generates scaffold and staple routing for polyhedral mesh surfaces to design DX-edge-based wireframe structures. Outputs from the software include a text file with the staple sequences required to fold the target geometry, as well as a 3D structure prediction following 3DNA convention (FIG. 1). As a test of the algorithm's performance, we generated designs for polyhedra with several different RNA sequences as the scaffold and compared the input geometry to the output predicted atomic model. The algorithm's predicted structures for its A-form scaffold and staple routing scheme match well with a range of input geometries, and a variety of sequence types. Biochemical stability of the scaffold-asymmetry designed EGFP mRNA-scaffolded tetrahedron with 66-bp edge length, using canonical nucleotides was characterized in triplicate, with RNase A-, RNase H-, and DNase I-nuclease treatment of the folded rT66 for 5 min at 37° C. Two of the three DNase I replicates showed the intact folded origami band.

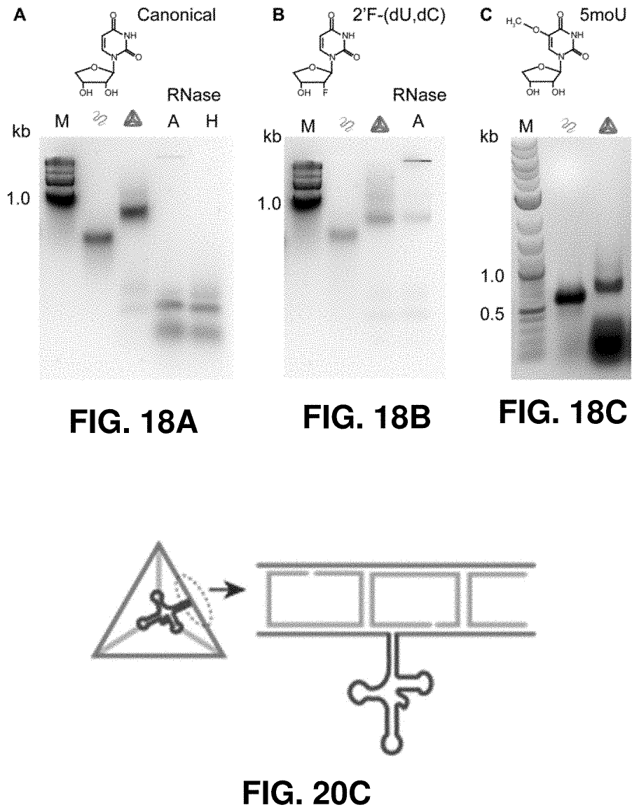

Biochemical stability of the scaffold-asymmetry-designed EGFP mRNA-scaffolded tetrahedron with 66-bp edge length modified with 2′-fluorinated deoxy-uridine and cytosine was characterized in presence of nucleases (FIGS. 18A-18C). Canonical unmodified nucleotides were used to fold nanoparticles and were subjected to treatment with RNase A and RNase H for 5 minutes at 37° C., showing complete degradation of the scaffold and release of the DNA staples (FIG. 18A). 2′-fluorinated deoxy-uridine and cytosine were folded using the A-form geometry staples, and additionally subjected to RNase A treatment, showing no detected degradation in the five minutes it took the unprotected nanoparticle to degrade, though a notable accumulation in the well suggests RNase binding without cleavage (FIG. 18B). 5-methoxyuridine can also fold to a single band using A-form staples (FIG. 18C).

TABLE 1
Staple sequences

| Staple Name | Sequence |
|---|---|
| rT66_EGFP_scaffold | GGTCGGGGTAGAGCTCCTCGCCCTTTTGTGTGGCTGCTTCATGT (SEQ ID |
| asymmetryAform_st1 | NO: 19) |
| rT66_EGFP_scaffold | CTGCACGCCGTGATGGGCACCACCCCGGTGAACCGGCTGAAGCA (SEQ ID |
| asymmetryAform_st2 | NO: 20) |
| rT66_EGFP_scaffold | CAGCTATGACCGTCGCCGATGGGGGTGTTCTGCCACACAGGAAA (SEQ ID |
| asymmetryAform_st3 | NO: 21) |
| rT66_EGFP_scaffold | AAGCTTGCATGTGTCGGGCAGCAGCACGGGGCCATGATTACGCC (SEQ ID |
| asymmetryAform_st4 | NO: 22) |
| rT66_EGFP_scaffold | CCAGCTTGACGTTGTGGCTGTTGTAGTCTTTGC (SEQ ID NO: 23) |
| asymmetryAform_st5 | |
| rT66_EGFP_scaffold | TCAGGGCGTCGCGCTTCTCGTTGGGGTTGTACT (SEQ ID NO: 24) |
| asymmetryAform_st6 | |
| rT66_EGFP_scaffold | TGCCCCAGGATCCATGATATAG (SEQ ID NO: 25) |
| asymmetryAform_st7 | |
| rT66_EGFP_scaffold | TCGATGTTGTGTCCTGGACGTAGCCTTCGGGCACGCTGCCGTCC (SEQ ID |
| asymmetryAform_st8 | NO: 26) |
| rT66_EGFP_scaffold | AGTTCACCTTGGTCCTTGAAGAAGATGGTGCGCGCGGATCTTGA (SEQ ID |
| asymmetryAform_st9 | NO: 27) |
| rT66_EGFP_scaffold | CTGAACTTATCGCCCTCGCCCTCGCCGCCGGCG (SEQ ID NO: 28) |
| asymmetryAform_st10 | |
| rT66_EGFP_scaffold | GCGGTCACGTCCATGCCGAGAGTGATCGACACG (SEQ ID NO: 29) |
| asymmetryAform_st11 | |
| rT66_EGFP_scaffold | GTGGCCGTTTACCGTAGGTGGC (SEQ ID NO: 30) |
| asymmetryAform_st12 | |
| rT66_EGFP_scaffold | CCCTCGAACTCGATGCGGTTCACCAGGTTGCCG (SEQ ID NO: 31) |
| asymmetryAform_st13 | |
| rT66_EGFP_scaffold | GTGGTGCAGGGCCAGGGCACGGGCAGCGTGTCG (SEQ ID NO: 32) |
| asymmetryAform_st14 | |
| rT66_EGFP_scaffold | CTTCACCTCGGATGCCCTTCAG (SEQ ID NO: 33) |
| asymmetryAform_st15 | |
| rT66_EGFP_scaffold | TGGTAGTGGTCTTTTTGGCGAGCTGCATGGCGGACTTGTTTTTAAGAAGT |
| asymmetryAform_st16 | CGTGAATTGTGAGCGTTTTTGATAACAATTT (SEQ ID NO: 34) |
| rT66_EGFP_scaffold | ATGCCGTTCTTTTTTTCTGCTTGTCGGGTTGCCGTCCTTTTTTCCTTGAA |
| asymmetryAform_st17 | GTCGCGCGGGTCTTGTTTTTTAGTTGCCGTC (SEQ ID NO: 35) |
| rT66_EGFP_scaffold | GAACTCCAGCATTTTTGGACCATGTGAGACTGGGTGCTTTTTTCAGGTAG |
| asymmetryAform_st18 | TGGTCCTGCAGTTACTTTTTTTGTACAGCTC (SEQ ID NO: 36) |
| rT66_EGFP_scaffold | GATGAACTTCATTTTTGGGTCAGCTTGCGTCGCCGTCCTTTTTAGCTCGA |
| asymmetryAform_st19 | CCAGAGGTCAGGGTGTTTTTGTCACGAGGGT (SEQ ID NO: 37) |
| rT66_EGFP_Hform_st1 | CGCTCCTGGAACTTGTGGCCGTTTACCCTCGCCG (SEQ ID NO: 38) |
| rT66_EGFP_Hform_st2 | GACACGCTGACGTAGCCTTCGGGCAGATGGTG (SEQ ID NO: 39) |
| rT66_EGFP_Hform_st3 | TGTCGGGCAGCTTTTTTTAGCACGGGGCCGCCGTAGGTGGTTTTTTTCAT |
| | CGCCCTCGCGTCGCCGTCCTTTTTTTAGCTCGACCAG (SEQ ID NO: 40) |
| rT66_EGFP_Hform_st4 | CGGTGAACGACTGGGTGCTCAGGTAGTGGTGATGGGCACCACCC (SEQ ID |
| | NO: 41) |
| rT66_EGFP_Hform_st5 | TTCAGGGTCAGCTTGTCGCCGATGGGGGTGTTCTGCAGATGAAC (SEQ ID |
| | NO: 42) |
| rT66_EGFP_Hform_st6 | ATGGCGGACTTGTCCTTGAAGA (SEQ ID NO: 43) |
| rT66_EGFP_Hform_st7 | TTGCTCAGGGCGAGCTCCTCGCCCTTTTGTGTGGGTTGGGGTCT (SEQ ID |
| | NO: 44) |
| rT66_EGFP_Hform_st8 | TTGCCGGTGGTGCTGGTAGTGGTCGGCGAGCTGCAACGGGCAGC (SEQ ID |
| | NO: 45) |
| rT66_EGFP_Hform_st9 | CGCTGCCGTCCTTTTTTTTCGATGTTGTGTGCTTGTCGGCTTTTTTTCAT |
| | GATATAGAGGTCACGAGGGTTTTTTTTGGGCCAGGGC (SEQ ID NO: 46) |
| rT66_EGFP_Hform_st10 | CCGTTCTTCGCGGATCTTGAAGTTCATCCCGGCG (SEQ ID NO: 47) |
| rT66_EGFP_Hform_st11 | GCGGTCACTCCATGCCGAGAGTGACCTTGATG (SEQ ID NO: 48) |
| rT66_EGFP_Hform_st12 | GAACTCCAGCATGTACAGCTCG (SEQ ID NO: 49) |
| rT66_EGFP_Hform_st13 | GCTGAAGCCTCCAGCTTGTGCCCCAGGATGTGGTCGGGGTAGCG (SEQ ID |
| | NO: 50) |
| rT66_EGFP_Hform_st14 | TTGCCGTCCTCTTTTTTTCTTGAAGTCGACGCGGGTCTTGTTTTTTTTAG |
| | TTGCCGTCGAAGAAGTCGTTTTTTTTGCTGCTTCATG (SEQ ID NO: 51) |
| rT66_EGFP_Hform_st15 | CCCTCGAATCGATGCGGTTCACCATGACCATG (SEQ ID NO: 52) |
| rT66_EGFP_Hform_st16 | ATTACGCCACACACAGGAAACAGCTAGGGTGTCG (SEQ ID NO: 53) |
| rT66_EGFP_Hform_st17 | AGCTTGCATGCTTTTTTTCTGCAGTTACTGGACCATGTGATTTTTTTTCG |
| | CGCTTCTCAATTGTGAGCGTTTTTTTGATAACAATTT (SEQ ID NO: 54) |
| rT66_EGFP_Hform_st18 | CTTCACCTCGGTGCCCTTCAGC (SEQ ID NO: 55) |
| rT66_EGFP_Hform_st19 | TAGTTGTAACTGCACGCCGTAGGTCAGGGTCGTTGTGGCTGTTG (SEQ ID |
| | NO: 56) |
| rPB66_23S_scaffold | ACAAAAGGTACCTGACTGCCAGGGCTGAGTCTCTGACCCATTAT (SEQ ID |
| asymmetryAform_st1 | NO: 57) |
| rPB66_23S_scaffold | CCTAAGCGTGCATTAGCACGTCCTTCATCGCCTGCAGTCACACG (SEQ ID |
| asymmetryAform_st2 | NO: 58) |
| rPB66_23S_scaffold | ACCTTACCGACTTTTTGCTTATCGCAGTCCCACTGCTTTTTTTGTACGTA |
| asymmetryAform_st3 | CACG (SEQ ID NO: 59) |
| rPB66_23S_scaffold | TTCATATCATTCGGAAATCGCCGGTTAAAGAGC (SEQ ID NO: 60) |
| asymmetryAform_st4 | |
| rPB66_23S_scaffold | TTCGCTTGGTTTACCGGGGCTTCGATCTAACGG (SEQ ID NO: 61) |
| asymmetryAform_st5 | |
| rPB66_23S_scaffold | TTCTTTTCGCCTTTTTTTTCCCTCACGTCGAAACACACTTTTTTGGGTTT |
| asymmetryAform_st6 | CCCC (SEQ ID NO: 62) |
| rPB66_23S_scaffold | CTATCGGTCAGGGATTCAGTTAATGATAGTGTGGTACTGGTTCA (SEQ ID |
| asymmetryAform_st7 | NO: 63) |
| rPB66_23S_scaffold | TAGCCTTGGAGCCGGTTCGCCTCATTAACCTATTCAGGAGTATT (SEQ ID |
| asymmetryAform_st8 | NO: 64) |
| rPB66_23S_scaffold | CACTGATTCAGTTTTTGCTCTGGGCTGGTACTTAGATGTTTTTTTTCAGT |
| asymmetryAform_st9 | TCCC (SEQ ID NO: 65) |
| rPB66_23S_scaffold | TTTTCCTCGGGCTCCCCGTTCG (SEQ ID NO: 66) |
| asymmetryAform_st10 | |
| rPB66_23S_scaffold | TTGATTTCCTCGCCGCTACTGGGGGAACGTCCA (SEQ ID NO: 67) |
| asymmetryAform_st11 | |
| rPB66_23S_scaffold | CTTTCGTGGCAGGCGTCACACCGTATATCTCGG (SEQ ID NO: 68) |
| asymmetryAform_st12 | |
| rPB66_23S_scaffold | AACACACAGCGCGCCTTTCCAGACGCTGTCGGG (SEQ ID NO: 69) |
| asymmetryAform_st13 | |
| rPB66_23S_scaffold | ATGACCCCGTTTGCATCGGGTTGGTAATCCACT (SEQ ID NO: 70) |
| asymmetryAform_st14 | |
| rPB66_23S_scaffold | GATGGTCCCCCTTTTTCATATTCAGACTACGGGGCTGTTTTTTCACCCTG |
| asymmetryAform_st15 | TATC (SEQ ID NO: 71) |
| rPB66_23S_scaffold | GCATTTTTGTGAGGATACCACG (SEQ ID NO: 72) |
| asymmetryAform_st16 | |
| rPB66_23S_scaffold | CAGCATGTTGTCCCGCCCTACTCATCGTCCGCT (SEQ ID NO: 73) |
| asymmetryAform_st17 | |
| rPB66_23S_scaffold | AATTTTTCCACCCCCAGCCACAAGTCAAGCTCA (SEQ ID NO: 74) |
| asymmetryAform_st18 | |
| rPB66_23S_scaffold | CGCCGGGGGTTTCAGGTTCTTTTTCACCATGGC (SEQ ID NO: 75) |
| asymmetryAform_st19 | |
| rPB66_23S_scaffold | TAGATCACACCCAACCTTCAACCTGCCTCCCCT (SEQ ID NO: 76) |
| asymmetryAform_st20 | |
| rPB66_23S_scaffold | TTTCACCCGCTTTTTTTTATCGTTACTCTTGCTACAGATTTTTATATAAG |
| asymmetryAform_st21 | TCGC (SEQ ID NO: 78) |
| rPB66_23S_scaffold | TCGCACTTCTGTCGGCTCCCCTATTCGGTTAACTATGTCAGCAT (SEQ ID |
| asymmetryAform_st22 | NO: 79) |
| rPB66_23S_scaffold | ATGCCTCACAGCCAGTTAAGACTCGGTTTCCCTATACCTCCAGC (SEQ ID |
| asymmetryAform_st23 | NO: 80) |
| rPB66_23S_scaffold | AACATTAGTCGTTTTTGTTCGGTCCTCTCTATACCCTGTTTTTCAACTTA |
| asymmetryAform_st24 | ACGCCACACCTTCGCTTTTTAGGCTTACAGA (SEQ ID NO: 81) |
| rPB66_23S_scaffold | CGGGTTTCGGGCAGTTAGTGTT (SEQ ID NO: 82) |
| asymmetryAform_st25 | |
| rPB66_23S_scaffold | TGCCGCAGCTTTTTTTCGGTGCATGGTTCTCCCGGTTTTTTTTGATTGGC |
| asymmetryAform_st26 | CTTT (SEQ ID NO: 83) |
| rPB66_23S_scaffold | ACATCTTCCGCAGCTTTCGGGGAGAACCAGCTATTAGCCCCGTT (SEQ ID |
| asymmetryAform_st27 | NO: 84) |
| rPB66_23S_scaffold | CGACCAGTGAGATTCACGAGGCGCTACCTAAATGCAGGCCGACT (SEQ ID |
| asymmetryAform_st28 | NO: 85) |
| rPB66_23S_scaffold | TGTCTCCCGTGTTTTTATAACATTCTCAGTGCTCTACCTTTTTCCCGGAG |
| asymmetryAform_st29 | ATGA (SEQ ID NO: 86) |
| rPB66_23S_scaffold | CTTGCCGAAACCGGTATTCGCA (SEQ ID NO: 87) |
| asymmetryAform_st30 | |
| rPB66_23S_scaffold | CACCCGCCGTGTAGCTGGCGGT (SEQ ID NO: 88) |
| asymmetryAform_st31 | |
| rPB66_23S_scaffold | GACGTTAGCTGGGTTGTTTCCCTCTTCTGGTAT (SEQ ID NO: 89) |
| asymmetryAform_st32 | |
| rPB66_23S_scaffold | CTTCGACTATAAACAGTTGCAGCCAGCACGACG (SEQ ID NO: 90) |
| asymmetryAform_st33 | |
| rPB66_23S_scaffold | CTATTACGCTTTTTTTTCTTTAAATGACTTAACCATGATTTTTCTTTGGG |
| asymmetryAform_st34 | ACCT (SEQ ID NO: 91) |
| rPB66_23S_scaffold | ATCGTTTCCCATGGCTGCTTCT (SEQ ID NO: 92) |
| asymmetryAform_st35 | |
| rPB66_23S_scaffold | CTTCCCACAAGCCAACATCCTGGCTGTAACATA (SEQ ID NO: 93) |
| asymmetryAform_st36 | |
| rPB66_23S_scaffold | GCCTTCTCGGACAACCGTCGCCCGGCCCTGGGC (SEQ ID NO: 94) |
| asymmetryAform_st37 | |
| rPB66_23S_scaffold | AGCGTCGCACGCTCCCCTACCCAACAACGACTA (SEQ ID NO: 95) |
| asymmetryAform_st38 | |
| rPB66_23S_scaffold | CGCCTTTCATATTAACCTGTTTCCCATCGCATA (SEQ ID NO: 96) |
| asymmetryAform_st39 | |
| rPB66_23S_scaffold | TTTTCCTGGAATTGGTCTTCCGGCGAGCGGGCTTGCTTAGAGGC (SEQ ID |
| asymmetryAform_st40 | NO: 332) |
| rPB66_23S_scaffold | GTTGCTTCAGCGATTAACGTTGGACAGGAACCCGCAGGGCATTT (SEQ ID |
| asymmetryAform_st41 | NO: 97) |
| rPB66_23S_scaffold | AGGGGTCGACTTTTTTCACCCTGCCCCACCGTAGTGCCTTTTTTCGTCAT |
| asymmetryAform_st42 | CACG (SEQ ID NO: 98) |
| rPB66_23S_scaffold | GGCCTCGCCTTCAAGTACAGGA (SEQ ID NO: 99) |
| asymmetryAform_st43 | |
| rPB66_23S_scaffold | ACCAGCCTACATTTTTCGCTTAAACCGCGTCCCCCCTTTTTTTCGCAGTA |
| asymmetryAform_st44 | ACAC (SEQ ID NO: 100) |
| rPB66_23S_scaffold | CCTGGAAACCTCAGCCTTGATTTTCCGCCGAAG (SEQ ID NO: 101) |
| asymmetryAform_st45 | |
| rPB66_23S_scaffold | TTACGGCACATATCAGCGTGCCTTCTCGATTTG (SEQ ID NO: 102) |
| asymmetryAform_st46 | |
| rPB66_23S_scaffold | GGGTGGAGACATTTTTGCCTGGCCATCGGTACGATTTGTTTTTATGTTAC |
| asymmetryAform_st47 | CTGA (SEQ ID NO: 103) |
| rPB66_23S_scaffold | CGTGCAGGTCGCTGACCACCTGTGTCGGTTTGGATTACGCCATT (SEQ ID |
| asymmetryAform_st48 | NO: 104) |
| rPB66_23S_scaffold | ACAAGGAATTTCAAGCGCCTTGGTATTCTCTACGAACTTACCCG (SEQ ID |
| asymmetryAform_st49 | NO: 105) |
| rPB66_23S_scaffold | AGTTCCTTCACTTTTTCCGAGTTCTCTCGCTACCTTAGTTTTTGACCGTT |
| asymmetryAform_st50 | ATAGTCAATTAACCTTTTTTTCCGGCACCGG (SEQ ID NO: 106) |
| rPB66_23S_scaffold | CCATTTTGCCTCGCTTCACCTA (SEQ ID NO: 107) |
| asymmetryAform_st51 | |
| rPB66_23S_scaffold | TTTGCACAGTGTTTTTCTGTGTTTTTAGATTTCAGCTCTTTTTCACGAGC |
| asymmetryAform_st52 | AAGT (SEQ ID NO: 108) |
| rPB66_23S_scaffold | CGCTAACCCCATTACGGCCGCC (SEQ ID NO: 109) |
| asymmetryAform_st53 | |
| rT66_EGFP_staple | GTGGTCGGGGTACAGCTCCTCGCCCTTTTGTGTTGCTGCTTCAT (SEQ ID |
| asymmetryAform_stap1 | NO: 110) |
| rT66_EGFP_staple | CACTGCACGCCAGGATGGGCACCACCCCGGTGAAGCGGCTGAAG (SEQ ID |
| asymmetryAform_stap2 | NO: 111) |
| rT66_EGFP_staple | TGGTAGTGGTCGGTTTTTCGAGCTGCACATGGCGGACTTGTTTTTAAGAA |
| asymmetryAform_stap3 | GTCGGGAATTGTGAGCGTTTTTGATAACAAT (SEQ ID NO: 112) |
| rT66_EGFP_staple | AACAGCTATGAGTCGCCGATGGGGGTGTTCTGCTTCACACAGGA (SEQ ID |
| asymmetryAform_stap4 | NO: 113) |
| rT66_EGFP_staple | CCAAGCTTGCATGTCGGGCAGCAGCACGGGGCCCCATGATTACG (SEQ ID |
| asymmetryAform_stap5 | NO: 114) |
| rT66_EGFP_staple | TCCAGCTTACGTTGTGGCTGTTGTAGTCTTTGC (SEQ ID NO: 115) |
| asymmetryAform_stap6 | |
| rT66_EGFP_staple | TCAGGGCGATCGCGCTTCTCGTTGGGGTTGTAC (SEQ ID NO: 116) |
| asymmetryAform_stap7 | |
| rT66_EGFP_staple | GTGCCCCAGGACCATGATATAG (SEQ ID NO: 117) |
| asymmetryAform_stap8 | |
| rT66_EGFP_staple | ATGCCGTTCTTCTTTTTTGCTTGTCGGTGTTGCCGTCCTCTTTTTCTTGA |
| asymmetryAform_stap9 | AGTCGGCGCGGGTCTTGTTTTTTAGTTGCCG (SEQ ID NO: 118) |
| rT66_EGFP_staple | TCGATGTTGTGGCTCCTGGACGTAGCCTTCGGGCGCTGCCGTCC (SEQ ID |
| asymmetryAform_stap10 | NO: 119) |
| rT66_EGFP_staple | AGTTCACCTTGTCGTCCTTGAAGAAGATGGTGCGCGGATCTTGA (SEQ ID |
| asymmetryAform_stap11 | NO: 120) |
| rT66_EGFP_staple | CGAACTCCAGCAGTTTTTGACCATGTGGACTGGGTGCTCATTTTTGGTAG |
| asymmetryAform_stap12 | TGGTTGCCTGCAGTTACTTTTTTTGTACAGC (SEQ ID NO: 121) |
| rT66_EGFP_staple | CGCTGAACCATCGCCCTCGCCCTCGCTCCCGGC (SEQ ID NO: 122) |
| asymmetryAform_stap13 | |
| rT66_EGFP_staple | GGCGGTCATCGTCCATGCCGAGAGTGACGGACA (SEQ ID NO: 123) |
| asymmetryAform_stap14 | |
| rT66_EGFP_staple | TTGTGGCCGTTGCCGTAGGTGG (SEQ ID NO: 124) |
| asymmetryAform_stap15 | |
| rT66_EGFP_staple | AGATGAACTTCAGTTTTTGGTCAGCTTTACGTCGCCGTCCTTTTTAGCTC |
| asymmetryAform_stap16 | GACCGTAGGTCAGGGTGTTTTTGTCACGAGG (SEQ ID NO: 125) |
| rT66_EGFP_staple | CGCCCTCGGCTCGATGCGGTTCACCAGCTTGCC (SEQ ID NO: 126) |
| asymmetryAform_stap17 | |
| rT66_EGFP_staple | GGTGGTGCGTGGGCCAGGGCACGGGCAGGGTGT (SEQ ID NO: 127) |
| asymmetryAform_stap18 | |
| rT66_EGFP_staple | AACTTCACCTCGATGCCCTTCA (SEQ ID NO: 128) |
| asymmetryAform_stap19 | |
| rPB66_23s_staple | TTTTCACCCGCTTTTTTTTATCGTTACAACCTTGCTACAGTTTTTAATAT |
| asymmetryAform_stap1 | AAGT (SEQ ID NO: 129) |
| rPB66_23s_staple | GCTTTTCCTGGCTTGGTCTTCCGGCGAGCGGGCGATGCTTAGAG (SEQ ID |
| asymmetryAform_stap2 | NO: 130) |
| rPB66_23s_staple | TTGTTGCTTCACGATTAACGTTGGACAGGAACCAAGCAGGGCAT (SEQ ID |
| asymmetryAform_stap3 | NO: 131) |
| rPB66_23s_staple | TTCGCACTTCTCCTTCGGCTCCCCTATTCGGTTTTATGTCAGCA (SEQ ID |
| asymmetryAform_stap4 | NO: 132) |
| rPB66_23s_staple | CATGCCTCACACGCCCAGTTAAGACTCGGTTTCGATACCTCCAG (SEQ ID |
| asymmetryAform_stap5 | NO: 133) |
| rPB66_23s_staple | TCAACATTAGTCGTTTTTGTTCGGTCCGGGTCTATACCCTTTTTTGCAAC |
| asymmetryAform_stap6 | TTAAGCACACCTTCGCATTTTTGGCTTACAG (SEQ ID NO: 134) |
| rPB66_23s_staple | TCACAGCACGTGTCCCGCCCTACTCATCATCCG (SEQ ID NO: 135) |
| asymmetryAform_stap7 | |
| rPB66_23s_staple | CTAATTTTTTTCACCCCCAGCCACAAGTCGAGC (SEQ ID NO: 136) |
| asymmetryAform_stap8 | |
| rPB66_23s_staple | TGTGCATTTTTACAGGATACCA (SEQ ID NO: 137) |
| asymmetryAform_stap9 | |
| rPB66_23s_staple | GGTTCTTTTCGCCTTTTTTTTCCCTCAGTGTCGAAACACATTTTTCTGGG |
| asymmetryAform_stap10 | TTTC (SEQ ID NO: 138) |
| rPB66_23s_staple | GGCTAGATTTACCCAACCTTCAACCTCACTCCC (SEQ ID NO: 139) |
| asymmetryAform_stap11 | |
| rPB66_23s_staple | CTCGCCGGACGGTTTCAGGTTCTTTTTGCCCAT (SEQ ID NO: 140) |
| ryAformasymmet_stap12 | |
| rPB66_23s_staple | CACCGGGTTTCTCCAGTTAGTG (SEQ ID NO: 141) |
| asymmetryAform_stap13 | |
| rPB66_23s_staple | TTCGTGCAGGTACCTGACCACCTGTGTCGGTTTTCATTACGCCA (SEQ ID |
| asymmetryAform_stap14 | NO: 142) |
| rPB66_23s_staple | CGACAAGGAATCTCAAGCGCCTTGGTATTCTCTCGGAACTTACC (SEQ ID |
| asymmetryAform_stap15 | NO: 143) |
| rPB66_23s_staple | TCGGGTGGAGACATTTTTGCCTGGCCAGGGGTACGATTTGTTTTTATGTT |
| asymmetryAform_stap16 | ACCT (SEQ ID NO: 144) |
| rPB66_23s_staple | TATACAAAAGGCTCTGACTGCCAGGGCTGAGTCCGCTGACCCAT (SEQ ID |
| asymmetryAform_stap17 | NO: 145) |
| rPB66_23s_staple | ACGCCTAAGCGAGATTAGCACGTCCTTCATCGCTACGCAGTCAC (SEQ ID |
| asymmetryAform_stap18 | NO: 146) |
| rPB66_23s_staple | TCACCTTACCGACTTTTTGCTTATCGCTGCTCCCACTGCTTTTTTTGTAC |
| asymmetryAform_stap19 | GTAC (SEQ ID NO: 147) |
| rPB66_23s_staple | AGCTTCGCCCGTTTACCGGGGCTTCGTTATAAC (SEQ ID NO: 148) |
| asymmetryAform_stap20 | |
| rPB66_23s_staple | GGTTCATACCCATTCGGAAATCGCCGGATCAAG (SEQ ID NO: 149) |
| asymmetryAform_stap21 | |
| rPB66_23s_staple | TTGCGCTAACCAGTTACGGCCG (SEQ ID NO: 150) |
| asymmetryAform_stap22 | |
| rPB66_23s_staple | AGGATGGTCCCCCTTTTTCATATTCAGGTGTACGGGGCTGTTTTTTCACC |
| asymmetryAform_stap23 | CTGT (SEQ ID NO: 151) |
| rPB66_23s_staple | CACTATCGGTCTATGGATTCAGTTAATGATAGTCGGTACTGGTT (SEQ ID |
| asymmetryAform_stap24 | NO: 152) |
| rPB66_23s_staple | TTTAGCCTTGGCCCCCGGTTCGCCTCATTAACCAGTCAGGAGTA (SEQ ID |
| asymmetryAform_stap25 | NO: 153) |
| rPB66_23s_staple | TGCCGCAGCTTCGTTTTTGTGCATGGTCTATCTCCCGGTTTTTTTTGATT |
| asymmetryAform_stap26 | GGCC (SEQ ID NO: 154) |
| rPB66_23s_staple | TAGGGGTCGACTCTTTTTACCCTGCCCGCACCGTAGTGCCTTTTTTCGTC |
| asymmetryAform_stap27 | ATCA (SEQ ID NO: 155) |
| rPB66_23s_staple | ACGCCTTTATATTAACCTGTTTCCCAACGCATA (SEQ ID NO: 156) |
| asymmetryAform_stap28 | |
| rPB66_23s_staple | AGCGTCGCAACGCTCCCCTACCCAACATCGACT (SEQ ID NO: 157) |
| asymmetryAform_stap29 | |
| rPB66_23s_staple | CGGCCTCGCCTCAAGTACAGGA (SEQ ID NO: 158) |
| asymmetryAform_stap30 | |
| rPB66_23s_staple | CCTTCCCAAAGCCAACATCCTGGCTGCAACATA (SEQ ID NO: 159) |
| asymmetryAform_stap31 | |
| rPB66_23s_staple | GCCTTCTCGGGACAACCGTCGCCCGGCTCTGGG (SEQ ID NO: 160) |
| asymmetryAform_stap32 | |
| rPB66_23s_staple | CATCGTTTCCCTGGCTGCTTCT (SEQ ID NO: 161) |
| asymmetryAform_stap33 | |
| rPB66_23s_staple | AACCAGCCTACACTTTTTGCTTAAACCCGTCCCCCCTTCGTTTTTCAGTA |
| asymmetryAform_stap34 | ACAC (SEQ ID NO: 162) |
| rPB66_23s_staple | CTAGTTCCTTCACTTTTTCCGAGTTCTTTCGCTACCTTAGTTTTTGACCG |
| asymmetryAform_stap35 | TTATCCATCAATTAACCTTTTTTTCCGGCAC (SEQ ID NO: 163) |
| rPB66_23s_staple | AGTTACGGACATATCAGCGTGCCTTCCGGATTT (SEQ ID NO: 164) |
| asymmetryAform_stap36 | |
| rPB66_23s_staple | GCCTGGAACGCCTCAGCCTTGATTTTCTCCCGA (SEQ ID NO: 165) |
| asymmetryAform_stap37 | |
| rPB66_23s_staple | CACCATTTTGCTCGCTTCACCT (SEQ ID NO: 166) |
| asymmetryAform_stap38 | |
| rPB66_23s_staple | TGTGTCTCCCGTGTTTTTATAACATTCAACAGTGCTCTACTTTTTCCCCG |
| asymmetryAform_stap39 | GAGA (SEQ ID NO: 167) |
| rPB66_23s_staple | CGGACGTTTCTGGGTTGTTTCCCTCTGCTGGTA (SEQ ID NO: 168) |
| asymmetryAform_stap40 | |
| rPB66_23s_staple | TCTTCGACTAATAAACAGTTGCAGCCATCACGA (SEQ ID NO: 169) |
| asymmetryAform_stap41 | |
| rPB66_23s_staple | AGCACCCGCCGTTAGCTGGCGG (SEQ ID NO: 170) |
| asymmetryAform_stap42 | |
| rPB66_23s_staple | CTATTACGCTTTCTTTTTTTTAAATGAACTTAACCATGACTTTTTTTTGG |
| asymmetryAform_stap43 | GACC (SEQ ID NO: 171) |
| rPB66_23s_staple | ACATCTTCCGCAATAGCTTTCGGGGAGAACCAGTTAGCCCCGTT (SEQ ID |
| asymmetryAform_stap44 | NO: 172) |
| rPB66_23s_staple | CGACCAGTGAGTGAATTCACGAGGCGCTACCTAGCAGGCCGACT (SEQ ID |
| asymmetryAform_stap45 | NO: 173) |
| rPB66_23s_staple | TGTTTGCACAGTGTTTTTCTGTGTTTTTGATTTCAGCTCCTTTTTACGAG |
| asymmetryAform_stap46 | CAAG (SEQ ID NO: 174) |
| rPB66_23s_staple | CGGTTGATCGCTCGCCGCTACTGGGGATACGTC (SEQ ID NO: 175) |
| asymmetryAform_stap47 | |
| rPB66_23s_staple | CACTTTCGCGGGCAGGCGTCACACCGTGAATCT (SEQ ID NO: 176) |
| asymmetryAform_stap48 | |
| rPB66_23s_staple | TTCTTTTCCTCTGCTCCCCGTT (SEQ ID NO: 177) |
| asymmetryAform_stap49 | |
| rPB66_23s_staple | CACACTGATTCAGTTTTTGCTCTGGGCGGGGTACTTAGATTTTTTGTTTC |
| asymmetryAform_stap50 | AGTT (SEQ ID NO: 178) |
| rPB66_23s_staple | GGGATGACCAGTTTGCATCGGGTTGGGCTTCCA (SEQ ID NO: 179) |
| asymmetryAform_stap51 | |
| rPB66_23s_staple | CTAACACAATCGCGCGCCTTTCCAGACTAAGTC (SEQ ID NO: 180) |
| asymmetryAform_stap52 | |
| rPB66_23s_staple | CCCCTTGCCGATCCGGTATTCG (SEQ ID NO: 181) |
| asymmetryAform_stap53 | |
| rO44_M13_staple | TGATAAATTAATGTTTTTCCGGAGAGGCAGGTCATTGCCTTTTTTGAGAG |
| asymmetryAform_stap1 | TCTG (SEQ ID NO: 182) |
| rO44_M13_staple | ACCGTTCTAGCAATATTTAAATTGTAAACGTTATATGATATTCA (SEQ ID |
| asymmetryAform_stap2 | NO: 183) |
| rO44_M13_staple | GAGACAGTCAAATTTTTTCACCATCAAATATTTTGTTAAATTTTTATTCG |
| asymmetryAform_stap3 | CATT (SEQ ID NO: 184) |
| rO44_M13_staple | GCCTGGGGTGCTTTTCACCAGTGAGACAGGCCGTAAAGTGTAAA (SEQ ID |
| asymmetryAform_stap4 | NO: 185) |
| rO44_M13_staple | TTGGGCGCCAGGGTTTTTTGGTTTTTCCTAATGAGTGAGCTTTTTTAACT |
| asymmetryAform_stap5 | CACA (SEQ ID NO: 186) |
| rO44_M13_staple | AAGGCTATGTAGCTATTTTTGAGAGACGTAGCGGTTTGTCTACA (SEQ ID |
| asymmetryAform_stap6 | NO: 187) |
| rO44_M13_staple | CCAGCTGGCGAAATTTTTGGGGGATGTGTTTTCCCAGTCATTTTTCGACG |
| asymmetryAform_stap7 | TTGT (SEQ ID NO: 188) |
| rO44_M13_staple | TCGCTATTACGTTGTTATCCGCTCACAATTCCATGCGGGCCTCT (SEQ ID |
| asymmetryAform_stap8 | NO: 189) |
| rO44_M13_staple | AACTGTTGGGAAGTTTTTGGCGATCGGCACAACATACGAGTTTTTCCGGA |
| asymmetryAform_stap9 | AGCA (SEQ ID NO: 190) |
| rO44_M13_staple | AAATCAGCTCACCATTCGCCATTCAGGCTGCGCAAATTTTTGTT (SEQ ID |
| asymmetryAform_stap10 | NO: 191) |
| rO44_M13_staple | GTGCCGGAAACCATTTTTGGCAAAGCGTTTTTTAACCAATTTTTTAGGAA |
| asymmetryAform_stap11 | CGCC (SEQ ID NO: 192) |
| rO44_M13_staple | ACGCCAGGGCTGCAAGGCGATTAAGTTCTGGCACCGCTTGGGTA (SEQ ID |
| asymmetryAform_stap12 | NO: 193) |
| rO44_M13_staple | TGAGCGAGTAACATTTTTACCCGTCGGAACAAACGGCGGATTTTTTTGAC |
| asymmetryAform_stap13 | CGTA (SEQ ID NO: 194) |
| rO44_M13_staple | CCTCAGGAAGACAGCTTTCATCAACATTAAATGGACAGTATCGG (SEQ ID |
| asymmetryAform_stap14 | NO: 195) |
| rO44_M13_staple | TCGCGTCTGGCCTTTTTTTCCTGTAGCTCGCACTCCAGCCTTTTTAGCTT |
| asymmetryAform_stap15 | TCCG (SEQ ID NO: 196) |
| rO44_M13_staple | AGCCCCAATGTACCCCGGTTGATAATTAATATCAAAAACAGAAA (SEQ ID |
| asymmetryAform_stap16 | NO: 197) |
| rO44_M13_staple | TAAAACTAGCATGTTTTTTCAATCATAAAACAGGAAGATTTTTTTGTATA |
| asymmetryAform_stap17 | AGCA (SEQ ID NO: 198) |
| rO44_M13_staple | GGTAATCGGAGCAAACAAGAGAATCGTGGGATTCTCCGATGAAC (SEQ ID |
| asymmetryAform_stap18 | NO: 199) |
| rO44_M13_staple | ACGTTGGTGTAGATTTTTTGGGCGCATTCTGCCAGTTTGATTTTTGGGGA |
| asymmetryAform_stap19 | CGAC (SEQ ID NO: 200) |
| rO44_M13_staple | ATTAATGACGGGAAACCTGTCGTGCCGGTCATGGGATAAGCTGC (SEQ ID |
| asymmetryAform_stap20 | NO: 201) |
| rO44_M13_staple | CGCTCACTGCCCGTTTTTCTTTCCAGTATCGGCCAACGCGTTTTTCGGGG |
| asymmetryAform_stap21 | AGAG (SEQ ID NO: 202) |
| rO44_M13_staple | ATCATGGTCCGGGTACCGAGCTCGAAGTTGTTAATTGCTTCGTA (SEQ ID |
| asymmetryAform_stap22 | NO: 203) |
| rO44_M13_staple | GCAGGTCGACTCTTTTTTAGAGGATCCCATAGCTGTTTCCTTTTTTGTGT |
| asymmetryAform_stap23 | GAAA (SEQ ID NO: 204) |
| rO44_M13_staple | GCATGCCTAAAACGACGGCCAGTGCCTGCACGTAACCGAAGCTT (SEQ ID |
| asymmetryAform_stap24 | NO: 205) |
| rO66_23s_staple | GCACCGTAGTGCCTTTTTTCGTCATCACCGGGACAACCGTTTTTTCGCCC |
| asymmetryAform_stap1 | GGCC (SEQ ID NO: 206) |
| rO66_23s_staple | GCTTTTCCTGGTTACTTATGTCAGCATTCGCACGATGCTTAGAG (SEQ ID |
| asymmetryAform_stap2 | NO: 207) |
| rO66_23s_staple | TTGTTGCTTCAGGGCTTTTCACCCGCTTTATCGAAGCAGGGCAT (SEQ ID |
| asymmetryAform_stap3 | NO: 208) |
| rO66_23s_staple | GGGGTACGATTTGTTTTTATGTTACCTTTCTGATACCTCCTTTTTAGCAT |
| asymmetryAform_stap4 | GCCT (SEQ ID NO: 209) |
| rO66_23s_staple | TTCCAGACGCTCTCTGACTGCCAGGGCCGGTTTATCGCGCGCCT (SEQ ID |
| asymmetryAform_stap5 | NO: 210) |
| rO66_23s_staple | ACACACTGATTAGATTAGCACGTCCTTCATCGCTCCACTAACAC (SEQ ID |
| asymmetryAform_stap6 | NO: 211) |
| rO66_23s_staple | TCACCTTACCGACTTTTTGCTTATCGCCAGGCTCTGGGCTTTTTTGCTCC |
| asymmetryAform_stap7 | CCGT (SEQ ID NO: 212) |
| rO66_23s_staple | ACCAGCCTTGATTTTCCGGATTTGCCTTATAAC (SEQ ID NO: 213) |
| asymmetryAform_stap8 | |
| rO66_23s_staple | GGTTCATACCCATTCGGAAATCGCCGGTGGAAA (SEQ ID NO: 214) |
| asymmetryAform_stap9 | |
| r066_23s_staple | ACACGCTTAAACGCCTCAGCCT (SEQ ID NO: 215) |
| asymmetryAform_stap10 | |
| rO66_23s_staple | TCGGTTTCCCTTCTTTTTGGCTCCCCTCGCAGTCACACGCTTTTTCTAAG |
| asymmetryAform_stap11 | CGTG (SEQ ID NO: 216) |
| rO66_23s_staple | TCTATACCCTGAGCTCACAGCATGTGCATTTTTCGGGTTTCGGG (SEQ ID |
| asymmetryAform_stap12 | NO: 217) |
| rO66_23s_staple | CCAGTTAAGACACGTGTCCCGCCCTACTCATCGCAACTTAACGC (SEQ ID |
| asymmetryAform_stap13 | NO: 218) |
| rO66_23s_staple | AACCTGCCCATGGTTTTTCTAGATCACGTGTACGGGGCTGTTTTTTCACC |
| asymmetryAform_stap14 | CTGT (SEQ ID NO: 219) |
| rO66_23s_staple | TTCGCAGGCTTCAGTTAGTGTTACCCAACCTTCCACAGCACACC (SEQ ID |
| asymmetryAform_stap15 | NO: 220) |
| rO66_23s_staple | CCCTACCCAACAACATTAGTCGGTTCGGTCCTCACAGAACGCTC (SEQ ID |
| asymmetryAform_stap16 | NO: 221) |
| rO66_23s_staple | ACAAGTCATCCGCTTTTTTAATTTTTCAACGCATAAGCGTTTTTTCGCTG |
| asymmetryAform_stap17 | CCGC (SEQ ID NO: 222) |
| rO66_23s_staple | ACCCATTACTTGCTACAGAATATAAGCTTTCAC (SEQ ID NO: 223) |
| asymmetryAform_stap18 | |
| rO66_23s_staple | CCCCAGCCATCTCCCGGTTTGATTGGCTCGCTG (SEQ ID NO: 224) |
| asymmetryAform_stap19 | |
| rO66_23s_staple | TACAAAAGGTAATTCGGTTAAC (SEQ ID NO: 225) |
| asymmetryAform_stap20 | |
| rO66_23s_staple | CATCCTGGCTGTCTTTTTTGGGCCTTCCCTTAGCTGGCGGTTTTTTCTGG |
| asymmetryAform_stap21 | GTTG (SEQ ID NO: 226) |
| rO66_23s_staple | CCCCGGAGATGATGATGGCTGCTTCTAAGCCAACAGTGCTCTAC (SEQ ID |
| asymmetryAform_stap22 | NO: 227) |
| rO66_23s_staple | CGCTACCTAAATGAGCTATTACGCTTTCTTTAAAATTCACGAGG (SEQ ID |
| asymmetryAform_stap23 | NO: 228) |
| rO66_23s_staple | CCGCGCAGGCCGATTTTTCTCGACCAGTAGCTTTCGGGGATTTTTGAACC |
| asymmetryAform_stap24 | AGCT (SEQ ID NO: 229) |
| rO66_23s_staple | CCGATTAACCTTAGGGGTCGACTCACGCCCCGT (SEQ ID NO: 230) |
| asymmetryAform_stap25 | |
| rO66_23s_staple | TACATCTTAGCTTCGGTGCATGGTTTACCTGCC (SEQ ID NO: 231) |
| asymmetryAform_stap26 | |
| rO66_23s_staple | CGTTGGACAGGTTTCGGCCTCG (SEQ ID NO: 232) |
| asymmetryAform_stap27 | |
| rO66_23s_staple | CCTGTTTCCCATCTTTTTGACTACGCCAACCCTTGGTCTTTTTTTCCGGC |
| asymmetryAform_stap28 | GAGC (SEQ ID NO: 233) |
| rO66_23s_staple | CCAAGTACCTCCGTCCCCCCTTCGCAACCATGA (SEQ ID NO: 234) |
| asymmetryAform_stap29 | |
| rO66_23s_staple | CTTTGGGACCACATCGTTTCCCACTTAGTAACA (SEQ ID NO: 235) |
| asymmetryAform_stap30 | |
| rO66_23s_staple | AGGAATATTAAAACATAGCCTT (SEQ ID NO: 236) |
| asymmetryAform_stap31 | |
| rO66_23s_staple | TGTGTCTCCCGTGTTTTTATAACATTCCGGGATGACCCCCTTTTTTTGCC |
| asymmetryAform_stap32 | (SEQ ID NO: 237) |
| rO66_23s_staple | GGATTCAGCCCCGGTTCGCCTCATTACGTTAGC (SEQ ID NO: 238) |
| asymmetryAform_stap33 | |
| rO66_23s_staple | ACCCGCCGTTTCCCTCTTCACGACGGAACCTAT (SEQ ID NO: 239) |
| asymmetryAform_stap34 | |
| rO66_23s_staple | TTAATGATAGTTGTTTCAGTTC (SEQ ID NO: 240) |
| asymmetryAform_stap35 | |
| rO66_23s_staple | TCTTTTCCTCGGGTTTTTGTACTTAGAGTGTCGAAACACATTTTTCTGGG |
| asymmetryAform_stap36 | TTTC (SEQ ID NO: 241) |
| rO66_23s_staple | TAGCCTTGCACTATCGGTCAGTCAGGGAATCTC (SEQ ID NO: 242) |
| asymmetryAform_stap37 | |
| rO66_23s_staple | GGTTGATTTCGCTCGCCGCTACTGGGGAGTATT (SEQ ID NO: 243) |
| asymmetryAform_stap38 | |
| rO66_23s_staple | GAGGATGGTCCCGGTACTGGTT (SEQ ID NO: 244) |
| asymmetryAform_stap39 | |
| rO66_23s_staple | GGTTCTTTTCGCCTTTTTTTTCCCTCACCCCATATTCAGATTTTTCAGGA |
| asymmetryAform_stap40 | TACC (SEQ ID NO: 245) |
| rO66_23s_staple | TTTTCACTTGTACGTACACGGTTTCAATCGGGT (SEQ ID NO: 246) |
| asymmetryAform_stap41 | |
| rO66_23s_staple | TGGTAAGTTCCGGTATTCGCAGTTTGCGGTTCT (SEQ ID NO: 247) |
| asymmetryAform_stap42 | |
| rO66_23s_staple | CCCCTCGCCGGCTCCCACTGCT (SEQ ID NO: 248) |
| asymmetryAform_stap43 | |
| rPB55_23s_staple | CTAAGCCAACATCTTTTTCTGGCTGTCCGGTTTCAGGTTCTTTTTTTTTT |
| asymmetryAform_stap1 | CACT (SEQ ID NO: 249) |
| rPB55_23s_staple | CCTTGGTCTTCTTTCTTTAAATGATGGCTGCTTTGGACAGGAAC (SEQ ID |
| asymmetryAform_stap2 | NO: 250) |
| rPB55_23s_staple | CGGCGAGCGGGAGCTATTACGC (SEQ ID NO: 251) |
| asymmetryAform_stap3 | |
| rPB55_23s_staple | ACATCGTTTCCGCTCCCACTGCTTGTACGTACATGGGCCTTCCC (SEQ ID |
| asymmetryAform_stap4 | NO: 252) |
| rPB55_23s_staple | CACTTAACCATCGCCTAAGCGT (SEQ ID NO: 253) |
| asymmetryAform_stap5 | |
| rPB55_23s_staple | CTTCGGCTCCCCTTTTTTATTCGGTTAATACAAAAGGTACTTTTTGCAGT |
| asymmetryAform_stap6 | CACAGACTTTGGGACCTTTTTTTAGCIGGCG (SEQ ID NO: 254) |
| rPB55_23s_staple | AGACGCTTGTCACCCTGTATCGCGCGCAAGACT (SEQ ID NO: 255) |
| asymmetryAform_stap7 | |
| rPB55_23s_staple | CGGTTTCCTGCAACTTAACGCCCAGTTCTTTCC (SEQ ID NO: 256) |
| asymmetryAform_stap8 | |
| rPB55_23s_staple | CCCATATTCAGACTTTTTAGGATACCAACTGGGTTTCCCCTTTTTATTCG |
| asymmetryAform_stap9 | GAAA (SEQ ID NO: 257) |
| rPB55_23s_staple | GACCCATTACCTTGCTACAGAATATAATGGAGG (SEQ ID NO: 258) |
| asymmetryAform_stap10 | |
| rPB55_23s_staple | ATGGTCCCAGTCAGGAGTATTTAGCCTGTCGCT (SEQ ID NO: 259) |
| asymmetryAform_stap11 | |
| rPB55_23s_staple | TGGTATTCTCTTCGGCCTCGCCTTAGGGGTCGACTCAAGCGCCT (SEQ ID |
| asymmetryAform_stap12 | NO: 260) |
| rPB55_23s_staple | ACCTGACCACCGACTACGCCTT (SEQ ID NO: 261) |
| asymmetryAform_stap13 | |
| rPB55_23s_staple | CTAGTTCCTTCACTTTTTCCGAGTTCTCTCACCCTGCCCCTTTTTGATTA |
| asymmetryAform_stap14 | ACGT (SEQ ID NO: 262) |
| rPB55_23s_staple | GGTTCTTTTCGCTCTGACTGCCAGGGCTTTTGCCCCCTCGCCGG (SEQ ID |
| asymmetryAform_stap15 | NO: 263) |
| rPB55_23s_staple | CCTTTCCCTCATCCTTCATCGC (SEQ ID NO: 264) |
| asymmetryAform_stap16 | |
| rPB55_23s_staple | ACGCTTATCGCAGTTTTTATTAGCACGCGGTACTGGTTCATTTTTCTATC |
| asymmetryAform_stap17 | GGTC (SEQ ID NO: 265) |
| rPB55_23s_staple | TTTCCTGGTGATGTTACCTGATGCTTAATATCA (SEQ ID NO: 266) |
| asymmetryAform_stap18 | |
| rPB55_23s_staple | CCTTACCGTCGCCGGTTATAACGGTTCGAGGCT (SEQ ID NO: 267) |
| asymmetryAform_stap19 | |
| rPB55_23s_staple | GTGCATTTTTGTGTTTTTTACGGGGCTCCACTAACACACATTTTTCACTG |
| asymmetryAform_stap20 | ATTC (SEQ ID NO: 268) |
| rPB55_23s_staple | CTACTCATCGATAATGATAGTGTGTCGAAACACCGTGTCCCGCC (SEQ ID |
| asymmetryAform_stap21 | NO: 269) |
| rPB55_23s_staple | GCTCACAGCATATGGATTCAGT (SEQ ID NO: 270) |
| asymmetryAform_stap22 | |
| rPB55_23s_staple | AGCACCCGCCGTGTTTTTTGTCTCCCGACCGGGTTTCGGGTTTTTTCTAT |
| asymmetryAform_stap23 | ACCC (SEQ ID NO: 271) |
| rPB55_23s_staple | GCGCAGGCCGACTTTTTTCGACCAGTGCTTTTCACCCGCTTTTTTTTATC |
| asymmetryAform_stap24 | GTTA (SEQ ID NO: 272) |
| rPB55_23s_staple | CATCTTCCTTCGGTGCATGGTTTAGCCTCACGA (SEQ ID NO: 273) |
| asymmetryAform_stap25 | |
| rPB55_23s_staple | CGGACGTTGTCTGGGTTGTTTCCCTCTCCGTTA (SEQ ID NO: 274) |
| asymmetryAform_stap26 | |
| rPB55_23s_staple | CCGGAGATCCCTTGCCGAAACAGTGCTCCCTAC (SEQ ID NO: 275) |
| asymmetryAform_stap27 | |
| rPB55_23s_staple | CCAACAACGCAGGCTTACAGAACGCTCCTACCC (SEQ ID NO: 276) |
| asymmetryAform_stap28 | |
| rPB55_23s_staple | GCATGCCTCACAGTTTTTCACACCTTCGCATAAGCGTCGCTTTTTTGCCG |
| asymmetryAform_stap29 | CAGC (SEQ ID NO: 277) |
| rPB55_23s_staple | GAATATTAACCTGTTTTTTTTCCCATCTGTGTCGGTTTGGTTTTTGGTAC |
| asymmetryAform_stap30 | GATTAAGCAGGGCATTTTTTTTGTTGCTTCA (SEQ ID NO: 278) |
| rPB55_23s_staple | AAGTACAGTCCGTCCCCCCTTCGCAGTTTCTGA (SEQ ID NO: 279) |
| asymmetryAform_stap31 | |
| rPB55_23s_staple | TACCTCCACTTATGTCAGCATTCGCACAACACC (SEQ ID NO: 280) |
| asymmetryAform_stap32 | |
| rPB55_23s_staple | TTGATTGGCCTTTTTTTTCACCCCCAGCGGTTCGGTCCTCTTTTTCAGTT |
| asymmetryAform_stap33 | AGTG (SEQ ID NO: 281) |
| rPB55_23s_staple | CTCCCGGTATAGCTTTCGGGGAGAACCAACCGG (SEQ ID NO: 282) |
| asymmetryAform_stap34 | |
| rPB55_23s_staple | GACAACCGAAACCAGCCTACACGCTTAAGCTAT (SEQ ID NO: 283) |
| asymmetryAform_stap35 | |
| rPB55_23s_staple | GGGTTGGTAAGTCTTTTTGGGATGACCGAATTCACGAGGCTTTTTGCTAC |
| asymmetryAform_stap36 | CTAA (SEQ ID NO: 284) |
| rPB55_23s_staple | TCCGGTATTCGTCAACCTGCCCATGGCTAGATCTGATAACATTC (SEQ ID |
| asymmetryAform_stap37 | NO: 285) |
| rPB55_23s_staple | CAGTTTGCATCTTACCCAACCT (SEQ ID NO: 286) |
| asymmetryAform_stap38 | |
| rPB55_23s_staple | TGATTTTCCGGATTTTTTTTGCCTGGATCGCCCGGCCAACTTTTTATAGC |
| asymmetryAform_stap39 | CTTC (SEQ ID NO: 287) |
| rPB55_23s_staple | TTCAGTTCTCTTTTCCTCGGGGTACTTTCACGC (SEQ ID NO: 288) |
| asymmetryAform_stap40 | |
| rPB55_23s_staple | CTCAGCCTGCACCGTAGTGCCTCGTCAAGATGT (SEQ ID NO: 289) |
| asymmetryAform_stap41 | |
| rPB55_23s_staple | ACTGGGGGAATCTTTTTTCGGTTGATTCCCCGGTTCGCCTTTTTTCATTA |
| asymmetryAform_stap42 | ACCT (SEQ ID NO: 290) |
| rPB55_23s_staple | ACATTAGTCCACAAGTCATCCGCTAATGTTCGC (SEQ ID NO: 291) |
| asymmetryAform_stap43 | |
| rPB55_23s_staple | TCGCCGCTAGGCTCTGGGCTGCTCCCCTTTTCA (SEQ ID NO: 292) |
| asymmetryAform_stap44 | |
| rT55_rsc1218_staple | CTACCTGCTGATGCACTCACTAGGCCCGTGGTAATCAAATAAGG (SEQ ID |
| asymmetryAform_stap1 | NO: 333) |
| rT55_rsc1218_staple | CTTTGGCTTGCTGAGTCTCTAA (SEQ ID NO: 334) |
| asymmetryAform_stap2 | |
| rT55_rsc1218_staple | AGTTCCTTCTGTCTTTTTCCTGAGGAGAATTTGGCTGTTGTTTTTGTTAA |
| asymmetryAform_stap3 | CTCCCACTTCCATACCATTTTTGGTTCTAAT (SEQ ID NO: 293) |
| rT55_rsc1218_staple | CAGGGCAGCAAAACATTAGATATGGGGTTGGCCGAATGGTGTCA (SEQ ID |
| asymmetryAform_stap4 | NO: 294) |
| rT55_rsc1218_staple | TGAAGTCTGGGGCAAGCCCAAC (SEQ ID NO: 295) |
| asymmetryAform_stap5 | |
| rT55_rsc1218_staple | GATGGGATTAGCAAGCAAGACCAAGGTACAGTT (SEQ ID NO: 296) |
| asymmetryAform_stap6 | |
| rT55_rsc1218_staple | ACAAGCCTAAGTAGCTAAGGGAGCATCCTTAGT (SEQ ID NO: 297) |
| asymmetryAform_stap7 | |
| rT55_rsc1218_staple | CTAGTGGTGTAATTTTTTGAAAAGTTCTATGCATAGCCCATTTTTTCAGC |
| asymmetryAform_stap8 | TTCAAGCCAGTCATCAGTTTTTGAGAAACCC (SEQ ID NO: 298) |
| rT55_rsc1218_staple | GGGCAGTCATAGGTTAATGGGCTCTGGGCAATACCTGCCATTAG (SEQ ID |
| asymmetryAform_stap9 | NO: 299) |
| rT55_rsc1218_staple | ACAAACTTCAGTCCTCTAGCAT (SEQ ID NO: 300) |
| asymmetryAform_stap10 | |
| rT55_rsc1218_staple | CAACACTGTGCATTTTTTATACATCAACCTCATAGATGGGTTTTTTACCC |
| asymmetryAform_stap11 | TACAGTGGGCCATGGACTTTTTTGCAAGATA (SEQ ID NO: 301) |
| rT55_rsc1218_staple | GTCCCCTCGAGCCCCTCATGTCCTTGGGCAAGG (SEQ ID NO: 302) |
| asymmetryAform_stap12 | |
| rT55_rsc1218_staple | TCCACTGCGCCATATGAATTGCATGGACCTAGG (SEQ ID NO: 303) |
| asymmetryAform_stap13 | |
| rT55_rsc1218_staple | CCTGACTAGACATTTTTTTAGTGCATATTCTGGTCATGGATTTTTTACAT |
| asymmetryAform_stap14 | GGCTATGATTCTCTCATTTTTTGAATCCCAA (SEQ ID NO: 304) |
| rT55_rsc1218_staple | TGTCAGGGAACCAATTTGAGGCCCAGAGACTAG (SEQ ID NO: 305) |
| asymmetryAform_stap15 | |
| rT55_rsc1218_staple | GGTGAAGGTTGTATTCCACATAGAGATCACAGA (SEQ ID NO: 306) |
| asymmetryAform_stap16 | |
| rT77_rsc1218_staple | AGCCAGTCATCTGCACTCACTAGGCCCCAAATAAGATGTCAGGG (SEQ ID |
| asymmetryAform_stap1 | NO: 307) |
| rT77_rsc1218_staple | TCCTCTAGCATGATACATGGCTTGAGTCTCTAAAGGAGAAACCC (SEQ ID |
| asymmetryAform_stap2 | NO: 308) |
| rT77_rsc1218_staple | GGTTAATGGGCTTCTGGTCATG (SEQ ID NO: 309) |
| asymmetryAform_stap3 | |
| rT77_rsc1218_staple | TGAAGTCTGGGGTTTTTTGGGCCATGGAACCAATTTGAGGTTTTTCCCAG |
| asymmetryAform_stap4 | ACACGATGCTTATAATGTTTTTGGACAGGTG (SEQ ID NO: 310) |
| rT77_rsc1218_staple | TTTCCCTCTAGGAATGGTGTCACAGGGCAGCAACCCATTGCCAA (SEQ ID |
| asymmetryAform_stap5 | NO: 311) |
| rT77_rsc1218_staple | CTATGGGGCTACACTTCCATACCAGGTTCTAATACCTTCCTTTT (SEQ ID |
| asymmetryAform_stap6 | NO: 312) |
| rT77_rsc1218_staple | GAAATTTACTCGCTCTGTGGTA (SEQ ID NO: 313) |
| asymmetryAform_stap7 | |
| rT77_rsc1218_staple | ACCCTACAGTTACAAGCCTCCTCATAGCCCTGG (SEQ ID NO: 314) |
| asymmetryAform_stap8 | |
| rT77_rsc1218_staple | ATTCTACATTTCATTTACAGCTCACCAATGGGT (SEQ ID NO: 315) |
| asymmetryAform_stap9 | |
| rT77_rsc1218_staple | AATTTCACCATGTTATCCAAAG (SEQ ID NO: 316) |
| asymmetryAform_stap10 | |
| rT77_rsc1218_staple | GCAAGCCCAACGGAGCATCACA (SEQ ID NO: 317) |
| asymmetryAform_stap11 | |
| rT77_rsc1218_staple | ATATACATCAAAATTTTTGTAGCTAAGAACATTAGATATGTTTTTGGGTT |
| asymmetryAform_stap12 | GGCCCTAGTGGTGTAATTTTTTGAAAAGTTC (SEQ ID NO: 318) |
| rT77_rsc1218_staple | GCCATATGAATTATGCATAGCCCATCAGCTTCAACTGCAAGATA (SEQ ID |
| asymmetryAform_stap13 | NO: 319) |
| rT77_rsc1218_staple | AGGTCCACTGCACCAAGGTCTTAGTGATGGGATTGCATGGAGCA (SEQ ID |
| asymmetryAform_stap14 | NO: 320) |
| rT77_rsc1218_staple | CAACACTGTGCTAGCAAGCAAG (SEQ ID NO: 321) |
| asymmetryAform_stap15 | |
| rT77_rsc1218_staple | GGAGGAAGGCTCTTTTTTAGAAAGCCAAAGTGGTCTACAATTTTTCCCCT |
| asymmetryAform_stap16 | TGCACTGGGGACACTCTTTTTTTTACCTGTA (SEQ ID NO: 322) |
| rT77_rsc1218_staple | AGTGCATATAGGGTGAAGGCCTGACTAGGGGCA (SEQ ID NO: 323) |
| asymmetryAform_stap17 | |
| rT77_rsc1218_staple | TGGCAAACAAGGGCAGTGCTTGCCCCTGACATT (SEQ ID NO: 324) |
| asymmetryAform_stap18 | |
| rT77_rsc1218_staple | AGGGGAGGGTGGTGAGAGTGCA (SEQ ID NO: 325) |
| asymmetryAform_stap19 | |
| rT77_rsc1218_staple | GAGCCCCTCATATAGAGATGAC (SEQ ID NO: 326) |
| asymmetryAform_stap20 | |
| rT77_rsc1218_staple | ATGAATCCCAATTTTTTTGTATTCCACGTCCTTGGCCTAGTTTTTGGTCC |
| asymmetryAform_stap21 | CCTCTCTGGGCAATAAATTTTTTTTGGCTGT (SEQ ID NO: 327) |
| rT77_rsc1218_staple | CAGTCATATCCCTGAGGAGCCTGCCATTGACTT (SEQ ID NO: 328) |
| asymmetryAform_stap22 | |
| rT77_rsc1218_staple | TGGCTTGCATCAAATAAGGCTACCTGCTAGGGG (SEQ ID NO: 329) |
| asymmetryAform_stap23 | |
| rT77_rsc1218_staple | ATGATTCTCTCTGGTTAACTCC (SEQ ID NO: 330) |
| asymmetryAform_stap24 | |
| rT77_rsc1218_staple | ACAAACTTCAGAGTTCCTTCTG (SEQ ID NO: 331) |
| asymmetryAform_stap25 | |

### SUMMARY

Folding of a variety of RNA scaffolds into several different 3D polyhedral wireframe geometries based on DX-edge designs was demonstrated.

In addition to the need to accommodate A-form helical geometry, one concern with using long RNA strands to scaffold origami is its tendency to be highly structured internally, which may occur by a combination of secondary and tertiary structure formation, as present in long natural RNAs from rRNA to viral genomes. However, the presence of native secondary and tertiary structure present in target RNA scaffolds did not prevent proper folding; the 23s rRNA fragment used to scaffold two objects has a variety of secondary structural motifs, and is approximately 58% base paired on its own, but after denaturing at high temperatures and re-annealing, the RNA scaffold bound preferentially to the staples and folded the target structure with high yield.

Scaffolding wireframe origami with RNA allowed the study of nucleic acid origami folding and stability with nucleotide-level precision. The DMS-MaPseq profiling presented in this study is the first application of chemical probing to large-scale scaffolded origami, made possible by the use of RNA scaffolds. this protocol can be applied to investigate base pairing stability for improved sequence design, for example, the data suggest that increasing the GC content just upstream of scaffold double crossovers may particularly benefit origami stability more than GC content in other areas. The approach also holds promise for kinetic studies of nucleic acid origami folding, since probing samples at various stages of the annealing ramp and comparing what sections of the scaffold are bound at each stage might improve mechanistic understanding of scaffolded origami folding.

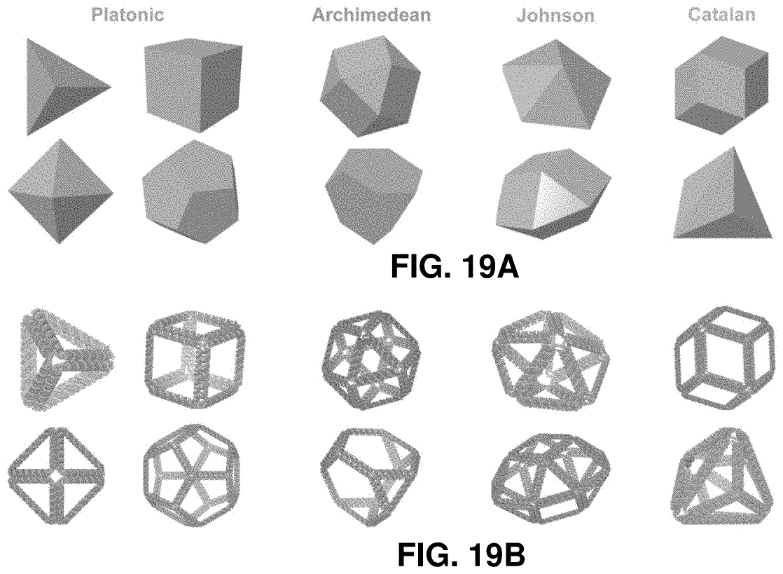

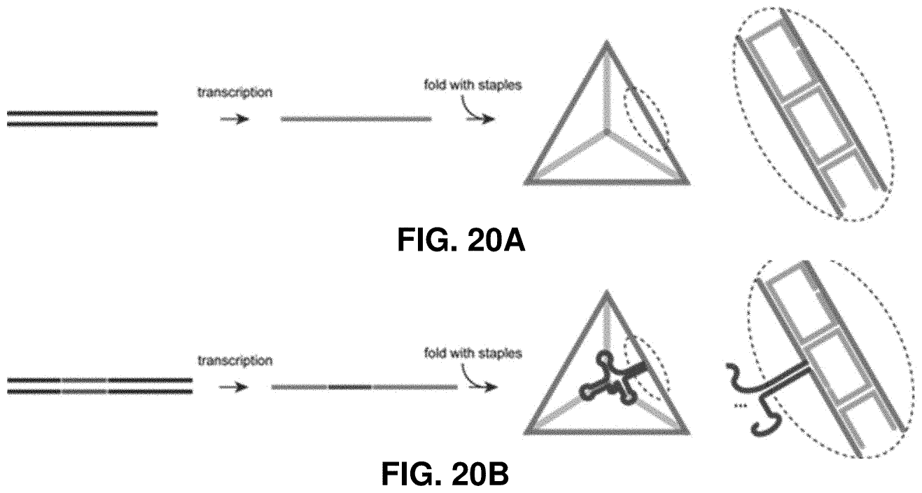

Beyond studies of nucleic acid origami itself, the use of RNA as scaffold for DX wireframe origami enables a variety of applications, depending on the particular scaffold used. RNA has a number of useful features distinct from standards DNA, including the ability to modify nucleotides for stability and translatability and to introduce riboswitches and aptamers, ribozymes, antisense oligos, and long RNAs. In particular, mRNA was used as a scaffold for the assembly of a nanoparticle containing the sequence encoding a fluorescent protein. Targeted cellular delivery of such a nanoparticle could offer important potential for nuclease-specific release of scaffold or staples within the cell, with applications in antisense oligonucleotide (ASO) therapy (Morcos, Biochem Biophys Res Commun 358, 521-527 (2007); Rinaldi & Wood, Nat Rev Neurol 14, 9-21 (2018)). multiplex automated genome engineering (MAGE) (Wang, et al., Nature 460, 894-898 (2009)), and homologous recombination template (Song & Stieger, Mol Ther Nucleic Acids 7, 53-60 (2017)). delivery. The tunable degradation rates introduced by the combined use of RNA, modified RNA and DNA prove useful for material templating and etching. Ribosomal RNA was additionally used to scaffold a pentagonal bipyramid that would leave domains V and VI of the 23S rRNA to fold freely (FIGS. 20A-20B). Engineering will enable generation of synthetic nucleic acid assemblies that can coordinate catalytic ribozymes (Sakai, et al., Genes (Basel) 9, (2018); Walter, et al. Nano Lett 17, 2467-2472 (2017)) test modification enzyme substrates (Punekar, et al., Nucleic Acids Res 41, 9537-9548 (2013); Punekar, et al., Nucleic Acids Res 40, 10507-10520 (2012)), and develop novel ribosomes and translation systems.

We claim:

1. A method for designing a scaffolded RNA nanostructure having a geometric shape comprising:
(a) determining the geometric parameters of an input,
wherein the input comprises a 3D polyhedral or 2D polygon geometric shape and optionally one or more of its physical dimensions;
(b) identifying a route for a single-stranded RNA scaffold that traces throughout the geometric shape based on A-form helical nucleic acid geometry; and
(c) generating the sequences of the single-stranded RNA scaffold and optionally the nucleic acid sequence of staple strands that combine to form a scaffolded RNA nanostructure having the geometric shape.

2. The method of claim 1, wherein generating the sequences of the single-stranded RNA scaffold in step (c) comprises staple crossover asymmetry, with 11 nucleotides per helical turn.

3. The method of claim 2, wherein the staple crossover asymmetry comprises a difference in the nucleotide position across two helices of an edge of from one to ten nucleotides, inclusive.

4. The method of claim 2, wherein the staple crossover asymmetry comprises a difference in the nucleotide position across two helices of an edge of four nucleotides.

5. The method of claim 1, wherein identifying a route for a single-stranded RNA scaffold that traces throughout the geometric shape in step (b) comprises scaffold crossover asymmetry, with 11 nucleotides per helical turn.

6. The method of claim 5, wherein the scaffold crossover asymmetry comprises a difference in the nucleotide position across two helices of an edge of from one to ten nucleotides, inclusive.

7. The method of claim 5, wherein the scaffold crossover asymmetry comprises a difference in the nucleotide position across two helices of an edge of six nucleotides.

8. The method of claim 1, wherein identifying a route for a single-stranded RNA scaffold that traces throughout the geometric shape in step (b) comprises no crossover asymmetry, with 11 nucleotides per helical turn.

9. The method of claim 1, wherein the input in step (a) further comprises one or more of the geometric shape's physical dimensions.

10. The method of claim 1, wherein the input in step (a) further comprises specifying an even number of parallel or anti-parallel helices within each edge of the nanostructure.

11. The method of claim 10, wherein each edge comprises four or more parallel or anti-parallel helices arranged in square cross-sectional morphology, or six or more parallel or anti-parallel helices arranged in honeycomb lattice morphology.

12. The method of claim 1, wherein the input in step (a) further comprises specifying for each vertex of the nanostructure that two or more edges come together in an aligned angle to create a bevel at the vertex.

13. The method of claim 1, wherein the geometric shape does not have spherical topology.

14. The method of claim 1, wherein the input in step (a) further comprises a template RNA scaffold sequence, or the sequence of one or more staples, or a template RNA scaffold sequence and the sequence of one or more staples.

15. The method of claim 1, wherein the input in step (a) further comprises the length of one or more of the edges spanning two vertices of the target structure.

16. The method of claim 15, wherein the length of each edge is between 22 base pairs and 1,100 base pairs, inclusive.

17. The method of claim 1, wherein the crossover type is anti-parallel crossover,
wherein the length of each edge is expressed as a multiple of 11 base pairs, and wherein the length of each edge is between 22 base pairs and 1,100 base pairs, inclusive.

18. The method of claim 17, wherein the length of each edge is 44 base pairs, 55 base pairs, 66 base pairs, or 77 base pairs.

19. The method of claim 1, wherein the staples are DNA.

20. The method of claim 1, wherein the staples are RNA.

21. The method of claim 1, wherein the input in step (a) comprises geometric parameters including vertex, face and edge information determined from a polygonal or polyhedral wire-mesh model of the target shape.

22. The method of claim 1, wherein identifying a route for a single-stranded RNA scaffold that traces throughout the geometric shape in step (b) comprises the steps of:
(i) rendering the geometric shape as a closed surface polyhedral network or an open surface polygonal network;
(ii) determining a spanning tree of the network, wherein the vertices and lines of the graph are the nodes and edges of the network, respectively;
(iii) classifying each edge of the network based on its membership in the spanning tree,
wherein edges that are members of the spanning tree do not have a scaffold double crossover, and edges that are not members of the spanning tree have a scaffold double crossover;
(iv) splitting each edge that is not a member of the spanning tree into two edges, each containing a pseudo-node at the point of the scaffold crossover;
(v) splitting each node at each of the vertices into two pseudo-nodes; and
(vi) calculating the Euler cycle of the network, wherein the Euler cycle represents the route of a single-stranded RNA scaffold that traces once along each edge in both directions throughout the entire geometric shape.

23. The method of claim 22, wherein the crossover type is parallel crossover, and wherein the length of each edge is between 22 base pairs and 1,100 base pairs, inclusive.

24. The method of claim 1, wherein identifying a route for a single-stranded RNA scaffold that traces throughout the geometric shape in step (b) comprises the steps of:
(i) rendering the geometric shape as a closed surface polyhedral network or an open surface polygonal network;
(ii) calculating a spanning tree of the network, wherein the vertices and lines of the graph are the nodes and edges of the network, respectively;
(iii) classifying each edge of the network as one of four types based on its membership in the spanning tree and on whether it employs anti-parallel or parallel crossovers; edges that are members of the spanning tree have each scaffold portion start and end at different vertices, and edges that are not members of the spanning tree have each scaffold portion start and end at the same vertex;
(iv) splitting each edge that is not a member of the spanning tree into two edges, each containing a pseudo-node at the point of the scaffold crossover;
(v) splitting each node at each of the vertices into two pseudo-nodes; and
(vi) calculating the Euler cycle of the network, wherein the Euler cycle represents the route of a single-stranded nucleic acid scaffold by superimposing and connecting units of partial scaffold routing within an edge based on its classification and length.

25. The method of claim 1, wherein identifying a route for a single-stranded RNA scaffold that traces throughout the geometric shape in step (b) comprises:
(i) rendering the geometric shape as a closed surface polyhedral network or an open surface polygonal network;
(ii) rendering each helix in the network as a line, based on the target cross-section of each edge;
(iii) calculating a loop-crossover structure, wherein two or more adjacent lines are connected to form loops and all possible double-crossover locations between two loops are calculated;
(iv) calculating a dual graph of the loop-crossover structure, wherein the loops and double-crossover locations of the network are converted to nodes and edges of the dual graph, respectively;
(v) calculating a spanning tree of the dual graph network;
(vi) calculating which of the locations of double-strand crossovers will be used, wherein a single double-strand crossover is placed at each edge that is the part of the spanning tree of the dual graph; and
(vii) calculating the Euler cycle of the network, wherein the Euler cycle represents the route of a single-stranded nucleic acid scaffold that traces once through each duplex throughout the entire geometric shape.

26. The method of claim 1, wherein the spanning tree of the network is determined using a breadth-first search or depth-first search.

27. The method of claim 26, wherein the spanning tree is calculated using Prim's formula or Kruskal's formula.

28. The method of claim 1, wherein the Euler circuit is the A-trail Euler circuit.

29. The method of claim 1, wherein rendering the geometric shape as polyhedral network comprises producing a node-edge network of the three-dimensional structure.

30. The method of claim 1, further comprising the step of:
(d) predicting the three-dimensional structure of the scaffolded RNA nanostructure.

31. The method of claim 1, further comprising the step of:
(e) assembling the scaffolded RNA nanostructure.

32. The method of claim 31, further comprising the step of:
(f) validating the scaffolded RNA nanostructure.

33. The method of claim 32, wherein the scaffolded RNA nanostructure is validated by comparison with a predicted three-dimensional structure.

34. A polyhedral scaffolded RNA nanostructure designed according to the method of claim 1.

35. A polyhedral scaffolded RNA nanostructure comprising two nucleic acid anti-parallel helices spanning each edge of the structure,
wherein the three-dimensional structure is formed from single stranded nucleic acid staple sequences hybridized to a single stranded RNA scaffold sequence,
wherein the RNA scaffold sequence is routed through the Euler cycle of the network defined by vertices and lines of a node-edge network of the polyhedral structure,
wherein the nanostructure comprises at least one edge including a double-strand crossover,
wherein the location of the double-strand crossover is determined by the spanning tree of the network of the polyhedral structure,
wherein the staple sequences are hybridized to the vertices, edges and double strand crossovers of the scaffold sequence to define the shape of the nanostructure, and
wherein the staples hybridized to the edges implement crossover asymmetry that comprises a difference in the nucleotide position across two helices of the edge of from one to ten nucleotides, inclusive.

36. A polyhedral scaffolded RNA nanostructure comprising two nucleic acid parallel helices spanning each edge of the structure,
wherein the three-dimensional structure is formed from a single stranded RNA scaffold sequence hybridized to itself and may also hybridize to single stranded nucleic acid staple sequences,
wherein the RNA scaffold sequence is routed through the Euler cycle of the network defined by vertices and lines of a node-edge network of the polyhedral structure,
wherein the RNA scaffold sequence hybridizes to itself in at least one edge using parallel crossovers,
wherein the staple sequences, if any, are hybridized to the edges and double strand crossovers of the scaffold sequence to define the shape of the nanostructure, and
wherein the staples hybridized to the edges implement crossover asymmetry that comprises a difference in the nucleotide position across two helices of the edge of from one to ten nucleotides, inclusive.

37. A polyhedral or polygonal scaffolded RNA nanostructure comprising four or more nucleic acid anti-parallel helices spanning each edge of the structure,
wherein the three-dimensional structure is formed from single stranded nucleic acid staple sequences hybridized to a single-stranded RNA scaffold sequence,
wherein the scaffold sequence is routed through the Euler cycle of the network defined by vertices and lines of a node-edge network of the polyhedral structure,
wherein the nanostructure comprises at least one edge including a double strand crossover,
wherein the location of the double strand crossover is determined by a spanning tree of the dual graph of the network of the polyhedral or polygonal structure,
wherein the helices comprising an edge are arranged as a square lattice of four or more helices, or honeycomb lattice of six or more helices,
wherein the helices meeting at a vertex can be beveled or non-beveled, and
wherein the staple sequences are hybridized to the vertices, edges and double strand crossovers of the scaffold sequence to define the shape of the nanostructure.

38. A polyhedral scaffolded RNA nanostructure comprising two nucleic acid anti-parallel helices spanning one or more edges of the structure,
wherein the three-dimensional structure is formed from single stranded nucleic acid staple sequences hybridized to a single stranded RNA scaffold sequence,
wherein the RNA scaffold sequence is routed through the Euler cycle of the network defined by vertices and lines of a node-edge network of the polyhedral structure,
wherein the nanostructure comprises at least one edge including a double-strand crossover,
wherein the location of the double-strand crossover is determined by the spanning tree of the network of the polyhedral structure,
wherein the staple sequences are hybridized to the vertices, edges and double strand crossovers of the scaffold sequence to define the shape of the nanostructure, and
wherein the staples hybridized to the edges implement crossover asymmetry that comprises a difference in the nucleotide position across two helices of the edge of from one to ten nucleotides, inclusive; and
wherein at least one part of the scaffold sequence is not hybridized to staples or itself, and
wherein the non-hybridized scaffold extends from an edge of the polyhedral nanostructure.

39. The polyhedral scaffolded RNA nanostructure of any one of claim 34, further comprising a molecule selected from the group consisting of PNA, protein, lipid, carbohydrate, a small-molecule, a dye, and RNA,
wherein the molecule is covalently or non-covalently bound to, or complexed with, or encapsulated within the nanostructure.

40. The polyhedral scaffolded RNA nanostructure of claim 34, further comprising a therapeutic, diagnostic or prophylactic agent.

41. A method of using the polyhedral scaffolded RNA nanostructure of claim 40 for the delivery of the therapeutic, diagnostic or prophylactic agent to a subject, the method comprising the step of administering the nanoparticle to the subject.

42. A method of programming 3D geometries of arbitrary compositions of one or more molecules selected from the group consisting of PNA, protein, lipid, carbohydrate, a small-molecule, a dye, and RNA,
wherein the molecules are conjugated to an underlying scaffolded RNA nanostructure,
wherein the 3D geometry of the one or more molecules is determined by the 3D geometry of the underlying scaffolded RNA nanostructure, and
wherein the scaffolded RNA nanostructure is designed according to the method of claim 1.

43. The scaffolded RNA nanostructure of claim 34, wherein single-stranded or double-stranded nucleic acid overhang sequences extend from nick positions from the oligonucleotide staple strands.

44. The scaffolded RNA nanostructure of claim 43, wherein the nucleic acid overhang sequences that extend from nick positions from the oligonucleotide staple strands form duplex reinforcements along one or more edges of the structure, or span between two vertices of the structure.

45. The scaffolded RNA nanostructure of claim 43, wherein the single-stranded or double-stranded nucleic acid overhangs comprise one or more sequences of nucleic acids that is complementary to a target RNA or DNA sequence.

46. The scaffolded RNA nanostructure of claim 43, wherein the single-stranded or double-stranded nucleic acid overhangs comprise one or more sequences of nucleic acids that interact with DNA binding proteins or RNA-binding proteins.

47. The scaffolded RNA nanostructure of claim 42, wherein the edge length and nanoparticle geometry is greater than the size of the target molecule that is to be captured, to allow for 1, 2, 3, or more than 3 molecules to be bound independently of any other.

48. The method of claim 1, wherein the RNA scaffold comprises one or more selected from the group consisting of messenger RNA (mRNA), replicating RNA (repRNA), guide-strand RNA (gsRNA), ribosomal RNA (rRNA), transfer RNA (tRNA), genomic transcript RNA, aptamer RNA and functional RNA(s).

49. The method or scaffolded RNA nanostructure of claim 48, wherein the RNA scaffold comprises messenger RNA (mRNA) encoding one or more polypeptides or proteins.

50. The method or scaffolded RNA nanostructure of claim 49, wherein the messenger RNA (mRNA) encodes one or more polypeptide or protein antigens.

51. The method or scaffolded RNA nanostructure of claim 50, wherein the antigen is selected from the group consisting of a viral antigen, bacterial antigen, protozoan antigen, environmental allergen, food allergen and tumor antigen.

52. The method or scaffolded RNA nanostructure of claim 49, wherein the messenger RNA (mRNA) encodes one or more enzymes, fluorescent proteins or antigen-binding proteins.

53. The method or scaffolded RNA nanostructure of claim 52, wherein the messenger RNA (mRNA) encodes the prokaryotic green fluorescent protein (GFP) protein.

54. The method or scaffolded RNA nanostructure of claim 48, wherein the RNA scaffold comprises a functional RNA selected from the group consisting of antisense molecules, silencing RNA (siRNA), micro RNA (miRNA), ribozymes, riboswitches, short hairpin RNA (shRNA), triplex forming RNA, and interfering RNA (RNAi).

55. The method of claim 1, wherein the RNA scaffold and/or staple sequences include one or more modified nucleotides.

56. The method or the scaffolded RNA nanostructure of claim 55, wherein the modified nucleotides reduce or prevent degradation of the modified RNA by RNAse enzymes.

57. The method or the scaffolded RNA nanostructure of claim 55, wherein the one or more modified nucleotides comprises 2′-fluorinated deoxy-uridine, or 2′-fluorinated deoxy-cytosine, or 5-methoxyuridine.

58. The method of claim 1, wherein nanostructure comprises one or more RNA/DNA hybrid regions.

59. The method or the scaffolded RNA nanostructure of claim 58, wherein one or more of the RNA/DNA hybrid regions facilitates release of the scaffold RNA and/or one or more cargo molecules in the presence of an RNA/DNA hybrid specific nuclease.

60. A vaccine comprising the scaffolded RNA nanostructure of claim 50.

61. The method of claim 31, wherein assembling the scaffolded RNA nanostructure comprises synthesizing the RNA scaffold sequence by a method comprising in vitro transcription.


SEQUENCE LISTING (334 sequences)

SEQ ID NO: 1 - RNA, 827 nt, synthetic construct
  source 1..827: mol_type = other RNA
ggtagctaaggaggtaaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcaggcatgcaagcttggcgtaatcatggtcatagctgtttcctgtgtgaaattgttatccgctcacaattccacacaacatacg

SEQ ID NO: 2 - RNA, 1985 nt, synthetic construct
  source 1..1985: mol_type = other RNA
gccctgtttttgcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaagcgacttgctcgtggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactca

SEQ ID NO: 3 - RNA, 1008 nt, synthetic construct
  source 1..1008: mol_type = other RNA
gggtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatca

SEQ ID NO: 4 - RNA, 660 nt, synthetic construct
  source 1..660: mol_type = other RNA
gggcctagtgagtgcattagagactcaagccatgtatccatgaccagaagaggggaccctaggccaaggacatgaggggctctatgcactaatgtctagtcaggccttcaccctagtcatctctatgtggaatacaattgggattcatgagagaatcatgcaagccaaagtcagcaggtagccttatttgatggagttaaccaacagccaaatttattgcccagagcccattaaccatgctagaggagggtttctcctgatgactggctccctgacatctgtgtctgggcctcaaattggtttgaagctgatgggctatgcataatcccatcactaagaccttggtcttgcttgctagaacttttcattacaccactagctgaagtttgttatgactgcccctaatggcaggctcctcagggacagaaggaactggccaaccccatatctaatgttgttgggcttgctgtagggtacccatctatgaggaggcttgtaactgtgatgctcccttagctacttttgatgtatatgcacagtgttggcagtggaccttgctccatgcaattcatatggctatcttgcagtccatggcccaccccagacttcattgctgccctgtgacaccattcattagaacctggtatggaagtgtaccac

SEQ ID NO: 5 - RNA, 924 nt, synthetic construct
  source 1..924: mol_type = other RNA
gggcctagtgagtgcattagagactcaagccatgtatccatgaccagaagaggggaccctaggccaaggacatgaggggctctatgcactaatgtctagtcaggccttcaccctagtcatctctatgtggaatacaattgggattcatgagagaatcatgcaagccaaagtcagcaggtagccttatttgatggagttaaccaacagccaaatttattgcccagagcccattaaccatgctagaggagggtttctcctgatgactggctccctgacatctgtgtctgggcctcaaattggtttgaagctgatgggctatgcataatcccatcactaagaccttggtcttgcttgctagaacttttcattacaccactagctgaagtttgttatgactgcccctaatggcaggctcctcagggacagaaggaactggccaaccccatatctaatgttgttgggcttgctgtagggtacccatctatgaggaggcttgtaactgtgatgctcccttagctacttttgatgtatatgcacagtgttggcagtggaccttgctccatgcaattcatatggctatcttgcagtccatggcccaccccagacttcattgctgccctgtgacaccattcattagaacctggtatggaagtgtaccacagagctgcaaggggttgtagaccacttatggtgaaatttgtagaatccagggtggtgagctgtaaatgaaactttggataactggctttctagagccttcctcccaccctcccctgtttgccatgccccaggggcaagcactgccctttgcactctcactacaggtaaagagtgtccccaggagtaaatttctagccccatagaaaaggaaggtctagagggaaattggcaatgggcacctgtcccattataagcatctatttg

SEQ ID NO: 6 - DNA, 34 bp, synthetic construct
  source 1..34: mol_type = other DNA
gaattctaatacgactcactataggtagctaagg

SEQ ID NO: 7 - DNA, 23 bp, synthetic construct
  source 1..23: mol_type = other DNA
cgtatgttgtgtggaattgtgag

SEQ ID NO: 8 - DNA, 40 bp, synthetic construct
  source 1..40: mol_type = other DNA
cttaagtaatacgactcactatagccctggcagtcagagg

SEQ ID NO: 9 - DNA, 20 bp, synthetic construct
  source 1..20: mol_type = other DNA
tgagtctcgggtggagacag

SEQ ID NO: 10 - DNA, 39 bp, synthetic construct
  source 1..39: mol_type = other DNA
taatacgactcactatagggtctcgctggtgaaaagaaa

SEQ ID NO: 11 - DNA, 19 bp, synthetic construct
  source 1..19: mol_type = other DNA
ggccggagacagtcaaatc

SEQ ID NO: 12 - DNA, 52 bp, synthetic construct
  source 1..52: mol_type = other DNA
ggcttatcgaaattaatacgactcactatagggcctagtgagtgcattagag

SEQ ID NO: 13 - DNA, 24 bp, synthetic construct
  source 1..24: mol_type = other DNA
gtggtacacttccataccaggttc

SEQ ID NO: 14 - DNA, 29 bp, synthetic construct
  source 1..29: mol_type = other DNA
caaatagatgcttataatgggacaggtgc

SEQ ID NO: 15 - DNA, 25 bp, synthetic construct
  source 1..25: mol_type = other DNA
ggagctttgttccttgggttcttgg

SEQ ID NO: 16 - DNA, 28 bp, synthetic construct
  source 1..28: mol_type = other DNA
ggcttatcgaaattaatacgactcacta

SEQ ID NO: 17 - DNA, 28 bp, synthetic construct
  source 1..28: mol_type = other DNA
ggagctgttgatcctttaggtatctttc

SEQ ID NO: 18 - RNA, 792 nt, synthetic construct
  source 1..792: mol_type = other RNA
aagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcaggcatgcaagcttggcgtaatcatggtcatagctgtttcctgtgtgaaattgttatccgctcacaattccacacaa

SEQ ID NO: 19 - DNA, 44 bp, synthetic construct
  source 1..44: mol_type = other DNA
ggtcggggtagagctcctcgcccttttgtgtggctgcttcatgt

SEQ ID NO: 20 - DNA, 44 bp, synthetic construct
  source 1..44: mol_type = other DNA
ctgcacgccgtgatgggcaccaccccggtgaaccggctgaagca

SEQ ID NO: 21 - DNA, 44 bp, synthetic construct
  source 1..44: mol_type = other DNA
cagctatgaccgtcgccgatgggggtgttctgccacacaggaaa

SEQ ID NO: 22 - DNA, 44 bp, synthetic construct
  source 1..44: mol_type = other DNA
aagcttgcatgtgtcgggcagcagcacggggccatgattacgcc

SEQ ID NO: 23 - DNA, 33 bp, synthetic construct
  source 1..33: mol_type = other DNA
ccagcttgacgttgtggctgttgtagtctttgc

SEQ ID NO: 24 - DNA, 33 bp, synthetic construct
  source 1..33: mol_type = other DNA
tcagggcgtcgcgcttctcgttggggttgtact

SEQ ID NO: 25 - DNA, 22 bp, synthetic construct
  source 1..22: mol_type = other DNA
tgccccaggatccatgatatag

SEQ ID NO: 26 - DNA, 44 bp, synthetic construct
  source 1..44: mol_type = other DNA
tcgatgttgtgtcctggacgtagccttcgggcacgctgccgtcc

SEQ ID NO: 27 - DNA, 44 bp, synthetic construct
  source 1..44: mol_type = other DNA
agttcaccttggtccttgaagaagatggtgcgcgcggatcttga

SEQ ID NO: 28 - DNA, 33 bp, synthetic construct
  source 1..33: mol_type = other DNA
ctgaacttatcgccctcgccctcgccgccggcg

SEQ ID NO: 29 - DNA, 33 bp, synthetic construct
  source 1..33: mol_type = other DNA
gcggtcacgtccatgccgagagtgatcgacacg

SEQ ID NO: 30 - DNA, 22 bp, synthetic construct
  source 1..22: mol_type = other DNA
gtggccgtttaccgtaggtggc

SEQ ID NO: 31 - DNA, 33 bp, synthetic construct
  source 1..33: mol_type = other DNA
ccctcgaactcgatgcggttcaccaggttgccg

SEQ ID NO: 32 - DNA, 33 bp, synthetic construct
  source 1..33: mol_type = other DNA
gtggtgcagggccagggcacgggcagcgtgtcg

SEQ ID NO: 33 - DNA, 22 bp, synthetic construct
  source 1..22: mol_type = other DNA
cttcacctcggatgcccttcag

SEQ ID NO: 34 - DNA, 81 bp, synthetic construct
  source 1..81: mol_type = other DNA
tggtagtggtctttttggcgagctgcatggcggacttgtttttaagaagtcgtgaattgtgagcgtttttgataacaattt

SEQ ID NO: 35 - DNA, 81 bp, synthetic construct
  source 1..81: mol_type = other DNA
atgccgttctttttttctgcttgtcgggttgccgtccttttttccttgaagtcgcgcgggtcttgttttttagttgccgtc

SEQ ID NO: 36 - DNA, 81 bp, synthetic construct
  source 1..81: mol_type = other DNA
gaactccagcatttttggaccatgtgagactgggtgcttttttcaggtagtggtcctgcagttactttttttgtacagctc

SEQ ID NO: 37 - DNA, 81 bp, synthetic construct
  source 1..81: mol_type = other DNA
gatgaacttcatttttgggtcagcttgcgtcgccgtcctttttagctcgaccagaggtcagggtgtttttgtcacgagggt

SEQ ID NO: 38 - DNA, 34 bp, synthetic construct
  source 1..34: mol_type = other DNA
cgctcctggaacttgtggccgtttaccctcgccg

SEQ ID NO: 39 - DNA, 32 bp, synthetic construct
  source 1..32: mol_type = other DNA
gacacgctgacgtagccttcgggcagatggtg

SEQ ID NO: 40 - DNA, 87 bp, synthetic construct
  source 1..87: mol_type = other DNA
tgtcgggcagctttttttagcacggggccgccgtaggtggtttttttcatcgccctcgcgtcgccgtcctttttttagctcgaccag

SEQ ID NO: 41 - DNA, 44 bp, synthetic construct
  source 1..44: mol_type = other DNA
cggtgaacgactgggtgctcaggtagtggtgatgggcaccaccc

SEQ ID NO: 42 - DNA, 44 bp, synthetic construct
  source 1..44: mol_type = other DNA
ttcagggtcagcttgtcgccgatgggggtgttctgcagatgaac

SEQ ID NO: 43 - DNA, 22 bp, synthetic construct
  source 1..22: mol_type = other DNA
atggcggacttgtccttgaaga

SEQ ID NO: 44 - DNA, 44 bp, synthetic construct
  source 1..44: mol_type = other DNA
ttgctcagggcgagctcctcgcccttttgtgtgggttggggtct

SEQ ID NO: 45 - DNA, 44 bp, synthetic construct
  source 1..44: mol_type = other DNA
ttgccggtggtgctggtagtggtcggcgagctgcaacgggcagc

SEQ ID NO: 46 - DNA, 87 bp, synthetic construct
  source 1..87: mol_type = other DNA
cgctgccgtccttttttttcgatgttgtgtgcttgtcggctttttttcatgatatagaggtcacgagggttttttttgggccagggc

SEQ ID NO: 47 - DNA, 34 bp, synthetic construct
  source 1..34: mol_type = other DNA
ccgttcttcgcggatcttgaagttcatcccggcg

SEQ ID NO: 48 - DNA, 32 bp, synthetic construct
  source 1..32: mol_type = other DNA
gcggtcactccatgccgagagtgaccttgatg

SEQ ID NO: 49 - DNA, 22 bp, synthetic construct
  source 1..22: mol_type = other DNA
gaactccagcatgtacagctcg

SEQ ID NO: 50 - DNA, 44 bp, synthetic construct
  source 1..44: mol_type = other DNA
gctgaagcctccagcttgtgccccaggatgtggtcggggtagcg

SEQ ID NO: 51 - DNA, 87 bp, synthetic construct
  source 1..87: mol_type = other DNA
ttgccgtcctctttttttcttgaagtcgacgcgggtcttgttttttttagttgccgtcgaagaagtcgttttttttgctgcttcatg

SEQ ID NO: 52 - DNA, 32 bp, synthetic construct
  source 1..32: mol_type = other DNA
ccctcgaatcgatgcggttcaccatgaccatg

SEQ ID NO: 53 - DNA, 34 bp, synthetic construct
  source 1..34: mol_type = other DNA
attacgccacacacaggaaacagctagggtgtcg

SEQ ID NO: 54 - DNA, 87 bp, synthetic construct
  source 1..87: mol_type = other DNA
agcttgcatgctttttttctgcagttactggaccatgtgattttttttcgcgcttctcaattgtgagcgtttttttgataacaattt

SEQ ID NO: 55 - DNA, 22 bp, synthetic construct
  source 1..22: mol_type = other DNA
cttcacctcggtgcccttcagc

SEQ ID NO: 56 - DNA, 44 bp, synthetic construct
  source 1..44: mol_type = other DNA
tagttgtaactgcacgccgtaggtcagggtcgttgtggctgttg

SEQ ID NO: 57 - DNA, 44 bp, synthetic construct
  source 1..44: mol_type = other DNA
acaaaaggtacctgactgccagggctgagtctctgacccattat

SEQ ID NO: 58 - DNA, 44 bp, synthetic construct
  source 1..44: mol_type = other DNA
cctaagcgtgcattagcacgtccttcatcgcctgcagtcacacg

SEQ ID NO: 59 - DNA, 54 bp, synthetic construct
  source 1..54: mol_type = other DNA
accttaccgactttttgcttatcgcagtcccactgctttttttgtacgtacacg

SEQ ID NO: 60 - DNA, 33 bp, synthetic construct
  source 1..33: mol_type = other DNA
ttcatatcattcggaaatcgccggttaaagagc

SEQ ID NO: 61 - DNA, 33 bp, synthetic construct
  source 1..33: mol_type = other DNA
ttcgcttggtttaccggggcttcgatctaacgg

SEQ ID NO: 62 - DNA, 54 bp, synthetic construct
  source 1..54: mol_type = other DNA
ttcttttcgccttttttttccctcacgtcgaaacacacttttttgggtttcccc

SEQ ID NO: 63 - DNA, 44 bp, synthetic construct
  source 1..44: mol_type = other DNA
ctatcggtcagggattcagttaatgatagtgtggtactggttca

SEQ ID NO: 64 - DNA, 44 bp, synthetic construct
  source 1..44: mol_type = other DNA
tagccttggagccggttcgcctcattaacctattcaggagtatt

SEQ ID NO: 65 - DNA, 54 bp, synthetic construct
  source 1..54: mol_type = other DNA
cactgattcagtttttgctctgggctggtacttagatgttttttttcagttccc

SEQ ID NO: 66 - DNA, 22 bp, synthetic construct
  source 1..22: mol_type = other DNA
ttttcctcgggctccccgttcg

SEQ ID NO: 67 - DNA, 33 bp, synthetic construct
  source 1..33: mol_type = other DNA
ttgatttcctcgccgctactgggggaacgtcca

SEQ ID NO: 68 - DNA, 33 bp, synthetic construct
  source 1..33: mol_type = other DNA
ctttcgtggcaggcgtcacaccgtatatctcgg

SEQ ID NO: 69 - DNA, 33 bp, synthetic construct
  source 1..33: mol_type = other DNA
aacacacagcgcgcctttccagacgctgtcggg

SEQ ID NO: 70 - DNA, 33 bp, synthetic construct
  source 1..33: mol_type = other DNA
atgaccccgtttgcatcgggttggtaatccact

SEQ ID NO: 71 - DNA, 54 bp, synthetic construct
  source 1..54: mol_type = other DNA
gatggtccccctttttcatattcagactacggggctgttttttcaccctgtatc

SEQ ID NO: 72 - DNA, 22 bp, synthetic construct
  source 1..22: mol_type = other DNA
gcatttttgtgaggataccacg

SEQ ID NO: 73 - DNA, 33 bp, synthetic construct
  source 1..33: mol_type = other DNA
cagcatgttgtcccgccctactcatcgtccgct

SEQ ID NO: 74 - DNA, 33 bp, synthetic construct
  source 1..33: mol_type = other DNA
aatttttccacccccagccacaagtcaagctca

SEQ ID NO: 75 - DNA, 33 bp, synthetic construct
  source 1..33: mol_type = other DNA
cgccgggggtttcaggttctttttcaccatggc

SEQ ID NO: 76 - DNA, 33 bp, synthetic construct
  source 1..33: mol_type = other DNA
tagatcacacccaaccttcaacctgcctcccct

SEQ ID NO: 77 - RNA, 232 nt, synthetic construct
  source 1..232: mol_type = other RNA
ggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcc

SEQ ID NO: 78 - DNA, 54 bp, synthetic construct
  source 1..54: mol_type = other DNA
tttcacccgcttttttttatcgttactcttgctacagatttttatataagtcgc

SEQ ID NO: 79 - DNA, 44 bp, synthetic construct
  source 1..44: mol_type = other DNA
tcgcacttctgtcggctcccctattcggttaactatgtcagcat

SEQ ID NO: 80 - DNA, 44 bp, synthetic construct
  source 1..44: mol_type = other DNA
atgcctcacagccagttaagactcggtttccctatacctccagc

SEQ ID NO: 81 - DNA, 81 bp, synthetic construct
  source 1..81: mol_type = other DNA
aacattagtcgtttttgttcggtcctctctataccctgtttttcaacttaacgccacaccttcgctttttaggcttacaga

SEQ ID NO: 82 - DNA, 22 bp, synthetic construct
  source 1..22: mol_type = other DNA
cgggtttcgggcagttagtgtt

SEQ ID NO: 83 - DNA, 54 bp, synthetic construct
  source 1..54: mol_type = other DNA
tgccgcagctttttttcggtgcatggttctcccggttttttttgattggccttt

SEQ ID NO: 84 - DNA, 44 bp, synthetic construct
  source 1..44: mol_type = other DNA
acatcttccgcagctttcggggagaaccagctattagccccgtt

SEQ ID NO: 85 - DNA, 44 bp, synthetic construct
  source 1..44: mol_type = other DNA
cgaccagtgagattcacgaggcgctacctaaatgcaggccgact

SEQ ID NO: 86 - DNA, 54 bp, synthetic construct
  source 1..54: mol_type = other DNA
tgtctcccgtgtttttataacattctcagtgctctacctttttcccggagatga

SEQ ID NO: 87 - DNA, 22 bp, synthetic construct
  source 1..22: mol_type = other DNA
cttgccgaaaccggtattcgca

SEQ ID NO: 88 - DNA, 22 bp, synthetic construct
  source 1..22: mol_type = other DNA
cacccgccgtgtagctggcggt

SEQ ID NO: 89 - DNA, 33 bp, synthetic construct
  source 1..33: mol_type = other DNA
gacgttagctgggttgtttccctcttctggtat

SEQ ID NO: 90 - DNA, 33 bp, synthetic construct
  source 1..33: mol_type = other DNA
cttcgactataaacagttgcagccagcacgacg

SEQ ID NO: 91 - DNA, 54 bp, synthetic construct
  source 1..54: mol_type = other DNA
ctattacgcttttttttctttaaatgacttaaccatgatttttctttgggacct

SEQ ID NO: 92 - DNA, 22 bp, synthetic construct
  source 1..22: mol_type = other DNA
atcgtttcccatggctgcttct

SEQ ID NO: 93 - DNA, 33 bp, synthetic construct
  source 1..33: mol_type = other DNA
cttcccacaagccaacatcctggctgtaacata

SEQ ID NO: 94 - DNA, 33 bp, synthetic construct
  source 1..33: mol_type = other DNA
gccttctcggacaaccgtcgcccggccctgggc

SEQ ID NO: 95 - DNA, 33 bp, synthetic construct
  source 1..33: mol_type = other DNA
agcgtcgcacgctcccctacccaacaacgacta

SEQ ID NO: 96 - DNA, 33 bp, synthetic construct
  source 1..33: mol_type = other DNA
cgcctttcatattaacctgtttcccatcgcata

SEQ ID NO: 97 - DNA, 44 bp, synthetic construct
  source 1..44: mol_type = other DNA
gttgcttcagcgattaacgttggacaggaacccgcagggcattt

SEQ ID NO: 98 - DNA, 54 bp, synthetic construct
  source 1..54: mol_type = other DNA
aggggtcgacttttttcaccctgccccaccgtagtgccttttttcgtcatcacg

SEQ ID NO: 99 - DNA, 22 bp, synthetic construct
  source 1..22: mol_type = other DNA
ggcctcgccttcaagtacagga

SEQ ID NO: 100 - DNA, 54 bp, synthetic construct
  source 1..54: mol_type = other DNA
accagcctacatttttcgcttaaaccgcgtcccccctttttttcgcagtaacac

SEQ ID NO: 101 - DNA, 33 bp, synthetic construct
  source 1..33: mol_type = other DNA
cctggaaacctcagccttgattttccgccgaag

SEQ ID NO: 102 - DNA, 33 bp, synthetic construct
  source 1..33: mol_type = other DNA
ttacggcacatatcagcgtgccttctcgatttg

SEQ ID NO: 103 - DNA, 54 bp, synthetic construct
  source 1..54: mol_type = other DNA
gggtggagacatttttgcctggccatcggtacgatttgtttttatgttacctga

SEQ ID NO: 104 - DNA, 44 bp, synthetic construct
  source 1..44: mol_type = other DNA
cgtgcaggtcgctgaccacctgtgtcggtttggattacgccatt

SEQ ID NO: 105 - DNA, 44 bp, synthetic construct
  source 1..44: mol_type = other DNA
acaaggaatttcaagcgccttggtattctctacgaacttacccg

SEQ ID NO: 106 - DNA, 81 bp, synthetic construct
  source 1..81: mol_type = other DNA
agttccttcactttttccgagttctctcgctaccttagtttttgaccgttatagtcaattaacctttttttccggcaccgg

SEQ ID NO: 107 - DNA, 22 bp, synthetic construct
  source 1..22: mol_type = other DNA
ccattttgcctcgcttcaccta

SEQ ID NO: 108 - DNA, 54 bp, synthetic construct
  source 1..54: mol_type = other DNA
tttgcacagtgtttttctgtgtttttagatttcagctctttttcacgagcaagt

SEQ ID NO: 109 - DNA, 22 bp, synthetic construct
  source 1..22: mol_type = other DNA
cgctaaccccattacggccgcc

SEQ ID NO: 110 - DNA, 44 bp, synthetic construct
  source 1..44: mol_type = other DNA
gtggtcggggtacagctcctcgcccttttgtgttgctgcttcat

SEQ ID NO: 111 - DNA, 44 bp, synthetic construct
  source 1..44: mol_type = other DNA
cactgcacgccaggatgggcaccaccccggtgaagcggctgaag

SEQ ID NO: 112 - DNA, 81 bp, synthetic construct
  source 1..81: mol_type = other DNA
tggtagtggtcggtttttcgagctgcacatggcggacttgtttttaagaagtcgggaattgtgagcgtttttgataacaat

SEQ ID NO: 113 - DNA, 44 bp, synthetic construct
  source 1..44: mol_type = other DNA
aacagctatgagtcgccgatgggggtgttctgcttcacacagga

SEQ ID NO: 114 - DNA, 44 bp, synthetic construct
  source 1..44: mol_type = other DNA
ccaagcttgcatgtcgggcagcagcacggggccccatgattacg

SEQ ID NO: 115 - DNA, 33 bp, synthetic construct
  source 1..33: mol_type = other DNA
tccagcttacgttgtggctgttgtagtctttgc

SEQ ID NO: 116 - DNA, 33 bp, synthetic construct
  source 1..33: mol_type = other DNA
tcagggcgatcgcgcttctcgttggggttgtac

SEQ ID NO: 117 - DNA, 22 bp, synthetic construct
  source 1..22: mol_type = other DNA
gtgccccaggaccatgatatag

SEQ ID NO: 118 - DNA, 81 bp, synthetic construct
  source 1..81: mol_type = other DNA
atgccgttcttcttttttgcttgtcggtgttgccgtcctctttttcttgaagtcggcgcgggtcttgttttttagttgccg

SEQ ID NO: 119 - DNA, 44 bp, synthetic construct
  source 1..44: mol_type = other DNA
tcgatgttgtggctcctggacgtagccttcgggcgctgccgtcc

SEQ ID NO: 120 - DNA, 44 bp, synthetic construct
  source 1..44: mol_type = other DNA
agttcaccttgtcgtccttgaagaagatggtgcgcggatcttga

SEQ ID NO: 121 - DNA, 81 bp, synthetic construct
  source 1..81: mol_type = other DNA
cgaactccagcagtttttgaccatgtggactgggtgctcatttttggtagtggttgcctgcagttactttttttgtacagc

SEQ ID NO: 122 - DNA, 33 bp, synthetic construct
  source 1..33: mol_type = other DNA
cgctgaaccatcgccctcgccctcgctcccggc

SEQ ID NO: 123 - DNA, 33 bp, synthetic construct
  source 1..33: mol_type = other DNA
ggcggtcatcgtccatgccgagagtgacggaca

SEQ ID NO: 124 - DNA, 22 bp, synthetic construct
  source 1..22: mol_type = other DNA
ttgtggccgttgccgtaggtgg

SEQ ID NO: 125 - DNA, 81 bp, synthetic construct
  source 1..81: mol_type = other DNA
agatgaacttcagtttttggtcagctttacgtcgccgtcctttttagctcgaccgtaggtcagggtgtttttgtcacgagg

SEQ ID NO: 126 - DNA, 33 bp, synthetic construct
  source 1..33: mol_type = other DNA
cgccctcggctcgatgcggttcaccagcttgcc

SEQ ID NO: 127 - DNA, 33 bp, synthetic construct
  source 1..33: mol_type = other DNA
ggtggtgcgtgggccagggcacgggcagggtgt

SEQ ID NO: 128 - DNA, 22 bp, synthetic construct
  source 1..22: mol_type = other DNA
aacttcacctcgatgcccttca

SEQ ID NO: 129 - DNA, 54 bp, synthetic construct
  source 1..54: mol_type = other DNA
ttttcacccgcttttttttatcgttacaaccttgctacagtttttaatataagt

SEQ ID NO: 130 - DNA, 44 bp, synthetic construct
  source 1..44: mol_type = other DNA
gcttttcctggcttggtcttccggcgagcgggcgatgcttagag

SEQ ID NO: 131 - DNA, 44 bp, synthetic construct
  source 1..44: mol_type = other DNA
ttgttgcttcacgattaacgttggacaggaaccaagcagggcat

SEQ ID NO: 132 - DNA, 44 bp, synthetic construct
  source 1..44: mol_type = other DNA
ttcgcacttctccttcggctcccctattcggttttatgtcagca

SEQ ID NO: 133 - DNA, 44 bp, synthetic construct
  source 1..44: mol_type = other DNA
catgcctcacacgcccagttaagactcggtttcgatacctccag

SEQ ID NO: 134 - DNA, 81 bp, synthetic construct
  source 1..81: mol_type = other DNA
tcaacattagtcgtttttgttcggtccgggtctatacccttttttgcaacttaagcacaccttcgcatttttggcttacag

SEQ ID NO: 135 - DNA, 33 bp, synthetic construct
  source 1..33: mol_type = other DNA
tcacagcacgtgtcccgccctactcatcatccg

SEQ ID NO: 136 - DNA, 33 bp, synthetic construct
  source 1..33: mol_type = other DNA
ctaatttttttcacccccagccacaagtcgagc

SEQ ID NO: 137 - DNA, 22 bp, synthetic construct
  source 1..22: mol_type = other DNA
tgtgcatttttacaggatacca

SEQ ID NO: 138 - DNA, 54 bp, synthetic construct
  source 1..54: mol_type = other DNA
ggttcttttcgccttttttttccctcagtgtcgaaacacatttttctgggtttc

SEQ ID NO: 139 - DNA, 33 bp, synthetic construct
  source 1..33: mol_type = other DNA
ggctagatttacccaaccttcaacctcactccc

SEQ ID NO: 140 - DNA, 33 bp, synthetic construct
  source 1..33: mol_type = other DNA
ctcgccggacggtttcaggttctttttgcccat

SEQ ID NO: 141 - DNA, 22 bp, synthetic construct
  source 1..22: mol_type = other DNA
caccgggtttctccagttagtg

SEQ ID NO: 142 - DNA, 44 bp, synthetic construct
  source 1..44: mol_type = other DNA
ttcgtgcaggtacctgaccacctgtgtcggttttcattacgcca

SEQ ID NO: 143 - DNA, 44 bp, synthetic construct
  source 1..44: mol_type = other DNA
cgacaaggaatctcaagcgccttggtattctctcggaacttacc

SEQ ID NO: 144 - DNA, 54 bp, synthetic construct
  source 1..54: mol_type = other DNA
tcgggtggagacatttttgcctggccaggggtacgatttgtttttatgttacct

SEQ ID NO: 145 - DNA, 44 bp, synthetic construct
  source 1..44: mol_type = other DNA
tatacaaaaggctctgactgccagggctgagtccgctgacccat

SEQ ID NO: 146 - DNA, 44 bp, synthetic construct
  source 1..44: mol_type = other DNA
acgcctaagcgagattagcacgtccttcatcgctacgcagtcac

SEQ ID NO: 147 - DNA, 54 bp, synthetic construct
  source 1..54: mol_type = other DNA
tcaccttaccgactttttgcttatcgctgctcccactgctttttttgtacgtac

SEQ ID NO: 148 - DNA, 33 bp, synthetic construct
  source 1..33: mol_type = other DNA
agcttcgcccgtttaccggggcttcgttataac

SEQ ID NO: 149 - DNA, 33 bp, synthetic construct
  source 1..33: mol_type = other DNA
ggttcatacccattcggaaatcgccggatcaag

SEQ ID NO: 150 - DNA, 22 bp, synthetic construct
  source 1..22: mol_type = other DNA
ttgcgctaaccagttacggccg

SEQ ID NO: 151 - DNA, 54 bp, synthetic construct
  source 1..54: mol_type = other DNA
aggatggtccccctttttcatattcaggtgtacggggctgttttttcaccctgt

SEQ ID NO: 152 - DNA, 44 bp, synthetic construct
  source 1..44: mol_type = other DNA
cactatcggtctatggattcagttaatgatagtcggtactggtt

SEQ ID NO: 153 - DNA, 44 bp, synthetic construct
  source 1..44: mol_type = other DNA
tttagccttggcccccggttcgcctcattaaccagtcaggagta

SEQ ID NO: 154 - DNA, 54 bp, synthetic construct
  source 1..54: mol_type = other DNA
tgccgcagcttcgtttttgtgcatggtctatctcccggttttttttgattggcc

SEQ ID NO: 155 - DNA, 54 bp, synthetic construct
  source 1..54: mol_type = other DNA
taggggtcgactctttttaccctgcccgcaccgtagtgccttttttcgtcatca

SEQ ID NO: 156 - DNA, 33 bp, synthetic construct
  source 1..33: mol_type = other DNA
acgcctttatattaacctgtttcccaacgcata

SEQ ID NO: 157 - DNA, 33 bp, synthetic construct
  source 1..33: mol_type = other DNA
agcgtcgcaacgctcccctacccaacatcgact

SEQ ID NO: 158 - DNA, 22 bp, synthetic construct
  source 1..22: mol_type = other DNA
cggcctcgcctcaagtacagga

SEQ ID NO: 159 - DNA, 33 bp, synthetic construct
  source 1..33: mol_type = other DNA
ccttcccaaagccaacatcctggctgcaacata

SEQ ID NO: 160 - DNA, 33 bp, synthetic construct
  source 1..33: mol_type = other DNA
gccttctcgggacaaccgtcgcccggctctggg

SEQ ID NO: 161 - DNA, 22 bp, synthetic construct
  source 1..22: mol_type = other DNA
catcgtttccctggctgcttct

SEQ ID NO: 162 - DNA, 54 bp, synthetic construct
  source 1..54: mol_type = other DNA
aaccagcctacactttttgcttaaacccgtccccccttcgtttttcagtaacac

SEQ ID NO: 163 - DNA, 81 bp, synthetic construct
  source 1..81: mol_type = other DNA
ctagttccttcactttttccgagttctttcgctaccttagtttttgaccgttatccatcaattaacctttttttccggcac

SEQ ID NO: 164 - DNA, 33 bp, synthetic construct
  source 1..33: mol_type = other DNA
agttacggacatatcagcgtgccttccggattt

SEQ ID NO: 165 - DNA, 33 bp, synthetic construct
  source 1..33: mol_type = other DNA
gcctggaacgcctcagccttgattttctcccga

SEQ ID NO: 166 - DNA, 22 bp, synthetic construct
  source 1..22: mol_type = other DNA
caccattttgctcgcttcacct

SEQ ID NO: 167 - DNA, 54 bp, synthetic construct
  source 1..54: mol_type = other DNA
tgtgtctcccgtgtttttataacattcaacagtgctctactttttccccggaga

SEQ ID NO: 168 - DNA, 33 bp, synthetic construct
  source 1..33: mol_type = other DNA
cggacgtttctgggttgtttccctctgctggta

SEQ ID NO: 169 - DNA, 33 bp, synthetic construct
  source 1..33: mol_type = other DNA
tcttcgactaataaacagttgcagccatcacga

SEQ ID NO: 170 - DNA, 22 bp, synthetic construct
  source 1..22: mol_type = other DNA
agcacccgccgttagctggcgg

SEQ ID NO: 171 - DNA, 54 bp, synthetic construct
  source 1..54: mol_type = other DNA
ctattacgctttcttttttttaaatgaacttaaccatgacttttttttgggacc

SEQ ID NO: 172 - DNA, 44 bp, synthetic construct
  source 1..44: mol_type = other DNA
acatcttccgcaatagctttcggggagaaccagttagccccgtt

SEQ ID NO: 173 - DNA, 44 bp, synthetic construct
  source 1..44: mol_type = other DNA
cgaccagtgagtgaattcacgaggcgctacctagcaggccgact

SEQ ID NO: 174 - DNA, 54 bp, synthetic construct
  source 1..54: mol_type = other DNA
tgtttgcacagtgtttttctgtgtttttgatttcagctcctttttacgagcaag

SEQ ID NO: 175 - DNA, 33 bp, synthetic construct
  source 1..33: mol_type = other DNA
cggttgatcgctcgccgctactggggatacgtc

SEQ ID NO: 176 - DNA, 33 bp, synthetic construct
  source 1..33: mol_type = other DNA
cactttcgcgggcaggcgtcacaccgtgaatct

SEQ ID NO: 177 - DNA, 22 bp, synthetic construct
  source 1..22: mol_type = other DNA
ttcttttcctctgctccccgtt

SEQ ID NO: 178 - DNA, 54 bp, synthetic construct
  source 1..54: mol_type = other DNA
cacactgattcagtttttgctctgggcggggtacttagattttttgtttcagtt

SEQ ID NO: 179 - DNA, 33 bp, synthetic construct
  source 1..33: mol_type = other DNA
gggatgaccagtttgcatcgggttgggcttcca

SEQ ID NO: 180 - DNA, 33 bp, synthetic construct
  source 1..33: mol_type = other DNA
ctaacacaatcgcgcgcctttccagactaagtc

SEQ ID NO: 181 - DNA, 22 bp, synthetic construct
  source 1..22: mol_type = other DNA
ccccttgccgatccggtattcg

SEQ ID NO: 182 - DNA, 54 bp, synthetic construct
  source 1..54: mol_type = other DNA
tgataaattaatgtttttccggagaggcaggtcattgccttttttgagagtctg

SEQ ID NO: 183 - DNA, 44 bp, synthetic construct
  source 1..44: mol_type = other DNA
accgttctagcaatatttaaattgtaaacgttatatgatattca

SEQ ID NO: 184 - DNA, 54 bp, synthetic construct
  source 1..54: mol_type = other DNA
gagacagtcaaattttttcaccatcaaatattttgttaaatttttattcgcatt

SEQ ID NO: 185 - DNA, 44 bp, synthetic construct
  source 1..44: mol_type = other DNA
gcctggggtgcttttcaccagtgagacaggccgtaaagtgtaaa

SEQ ID NO: 186 - DNA, 54 bp, synthetic construct
  source 1..54: mol_type = other DNA
ttgggcgccagggttttttggtttttcctaatgagtgagcttttttaactcaca

SEQ ID NO: 187 - DNA, 44 bp, synthetic construct
  source 1..44: mol_type = other DNA
aaggctatgtagctatttttgagagacgtagcggtttgtctaca

SEQ ID NO: 188 - DNA, 54 bp, synthetic construct
  source 1..54: mol_type = other DNA
ccagctggcgaaatttttgggggatgtgttttcccagtcatttttcgacgttgt

SEQ ID NO: 189 - DNA, 44 bp, synthetic construct
  source 1..44: mol_type = other DNA
tcgctattacgttgttatccgctcacaattccatgcgggcctct

SEQ ID NO: 190 - DNA, 54 bp, synthetic construct
  source 1..54: mol_type = other DNA
aactgttgggaagtttttggcgatcggcacaacatacgagtttttccggaagca

SEQ ID NO: 191 - DNA, 44 bp, synthetic construct
  source 1..44: mol_type = other DNA
aaatcagctcaccattcgccattcaggctgcgcaaatttttgtt

SEQ ID NO: 192 - DNA, 54 bp, synthetic construct
  source 1..54: mol_type = other DNA
gtgccggaaaccatttttggcaaagcgttttttaaccaattttttaggaacgcc

SEQ ID NO: 193 - DNA, 44 bp, synthetic construct
  source 1..44: mol_type = other DNA
acgccagggctgcaaggcgattaagttctggcaccgcttgggta

SEQ ID NO: 194 - DNA, 54 bp, synthetic construct
  source 1..54: mol_type = other DNA
tgagcgagtaacatttttacccgtcggaacaaacggcggatttttttgaccgta

SEQ ID NO: 195 - DNA, 44 bp, synthetic construct
  source 1..44: mol_type = other DNA
cctcaggaagacagctttcatcaacattaaatggacagtatcgg

SEQ ID NO: 196 - DNA, 54 bp, synthetic construct
  source 1..54: mol_type = other DNA
tcgcgtctggcctttttttcctgtagctcgcactccagcctttttagctttccg

SEQ ID NO: 197 - DNA, 44 bp, synthetic construct
  source 1..44: mol_type = other DNA
agccccaatgtaccccggttgataattaatatcaaaaacagaaa

SEQ ID NO: 198 - DNA, 54 bp, synthetic construct
  source 1..54: mol_type = other DNA
taaaactagcatgttttttcaatcataaaacaggaagatttttttgtataagca

SEQ ID NO: 199 - DNA, 44 bp, synthetic construct
  source 1..44: mol_type = other DNA
ggtaatcggagcaaacaagagaatcgtgggattctccgatgaac

SEQ ID NO: 200 - DNA, 54 bp, synthetic construct
  source 1..54: mol_type = other DNA
acgttggtgtagattttttgggcgcattctgccagtttgatttttggggacgac

SEQ ID NO: 201 - DNA, 44 bp, synthetic construct
  source 1..44: mol_type = other DNA
attaatgacgggaaacctgtcgtgccggtcatgggataagctgc

SEQ ID NO: 202 - DNA, 54 bp, synthetic construct
  source 1..54: mol_type = other DNA
cgctcactgcccgtttttctttccagtatcggccaacgcgtttttcggggagag

SEQ ID NO: 203 - DNA, 44 bp, synthetic construct
  source 1..44: mol_type = other DNA
atcatggtccgggtaccgagctcgaagttgttaattgcttcgta

SEQ ID NO: 204 - DNA, 54 bp, synthetic construct
  source 1..54: mol_type = other DNA
gcaggtcgactcttttttagaggatcccatagctgtttccttttttgtgtgaaa

SEQ ID NO: 205 - DNA, 44 bp, synthetic construct
  source 1..44: mol_type = other DNA
gcatgcctaaaacgacggccagtgcctgcacgtaaccgaagctt

SEQ ID NO: 206 - DNA, 54 bp, synthetic construct
  source 1..54: mol_type = other DNA
gcaccgtagtgccttttttcgtcatcaccgggacaaccgttttttcgcccggcc

SEQ ID NO: 207 - DNA, 44 bp, synthetic construct
  source 1..44: mol_type = other DNA
gcttttcctggttacttatgtcagcattcgcacgatgcttagag

SEQ ID NO: 208 - DNA, 44 bp, synthetic construct
  source 1..44: mol_type = other DNA
ttgttgcttcagggcttttcacccgctttatcgaagcagggcat

SEQ ID NO: 209 - DNA, 54 bp, synthetic construct
  source 1..54: mol_type = other DNA
ggggtacgatttgtttttatgttacctttctgatacctcctttttagcatgcct

SEQ ID NO: 210 - DNA, 44 bp, synthetic construct
  source 1..44: mol_type = other DNA
ttccagacgctctctgactgccagggccggtttatcgcgcgcct

SEQ ID NO: 211 - DNA, 44 bp, synthetic construct
  source 1..44: mol_type = other DNA
acacactgattagattagcacgtccttcatcgctccactaacac

SEQ ID NO: 212 - DNA, 54 bp, synthetic construct
  source 1..54: mol_type = other DNA
tcaccttaccgactttttgcttatcgccaggctctgggcttttttgctccccgt

SEQ ID NO: 213 - DNA, 33 bp, synthetic construct
  source 1..33: mol_type = other DNA
accagccttgattttccggatttgccttataac

SEQ ID NO: 214 - DNA, 33 bp, synthetic construct
  source 1..33: mol_type = other DNA
ggttcatacccattcggaaatcgccggtggaaa

SEQ ID NO: 215 - DNA, 22 bp, synthetic construct
  source 1..22: mol_type = other DNA
acacgcttaaacgcctcagcct

SEQ ID NO: 216 - DNA, 54 bp, synthetic construct
  source 1..54: mol_type = other DNA
tcggtttcccttctttttggctcccctcgcagtcacacgctttttctaagcgtg

SEQ ID NO: 217 - DNA, 44 bp, synthetic construct
  source 1..44: mol_type = other DNA
tctataccctgagctcacagcatgtgcatttttcgggtttcggg

SEQ ID NO: 218 - DNA, 44 bp, synthetic construct
  source 1..44: mol_type = other DNA
ccagttaagacacgtgtcccgccctactcatcgcaacttaacgc

SEQ ID NO: 219 - DNA, 54 bp, synthetic construct
  source 1..54: mol_type = other DNA
aacctgcccatggtttttctagatcacgtgtacggggctgttttttcaccctgt

SEQ ID NO: 220 - DNA, 44 bp, synthetic construct
  source 1..44: mol_type = other DNA
ttcgcaggcttcagttagtgttacccaaccttccacagcacacc

SEQ ID NO: 221 - DNA, 44 bp, synthetic construct
  source 1..44: mol_type = other DNA
ccctacccaacaacattagtcggttcggtcctcacagaacgctc

SEQ ID NO: 222 - DNA, 54 bp, synthetic construct
  source 1..54: mol_type = other DNA
acaagtcatccgcttttttaatttttcaacgcataagcgttttttcgctgccgc

SEQ ID NO: 223 - DNA, 33 bp, synthetic construct
  source 1..33: mol_type = other DNA
acccattacttgctacagaatataagctttcac

SEQ ID NO: 224 - DNA, 33 bp, synthetic construct
  source 1..33: mol_type = other DNA
ccccagccatctcccggtttgattggctcgctg

SEQ ID NO: 225 - DNA, 22 bp, synthetic construct
  source 1..22: mol_type = other DNA
tacaaaaggtaattcggttaac

SEQ ID NO: 226 - DNA, 54 bp, synthetic construct
  source 1..54: mol_type = other DNA
catcctggctgtcttttttgggccttcccttagctggcggttttttctgggttg

SEQ ID NO: 227 - DNA, 44 bp, synthetic construct
  source 1..44: mol_type = other DNA
ccccggagatgatgatggctgcttctaagccaacagtgctctac

SEQ ID NO: 228 - DNA, 44 bp, synthetic construct
  source 1..44: mol_type = other DNA
cgctacctaaatgagctattacgctttctttaaaattcacgagg

SEQ ID NO: 229 - DNA, 54 bp, synthetic construct
  source 1..54: mol_type = other DNA
ccgcgcaggccgatttttctcgaccagtagctttcggggatttttgaaccagct

SEQ ID NO: 230 - DNA, 33 bp, synthetic construct
  source 1..33: mol_type = other DNA
ccgattaaccttaggggtcgactcacgccccgt

SEQ ID NO: 231 - DNA, 33 bp, synthetic construct
  source 1..33: mol_type = other DNA
tacatcttagcttcggtgcatggtttacctgcc

SEQ ID NO: 232 - DNA, 22 bp, synthetic construct
  source 1..22: mol_type = other DNA
cgttggacaggtttcggcctcg

SEQ ID NO: 233 - DNA, 54 bp, synthetic construct
  source 1..54: mol_type = other DNA
cctgtttcccatctttttgactacgccaacccttggtctttttttccggcgagc

SEQ ID NO: 234 - DNA, 33 bp, synthetic construct
  source 1..33: mol_type = other DNA
ccaagtacctccgtccccccttcgcaaccatga

SEQ ID NO: 235 - DNA, 33 bp, synthetic construct
  source 1..33: mol_type = other DNA
ctttgggaccacatcgtttcccacttagtaaca

SEQ ID NO: 236 - DNA, 22 bp, synthetic construct
  source 1..22: mol_type = other DNA
aggaatattaaaacatagcctt

SEQ ID NO: 237 - DNA, 54 bp, synthetic construct
  source 1..54: mol_type = other DNA
tgtgtctcccgtgtttttataacattccgggatgaccccctttttttgccgaaa

SEQ ID NO: 238 - DNA, 33 bp, synthetic construct
  source 1..33: mol_type = other DNA
ggattcagccccggttcgcctcattacgttagc

SEQ ID NO: 239 - DNA, 33 bp, synthetic construct
  source 1..33: mol_type = other DNA
acccgccgtttccctcttcacgacggaacctat

SEQ ID NO: 240 - DNA, 22 bp, synthetic construct
  source 1..22: mol_type = other DNA
ttaatgatagttgtttcagttc

SEQ ID NO: 241 - DNA, 54 bp, synthetic construct
  source 1..54: mol_type = other DNA
tcttttcctcgggtttttgtacttagagtgtcgaaacacatttttctgggtttc

SEQ ID NO: 242 - DNA, 33 bp, synthetic construct
  source 1..33: mol_type = other DNA
tagccttgcactatcggtcagtcagggaatctc

SEQ ID NO: 243 - DNA, 33 bp, synthetic construct
  source 1..33: mol_type = other DNA
ggttgatttcgctcgccgctactggggagtatt

SEQ ID NO: 244 - DNA, 22 bp, synthetic construct
  source 1..22: mol_type = other DNA
gaggatggtcccggtactggtt

SEQ ID NO: 245 - DNA, 54 bp, synthetic construct
  source 1..54: mol_type = other DNA
ggttcttttcgccttttttttccctcaccccatattcagatttttcaggatacc

SEQ ID NO: 246 - DNA, 33 bp, synthetic construct
  source 1..33: mol_type = other DNA
ttttcacttgtacgtacacggtttcaatcgggt

SEQ ID NO: 247 - DNA, 33 bp, synthetic construct
  source 1..33: mol_type = other DNA
tggtaagttccggtattcgcagtttgcggttct

SEQ ID NO: 248 - DNA, 22 bp, synthetic construct
  source 1..22: mol_type = other DNA
cccctcgccggctcccactgct

SEQ ID NO: 249 - DNA, 54 bp, synthetic construct
  source 1..54: mol_type = other DNA
ctaagccaacatctttttctggctgtccggtttcaggttcttttttttttcact

SEQ ID NO: 250 - DNA, 44 bp, synthetic construct
  source 1..44: mol_type = other DNA
ccttggtcttctttctttaaatgatggctgctttggacaggaac

SEQ ID NO: 251 - DNA, 22 bp, synthetic construct
  source 1..22: mol_type = other DNA
cggcgagcgggagctattacgc

SEQ ID NO: 252 - DNA, 44 bp, synthetic construct
  source 1..44: mol_type = other DNA
acatcgtttccgctcccactgcttgtacgtacatgggccttccc

SEQ ID NO: 253 - DNA, 22 bp, synthetic construct
  source 1..22: mol_type = other DNA
cacttaaccatcgcctaagcgt

SEQ ID NO: 254 - DNA, 81 bp, synthetic construct
  source 1..81: mol_type = other DNA
cttcggctccccttttttattcggttaatacaaaaggtactttttgcagtcacagactttgggacctttttttagctggcg

SEQ ID NO: 255 - DNA, 33 bp, synthetic construct
  source 1..33: mol_type = other DNA
agacgcttgtcaccctgtatcgcgcgcaagact

SEQ ID NO: 256 - DNA, 33 bp, synthetic construct
  source 1..33: mol_type = other DNA
cggtttcctgcaacttaacgcccagttctttcc

SEQ ID NO: 257 - DNA, 54 bp, synthetic construct
  source 1..54: mol_type = other DNA
cccatattcagactttttaggataccaactgggtttcccctttttattcggaaa

SEQ ID NO: 258 - DNA, 33 bp, synthetic construct
  source 1..33: mol_type = other DNA
gacccattaccttgctacagaatataatggagg

SEQ ID NO: 259 - DNA, 33 bp, synthetic construct
  source 1..33: mol_type = other DNA
atggtcccagtcaggagtatttagcctgtcgct

SEQ ID NO: 260 - DNA, 44 bp, synthetic construct
  source 1..44: mol_type = other DNA
tggtattctcttcggcctcgccttaggggtcgactcaagcgcct

SEQ ID NO: 261 - DNA, 22 bp, synthetic construct
  source 1..22: mol_type = other DNA
acctgaccaccgactacgcctt

SEQ ID NO: 262 - DNA, 54 bp, synthetic construct
  source 1..54: mol_type = other DNA
ctagttccttcactttttccgagttctctcaccctgcccctttttgattaacgt

SEQ ID NO: 263 - DNA, 44 bp, synthetic construct
  source 1..44: mol_type = other DNA
ggttcttttcgctctgactgccagggcttttgccccctcgccgg

SEQ ID NO: 264 - DNA, 22 bp, synthetic construct
  source 1..22: mol_type = other DNA
cctttccctcatccttcatcgc

SEQ ID NO: 265 - DNA, 54 bp, synthetic construct
  source 1..54: mol_type = other DNA
acgcttatcgcagtttttattagcacgcggtactggttcatttttctatcggtc

SEQ ID NO: 266 - DNA, 33 bp, synthetic construct
  source 1..33: mol_type = other DNA
tttcctggtgatgttacctgatgcttaatatca

SEQ ID NO: 267 - DNA, 33 bp, synthetic construct
  source 1..33: mol_type = other DNA
ccttaccgtcgccggttataacggttcgaggct

SEQ ID NO: 268 - DNA, 54 bp, synthetic construct
  source 1..54: mol_type = other DNA
gtgcatttttgtgttttttacggggctccactaacacacatttttcactgattc

SEQ ID NO: 269 - DNA, 44 bp, synthetic construct
  source 1..44: mol_type = other DNA
ctactcatcgataatgatagtgtgtcgaaacaccgtgtcccgcc

SEQ ID NO: 270 - DNA, 22 bp, synthetic construct
  source 1..22: mol_type = other DNA
gctcacagcatatggattcagt

SEQ ID NO: 271 - DNA, 54 bp, synthetic construct
  source 1..54: mol_type = other DNA
agcacccgccgtgttttttgtctcccgaccgggtttcgggttttttctataccc

SEQ ID NO: 272 - DNA, 54 bp, synthetic construct
  source 1..54: mol_type = other DNA
gcgcaggccgacttttttcgaccagtgcttttcacccgcttttttttatcgtta

SEQ ID NO: 273 - DNA, 33 bp, synthetic construct
  source 1..33: mol_type = other DNA
catcttccttcggtgcatggtttagcctcacga

SEQ ID NO: 274 - DNA, 33 bp, synthetic construct
  source 1..33: mol_type = other DNA
cggacgttgtctgggttgtttccctctccgtta

SEQ ID NO: 275 - DNA, 33 bp, synthetic construct
  source 1..33: mol_type = other DNA
ccggagatcccttgccgaaacagtgctccctac

SEQ ID NO: 276 - DNA, 33 bp, synthetic construct
  source 1..33: mol_type = other DNA
ccaacaacgcaggcttacagaacgctcctaccc

SEQ ID NO: 277 - DNA, 54 bp, synthetic construct
  source 1..54: mol_type = other DNA
gcatgcctcacagtttttcacaccttcgcataagcgtcgcttttttgccgcagc

SEQ ID NO: 278 - DNA, 81 bp, synthetic construct
  source 1..81: mol_type = other DNA
gaatattaacctgttttttttcccatctgtgtcggtttggtttttggtacgattaagcagggcattttttttgttgcttca

SEQ ID NO: 279 - DNA, 33 bp, synthetic construct
  source 1..33: mol_type = other DNA
aagtacagtccgtccccccttcgcagtttctga

SEQ ID NO: 280 - DNA, 33 bp, synthetic construct
  source 1..33: mol_type = other DNA
tacctccacttatgtcagcattcgcacaacacc

SEQ ID NO: 281 - DNA, 54 bp, synthetic construct
  source 1..54: mol_type = other DNA
ttgattggccttttttttcacccccagcggttcggtcctctttttcagttagtg

SEQ ID NO: 282 - DNA, 33 bp, synthetic construct
  source 1..33: mol_type = other DNA
ctcccggtatagctttcggggagaaccaaccgg

SEQ ID NO: 283 - DNA, 33 bp, synthetic construct
  source 1..33: mol_type = other DNA
gacaaccgaaaccagcctacacgcttaagctat

SEQ ID NO: 284 - DNA, 54 bp, synthetic construct
  source 1..54: mol_type = other DNA
gggttggtaagtctttttgggatgaccgaattcacgaggctttttgctacctaa

SEQ ID NO: 285 - DNA, 44 bp, synthetic construct
  source 1..44: mol_type = other DNA
tccggtattcgtcaacctgcccatggctagatctgataacattc

SEQ ID NO: 286 - DNA, 22 bp, synthetic construct
  source 1..22: mol_type = other DNA
cagtttgcatcttacccaacct

SEQ ID NO: 287 - DNA, 54 bp, synthetic construct
  source 1..54: mol_type = other DNA
tgattttccggattttttttgcctggatcgcccggccaactttttatagccttc

SEQ ID NO: 288 - DNA, 33 bp, synthetic construct
  source 1..33: mol_type = other DNA
ttcagttctcttttcctcggggtactttcacgc

SEQ ID NO: 289 - DNA, 33 bp, synthetic construct
  source 1..33: mol_type = other DNA
ctcagcctgcaccgtagtgcctcgtcaagatgt

SEQ ID NO: 290 - DNA, 54 bp, synthetic construct
  source 1..54: mol_type = other DNA
actgggggaatcttttttcggttgattccccggttcgccttttttcattaacct

SEQ ID NO: 291 - DNA, 33 bp, synthetic construct
  source 1..33: mol_type = other DNA
acattagtccacaagtcatccgctaatgttcgc

SEQ ID NO: 292 - DNA, 33 bp, synthetic construct
  source 1..33: mol_type = other DNA
tcgccgctaggctctgggctgctccccttttca

SEQ ID NO: 293 - DNA, 81 bp, synthetic construct
  source 1..81: mol_type = other DNA
agttccttctgtctttttcctgaggagaatttggctgttgtttttgttaactcccacttccataccatttttggttctaat

SEQ ID NO: 294 - DNA, 44 bp, synthetic construct
  source 1..44: mol_type = other DNA
cagggcagcaaaacattagatatggggttggccgaatggtgtca

SEQ ID NO: 295 - DNA, 22 bp, synthetic construct
  source 1..22: mol_type = other DNA
tgaagtctggggcaagcccaac

SEQ ID NO: 296 - DNA, 33 bp, synthetic construct
  source 1..33: mol_type = other DNA
gatgggattagcaagcaagaccaaggtacagtt

SEQ ID NO: 297 - DNA, 33 bp, synthetic construct
  source 1..33: mol_type = other DNA
acaagcctaagtagctaagggagcatccttagt

SEQ ID NO: 298 - DNA, 81 bp, synthetic construct
  source 1..81: mol_type = other DNA
ctagtggtgtaattttttgaaaagttctatgcatagcccattttttcagcttcaagccagtcatcagtttttgagaaaccc

SEQ ID NO: 299 - DNA, 44 bp, synthetic construct
  source 1..44: mol_type = other DNA
gggcagtcataggttaatgggctctgggcaatacctgccattag

SEQ ID NO: 300 - DNA, 22 bp, synthetic construct
  source 1..22: mol_type = other DNA
acaaacttcagtcctctagcat

SEQ ID NO: 301 - DNA, 81 bp, synthetic construct
  source 1..81: mol_type = other DNA
caacactgtgcattttttatacatcaacctcatagatgggttttttaccctacagtgggccatggacttttttgcaagata

SEQ ID NO: 302 - DNA, 33 bp, synthetic construct
  source 1..33: mol_type = other DNA
gtcccctcgagcccctcatgtccttgggcaagg

SEQ ID NO: 303 - DNA, 33 bp, synthetic construct
  source 1..33: mol_type = other DNA
tccactgcgccatatgaattgcatggacctagg

SEQ ID NO: 304 - DNA, 81 bp, synthetic construct
  source 1..81: mol_type = other DNA
cctgactagacatttttttagtgcatattctggtcatggattttttacatggctatgattctctcattttttgaatcccaa

SEQ ID NO: 305 - DNA, 33 bp, synthetic construct
  source 1..33: mol_type = other DNA
tgtcagggaaccaatttgaggcccagagactag

SEQ ID NO: 306 - DNA, 33 bp, synthetic construct
  source 1..33: mol_type = other DNA
ggtgaaggttgtattccacatagagatcacaga

SEQ ID NO: 307 - DNA, 44 bp, synthetic construct
  source 1..44: mol_type = other DNA
agccagtcatctgcactcactaggccccaaataagatgtcaggg

SEQ ID NO: 308 - DNA, 44 bp, synthetic construct
  source 1..44: mol_type = other DNA
tcctctagcatgatacatggcttgagtctctaaaggagaaaccc

SEQ ID NO: 309 - DNA, 22 bp, synthetic construct
  source 1..22: mol_type = other DNA
ggttaatgggcttctggtcatg

SEQ ID NO: 310 - DNA, 81 bp, synthetic construct
  source 1..81: mol_type = other DNA
tgaagtctggggttttttgggccatggaaccaatttgaggtttttcccagacacgatgcttataatgtttttggacaggtg

SEQ ID NO: 311 - DNA, 44 bp, synthetic construct
  source 1..44: mol_type = other DNA
tttccctctaggaatggtgtcacagggcagcaacccattgccaa

SEQ ID NO: 312 - DNA, 44 bp, synthetic construct
  source 1..44: mol_type = other DNA
ctatggggctacacttccataccaggttctaataccttcctttt

SEQ ID NO: 313 - DNA, 22 bp, synthetic construct
  source 1..22: mol_type = other DNA
gaaatttactcgctctgtggta

SEQ ID NO: 314 - DNA, 33 bp, synthetic construct
  source 1..33: mol_type = other DNA
accctacagttacaagcctcctcatagccctgg

SEQ ID NO: 315 - DNA, 33 bp, synthetic construct
  source 1..33: mol_type = other DNA
attctacatttcatttacagctcaccaatgggt

SEQ ID NO: 316 - DNA, 22 bp, synthetic construct
  source 1..22: mol_type = other DNA
aatttcaccatgttatccaaag

SEQ ID NO: 317 - DNA, 22 bp, synthetic construct
  source 1..22: mol_type = other DNA
gcaagcccaacggagcatcaca

SEQ ID NO: 318 - DNA, 81 bp, synthetic construct
  source 1..81: mol_type = other DNA
atatacatcaaaatttttgtagctaagaacattagatatgtttttgggttggccctagtggtgtaattttttgaaaagttc

SEQ ID NO: 319 - DNA, 44 bp, synthetic construct
  source 1..44: mol_type = other DNA
gccatatgaattatgcatagcccatcagcttcaactgcaagata

SEQ ID NO: 320 - DNA, 44 bp, synthetic construct
  source 1..44: mol_type = other DNA
aggtccactgcaccaaggtcttagtgatgggattgcatggagca

SEQ ID NO: 321 - DNA, 22 bp, synthetic construct
  source 1..22: mol_type = other DNA
caacactgtgctagcaagcaag

SEQ ID NO: 322 - DNA, 81 bp, synthetic construct
  source 1..81: mol_type = other DNA
ggaggaaggctcttttttagaaagccaaagtggtctacaatttttccccttgcactggggacactcttttttttacctgta

SEQ ID NO: 323 - DNA, 33 bp, synthetic construct
  source 1..33: mol_type = other DNA
agtgcatatagggtgaaggcctgactaggggca

SEQ ID NO: 324 - DNA, 33 bp, synthetic construct
  source 1..33: mol_type = other DNA
tggcaaacaagggcagtgcttgcccctgacatt

SEQ ID NO: 325 - DNA, 22 bp, synthetic construct
  source 1..22: mol_type = other DNA
aggggagggtggtgagagtgca

SEQ ID NO: 326 - DNA, 22 bp, synthetic construct
  source 1..22: mol_type = other DNA
gagcccctcatatagagatgac

SEQ ID NO: 327 - DNA, 81 bp, synthetic construct
  source 1..81: mol_type = other DNA
atgaatcccaatttttttgtattccacgtccttggcctagtttttggtcccctctctgggcaataaattttttttggctgt

SEQ ID NO: 328 - DNA, 33 bp, synthetic construct
  source 1..33: mol_type = other DNA
cagtcatatccctgaggagcctgccattgactt

SEQ ID NO: 329 - DNA, 33 bp, synthetic construct
  source 1..33: mol_type = other DNA
tggcttgcatcaaataaggctacctgctagggg

SEQ ID NO: 330 - DNA, 22 bp, synthetic construct
  source 1..22: mol_type = other DNA
atgattctctctggttaactcc

SEQ ID NO: 331 - DNA, 22 bp, synthetic construct
  source 1..22: mol_type = other DNA
acaaacttcagagttccttctg

SEQ ID NO: 332 - DNA, 44 bp, synthetic construct
  source 1..44: mol_type = other DNA
ttttcctggaattggtcttccggcgagcgggcttgcttagaggc

SEQ ID NO: 333 - DNA, 44 bp, synthetic construct
  source 1..44: mol_type = other DNA
ctacctgctgatgcactcactaggcccgtggtaatcaaataagg

SEQ ID NO: 334 - DNA, 22 bp, synthetic construct
  source 1..22: mol_type = other DNA
ctttggcttgctgagtctctaa